(19) United States
(12) Patent Application Publication
(10) Pub. No.: US 2014/0017678 A1
(43) Pub. Date: Jan. 16, 2014

(54) PATHWAY CHARACTERIZATION OF CELLS

(71) Applicant: Nodality Inc., (US)

(72) Inventors: Alessandra Cesano, Redwood City, CA (US); David B. Rosen, Mountain View, CA (US); Wendy J. Fantl, San Francisco, CA (US); Rachael Hawtin, San Carlos, CA (US)

(73) Assignee: Nodality, Inc., South San Francisco, CA (US)

(21) Appl. No.: 13/801,420

(22) Filed: Mar. 13, 2013

Related U.S. Application Data
(60) Provisional application No. 61/658,092, filed on Jun. 11, 2012.
(60) Provisional application No. 61/728,981, filed on Nov. 21, 2012.

(51) Int. Cl. G01N 33/68

(52) U.S. Cl. CPC G01N 33/6893
USPC 435/6.11; 435/7.1

(57) ABSTRACT

The present invention provides methods, compositions and kits for the characterization of cellular pathways in cells containing genetic alterations.

### CROSS-REFERENCE

This application claims the benefit of U.S. Provisional Application No. 61/658,092, entitled “Pathway Characterization of Cells,” and filed Jun. 11, 2012, and U.S. Provisional Application No. 61/728,981, entitled “Pathway Characterization of Cells,” and filed Nov. 21, 2012, which applications are incorporated herein by reference.

This application is related to the following co-pending patent applications: U.S. Provisional Application No. 61/353,155 filed Jun. 9, 2010, and U.S. Ser. No. 13/384,181, filed Jan. 13, 2012, which applications are incorporated herein by reference.

### BACKGROUND OF THE INVENTION

Genomic instability is a characteristic of most cancers. In hereditary cancers, genomic instability results from mutations in DNA repair genes and mitotic checkpoint genes which drive cancer progression by increasing the rate of spontaneous mutations. Caretaker proteins protect the genome against mutations, while gatekeepers induce cell death or cell cycle arrest of potentially tumorigenic cells (Negrini et al. (2010) Nat. Reviews Mol. Cell Biol 11:220-8).

A variety of genes are involved in the control of cell growth and division. The cell cycle, or cell-division cycle, is the series of events that ensures faithful, error-free duplication of the cellular genome (replication) and subsequent physical division into two daughter cells. Tight regulation of this process ensures that the DNA in a dividing cell is copied correctly, any damage in the DNA is repaired, and that each daughter cell receives a full set of chromosomes. The cell cycle has checkpoints, which ensure that a cell cannot advance from one phase to another if the genome is in need of repair. Genes involved in this process are referred to as being part of the cellular DNA damage response (DDR) machinery. Germline (hereditary) mutations in DDR genes cause an increased risk for developing cancer, described in FIG. 3.

If a cell has an error in its DNA that cannot be repaired, it may undergo apoptosis, also known as programmed cell death. Conditions such as cancer can result from deregulation of the DDR, cell cycle, apoptosis, or any combination of these pathways. In the event of DNA damage, cells have evolved numerous mechanisms to halt cell cycle progression. This process of halting cell cycle progression is known in the art as a cell cycle checkpoint. When the cell cycle evades checkpoint control, cells can divide without repairing genomic damage and thereby accumulate genetic defects that can lead to cancer (sometimes referred to as neoplasia).

In sporadic (non-hereditary) cancers, the molecular bases of genomic instability remains unclear, but recent high-throughput sequencing studies suggest that mutation in DNA repair genes are infrequent before cancer therapy arguing against this mechanism. Genomic instability in sporadic cancer is most likely attributed to oncogene-induced DNA damage caused by mutations in ataxia telangiectasia mutated (ATM), cyclin-dependent kinase inhibitor 2A (which encodes p16INK4A and TP53 which encodes p53.

Gene sequencing is the more common way to identify gene mutations. However, this may underestimate the presence of gene inactivation because gene function can be altered by other mechanisms such as epigenetic mechanisms.

A method which allows for a functional assessment of carekeeper genes in germline cells will provide a tool to identify subjects at risk for tumor development and will inform appropriate medical management/interventions.

For instance, carriers of a germline BRCA1 or BRCA2 heterozygous mutation are at increased risk in developing breast, ovarian, prostate and pancreatic cancers when the wild type allele is inactivated, whether by somatic loss, a second mutation, or an epigenetic event. Currently, individuals with a family history of pre-menopausal breast cancer are tested using gene sequencing for the presence of germline BRCA1/2 mutations and if positive they are counseled to consider and may undergo preventive bilateral mastectomy and/or oophorectomy. However, there is a subset of women whose breast cancers are estrogen receptor negative, progesterone receptor negative, and herceptin negative (termed triple negative) and whose tumors bear similarities in clinico-pathologic features and response to therapeutic agents (resistance to hormonal therapy and Her-2 inhibitor, sensitivity to chemotherapy) to tumors from patients that carry germ line mutations in BRCA1/2 genes. Accordingly, there is a need to develop tools which allow for a functional evaluation of DNA repair mechanisms and mitotic checkpoints at the single cell level.

### SUMMARY OF THE INVENTION

In some embodiments the invention provides methods of classification, diagnosis, prognosis and/or prediction of an outcome of a condition in an individual, the method comprising: a) contacting a cell population from the individual with a DNA damage or apoptosis inducing agent, where the cell population comprises a genetic and/or epigenetic alteration, where the alteration is associated with the development of the condition; b) characterizing a plurality of DNA damage repair pathways in one or more cells from the cell population by determining an activation level of at least one activatable element within the plurality of DNA damage repair pathways; c) determining whether the plurality of DNA damage pathways are functional in the individual based on the activation levels of the activatable elements; and d) making a decision regarding classification, diagnosis, prognosis and/or prediction of an outcome of the condition in the individual, where the decision is based on the determination on step (c). In some embodiments, step (c) further comprises a correlation between the activation levels of the activatable elements within the plurality of DNA damage repair pathways. In some embodiments, the methods further comprise correlating the activation levels of the activatable elements within the plurality of DNA damage repair pathways with apoptosis induced by the DNA damage or apoptosis inducing agent on the cell population. In some embodiments, the DNA damage repair pathway is selected from the group consisting of nucleotide excision repair, checkpoint activation, homologous recombination, non-homologous end joining, base excision repair, mismatch repair, double strand DNA damage repair and fanconi anaemia pathway. In some embodiments, a homologous recombination, a double strand DNA damage repair and a non-homologous end joining, base excision repair are characterized

In some embodiments, the invention is a method of classification, diagnosis, prognosis and/or prediction of an outcome of a condition in an individual, the method comprising: a) contacting a cell population from the individual with a DNA damage or apoptosis inducing agent, where the cell population comprises a genetic and/or epigenetic alteration, and where the cell population is not associated and/or is not causative of the condition; b) determining an activation level of at least one activatable element within a DNA damage pathway, an apoptosis pathway, and/or a cell cycle pathway in one or more cells from the cell population; and c) making a decision regarding classification, diagnosis, prognosis and/or prediction of an outcome of the condition in the individual, where the decision is based on the activation levels of the at least one activatable element within the DNA damage pathway, an apoptosis pathway, and/or a cell cycle pathway.

In other embodiment, the invention is a method of determine a signaling phenotype of a cell population, where the cell population comprises a genetic/epigenetic alteration of interest, the method comprising: a) subjecting the cell population comprising the genetic alteration to a plurality of modulators in separate of cultures; b) characterizing at least one pathway in the cell population from separate plurality of cultures by determining an activation level of at least one activatable element within the at least one pathway; c) creating a response panel for the comprising the characterization of the at least one pathway from the separate cultures; and d) determining a signaling phenotype, where the signaling phenotype is based on the response panel.

In yet other embodiments, the invention is a method for detecting p53 levels in a cell population, the method comprising: a) subjecting the cell population to a modulator; b) contacting the cell population with a binding element specific for p53; and c) using flow cytometry to detect presence or absence of binding of the binding element to p53, where the presence or absence of binding of the binding element is indicative of the p53 levels in the population.

In some embodiments, the condition is selected from the group consisting of acute leukemia, myelodysplastic syndrome and myeloproliferative neoplasms.

In some embodiments, the methods further comprise performing a molecular analysis to detect the genetic alteration is the cell population.

In some embodiments, the individual has a predefined clinical parameter. In some embodiments, the predefined clinical parameter is selected from the group consisting of age, de novo acute myeloid leukemia patient, secondary acute myeloid leukemia patient, or a biochemical/molecular marker. In some embodiments, making a decision regarding classification, diagnosis, prognosis and/or prediction of an outcome of the condition in the individual is based on determination of the methods described herein in combination with the predefined clinical parameter.

In some embodiments, DNA damage or apoptosis inducing agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, temozolomide, teniposide, Thioguanine, topotecan, troxacitabine, Abraxane, Adriamycin, carboplatin, Cytoxan, Doxil, Ellence, fluorouracil, Gemzar, Ixempra, methotrexate, Mitomycin, mitoxantrone, Navelbine, Taxol, Taxotere, thiotepa, vincristine, Xeloda, Herceptin, Tykerb, Avastin, mitotic inhibitors, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

In some embodiments, at least one activatable element is selected from the group consisting of p-BRCA1, p-DNA-PKcs, pKu70, pKu80, p-Akt, p-Rad51, pRad52, pRPA32, p-ATR, p53BP1, p-Chk1, p-Chk2, p-53, p-ATM, and p-H2AX.

In some embodiments, the characterization step further comprises characterizing an apoptosis pathway by determining an activation level of at least one activatable element within the apoptosis pathway. In some embodiments, the activatable element within the apoptosis pathway is selected from the group consisting of Cleaved PARP, Cleaved Caspase 3, Cleaved Caspase 8, BAX, Bak, and Cytochrome C. In some embodiments, the characterization step further comprises characterizing a cell cycle pathway by determining an activation level of at least one activatable element within the cell cycle pathway. In some embodiments, at least one activatable element within a cell cycle pathway is selected from the group consisting of Cdc25, p-p53, cCdk1, CyclinB1, p16, p21, p-Histone H3 and Gadd45.

In some embodiments, the methods further comprise determining guide selection of a therapeutic treatment for the individual.

In some embodiments, the methods further comprise contacting the cell population with additional modulators and characterizing additional pathways by determining the activation level of at least one activatable element within the additional pathway. In some embodiments, the additional pathway is selected from the group consisting of drug conversion into an active agent, internal cellular pH, redox potential environment, phosphorylation state of ITIM; drug activation; and signaling pathways. In some embodiments, the additional pathway is selected from the group consisting of Jak/Stat, PI3K/Akt, and MAPK pathways. In some embodiments, the activatable element within the PI3K/AKT or MAPK pathways is selected from the group consisting of Akt, p-ERK, p-SyK, p38 and pS6 and the modulator is selected from the group consisting of FLT3L, SCF, G-CSF, GM-CSF, SCF, SDF1a, LPS, PMA, and Thapsigargin. In some embodiments, the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, GM-CSF and G-CSF.

In some embodiments, the methods further comprise determining the presence or absence of one or more cell surface markers, intracellular markers, or combination thereof. In some embodiments, the cell surface markers and the intracellular markers are independently selected from the group consisting of proteins, carbohydrates, lipids, nucleic acids and metabolites. In some embodiments, determining of the presence or absence of one or more cell surface markers or intracellular markers comprises determining the presence or absence of an epitope in both activated and non-activated forms of the cell surface markers or the intracellular markers. In some embodiments, the classification, diagnosis, prognosis and/or prediction of outcome of the condition in an individual is based on both the activation levels of the activatable element and the presence or absence of the one or more cell surface markers, intracellular markers, or combination thereof.

In some embodiments, the activation level is determined by a process comprising the binding of a binding element which is specific to a particular activation state of the particular activatable element. In some embodiments, the binding element comprises an antibody, recombinant protein, or fluorescent dye. In some embodiments, the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

In some embodiments, the activation level is determined by a process comprising the binding of a binding element which is specific to a particular activation state of the particular activatable element, and where the level of binding of the binding element is detected at a single cell level.

In some embodiments, the genetic alteration is a germline alteration. In some embodiments, the genetic alteration is an alteration in a gene selected from the group consisting of APC, AXIN2, ARF, ATM, BLM, CDH1, GPC3, CYLD, EXT1, EXT2, PTCH, SUFU, FH, SDHB, SDHC, SDHD, VHL, TP53, WT1, STK11, PTEN, TSC1, TSC2, CDKN2A, CDK4, RB1, RAD50, NF1, BMPR1A, MEN1, SMAD4, BHD, HRPT2, NF2, MUTYH, ATM, BLM, BRCA1, BRCA2, FANCA, FANCC, FANCD2, FANCE, FANCF, FANCG, NBS1, RECQL4, WRN, MSH2, MLH1, MSH6, MDM2, MRE11, NBS1, RAS, RHO, RAN, RAB, PMS2, p53, XPA, XPC, ERCC2, ERCC3, ERCC4, ERCC5, DDB2, KIT, MET, PDGFRA, RET, and DNA replication factor C. In some embodiments, the genetic alteration is in a gene from Table 1. In some embodiments, the genetic alteration is in a BRCA gene.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the functional domains of p53.

FIG. 2 depicts DNA damage response and repair pathways and activatable elements for detection of genomic instability in wild type (wt).

FIG. 3 depicts DNA damage response and repair pathways and activatable elements for detection of genomic instability in a BRCA mutant model.

FIG. 4 depicts DNA damage response and repair pathways and activatable elements for detection of genomic instability in wild type (wt) with inhibition of PARP.

FIG. 5 depicts DNA damage response and repair pathways and activatable elements for detection of genomic instability in a BRCA mutant model with inhibition of PARP.

FIG. 6 depicts a nucleotide excision repair model and enzymes involved that can be targets for use with the invention.

FIG. 7 depicts the cellular DNA damage response (DDR) machinery and agents useful for treatment of tumorigenesis and cancer by targeting components of DNA damage signaling.

FIG. 8 depicts the cellular DNA damage response (DDR) machinery and agents useful for treatment of tumorigenesis and cancer by targeting components of DNA damage signaling.

FIG. 9 shows that an ATM mutant cell lines display attenuated DNA damage response to etoposide.

FIG. 10 shows that Etoposide induced DNA damage (pH2AX) and apoptosis identified distinct subgroups in AML samples.

FIG. 11 shows that Etoposide-induced HR (pBRCA1) and Apoptosis identify distinct subgroups in AML samples.

FIG. 12 shows that induced phosphorylation of NHEJ (pDNA-PKcs) identifies Etoposide sensitive samples.

FIG. 13 shows that pediatric AML samples display distinct kinetics of Etoposide induced pDNA-PK downregulation.

FIG. 14 shows correlations between DDR nodes in non-apoptotic cells in AML samples (2 h, 6 h) highlight unique patient biology.

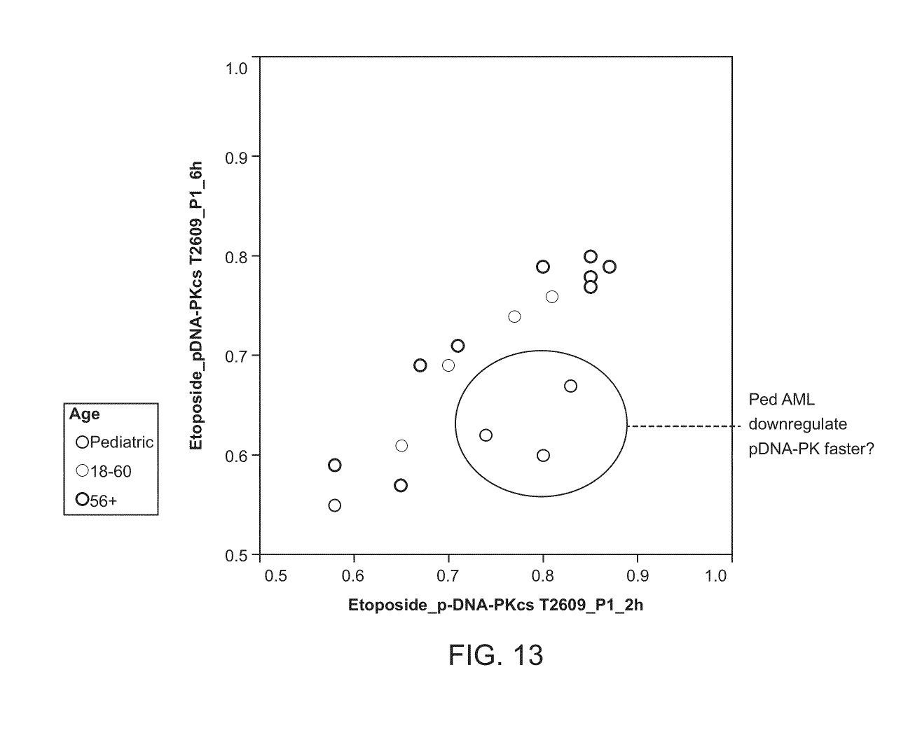

FIG. 13 shows that treatment with PARP inhibitors induces DNA damage in cycling cells.

FIG. 15 shows that focusing on cycling cells reveals PARPi induced DNA damage in cell lines

FIG. 16 shows that adjusting apoptosis by % Cyclin+ reveals PARPi induced apoptosis in HR defective cell lines.

FIG. 17 shows that adjusting apoptosis by % Cyclin+ reveals PARPi induced apoptosis in HR defective cell lines.

FIG. 18 shows that focusing on cycling cells reveals PARPi induced DNA damage in AML samples

FIG. 19 shows that SCNP detects AML samples sensitive to and in vitro PARP inhibitor treatment.

FIG. 20 shows PARPi+Temodar combination induced Apoptosis in AML samples. The figure shows unique patient trends of Temodar and PARP sensitivity.

FIG. 21 shows that SCNP can detect activation of multiple DDR pathways after PARP inhibition.

FIG. 22 shows an overview of BRCA1 DDR data.

FIG. 23 shows the Table of Patient Characteristics.

FIG. 24 shows Higher PARPi induced DNA Damage in HR mutants and different levels of HR deficiency, particularly in Cyclin A2+ cells.

FIG. 25 shows that for PBMC T cells, Induced proliferation is required to measure PARPi effect.

FIG. 26 shows the Sample Overview+Study Design For Assaying HR function in PBMC.

FIG. 27 shows that Associations still observed in CyclinA2+ cells suggest technical variance is affecting proliferation which is affecting DDR readouts.

FIG. 28 shows Approach #1 for Controlling Proliferation: Focus analysis on samples within the middle range of proliferation, where a large dynamic range for DDR readouts still exists.

FIG. 29 shows Approach #2 for Controlling Proliferation: Adjust batches to make data more comparable across all batches.

FIG. 30 shows Controlling for proliferation improves BRCA1 stratification and demonstrates higher induced p21, p53, p-H2AX in BRCA1+/−PBMC vs. BRCA1+/+ samples.

FIG. 31 shows Multivariate models stratify HR impaired BRCA1+/−PBMCs.

FIG. 32 shows Combining multiple DNA Damage pathway measurements (Multivariate models) improves BRCA1 stratification.

FIG. 33 shows a model.

FIG. 34 shows a model.

FIG. 35 shows a Gating Scheme for PBMC BRCA1 Study.

FIG. 36 shows Low-proliferating non-evaluable samples (<7.5% CyclinA2+), display low DDR signaling and mask BRCA1 stratification.

FIG. 37 shows DDR nodes (Y) are associated with proliferation (X) and gating on CyclinA2− or CyclinA2+ subsets reduces (but does not eliminate) the association (R2) of DDR nodes (Y) with proliferation (X).

FIG. 38 shows Lower basal p-BRCA1, higher induced p-BRCA1 observed in BRCA1 MUT PBMC vs BRCA1 WT PBMC.

FIG. 39 shows Controlling for proliferation and technical variance via batch adjustment or analysis of middle proliferation samples. reveals BRCA1 stratification in nodes associated with proliferation (48 h data).

FIG. 40 shows BRCA1+/− with higher induction of p53, p-H2AX compared to BRCA1+/+ in 1) all samples or within 2) cancer samples or 3) healthy subjects.

FIGS. 41 and 42 show two methods for controlling for proliferation+technical variance to reveal similar BRCA1 stratification: (Batch adjustment, or analysis of samples within the middle range of proliferation) Uu metric.

FIG. 43 shows evidence of lower p-BRCA1 basal levels in BRCA1+/− samples and higher induction (less consistent among metrics, prolif adjustment method vs. other stratifying trends).

FIG. 44 shows a Summary Table (4 stratifying DDR nodes in PARP conditions).

FIG. 45 shows a Summary Table v1 (all 6 DDR nodes in PARP conditions).

FIG. 46 shows Gating on Cyclin− or Cyclin+ reduces the association (R2 and slope) with proliferation.

FIG. 47 shows BRCA1+/−PBMC show higher p-H2AX, p53, p21 responses to PARPi (AZD2281)+/−TMZ compared to BRCA1+/+ PBMC.

FIG. 48 shows Summary of Conditions.

FIG. 49 shows Technical Methods for DDR.

FIG. 50 shows Technical Methods DDR: Samples Used.

FIG. 51 shows Overview of DNA Damage Response Pathways Measured.

FIG. 52 shows Genetic controls validates SCNP DDR readouts: Muted DDR in etoposide treated ATM−/− cell lines.

FIG. 53 shows Correlations Between Etoposide induced DDR Nodes In AML Samples Highlight Unique Patient Biology.

FIG. 54 shows Analyzing DNA Damage in Cycling and Non-Cycling cells demonstrates cell-cycle specific effects of individual genotoxins on AML samples.

FIG. 55 shows Higher PARPi Induced DDR readouts in Cyclin+vs Cyclin− Cells

FIG. 56 shows Lower CVs (Better Reproducibility) of DDR readouts in Cyclin+vs. Cyclin− cells.

FIG. 57 shows Gating on CyclinA2+ cells reveals HR defective samples.

FIG. 58 shows Gating on CyclinA2+ cells reveals distinct levels of HR deficiency including carrier status: BRCA2−/−>BRCA1+/−>BRCA1+/+ for PARPi induced pH2AX in CyclinA2+ cells.

FIG. 59 shows Clinical Characteristics of AML samples.

FIG. 60 shows Cell Lines Tables.

FIG. 61 shows Tools for Assaying DNA Damage Response and Repair Pathways

FIG. 62 shows proliferation rate information.

FIG. 63 shows Analyzing DNA Damage in Cycling and Non-Cycling cells demonstrates cell-cycle specific effects of individual genotoxins on Cell Lines.

FIG. 64 shows Gating on Cyclin+ reduces the affect of proliferation on DDR Readouts.

### DETAILED DESCRIPTION OF THE INVENTION

The present invention incorporates information disclosed in other applications and texts. The following patent and other publications are hereby incorporated by reference in their entireties: Haskell et al, Cancer Treatment, 5th Ed., W.B. Saunders and Co., 2001; Alberts et al., The Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.; and Sambrook, Fritsche and Maniatis. “Molecular Cloning A laboratory Manual” 3rd Ed. Cold Spring Harbor Press (2001), Andrew Murray & Tim Hunt, “The Cell Cycle: An Introduction” (1993) all of which are herein incorporated in their entirety by reference for all purposes.

Also, patents and applications that are incorporated by reference include U.S. Pat. Nos. 7,381,535; 7,393,656; 7,695,924; 7,695,926; and 8,187,885; U.S. patent application Ser. Nos. 10/193,462; 11/655,785; 11/655,789; 11/655,821; 11/338,957; 12/877,998; 12/784,478; 12/730,170; 12/703,741; 12/687,873; 12/617,438; 12/606,869; 12/713,165; 12/293,081; 12/581,536; 12/538,643; 12/501,274; 61/079,537; 12/501,295; 12/688,851; 12/471,158; 12/910,769; 12/460,029; 12/432,239; 12/432,720; 12/229,476, 12/877,998; 13/083,156; 61/469,812; 61/436,534; 61/317,187; 61/353,155; 61/542,910; 61/557,831; and 61/640,794 and PCT Application Nos. PCT/US2011/029845; PCT/US2010/048181; PCT/US2011/01565; and PCT/US2011/065675. The present application is especially related to U.S. Ser. No. 13/384,181 which is incorporated by reference in its entirety.

Some commercial reagents, protocols, software and instruments that are useful in some embodiments of the present invention are available at the Becton Dickinson Website http(double slash)www.bdbiosciences.com(slash)features(slash)products(slash), and the Beckman Coulter website, http:(double slash)www.beckmancoulter.com(slash)Default.asp?bhfv=7. Relevant articles include High-content single-cell drug screening with phosphospecific flow cytometry, Krutzik et al., Nature Chemical Biology, 23 Dec. 2007; Irish et al., FLt3 ligand Y591 duplication and Bcl-2 over expression are detected in acute myeloid leukemia cells with high levels of phosphorylated wild-type p53, Neoplasia, 2007, Irish et al. Mapping normal and cancer cell signaling networks: towards single-cell proteomics, Nature, Vol. 6 146-155, 2006; and Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol, 2007, 78:8 8.17.1-20; Krutzik, P. O., et al., Coordinate analysis of murine immune cell surface markers and intracellular phosphoproteins by flow cytometry, J Immunol. 2005 Aug. 15; 175(4):2357-65; Krutzik, P. O., et al., Characterization of the murine immunological signaling network with phosphospecific flow cytometry, J Immunol. 2005 Aug. 15; 175(4):2366-73; Shulz et al., Current Protocols in Immunology 2007, 78:8.17.1-20; Stelzer et al. Use of Multiparameter Flow Cytometry and Immunophenotyping for the Diagnosis and Classfication of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. 2003 October; 55(2):61-70; Hanahan D., Weinberg, The Hallmarks of Cancer, CELL, 2000 Jan. 7; 100(1) 57-70; Krutzik et al, High content single cell drug screening with phosphospecific flow cytometry, Nat Chem Biol. 2008 February; 4(2):132-42. Experimental and process protocols and other helpful information can be found at http:/proteomices.stanford.edu. The articles and other references cited below are also incorporated by reference in their entireties for all purposes.

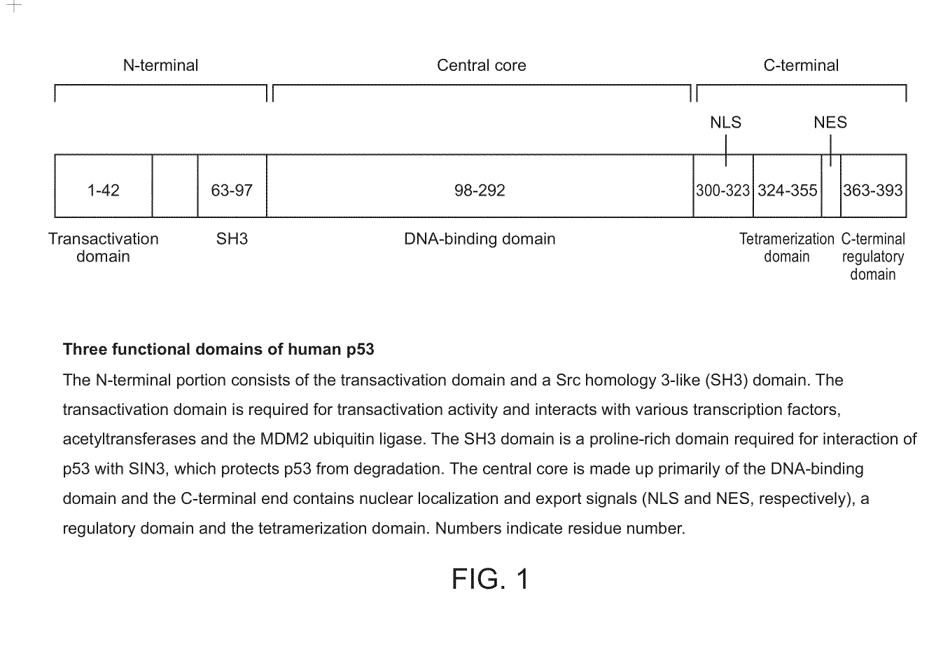

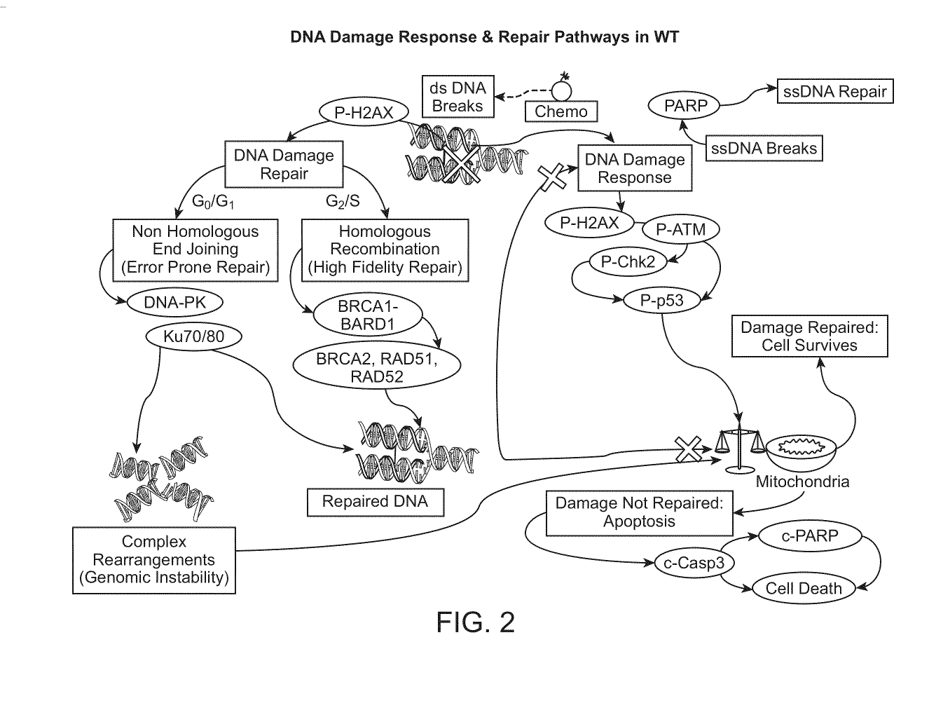

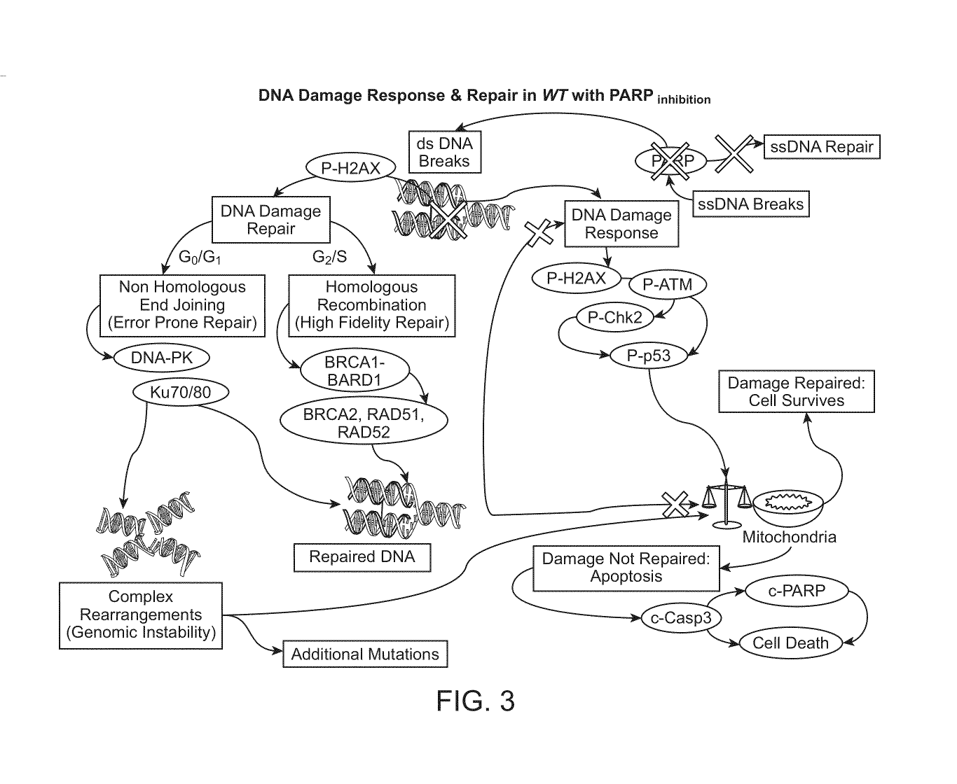

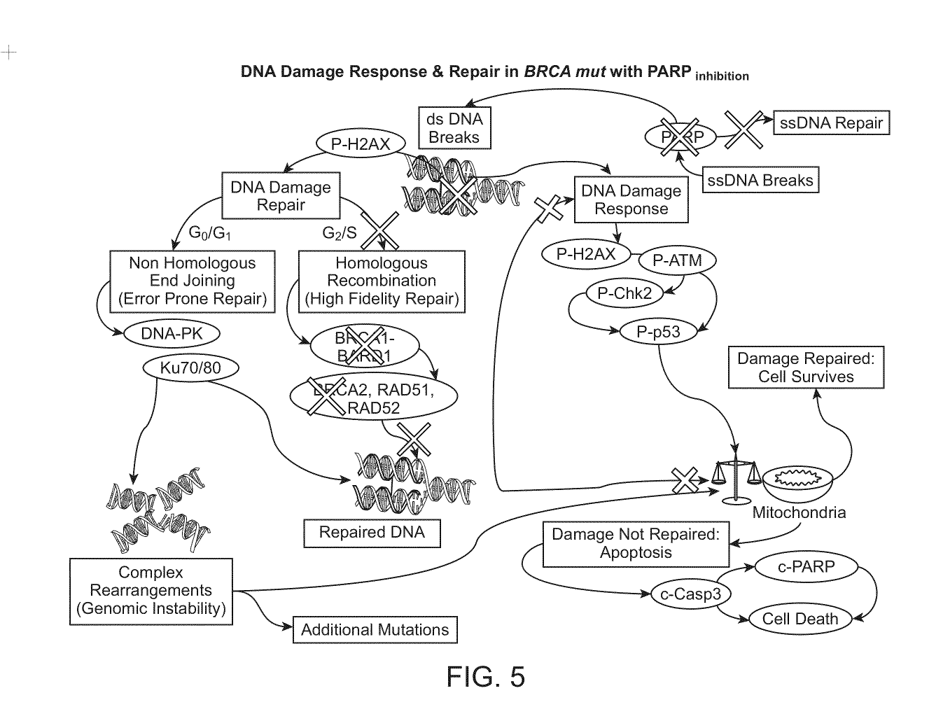

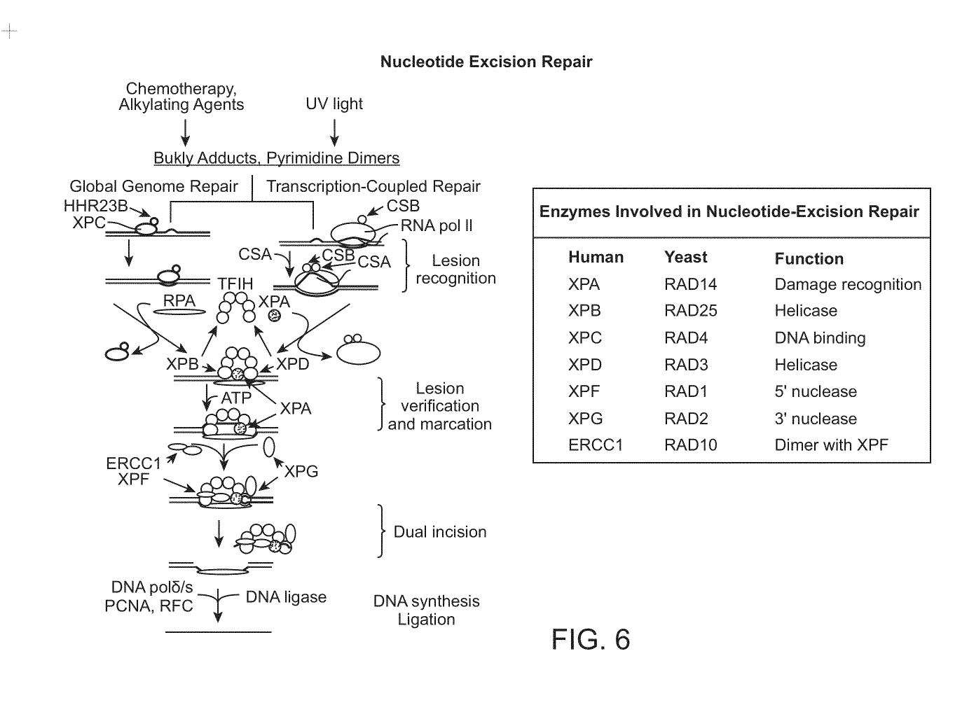

In some embodiments, the invention provides methods and compositions for determining genomic instability in one or more cells and/or one or more cell populations. In some embodiments, the invention provides methods and compositions for determining the activation level of an activatable element of a cell having genomic instability. The genomic instability can be from a mutation in a DNA repair gene, a gene that functions to maintain genomic stability or a gene involved in the control of cell cycle growth and/or division. In some embodiments, the genomic instability can be from a mutation in any one or more of the cellular DNA damage response (DDR) genes listed in FIG. 3. In some embodiments, the invention provides compositions and methods to indentify one or more germline mutations and/or one or more somatic mutation. In some embodiment, cellular pathway deregulations caused by the mutations are identified by performing functional assays. In some embodiments, the invention provides methods to indentify cellular pathway deregulations caused by one or more germline mutations and/or one or more somatic mutation by measuring the activation level of an activatable element listed in FIGS. 1 to 8. In some embodiments, the activation levels of an activatable element resulting from a mutation are measured in response to a modulator. In some embodiments, the activation level of an activatable element that is within the same pathway as an activatable element resulting from a mutation is measured in response to a modulator. In some embodiments, the mutation is associated with a condition (e.g. cancer) and/or the activation level of an activatable element that is affected by a gene or a gene product whose genetic alteration is associated with a condition. In some embodiments, the methods of the present invention can be used following genomic testing for genetic mutations. In one embodiment, cells that are not associated with the diseased tissue may be tested for a disease associated mutation. Then an additional test may be used to measure activatable elements in cells associated with a condition. In other embodiments, the methods of the present invention can be used independently from genomic testing for genetic mutations. In some embodiments, the invention provides methods for determining the changes involved in gene expression. Those changes include changes by mutations, epigenetic modifications, cytogenetic modifications, chromosomal changes, induced translocation of genes, and DNA damage. In other embodiments, the methods of the invention can be used on cells treated with a modulator to examine reexpression of genes previously silenced by epigenetic modification.

In some embodiments, the present invention provides compositions and methods for determining the activation level of an activatable element wherein the activatable element has a genetic alteration associated with a condition (e.g. cancer) in individual cells. In some embodiments, the invention provides methods and compositions for determining the activation level of an activatable element that is affected by a gene or gene product that has a genetic alteration associated with a condition (e.g. cancer). In some embodiments, the invention provides methods and compositions for determining the activation level of an activatable element that is in the same pathway of a gene product that has a genetic alteration associated with a condition (e.g. cancer). In some embodiments, the genetic alteration is in a caretaker gene. The term “genetic alteration” as used herein includes mutations, deletions, insertions, base substitutions, breakage, rearangement, transpositions, transitions, transversions, amplifications, inversions, loss of heterozygosity, tautomerism, depurination, deamination, changes in methylation, changes in acetylation, chemical induced mutations, viral induced mutations, and radiation induced mutations. The term “genetic alteration” is also meant to include epigenetic changes. In some embodiments, the invention provides methods and compositions for determining the activation level of an activatable element in a cell suspected of being cancerous, wherein the activatable element has a genetic alteration. In some embodiments, the invention provides methods and compositions for determining the activation level of an activatable element in a cell suspected of being cancerous, wherein the activatable element is affected by and/or is in the same pathway as a gene or gene product that has a genetic alteration associated with a condition (e.g. cancer). In some embodiments, the invention provides methods and compositions for determining the activation level of an activatable element in a cell, wherein the activatable element has a genetic alteration associated with a predisposition for cancer. In some embodiments, the invention provides methods and compositions for determining the activation level of an activatable element in a cell, wherein the activatable element is affected by and/or is in the same pathway as a gene or gene product that has a genetic alteration associated with a condition (e.g. cancer). Genetic alterations can occur due to heritable mutations known in the art as germ line alterations or germ line mutations. Such inherited mutations are carried by all nucleated cells of an individual, and are thus assayable in any tissue, not just tissues associated with the development of cancer resulting from such mutations or tumor tissue itself. Because the presence of a germline mutation and/or its functional effects can be monitored in any nucleated cell, cellular pathway deregulation—caused by a mutation—may predispose, indicate, cause or contribute to a condition or phenotype, (for example, cancer) can be determined by assessing at least one signaling response in any sample (e.g. even samples not associated with a condition), such as blood samples, collected from patients to evaluate risk of developing a certain condition, or diagnose, prognose, and/or select a method of treatment based on the diagnosis and/or prognosis of the condition. In some embodiments, the activation level of an activatable element resulting from a genetic alteration that is associated with a condition (e.g. cancer or predisposition to develop cancer) is tested in cells other than the cells associated with the condition. For example, the methods of the invention can use peripheral blood mononuclear cells (PBMCs). In some embodiments, PBMCs can be purified from blood samples taken from women at high risk of developing breast cancer based on family history, such as to evaluate the risk of developing premenopausal breast/ovarian cancer, diagnose, prognose and/or select a method of treatment for an individual carrying the functional alterations. In some embodiments, the activation level of an activatable element within a DNA damage repair pathway is used to identify a genetic alteration that is associated with a condition (e.g. cancer or predisposition to develop cancer) in cells associated with a condition and/or in cells other than the cells associated with the condition. In some embodiments, the DNA damage repair pathway is selected from the group consisting of double strand DNA damage repair pathway and homologous recombination pathway.

One embodiment of the invention is a method to measure the genetic instability both at the individual level (e.g. cell having germline-based mutations of keeper genes) and at the tumor level (i.e. tumor cell having somatic mutations in the same genes). In one embodiment, one or more cell signaling pathways in a cell from a normal individual, for example those that do not have one or more genetic mutation of interest, can be analyzed and recorded, such as in a database. One or more cell signaling pathways from a cell in an individual having one or more mutations of interest can be analyzed and compared to the pathways that are classified as normal. These methods can utilize the process of activatable element analysis described below.

Another embodiment of the invention permits the measurement of genomic instability in predisposing to cancer as well as in cancer and the response to certain drugs in that cancer.

Caretaker Genes and Downstream Genes

In some embodiments, the compositions and methods of the present invention can be used for determining the activation level of an activatable element where the activatable element is the product (e.g. protein) of a caretaker gene or a gene downstream from a caretaker gene. In some embodiments, the caretaker genes can be genes involved in DNA damage repair, genes involved in homologous recombination, genes involved in non-homologous recombination, such as non-homologous end-joining, or genes involved in double-stranded break repair. In some embodiments, the compositions and methods of the present invention can be used for determining the activation level of an activatable element in response to a modulator.

BRCA1/2 and Related Genes

In some embodiments, the compositions and methods of the invention measure one or more activatable elements associated with the BRCA1 and/or BRCA2 gene and/or BRCA1 and/or BRCA2 related pathways. Mutations in either BRCA1 or BRCA2 genes are associated with certain breast cancer tumors, i.e. triple-negative/basal-like carcinomas, where triple negative refers to breast cancer tumors that are negative by immunohistochemistry for estrogen receptor (ER), progesterone receptor (PgR) and human epidermal growth factor receptor 2 (HER2). BRCA1 is a tumor suppressor that resides in a large multi-subunit protein complex of tumor suppressors, DNA damage sensors and signal transducers named BASC for BRCA1-associated geneome surveillance complex. BRCA1 has many functions in the normal cell, including maintenance of geneomic integrity, chromatin remodeling, transcriptional regulation, cell cycle checkpoint regulation, repair of double-stranded DNA breaks, and homologous recombination mediated DNA repair. Mutations in this gene are responsible for approximately 40% of inherited breast cancers (which as a group constitutes ˜4% of human breast cancer). In some embodiments, compositions and methods for deriving a proteomic profile by measuring one or more activatable elements associated with one or more of the other members of BASC, such as MSH2, MSH6, MLH1, ATM, BLM, the RAD50-MRE11-NBS1 or DNA replication factor C (Genes & Dev. 2000. 14: 927-939). Each of these genes can cause genomic instability and can result in the loss or attenuation of protein function, which can in turn have a correspondingly variable degree of impact on the pathways in which these genes and their protein products function.

About 3-8% of breast cancer patients have been shown to have mutations in their BRCA genes. Individuals with BRCA1 or 2 mutations have a very high risk (up to 80%) of developing bilateral breast cancers at a relatively young age (<50 years) and also a high risk of developing ovarian cancer. (˜60%) Currently individuals with a family history of breast cancer are tested for the presence of BRCA1 and BRCA2 mutations and if positive they are counseled to undergo preventive bilateral mastectomy and oophorectomy. Also if a young woman even without family history of breast cancer develops triple negative breast cancer before menopause she will be tested for the presence of a germline mutation in those two genes; if positive other family members such as daughters, sisters, nieces etc. are also tested.

The breast cancer tumors in individuals with alterations in BRCA1 or BRCA2 genes also tend to be “triple-negative” tumors and or basal-like and are non-responsive to hormone therapy or treatment with the HER2 receptor antagonist Herceptin. While the increased presence of mutations, translocations and chromosomal instability are often correlated with the absence or mutation of BRCA1 and/or BRCA2, such genomic instability is also found in about one third of all breast cancers that lack any genetic alterations in the BRCA 1 or 2 genes i.e. sporadic tumors. Genomic instability in sporadic cancers is thought to be primarily the results of an oncogene-induced collapse of DNA replication forks, which in turns leads to DNA double-strand breaks and genomic instability.

The majority of BRCA1-associated breast cancers share many phenotypic features with triple negative and basal-like breast cancer (BLBC). BRCA1-associated tumors generally cluster with the basal-like subtype in gene expression profiling studies. Approximately 80-90% of these tumors are triple negative. Furthermore, there are morphologic similarities between BRCA1-associated, triple negative and basal-like breast cancers. 11-30% of breast cancer patients with triple negative tumors do not have germ line alterations in BRCA1 or 2 genes, yet in terms of clinico-pathologic features (time of development, aggressiveness) and therapeutic response their tumors behave like tumors from patients which carry germ line alterations in the BRCA1/2 genes. These observations suggest that tumors from these patients could be characterized by the presence of at least one functional abnormality mediated by or in the BRCA-encoded protein rather than any mutation within the BRCA1/2 genes. For patients with BRCA1 and/or 2 mutations the improper DNA sequence causes the functional abnormality. In the remaining majority of patients, functional insufficiency most likely arises because of aberrant regulation of BRCA1/2 expression, disruption of BRCA1/2 subcellular localization, or improper post-translational modifications. In one embodiment, compositions and methods for deriving a proteomic profile in these subgroups of breast cancer by measuring pathway activity will provide useful information regarding the behavior of cancerous or precancerous cells that display an identical phenotype as cancer cells that carry germline mutations in the BRCA1 and/or 2 genes.

Inherited BRCA mutations are carried by all nucleated cells of the body. In some embodiments, BRCA protein activity is tested in cells other than cells derived from a primary breast tumor or cells associated with breast cancer (e.g. PBMCs obtained from blood samples) to diagnose, prognose and/or select a method of treatment for an individual carrying the mutation. In some embodiments, cellular pathway deregulations caused by genetic alterations other than BRCA genetic alterations are tested in cells other than cells associated with breast cancer (e.g. PBMCs obtained from blood samples) to diagnose, prognose and/or select a method of treatment for an individual carrying the mutation. In some embodiments, protein activity of proteins other than BRCA is tested in cells other than cells associated with breast cancer (e.g. PBMCs obtained from blood samples) to diagnose, prognose and/or select a method of treatment for an individual carrying the mutation. In some embodiments, protein activity of proteins is tested in cells other than cells associated with breast cancer (e.g. PBMCs obtained from blood samples) that have genetic alterations other than BRCA but that phenocopy, or behave substantially similar to cells having BRCA alterations to diagnose, prognose and/or select a method of treatment for an individual carrying the mutation. In some embodiments, the activation level of an activatable element within a DNA damage repair pathway is measured in cells other than cells derived from a primary breast tumor or cells associated with breast cancer (e.g. PBMCs obtained from blood samples) to diagnose, prognose and/or select a method of treatment for an individual carrying the mutation. In some embodiments, the DNA damage repair pathway is selected from the group consisting of double strand DNA damage repair pathway and homologous recombination pathway.

In some embodiments, BRCA protein activity is tested in cells derived from a primary breast tumor or cells associated with breast cancer to diagnose, prognose and/or select a method of treatment for an individual carrying the mutation. In some embodiments BRCA protein activity is tested in cells derived from a primary breast tumor or cells associated with breast cancer using flow cytometry. In some embodiments, genetic alterations other than BRCA genetic alterations are tested in cells associated with breast cancer to diagnose, prognose and/or select a method of treatment for an individual carrying the mutation. In some embodiments, protein activity of proteins other than BRCA is tested in cells associated with breast cancer to diagnose, prognose and/or select a method of treatment for an individual carrying the mutation. In some embodiments, protein activity of proteins is tested in cells associated with breast cancer that have genetic alterations other than BRCA but that phenocopy behave substantially similar to cells having BRCA alterations to diagnose, prognose and/or select a method of treatment for an individual carrying the mutation. In some embodiments, the activation level of an activatable element within a DNA damage repair pathway is measured in cells derived from a primary breast tumor or cells associated with breast cancer to diagnose, prognose and/or select a method of treatment for an individual carrying a BRCA mutation. In some embodiments, the DNA damage repair pathway is selected from the group consisting of double strand DNA damage repair pathway and homologous recombination pathway.

In some embodiments, the activity of BRCA1 or 2 in response to a modulator can be assessed directly by monitoring the activation levels of BRCA in individual cells. BRCA1/2 activity can be further assessed by evaluating changes in the levels and activities of activatable elements downstream or upstream of BRCA1 activation in affected pathways. Examples of downstream changes include, but are not limited to, phosphorylation of Chk1 and transcriptional activation of GADD45 and p21WAF/CIP. In some embodiments, the activation level of BRCA1 and/or activatable elements downstream or upstream of BRCA1 activation in response to DNA damage induced by a modulator is measured. In some embodiments, DNA repair deficiencies are detected in (1) cells with BRCA mutations giving rise to abnormal protein products, and/or (2) cells with DNA repair functional insufficiencies that predispose patients to cancer. In some embodiments, the methods of the present invention predict the response of a cancer cell or tumor to a therapeutic agent (e.g. PARP-inhibitor). In some embodiments, cells from patients having at least one BRCA mutation and cells responding to the BRCA mutation-targeted chemotherapy regime are compared to wild type cells. In some embodiments, the cells are blood cells.

In some embodiments, the compositions and methods are useful for diagnosing a patient that has a genomic instability associated with BRCA1 or BRCA2, or a gene associated with BRCA1/2. In some embodiments, the compositions and methods are useful for diagnosing a patient that has a genomic instability associated with a gene other than BRCA1/2. In some embodiments, a patient may have a mutation or other functional insufficiency in at least one other gene or gene product that cooperates with BRCA mutations to promote neoplasia. For example, a mutation in the adenomatous polyposis coli (APC) gene, or in genes encoding proteins including but not limited to β-catenin and axin, affecting or affected by APC. Mutations in this gene correlate with the development of colorectal cancer (Curr Opin Genet Dev. 2007, EMBO Reports, 2005, 6(2): 184-190). The APC protein interacts with proteins acting in the Wnt signaling pathway, activation of which leads to beta-catenin/lymphoid-enhancing factor 1-dependent activation of other transformation-inducing genes. This activation can result from APC mutations that lead to a decrease in or loss of function, as APC acts as a negative regulator of beta-catenin. In one embodiment, the level and/or activation level of APC, or levels and/or activation level of beta-catenin or other activatable elements downstream or upstream of APC in participating pathways, may be measured in individual cells. In some embodiments, the compositions and methods can be used to determine if the patient has cancer or a predisposition for cancer. In some embodiments, the level and/or activation level of APC, or levels and/or activation levels of beta-catenin or other activatable elements downstream of APC in participating pathways, is measured in cells not associated with colorectal cancer (e.g. PBMCs obtained from blood samples). Analysis of these measurements can be used as a diagnostic, prognostic, or theranostic indicator of the development or progression of disease.

Ras Family Pathway

In another embodiment, a patient may have a mutation in the Ras gene, or in genes encoding proteins affecting or affected by Ras activity. The Ras superfamily of GTPases is a class of 21 KDa proteins that regulate multiple cellular functions including but not limited to cell growth, cell cycle control, protein secretion, cell motility, and intracellular vesicle transport and interaction. In particular, members of the Ras family regulate cellular functions by transducing mitogenic signals emanating from activated receptors (Tavitian, A. (1995) C. R. Seances Soc. Biol. Fil. 189:7-12). During this process, the hydrolysis of GTP acts as an energy source as well as an on-off switch for the GTPase activity of the LMW GTP-binding proteins.

The Ras superfamily is comprised of five subfamilies: Ras, Rho, Ran, Rab, and ADP-ribosylation factor (ARF). Ras superfamily members are necessary for the coordinated control of cell growth and proliferation. Mutations in Ras genes are associated with many types of cancer. Each Ras subfamily has distinct, yet partially overlapping functions. Rho proteins transduce proliferative signals from stimulated growth factor receptors to the actin cytoskeleton. Rho activation ultimately promotes actin polymerization and cytoskeletal remodeling necessary for cell division. Rab proteins control intracellular vesicle translocation and thereby regulate protein localization, protein processing, and secretion. Ran shuttles between the cell nucleus and the cytosol and is necessary for nuclear protein import, DNA replication, and cell-cycle progression. ARF and ARF-like proteins participate in a wide variety of cellular functions including vesicle trafficking, exocrine secretion, regulation of phospholipase activity, and endocytosis. In some embodiments, the invention provides methods and compositions for determining the activation level of these and other members of participating pathways in individual cells. In some embodiments, the measurement of the activation levels of these and other members of participating pathways are made in cells not associated with a cancer (e.g. PBMCs obtained from blood samples). In some embodiments, the compositions and methods are useful for determining genomic instability in cells having Ras mutations, or a mutation in a gene associated with Ras, such as growth factor receptors or FLT3. In some embodiments, the activation level of an activatable element within a DNA damage repair pathway is measured: (i) in cells derived from a primary tumor or cells associated with cancer, or (ii) cells not associated with a cancer (e.g. PBMCs obtained from blood samples), from an individual carrying a Ras mutation, or a mutation in a gene associated with Ras. In some embodiments, the DNA damage repair pathway is selected from the group consisting of double strand DNA damage repair pathway and homologous recombination pathway. Analysis of these measurements can be used as a diagnostic, prognostic, or theranostic indicator of the development or progression of disease.

P53 Pathway

In some embodiments, a patient may have at least one mutation in the gene coding for p53, or in genes encoding proteins affecting or affected by p53. The p53 tumor suppressor exerts anti-proliferative effects, including growth arrest, apoptosis, and cell senescence, in response to various types of stress (Levine, A. J., Cell 88:323-31, 1997; Oren, M., J. Biol. Chem. 274: 36031-034, 1999). p53 may represent the central node of a regulatory circuit that monitors diverse signaling pathways of disparate function, including DNA damage responses (e.g., ATM/ATR activation), abnormal oncogenic events (e.g., Myc or Ras activation) and cellular homeostasis. While p53 mutations occur in more than half of all human tumors (Hollstein et al., Mutat Res. 431:199-209, 1999), defects in other components of various p53 modulated pathways, such as the ARF tumor suppressor pathway, are observed in tumor cells that retain wildtype p53 (Shen, C. J., Nat Rev Mol Cell Biol 2:731-737, 2001; Sharpless, N. E., DePinho, R. A., J Clin Invest 113:160-8, 2004). Functional Inactivation of the p53 pathway appears to be a common, if not universal, feature of human cancer.

Paradoxically, functionally aberrant p53 mutants observed in human cancer may be stabilized and persist within tumor cells for a longer time than wild-type, functionally normal p53. p53 mutants observed in human tumors are often phosphorylated and acetylated at sites known to stabilize the protein by preventing its degradation by the 26S proteasome. In one embodiment, the activation level of p53, or activation levels of activatable elements downstream or upstream of p53 in affected pathways, is measured in individual cells, wherein the amount of total p53 or p53 recognized by a binding element with specificity for a particular p53 subspecies, such as phospho-serine 15 p53, serves as a diagnostic, prognostic, or theranostic indicator of the development or progression of a condition such as cancer. For example, in chronic lymphocytic leukemia (CLL), the percentage of p53 positivity is positively correlated with progressively later stages of the disease—Binet stage A (8/100 7.4%) to Binet stage B (12/49 24.4%) and to Binet stage C (7/25 29.2%). p53 correlated with the phase of the disease showing low expression at diagnosis (8/112 7.1%) and a higher level as the disease progressed (7/35 20%) (Cordone et al., Blood (1998) Vol. 91 p. 4342).

In some embodiments, the levels and/or activation levels of p53 are measured in single cells. In some embodiments, the levels and/or activation levels of p53 are measured in single cells using flow cytometry. For example, in a CLL sample the levels and/or activation levels of p53 are measured in B cells. In the same CLL sample p53 levels can be measured in other cell types, such as T cells and myeloid cells. p53 levels can also be measured simultaneously or sequentially with other activatable elements within one or more signaling pathways in distinct cell subsets within a CLL sample. In some embodiments, p53 levels are measured simultaneously or sequentially with other activatable elements within one or more signaling pathways in response to a modulator in distinct cell subsets within a CLL sample. This will be advantageous for monitoring levels of p53 expression in specific cell subsets within diagnostic CLL patient samples as well as changes in p53 that occur in the same patient over time. Measurements of p53 using the methods described herein can be made in other hematologic malignancies as well as in other tumor types.

In some embodiments, the levels and/or activation levels of functional, wild type p53 in leukemic cells is measured. For example, it is known that drugs such as etoposide stabilize p53 through ATM-mediated phosphorylation of serine 15. Phosphoryation at serine 15 prevents MDM2 binding and subsequent targeting of p53 for degradation by the 26S proteosome. In some embodiments, if mutant p53 protein is not detected then cells would be treated with a modulator to evaluate p-p53 (S15); this would indicate the presence of functional wild type protein. Other sites for post translational modification of p53 can be monitored with the methods described herein. Levels of MDM2, post translational modifications of MDM2, and p-Arf levels could also be measured by the methods described herein either alone or combined with each other or with the other modulated signaling assays.

In some embodiments, the compositions and methods of the invention can be used to measure the activation level of activatable elements associated with homologous recombination or activatable elements downstream from activatable elements associated with homologous recombination. Examples of activatable elements associated with homologous recombination include Rad51 and Rad52.

In some embodiments, the compositions and methods of the invention can be used to measure the activation level of activatable elements associated with non-homologous recombination or activatable elements downstream from activatable elements associated with non-homologous recombination. Examples of activatable elements associated with homologous recombination include DNA-pk, Ku70, Ku80 or ATM.

In some embodiments, the current method encompasses assaying the activation level of activatable elements, or activatable elements encoded by genes affecting or affected by a genetic alteration including but not limited to APC, AXIN2, ARF, ATM, ATR, a1sBLM, CDH1, GPC3, CYLD, EXT1, EXT2, PTCH, SUFU, FH, SDHB, SDHC, SDHD, VHL, TP53, WT1, STK11, PTEN, TSC1, TSC2, CDKN2A, CDK4, RB1, RAD50, NF1, BMPR1A, MEN1, SMAD4, BHD, HRPT2, NF2, MUTYH, ATM, BLM, BRCA1, BRCA2, FANCA, FANCC, FANCD2, FANCE, FANCF, FANCG, NBS1, RECQL4, WRN, MSH2, MLH1, MSH6, MDM2, MRE11, NBS1, RAS, RHO, RAN, RAB, PMS2, p53, XPA, XPC, ERCC2, ERCC3, ERCC4, ERCC5, DDB2, KIT, MET, PDGFRA, RET, and DNA replication factor C.

In some embodiments, the methods described herein are used in cells carrying germline and/or somatic mutations in the genes described in table 1, or genes that affect the genes listed in table 1, or genes that might be affected by the genes listed in table 1. The cells can be cells associated with a condition, or the cells can be cells not associated with a condition. The cells can be cells associated with a condition that have not yet developed the condition. Other germline or somatic mutations are shown in Negrini, Nature Reviews, Molecular Cell Biology, March 2010, p 220, vol 11 which is hereby incorporated by reference.

TABLE 1

| | | Hereditary | | Major Heredity Cancer |
|---|---|---|---|---|
| Gene | Syndrome | pattern | Pathway | types |
| APC | FAP | Dominant | APC | Colon, thyroid, stomach, |
| | | | | intestine |
| AXIN2 | Attenuated | Dominant | APC | Colon |
| | polyposis | | | |
| CDH1 | Familial | Dominant | APC | Stomach |
| | gastric | | | |
| | carcinoma | | | |
| GPC3 | Simpson- | X-linked | APC | Emryonal |
| | Golabi- | | | |
| | Behmel | | | |
| | syndrome | | | |
| CYLD | Familial | Dominant | Apoptosis | Pilotrichomas |
| | cylindromatosis | | | |
| EXT1, 2 | Hereditary | Dominant | GLI | Bone |
| | multiple | | | |
| | exostoses | | | |
| PTCH | Gorlin | Dominant | GLI | Skin, medulloblastoma |
| | syndrome | | | |
| SUFU | Medulloblastoma | Dominant | GLI | Skin, medulloblastoma |
| | predisposition | | | |
| FH | Hereditary | Dominant | HIF1 | Leiomyomas |
| | leiomyomatosis | | | |
| SDHB, C, | Familial | Dominant | HIF1 | Paragangliomas, |
| D | paraganglioma | | | pheochromocytomas |
| VHL | Von Hippel- | Dominant | HIF1 | Kidney |
| | Lindau | | | |
| | syndrome | | | |
| TP53 | Li-Fraumeni | Dominant | p53 | Breast, sarcoma, adrenal, |
| | syndrome | | | brain, others |
| WT1 | Familial | Dominant | p53 | Wilms' |
| | Wilms tumor | | | |
| STK11 | Peutz- | Dominant | PI3K | Intestinal, ovarian, |
| | Jeghers | | | pancreatic |
| | syndrome | | | |
| PTEN | Cowden | Dominant | PI3K | Hamartoma, glioma, uterus |
| | syndrome | | | |
| TSC1, | Tuberous | Dominant | PI3K | Hamartoma, kidney |
| TSC2 | sclerosis | | | |
| CDKN2A | Familial | Dominant | RB | Melanoma, pancreas |
| | malignant | | | |
| | melanoma | | | |
| CDK4 | Familial | Dominant | RB | Melanoma |
| | malignant | | | |
| | melanoma | | | |
| RB1 | Hereditary | Dominant | RB | Eye |
| | retinoblastoma | | | |
| NF1 | Neurofibromatosis | Dominant | Receptor | Neurofibroma |
| | type 1 | | tyrosine | |
| | | | kinase | |
| BMPR1A | Juvenile | Dominant | SMAD | Gastrointestinal |
| | polyposis | | | |
| MEN1 | Multiple | Dominant | SMAD | Parathyroid, pituitary, islet |
| | endocrine | | | cell, carcinoid |
| | neoplasia | | | |
| | type I | | | |
| SMAD4 | Juvenile | Dominant | SMAD | Gastrointestinal |
| | polyposis | | | |
| BHD | Birt-Hogg- | Dominant | | Renal, hair follicle |
| | Dube | | | |
| | syndrome | | | |
| HRPT2 | Hyperparathy- | Dominant | | Parathyroid, jaw fibroma |
| | roidism, Jaw- | | | |
| | tumor | | | |
| | syndrome | | | |
| NF2 | Neurofibromatosis | Dominant | | Meningioma, acousic |
| | type 2 | | | neuroma |
| MUTYH | Attenuated | Recessive | BER | Colon |
| | polyposis | | | |
| ATM | Ataxia | Recessive | Chromosomal | Leukemias, lymphomas, |
| | telangiectasia | | instability | brain |
| BLM | Bloom | Recessive | Chromosomal | Leukemias, lymphomas, skin |
| | syndrome | | instability | |
| BRCA1, | Hereditary | Dominant | Chromosomal | Breast, ovary |
| BRCA2 | breast cancer | | instability | |
| FANCA, | Fanconi | Recessive | Chromosomal | Leukemias |
| C, D2, E, | anemia | | instability | |
| F, G | | | | |
| NBS1 | Nijmegen | Recessive | Chromosomal | Lymphomas, brain |
| | breakage | | instability | |
| | syndrome | | | |
| RECQL4 | Rothmund- | Recessive | Chromosomal | Bone, skin |
| | Thomson | | instability | |
| | syndrome | | | |
| WRN | Werner | Recessive | Chromosomal | Bone, brain |
| | syndrome | | instability | |
| MSH2, | HNPCC | Dominant | Mismatch | Colon, uterus |
| MLH1, | | | repair | |
| MSH6, | | | | |
| PMS2 | | | | |
| XPA, C; | Xeroderma | Recessive | Nucleotide- | Skin |
| ERCC2-5; | pigmentosum | | excision | |
| DDB2 | | | repair | |
| KIT | Familial | Dominant | Receptor | Gastrointestinal stromal |
| | gastrointestinal | | tyrosine | tumors |
| | stromal | | kinase | |
| | tumors | | | |
| MET | Hereditary | Dominant | Receptor | Kidney |
| | papillary renal | | tyrosine | |
| | cell | | kinase | |
| | carcinoma | | | |
| PDGFRA | Familial | Dominant | Receptor | Gastrointestinal stromal |
| | gastrointestinal | | tyrosine | tumors |
| | stromal | | kinase | |
| | tumors | | | |
| RET | Multiple | Dominant | Receptor | Thyroid, parathyroid, adrenal |
| | endocrine | | tyrosine | |
| | neoplasia | | kinase | |
| | type II | | | |

Examples of cancers that can be studied by the methods described herein include, but are not limited to, colon cancers, thyroid cancers, stomach cancers, intestinal cancers, embryonal cancers, pilotrichomas, bone cancers, skin cancers, medulloblastoma, leiomyomas, paragangliomas, pheochromocytomas, kidney cancers, breast cancers, adrenal cancers, brain cancers, Wilms' cancers, ovarian cancers, pancreatic cancers, hamartoma, glioma, uterine cancers, melanoma, cancers of the eye, neurofibroma, gastrointestinal cancers, parathyroid cancers, pituitary cancers, islet cell cancers, carcinoid cancers, hair follicle cancers, jaw fibroma, meningioma, acoustic neuroma, leukemias, and lymphomas.

### Samples and Sampling

The methods involve analysis of one or more samples from an individual. An individual or a patient is any multicellular organism; in some embodiments, the individual is an animal, e.g., a mammal. In some embodiments, the individual is a human.

The sample may be any suitable type that allows for the analysis of different populations of cells. The sample may be any suitable type that allows for the analysis of single populations of cells. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement of treatment response and also the monitoring for disease.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

Samples may include circulating tumor cells (CTC). Methods for isolating CTC are known in the art. See for example: Toner M et al. Nature 450, 1235-1239 (20 Dec. 2007); Lustenberger P et al. Int J Cancer. 1997 Oct. 21; 74(5):540-4; Reviews in Clinical Laboratory Sciences, Volume 42, Issue 2 Mar. 2005, pages 155-196; and Biotechno, pp. 109-113, 2008 International Conference on Biocomputation, Bioinformatics, and Biomedical Technologies, 2008.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

In one embodiment, a sample may be obtained from an apparently healthy individual during a routine checkup and analyzed so as to provide an assessment of the individual's general health status and risk of developing certain cancers. In another embodiment, a sample may be taken to screen for commonly occurring diseases. Such screening may encompass testing for a single disease, a family of related diseases or a general screening for multiple, unrelated diseases. Screening can be performed weekly, bi-weekly, monthly, bi-monthly, every several months, annually, or in several year intervals and may replace or complement existing screening modalities.

In another embodiment, an individual with a known increased probability of disease occurrence may be monitored regularly to detect the appearance of a particular disease or class of diseases. An increased probability of disease occurrence can be based on familial association, age, previous genetic testing results, or occupational, environmental or therapeutic exposure to disease causing agents. Breast and ovarian cancer in which patients have inherited germline mutations in the BRCA1 and BRCA2genes are examples of diseases with a familial association wherein susceptible individuals can be identified through genetic testing. Another example is the presence of inherited mutations in the APC gene predisposing individuals to colorectal cancer. Examples of environmental or therapeutic exposure include individuals occupationally exposed to benzene that have increased risk for the development of various forms of leukemia, and individuals therapeutically exposed to alkylating agents for the treatment of earlier malignancies. Individuals with increased risk for specific diseases can be monitored regularly for the first signs of an appearance of an abnormal cell population. Monitoring can be performed weekly, bi-weekly, monthly, bi-monthly, every several months, annually, or in several year intervals, or any combination thereof. Monitoring may replace or complement existing screening modalities. Through routine monitoring, early detection of the presence of disease causative or associated cells may result in increased treatment options including treatments with lower toxicity and increased chance of disease control or cure.

In a further embodiment, testing can be performed to confirm or refute the presence of a suspected genetic alteration associated with increased risk of disease. Such methodologies are known in the art. Such testing methodologies include, but are not limited to, techniques like cytogenetic analysis, fluorescent in situ histochemistry (FISH), PCR, DNA arrays and sequencing.

In instances where an individual has a known pre-pathologic or pathologic condition, one or a plurality of cell populations from the appropriate location can be sampled and analyzed to predict the response of the individual to available treatment options. In one embodiment, an individual treated with the intent to reduce in number or ablate cells that are causative or associated with a pre-pathological or pathological condition can be monitored to assess the decrease in such cells over time. A reduction in causative or associated cells may or may not be associated with the disappearance or lessening of disease symptoms. If the anticipated decrease in cell number does not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another cell population(s) that are associated with a good prognosis. If a beneficial, population of cells is observed, measures can be taken to further increase their numbers, such as the administration of growth factors. Alternatively, individuals may be monitored for the appearance or increase in cell number of another cell population(s) associated with a poor prognosis. In such a situation, renewed therapy can be considered including continuing, modifying the present therapy or initiating another type of therapy.

In these embodiments, one or more samples may be taken from the individual, and subjected to a modulator, as described herein. In some embodiments, the sample is divided into subsamples that are each subjected to a different modulator. After treatment with the modulator, one or more different populations of cells in the sample or subsample are analyzed to determine their activation level(s). In some embodiments, single cells in one or more different population are analyzed. Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the activation level may be achieved by the use of activation state-specific binding elements, such as antibodies, as described herein. A plurality of activatable elements may be examined in one or more of the different cell populations.

Certain fluid samples can be analyzed in their native state with or without the addition of a diluent or buffer. Alternatively, fluid samples may be further processed to obtain enriched or purified cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 μm, as disclosed in U.S. patent application Ser. No. 09/790,673. Alternatively, whole blood can be separated into its constituent cells based on size, shape, deformability or surface receptors or surface antigens by the use of a microfluidic device as disclosed in U.S. patent application Ser. No. 10/529,453.

Select cell populations can also be enriched for or isolated from whole blood through positive or negative selection based on the binding of antibodies or other entities that recognize cell surface or cytoplasmic constituents. For example, U.S. Pat. No. 6,190,870 to Schmitz et al. discloses the enrichment of tumor cells from peripheral blood by magnetic sorting of tumor cells that are magnetically labeled with antibodies directed to tissue specific antigens.

Solid tissue samples may require the disruption of the extracellular matrix or tissue stroma and the release of single cells for analysis. Various techniques are known in the art including enzymatic and mechanical degradation employed separately or in combination. An example of enzymatic dissociation using collagenase and protease can be found in Wolters G H J et al. An analysis of the role of collagenase and protease in the enzymatic dissociation of the rat pancrease for islet isolation. Diabetologia 35:735-742, 1992. Examples of mechanical dissociation can be found in Singh, N P. Technical Note: A rapid method for the preparation of single-cell suspensions from solid tissues. Cytometry 31:229-232 (1998). Alternately, single cells may be removed from solid tissue through microdissection including laser capture microdissection as disclosed in Laser Capture Microdissection, Emmert-Buck, M. R. et al. Science, 274(8):998-1001, 1996.

In some embodiments, single cells can be analyzed within a tissue sample, such as a tissue section or slice, without requiring the release of individual cells before determining step is performed.

The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 61/048,886; 61/048,920; and 61/048,657. See also, the commercial products from companies such as BD and BCI as identified above.

See also U.S. Pat. Nos. 7,381,535 and 7,393,656. All of the above patents and applications are incorporated by reference as stated above.

In some embodiments, the cells are cultured post collection in a media suitable for revealing the activation level of an activatable element (e.g. RPM I, DMEM) in the presence, or absence, of serum such as fetal bovine serum, bovine serum, human serum, porcine serum, horse serum, or goat serum. When serum is present in the media it could be present at a level ranging from 0.0001% to 30%.

### Modulators

In some embodiments, the methods and composition utilize a modulator. A modulator can be an activator, a therapeutic compound, an inhibitor or a compound capable of impacting a cellular pathway. Modulators can also take the form of environmental cues and inputs. Modulators can be uncharacterized or characterized as known compounds. A modulator can be a biological specimen or sample of a cellular or physiological environment from an individual, which may be a heterogeneous sample without complete chemical or biological characterization. Collection of the modulator specimen may occur directly from the individual, or be obtained indirectly. An illustrative example would be to remove a cellular sample from the individual, and then culture that sample to obtain modulators.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. See U.S. Patent Application 61/048,657 which is incorporated by reference.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments the modulator is selected from the group consisting of growth factors, cytokines, adhesion molecules, drugs, hormones, small molecules, polynucleotides, antibodies, natural compounds, lactones, chemotherapeutic agents, immune modulators, carbohydrates, proteases, ions, reactive oxygen species, peptides, and protein fragments, either alone or in the context of cells, cells themselves, viruses, and biological and non-biological complexes (e.g. beads, plates, viral envelopes, antigen presentation molecules such as major histocompatibility complex). In some embodiments, the modulator is a physical stimulus such as heat, cold, UV radiation, and radiation. Examples of modulators, include but are not limited to EGF, amphiregulin, TGFα, TGFβ, PDGFs, FGFs IGF-1, insulin, Ephs, VEGFs, Wnts, notch ligands, hedgehogs, angiopoietins, SDF-1α, IFN-α, IFN-γ, IL-10, IL-6, IL-27, G-CSF, FLT-3L, IGF-1, M-CSF, SCF, PMA, Thapsigargin, H₂O₂, etoposide, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L.

In some embodiments, the modulator is an activator. In some embodiments the modulator is an inhibitor. In some embodiments, cells are exposed to one or more modulator. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the cross-linker is a molecular binding entity. In some embodiments, the molecular binding entity is a monovalent, bivalent, or multivalent is made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor. Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride. In some embodiments, the phosphatase inhibitor is H₂O₂.

In some embodiments, the activation level of an activatable element in a cell is determined by contacting the cell with at least 2, 3, 4, 5, 6, 7, 8, 9, or 10 modulators. In some embodiments, the activation level of an activatable element in a cell is determined by contacting the cell with at least 2, 3, 4, 5, 6, 7, 8, 9, or 10 modulators where at least one of the modulators is an inhibitor. In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In some embodiments, the physiological status a population of cells is determined by measuring the activation level of an activatable element when the population of cells is exposed to one or more modulators. The population of cells can be divided into a plurality of samples, and the physiological status the population is determined by measuring the activation level of at least one activatable element in the samples after the samples have been exposed to one or more modulators. In some embodiments, the physiological status of different populations of cells is determined by measuring the activation level of an activatable element in each population of cells when each of the populations of cells is exposed to a modulator. The different populations of cells can be exposed to the same or different modulators.

In some embodiments, the modulators include SDF-1α, IFN-α, IFN-γ, IL-10, IL-6, IL-27, G-CSF, FLT-3L, IGF-1, M-CSF, SCF, PMA, Thapsigargin, H₂O₂, etoposide, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L. For instance a population of cells can be exposed to one or more, all or a combination of the following combination of modulators: SDF-1a, IFN-α, IFN-γ, IL-10, IL-6, IL-27, G-CSF, FLT-3L, IGF-1, M-CSF, SCF, PMA, Thapsigargin, H₂O₂, etoposide, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L. In some embodiments, the physiological status of different cell populations is used for the diagnosis, prognosis, and/or selection of treatment of an individual as described herein.

### Determination of Physiological Status of a Cell Population

After treatment with one or more modulators, if used, in some embodiments the sample is analyzed to determine the physiological status of at least one cell or at least one cell population. In some embodiments, the physiological status of a cell population is determined by contacting the cell population with one or more modulators and determining the activation level of an activatable element of at least one cell in the cell population. The population of cells can be divided into a plurality of samples, and the physiological status the population is determined by measuring the activation level of at least one activatable element in the samples after the samples have been exposed to one or more modulators. In some embodiments the physiological status is measured for: (i) cells derived from a primary tumor or cells associated with cancer, (ii) cells not associated with a cancer (e.g. PBMCs obtained from blood samples), or (iii) cells associated with cancer that have not yet developed cancer, from an individual carrying a germline and/or somatic mutation. In some embodiments, the physiological status of a cell is determined by measuring DNA repair levels in cells containing a genetic alteration. Thus, in some embodiments, determining the physiological status of a cell involves determining DNA repair deficiencies in the cell. In some embodiments, DNA repair deficiencies are determined by measuring the activation level of an activatable element within a DNA damage repair pathway. In some embodiments, the DNA damage repair pathway is selected from the group consisting of double strand DNA damage repair pathway and homologous recombination pathway. In some embodiments, the analysis is performed in single cells. Any suitable analysis that allows determination of the activation level of an activatable element within single cells, which provides information useful for determining the physiological status of a cell population from whom the sample was taken, may be used. Examples include flow cytometry, immunohistochemistry, immunofluorescent histochemistry with or without confocal microscopy, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, Inductively Coupled Plasma Mass Spectrometer (ICP-MS) and label-free cellular assays. Additional information for the further discrimination between single cells can be obtained by many methods known in the art including the determination of the presence of absence of extracellular and/or intracellular markers, the presence of metabolites, gene expression profiles, DNA sequence analysis, and karyotyping. In some embodiments, the methods describe herein measure the functional consequences of genetic and epigenetic alterations (e.g., in genes and their expression) affecting proteins which are part of a DDR pathway. Thus the methods describe herein measure functionally deregulation in DDR pathways caused by any genetic and epigenetic alteration. In some embodiments, deregulation in DDR pathways caused by the germline mutation can be used as predisposition factor to cancer. In some embodiments, deregulation in DDR pathways caused by somatic mutations can be used as an indicator of possible response to treatment, e.g., hypersensitivity to PARP inhibitors.

For information on PARP, see U.S. Ser. No. 61/436,534 and PCT/US2011/48322. PARP inhibitors (PARPi) are a group of pharmacological inhibitors of the enzyme poly ADP ribose polymerase (PARP). They are developed for multiple indications; the most important is the treatment of cancer.[1] Several forms of cancer are more dependent on PARP than regular cells, making PARP an attractive target for chemotherapeutic cancer therapy. DNA is damaged thousands of times during each cell cycle, and that damage must be repaired.

BRCA1, BRCA2 and PALB2 are proteins that are important for the repair of double-strand DNA breaks by the error-free homologous recombinational repair, or HR, pathway. When the gene for either protein is mutated, the change can lead to errors in DNA repair that can eventually cause breast cancer. When subjected to enough damage at one time, the altered gene can cause the death of the cells. PARP1 is a protein that is important for repairing single-strand breaks (‘nicks’ in the DNA). If such nicks persist unrepaired until DNA is replicated (which must precede cell division), then the replication itself will cause double strand breaks to form. Drugs that inhibit PARP1 cause multiple double strand breaks to form in this way, and in tumours with BRCA1, BRCA2 or PALB2 mutations these double strand breaks cannot be efficiently repaired, leading to the death of the cells. Normal cells that don't replicate their DNA as often as cancer cells, and that lacks any mutated BRCA1 or BRCA2 still have homologous repair operating, which allows them to survive the inhibition of PARP. Some cancer cells that lack the tumor suppressor PTEN may be sensitive to PARP inhibitors because of downregulation of Rad51, a critical homologous recombination component, although other data suggest PTEN may not regulate Rad51. Hence PARP inhibitors may be effective against many PTEN-defective tumours (e.g. some aggressive prostate cancers). See Wikipedia for further information for PARP inhibitors.

PARP inhibitors can include Olaparib (AZD2281); Iniparib (BSI 201); Rucaparib (AG014699); Velparib (ABT-888); CEP 9722; MK 4827; BMN-673 and 3-aminobenzamide.

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously).

As will be appreciated by those in the art, a wide variety of activation events can find use in the present invention. In general, the basic requirement is that the activation results in a change in the activatable protein that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). What is important is to differentiate, using detectable events or moieties, between two or more activation states. However, in other instances an activatable element gets activated by increased expression. Thus, in those instances the increased expression of the activatable element will be measured whether or not there is a differentiating moiety between two or more activation states of the cells.

In some instances, the activation state of an individual activatable element is either in the on or off state. As an illustrative example, and without intending to be limited to any theory, an individual phosphorylatable site on a protein will either be phosphorylated and then be in the “on” state or it will not be phosphorylated and hence, it will be in the “off” state. See Blume-Jensen and Hunter, Nature, vol 411, 17 May 2001, p 355-365. The terms “on” and “off,” when applied to an activatable element that is a part of a cellular constituent, are used here to describe the state of the activatable element (e.g., phosphorylated is “on” and non-phosphorylated is “off”), and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis determining the activation levels of one or more activatable elements in cells may use the distribution of activation levels for one or more specific activatable elements which will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to determine the physiological status of a cell in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may not contain activatable elements, of one or more cells in a population of cells may be used to determine the physiological status of the cell population.

In some embodiments, the basis for determining the physiological status of a population of cells may use the position of a cell in a contour or density plot. The contour or density plot represents the number of cells that share a characteristic such as the activation level of activatable proteins in response to a modulator. For example, when referring to activation levels of activatable elements in response to one or more modulators, normal individuals and patients with a condition might show populations with increased activation levels in response to the one or more modulators. However, the number of cells that have a specific activation level (e.g. specific amount of an activatable element) might be different between normal individuals and patients with a condition. Thus, the physiological status of a cell can be determined according to its location within a given region in the contour or density plot.

In addition to activation levels of intracellular activatable elements, expression levels of intracellular or extracellular biomolecules, e.g., proteins may be used alone or in combination with activation states of activatable elements to determine the physiological status of a population of cells. Further, additional cellular elements, e.g., biomolecules or molecular complexes such as RNA, DNA, carbohydrates, metabolites, and the like, may be used in conjunction with activatable states, expression levels or any combination of activatable states and expression levels in the determination of the physiological status of a population of cells encompassed here.

In some embodiments, other characteristics that affect the status of a cellular constituent may also be used to determine the physiological status of a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to determine the physiological status of a cell, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, myeloid lineage cells can be further subdivided based on the expression of cell surface markers such as CD14, CD15, or CD33, CD34 and CD45.

Alternatively, populations of cells can be aggregated based upon shared characteristics that may include inclusion in one or more additional cell populations or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing characteristics.

In some embodiments, the physiological status of one or more cells is determined by examining and profiling the activation level of one or more activatable elements in a cellular pathway. In some embodiments, the physiological status of one or more cells is determined by examining and profiling the activation level of one or more activatable elements in a DNA damage repair pathway. In some embodiments, the physiological status of one or more cells is determined by examining and profiling the activation level of one or more activatable elements in a plurality of cellular pathways. In some embodiments, one of the cellular pathways in the plurality of pathway is DNA damage repair pathway. In some embodiments, the physiological status of one or more cells is determined by examining and profiling the activation level of one or more activatable elements in a plurality of DNA damage repair pathways. In some embodiments, the activation levels of one or more activatable elements of a cell from a first population of cells and the activation levels of one or more activatable elements of cell from a second population of cells are correlated with a condition. In some embodiments, the first population of cells and second population of cells are hematopoietic cell populations. In some embodiments, the activation levels of one or more activatable elements of a cell from a first population of hematopoietic cells and the activation levels of one or more activatable elements of cell from a second population of hematopoietic cells are correlated with a neoplastic, autoimmune or hematopoietic condition as described herein. Examples of different populations of hematopoietic cells include, but are not limited to, pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of activatable elements in single cells are determined. The term “plurality” as used herein refers to two or more. In some embodiments, at least about 2, 3, 4, 5, 6, 7, 8, 9, 10, or more than 10 intracellular activatable elements are determined. The plurality of activatable elements can be part of the same cellular pathway or can be part of different cellular pathways. In some embodiments, at least one of the activatable elements is an activatable element within a DNA damage repair pathway. In some embodiments, at least one of the activatable elements is measured in a plurality of DNA damage repair pathways.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

In some embodiments, the activatable element is a protein. Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886, 61/048,920 and Shulz et al, Current Protocols in Immunology 2007, 7:8.17.1-20.

In some embodiments, the protein that may be activated is selected from the group consisting of HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon a, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pinl prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors. In some embodiments, the protein that may be activated is selected from the proteins listed in FIGS. 1-8.

In some embodiments of the invention, the methods described herein are employed to determine the activation level of an activatable element, e.g., in a cellular pathway. Methods and compositions are provided for the determination of the physiological status of a cell according to the activation level of an activatable element in a cellular pathway. Methods and compositions are provided for the determination of the physiological status of a cell in a first cell population and a cell in a second cell population according to the activation level of an activatable element in a cellular pathway in each cell. The cells can be a hematopoietic cell and examples are shown above.

In some embodiments, the determination of the physiological status cells in different populations according to activation level of an activatable element, e.g., in a cellular pathway comprises classifying at least one of the cells as a cell that is correlated with a clinical outcome. Examples of clinical outcomes, staging, as well as patient responses are also shown above.

### Signaling Pathways

In some embodiments, the methods of the invention are employed to determine the status of an activatable element in a signaling pathway. In some embodiments, a cell is classified, as described herein, according to the activation level of one or more activatable elements in one or more signaling pathways. Signaling pathways and their members have been described. See (Hunter T. Cell Jan. 7, 2000; 100(1): 13-27). Exemplary signaling pathways include the following pathways and their members: The MAP kinase pathway including Ras, Raf, MEK, ERK and elk; the PI3K/Akt pathway including PI-3-kinase, PDK1, Akt and Bad; the NF-κB pathway including IKKs, IkB and the Wnt pathway including frizzled receptors, beta-catenin, APC and other co-factors and TCF (see Cell Signaling Technology, Inc. 2002 Catalog pages 231-279 and Hunter T., supra.). In some embodiments of the invention, the correlated activatable elements being assayed (or the signaling proteins being examined) are members of the MAP kinase, Akt, NFkB, WNT, RAS/RAF/MEK/ERK, JNK/SAPK, p38 MAPK, Src Family Kinases, JAK/STAT and/or PKC signaling pathways.

In some embodiments, the methods of the invention are employed to determine the activation level of a signaling protein in a signaling pathway known in the art including, but not limited to those described herein. Exemplary types of signaling proteins within the scope of the present invention include, but are not limited to kinases, kinase substrates (i.e. phosphorylated substrates), phosphatases, phosphatase substrates, binding proteins (such as 14-3-3), receptor ligands, receptors (cell surface receptor tyrosine kinases and nuclear receptors), proteases (e.g. caspases), and membrane associated proteins (e.g. Bax). Kinases and protein binding domains, for example, have been well described (see, e.g., Cell Signaling Technology, Inc., 2002 Catalogue “The Human Protein Kinases” and “Protein Interaction Domains” pgs. 254-279).

Nuclear Factor-kappaB (NF-κB) Pathway:

Nuclear factor-kappaB (NF-kappaB) transcription factors and the signaling pathways that activate them are central coordinators of innate and adaptive immune responses. More recently, it has become clear that NF-kappaB signaling also has a critical role in cancer development and progression. NF-kappaB provides a mechanistic link between inflammation and cancer, and is a major factor controlling the ability of both pre-neoplastic and malignant cells to resist apoptosis-based tumor-surveillance mechanisms. In mammalian cells, there are five NF-κB family members, RelA (p65), RelB, c-Rel, p50/p105 (NF-κB1) and p52/p100 (NF-κB2) and different NF-κB complexes are formed from their homo and heterodimers. In most cell types, NF-κB complexes are retained in the cytoplasm by a family of inhibitory proteins known as inhibitors of NF-κB (IκBs). Activation of NF-κB typically involves the phosphorylation of IκB by the IκB kinase (IKK) complex, which results in IκB polyubiquitination and subsequent proteosome dependent degradation. This releases NF-κB and allows it to translocate into the nucleus. The genes regulated by NF-κB include those controlling programmed cell death, cell adhesion, proliferation, the innate- and adaptive-immune responses, inflammation, the cellular-stress response, and tissue remodeling. However, the expression of these genes is tightly coordinated with the activity of many other signaling pathways. Therefore, the ultimate phenotypic response induced by NF-κB activation depends on the amplitude, duration, and cellular context of its induction. For example, it has become apparent that NF-κB activity can be regulated by both oncogenes and tumor suppressors, resulting in either stimulation or inhibition of apoptosis and proliferation. See Perkins, N. Integrating cell-signaling pathways with NF-κB and IKK function. Reviews: Molecular Cell Biology. January, 2007; 8(1): 49-62, hereby fully incorporated by reference in its entirety for all purposes. Hayden, M. Signaling to NF-κB. Genes & Development. 2004; 18: 2195-2224, hereby fully incorporated by reference in its entirety for all purposes. Perkins, N. Good Cop, Bad Cop: The Different Faces of NF-κB. Cell Death and Differentiation. 2006; 13: 759-772, hereby fully incorporated by reference in its entirety for all purposes.

Phosphatidylinositol 3-kinase (PI3-K)/AKT Pathway:

PI3-Ks are activated by a wide range of cell surface receptors, including but not limited to FLT3 LIGAND, EGFR, IGF-1R, HER2/neu, VEGFR, and PDGFR, to generate the lipid second messengers phosphatidylinositol 3,4-biphosphate (PIP₂) and phosphatidylinositol 3,4,5-trisphosphate (PIP₃). The lipid second messengers generated by PI3K regulate a diverse array of cellular functions. The specific binding of PIP2 and PIP3 to target proteins is mediated through the pleckstrin homology (PH) domain present in these target proteins. One key downstream effector of PI3-K is Akt, a serine/threonine kinase, which is activated when its PH domain interacts with PIP2 and PIP3 resulting in recruitment of Akt to the plasma membrane. Membrane bound, Akt is phosphorylated at threonine 308 by 3-phosphoinositide-dependent protein kinase-1 (PDK-1) and at serine 473 by several PDK2 kinases and thereby become fully active. Akt then acts downstream of PI3K to regulate the phosphorylation of numerous substrates, including but not limited to forkhead box 0 transcription factors, Bad, GSK-3β, I-κB, mTOR, MDM-2, and S6 ribosomal subunit. These phosphorylation events in turn mediate cell survival, cell proliferation, vesicle trafficking, glucose homeostasis, cellular metabolism, and cell motility. Disregulation of the PI3K pathway occurs by activating mutations in growth factor receptors, activating mutations in PI3-K genes (e.g. PIK3CA), loss of function mutations in a lipid phosphatase (e.g. PTEN), up-regulation of Akt expression and/or activity, or any functional impairment of the tuberous sclerosis complex (TSC1/2). All these events promote cell survival and proliferation. See Vivanco, I. The Phosphatidylinositol 3-Kinase-AKT Pathway in Human Cancer. Nature Reviews: Cancer. July, 2002; 2: 489-501 and Shaw, R. Ras, PI(3)K and mTOR signaling controls tumor cell growth. Nature. May, 2006; 441: 424-430, Marone et al., Biochimica et Biophysica Acta, 2008; 1784, p159-185 hereby fully incorporated by reference in their entirety for all purposes.

Wnt Pathway:

The complex Wnt signaling pathway comprises many proteins known for their roles in embryogenesis, tissue homeostasis, and cancer. The Wnt pathway also regulates self-renewal of hematopoietic stem cells (Reya T et al., Nature. 2003 May 22; 423(6938):409-14). Cytoplasmic levels of β-catenin are normally suppressed through the continuous proteosome mediated degradation of β-catenin controlled by a complex of glycogen synthase kinase 3β (GSK-3β), axin, and APC. Wnt pathway activation abrogates β-catenin degradation. Upon Wnt binding to a receptor complex composed of the Frizzled receptor (Fz) and low density lipoprotein receptor-related protein (LRP) at the cell surface, the GSK-3/axin/APC complex is inhibited. Key intermediate events during Wnt pathway activation include disheveled (Dsh) and axin binding the cytoplasmic tail of LRP. As β-catenin levels increase, it accumulates in the cytoplasm and nucleus. Nuclear β-catenin interacts with transcription factors such as lymphoid enhanced-binding factor 1 (LEF) and T cell-specific transcription factor (TCF) to affect transcription of target genes. See Gordon, M. Wnt Signaling: Multiple Pathways, Multiple Receptors, and Multiple Transcription Factors. J of Biological Chemistry. June, 2006; 281(32): 22429-22433, Logan C Y, Nusse R: The Wnt signaling pathway in development and disease. Annu Rev Cell Dev Biol 2004, 20:781-810, Clevers H: Wnt/beta-catenin signaling in development and disease. Cell 2006, 127:469-480. hereby fully incorporated by reference in its entirety for all purposes.

Protein Kinase C (PKC) Signaling:

The PKC family of serine/threonine kinases mediates signaling following activation of receptor tyrosine kinases, G-protein coupled receptors and cytoplasmic tyrosine kinases. Activation of PKC family members is associated with cell proliferation, differentiation, survival, immune function, invasion, migration, and angiogenesis. Disruption of PKC signaling has been implicated in tumorigenesis and chemotherapeutic drug resistance. PKC isoforms have distinct and overlapping functional roles. PKC was originally identified as a phospholipid and calcium-dependent protein kinase. The mammalian PKC superfamily consists of 13 different isoforms that are divided into four subgroups on the basis of their structural differences and related cofactor requirements cPKC (classical PKC) isoforms (α, βI, βII and γ), which respond both to Ca2+ and the lipid diacylglycerol (DAG), nPKC (novel PKC) isoforms (δ, ε, θ and η), which are insensitive to Ca2+, but dependent on DAG, atypical PKCs (aPKCs, ι/λ, ζ), which are responsive to neither co-factor, but may be activated by other lipids and through protein-protein interactions, and the related PKN (protein kinase N) family (e.g. PKN1, PKN2 and PKN3), members of which are subject to regulation by small GTPases. Consistent with their different biological functions, PKC isoforms differ in their tissue distribution, subcellular localization, mode of activation and substrate specificity. Maximal activation of PKC requires a priming phosphorylation provided by the constitutively active phosphoinositide-dependent kinase 1 (PDK-1). DAG plays a central role in PKC activation by causing an increase in the affinity of classical PKCs for the inner cell membrane. Membrane association further activates PKC by promoting the release of an inhibitory pseudo-substrate substrate which binds inactive PKC. Fully active PKC then phosphorylates and activates a range of kinases to transduce downstream signals.

The precise downstream events following PKC activation are poorly understood, although the MEK-ERK (mitogen activated protein kinase kinase-extracellular signal-regulated kinase) pathway is thought to have an important role. There is also evidence to support a role for PKC during activation of the PI3K-Akt pathway. Many reports describe dysregulation of several PKC family members. For example alterations in PKCε have been detected in thyroid cancer, and have been correlated with aggressive, metastatic breast cancer and PKCζ was shown to be associated with poor outcome in ovarian cancer. (Knauf J A, et al. Isozyme-Specific Abnormalities of PKC in Thyroid Cancer: Evidence for Post-Transcriptional Changes in PKC Epsilon. The Journal of Clinical Endocrinology & Metabolism. Vol. 87, No. 5, pp 2150-2159; Zhang L et al. Integrative Genomic Analysis of Protein Kinase C (PKC) Family Identifies PKC{iota} as a Biomarker and Potential Oncogene in Ovarian Carcinoma. Cancer Res. 2006, Vol 66, No. 9, pp 4627-4635)

Mitogen Activated Protein (MAP) Kinase Pathways:

MAP kinases transduce signals that are involved in a multitude of cellular pathways and are activated in response to a variety of ligands and cell stimuli. (Lawrence et al., Cell Research (2008) 18: 436-442). MAPKsignaling regulates several cellular processes such as protein relocalization, downstream kinase activation, transcription upregulation, and cell proliferation. MAPK also promotes complex processes such as embryogenesis and differentiation. Aberrant MAPK signaling is observed in diseases such as cancer, inflammatory disease, obesity, and diabetes. MAPKs are activated by upstream protein kinase cascades consisting of three or more protein kinases in series. MAPK kinase kinases (MAP3Ks) activate MAPK kinases (MAP2Ks) by dual phosphorylation on S/T residues; MAP2Ks then activate MAPKs by dual phosphorylation on Y and T residues MAPKs then phosphorylate target substrates on select S/T residues typically followed by a proline residue. In the ERK1/2 cascade the MAP3K is usually a member of the Raf family. Many diverse MAP3Ks are upstream of the p38 and the c-Jun N-terminal kinase/stress-activated protein kinase (JNK/SAPK) MAPK groups, which have generally been associated with responses to cellular stress. In an additional layer of complexity, the various kinases situated at several points along the kinase cascades described above may be directly stimulated by small G proteins, MAP4Ks, scaffolds, or oligomerization of a MAP3K.

Ras/RAF/MEK/ERK Pathway:

Classic activation of the RAS/Raf/MAPK cascade occurs following ligand binding to a receptor tyrosine kinase at the cell surface, but a vast array of other receptors have the ability to activate the cascade as well, such as integrins, serpentine receptors, heterotrimeric G-proteins, and cytokine receptors. Although conceptually linear, considerable cross talk occurs between the Ras/Raf/MAPK/Erk kinase (MEK)/Erk MAPK pathway and other MAPK pathways as well as many other signaling cascades. Ras signaling is further complicated by the presence of at least three Ras isoforms in human cells, including K-Ras, N-Ras, and H-Ras. The pivotal role of the Ras/Raf/MEK/Erk MAPK pathway in multiple cellular functions underlies the importance of this kinase cascade in oncogenesis and maintenance of transformed cells. As such, the MAPK pathway has been a focus of intense investigation for therapeutic targeting. Many receptor tyrosine kinases may initiate MAPK signaling. Receptor tyrosine kinases activate MAPK signaling by auto-phosphorylating their cytoplasmic domains in response to binding extracellular growth factors. Auto-phosphorylation sites within the cytoplasmic domains of receptor tyrosine kinases provide docking sites for src-homology 2 (SH2) domain-containing signaling molecules. One class of molecules that contain SH2 domains are known as adaptor proteins. The adaptor protein Grb2 recruits guanine nucleotide exchange factors such as SOS-1 to the cell membrane. The guanine nucleotide exchange factor interacts with Ras at the cell membrane to promote the exchange of GDP for GTP bound to Ras. GTP-bound Ras is active and competent to transducer a signal to downstream effectors. Ras inactivation occurs upon hydrolysis of RasGTP to RasGDP. Ras proteins have intrinsically low GTPase activity. However, GTPase activity is stimulated by GTPase-activating proteins such as NF-1 GTPase-activating protein/neurofibromin and p120 GTPase activating protein thereby preventing prolonged Ras activation and thus Ras-mediated signaling. Ras activation is the first step in activation of the MAPK cascade. Following Ras activation, Raf (A-Raf, B-Raf, or Raf-1) is recruited to the cell membrane through binding to Ras and activated in a complex process involving phosphorylation and multiple cofactors that is not completely understood. Raf proteins directly activate MEK1 and MEK2 via phosphorylation of multiple serine residues. MEK1 and MEK2 are themselves tyrosine and threonine/serine dual-specificity kinases that subsequently phosphorylate threonine and tyrosine residues in Erk1 and Erk2 resulting in activation. Although MEK1/2 have no known targets besides Erk proteins, Erk has multiple targets including Elk-1, c-Ets1, c-Ets2, p90RSK1, MNK1, MNK2, and TOB. The cellular functions of Erk are diverse and include regulation of cell proliferation, survival, mitosis, and migration. McCubrey, J. Roles of the Raf/MEK/ERK pathway in cell growth, malignant transformation and drug resistance. Biochimica et Biophysica Acta. 2007; 1773: 1263-1284, hereby fully incorporated by reference in its entirety for all purposes, Friday and Adjei, Clinical Cancer Research (2008) 14, p342-346.

c-Jun N-Terminal Kinase (JNK)/Stress-Activated Protein Kinase (SAPK) Pathway:

The c-Jun N-terminal kinases (JNKs) were initially described as a family of serine/threonine protein kinases, activated by a range of stress stimuli and able to phosphorylate the N-terminal transactivation domain of the c-Jun transcription factor. This phosphorylation enhances c-Jun dependent transcriptional events in mammalian cells. Further research has revealed three JNK genes (JNK1, JNK2 and JNK3) and their splice-forms as well as the range of external stimuli that lead to JNK activation. JNK1 and JNK2 are ubiquitous, whereas JNK3 is relatively restricted to brain. The predominant MAP2Ks upstream of JNK are MEK4 (MKK4) and MEK7 (MKK7). MAP3Ks with the capacity to activate JNK/SAPKs include MEKKs (MEKK1, -2, -3 and -4), mixed lineage kinases (MLKs, including MLK1-3 and DLK), Tp12, ASKs, TAOs and TAK1. Knockout studies in several organisms indicate that different MAP3Ks predominate in JNK/SAPK activation in response to different upstream stimuli. The wiring may be comparable to, but perhaps even more complex than, MAP3K selection and control of the ERK1/2 pathway. JNK/SAPKs are activated in response to inflammatory cytokines; environmental stresses, such as heat shock, ionizing radiation, oxidant stress and DNA damage; DNA and protein synthesis inhibition; and growth factors. JNKs phosphorylate transcription factors c-Jun, ATF-2, p53, Elk-1, and nuclear factor of activated T cells (NFAT), which in turn regulate the expression of specific sets of genes to mediate cell proliferation, differentiation or apoptosis. INK proteins are involved in cytokine production, the inflammatory response, stress-induced and developmentally programmed apoptosis, actin reorganization, cell transformation and metabolism. Raman, M. Differential regulation and properties of MAPKs. Oncogene. 2007; 26: 3100-3112, hereby fully incorporated by reference in its entirety for all purposes.

p38 MAPK Pathway:

Several independent groups identified the p38 Map kinases, and four p38 family members have been described (α, β, γ, δ). Although the p38 isoforms share about 40% sequence identity with other MAPKs, they share only about 60% identity among themselves, suggesting highly diverse functions. p38 MAPKs respond to a wide range of extracellular cues particularly cellular stressors such as UV radiation, osmotic shock, hypoxia, pro-inflammatory cytokines and less often growth factors. Responding to osmotic shock might be viewed as one of the oldest functions of this pathway, because yeast p38 activates both short and long-term homeostatic mechanisms to osmotic stress. p38 is activated via dual phosphorylation on the TGY motif within its activation loop by its upstream protein kinases MEK3 and MEK6. MEK3/6 are activated by numerous MAP3Ks including MEKK1-4, TAOs, TAK and ASK. p38 MAPK is generally considered to be the most promising MAPK therapeutic target for rheumatoid arthritis as p38 MAPK isoforms have been implicated in the regulation of many of the processes, such as migration and accumulation of leucocytes, production of cytokines and pro-inflammatory mediators and angiogenesis, that promote disease pathogenesis. Further, the p38 MAPK pathway plays a role in cancer, heart and neurodegenerative diseases and may serve as promising therapeutic target. Cuenda, A. p38 MAP-Kinases pathway regulation, function, and role in human diseases. Biochimica et Biophysica Acta. 2007; 1773: 1358-1375; Thalhamer et al., Rheumatology 2008; 47:409-414; Roux, P. ERK and p38 MAPK-Activated Protein Kinases: a Family of Protein Kinases with Diverse Biological Functions. Microbiology and Molecular Biology Reviews. June, 2004; 320-344 hereby fully incorporated by reference in its entirety for all purposes.

Src Family Kinases:

Src is the most widely studied member of the largest family of nonreceptor protein tyrosine kinases, known as the Src family kinases (SFKs). Other SFK members include Lyn, Fyn, Lck, Hck, Fgr, Blk, Yrk, and Yes. The Src kinases can be grouped into two sub-categories, those that are ubiquitously expressed (Src, Fyn, and Yes), and those which are found primarily in hematopoietic cells (Lyn, Lck, Hck, Blk, Fgr). (Benati, D. Src Family Kinases as Potential Therapeutic Targets for Malignancies and Immunological Disorders. Current Medicinal Chemistry. 2008; 15: 1154-1165) SFKs are key messengers in many cellular pathways, including those involved in regulating proliferation, differentiation, survival, motility, and angiogenesis. The activity of SFKs is highly regulated intramolecularly by interactions between the SH2 and SH3 domains and intermolecularly by association with cytoplasmic molecules. This latter activation may be mediated by focal adhesion kinase (FAK) or its molecular partner Crk-associated substrate (CAS), which plays a prominent role in integrin signaling, and by ligand activation of cell surface receptors, e.g. epidermal growth factor receptor (EGFR). These interactions disrupt intramolecular interactions within Src, leading to an open conformation that enables the protein to interact with potential substrates and downstream signaling molecules. Src can also be activated by dephosphorylation of tyrosine residue Y530. Maximal Src activation requires the autophosphorylation of tyrosine residue Y419 (in the human protein) present within the catalytic domain. Elevated Src activity may be caused by increased transcription or by deregulation due to overexpression of upstream growth factor receptors such as EGFR, HER2, platelet-derived growth factor receptor (PDGFR), fibroblast growth factor receptor (FGFR), vascular endothelial growth factor receptor, ephrins, integrin, or FAK. Alternatively, some human tumors show reduced expression of the negative Src regulator, Csk. Increased levels, increased activity, and genetic abnormalities of Src kinases have been implicated in both solid tumor development and leukemias. Ingley, E. Src family kinases: Regulation of their activities, levels and identification of new pathways. Biochimica et Biophysica Acta. 2008; 1784 56-65, hereby fully incorporated by reference in its entirety for all purposes. Benati and Baldari., Curr Med Chem. 2008; 15(12):1154-65, Finn (2008) Ann Oncol. May 16, hereby fully incorporated by reference in its entirety for all purposes.

Janus Kinase (JAK)/Signal Transducers and Activators of Transcription (STAT) Pathway:

The JAK/STAT pathway plays a crucial role in mediating the signals from a diverse spectrum of cytokine receptors, growth factor receptors, and G-protein-coupled receptors. Signal transducers and activators of transcription (STAT) proteins play a crucial role in mediating the signals from a diverse spectrum of cytokine receptors growth factor receptors, and G-protein-coupled receptors. STAT directly links cytokine receptor stimulation to gene transcription by acting as both a cytosolic messenger and nuclear transcription factor. In the Janus Kinase (JAK)-STAT pathway, receptor dimerization by ligand binding results in JAK family kinase (JFK) activation and subsequent tyrosine phosphorylation of the receptor, which leads to the recruitment of STAT through the SH2 domain, and the phosphorylation of conserved tyrosine residue. Tyrosine phosphorylated STAT forms a dimer, translocates to the nucleus, and binds to specific DNA elements to activate target gene transcription, which leads to the regulation of cellular proliferation, differentiation, and apoptosis. The entire process is tightly regulated at multiple levels by protein tyrosine phosphatases, suppressors of cytokine signaling and protein inhibitors of activated STAT. In mammals seven members of the STAT family (STAT1, STAT2, STAT3, STAT4, STAT5a, STAT5b and STATE) have been identified. JAKs contain two symmetrical kinase-like domains; the C-terminal JAK homology 1 (JH1) domain possesses tyrosine kinase function while the immediately adjacent JH2 domain is enzymatically inert but is believed to regulate the activity of JH1. There are four JAK family members: JAK1, JAK2, JAK3 and tyrosine kinase 2 (Tyk2). Expression is ubiquitous for JAK1, JAK2 and TYK2 but restricted to hematopoietic cells for JAK3. Mutations in JAK proteins have been described for several myeloid malignancies. Specific examples include but are not limited to: Somatic JAK3 (e.g. JAK3A572V, JAK3V722I, JAK3P132T) and fusion JAK2 (e.g. ETV6-JAK2, PCM1-JAK2, BCR-JAK2) mutations have respectively been described in acute megakaryocytic leukemia and acute leukemia/chronic myeloid malignancies, JAK2 (V617F, JAK2 exon 12 mutations) and MPL MPLW515L/K/S, MPLS505N) mutations associated with myeloproliferative disorders and myeloproliferative neoplasms. JAK2 mutations, primarily JAK2V617F, are invariably associated with polycythemia vera (PV). This mutation also occurs in the majority of patients with essential thrombocythemia (ET) or primary myelofibrosis (PMF) (Tefferi n., Leukemia & Lymphoma, March 2008; 49(3): 388-397). STATs can be activated in a JAK-independent manner by src family kinase members and by oncogenic FLt3 ligand-ITD (Hayakawa and Naoe, Ann N Y Acad Sci. 2006 November; 1086:213-22; Choudhary et al. Activation mechanisms of STAT5 by oncogenic FLt3 ligand-ITD. Blood (2007) vol. 110 (1) pp. 370-4). Although mutations of STATs have not been described in human tumors, the activity of several members of the family, such as STAT1, STAT3 and STAT5, is dysregulated in a variety of human tumors and leukemias. STAT3 and STAT5 acquire oncogenic potential through constitutive phosphorylation on tyrosine, and their activity has been shown to be required to sustain a transformed phenotype. This was shown in lung cancer where tyrosine phosphorylation of STAT3 was JAK-independent and mediated by EGF receptor activated through mutation and Src. (Alvarez et al., Cancer Research, Cancer Res 2006; 66) STAT5 phosphorylation was also shown to be required for the long-term maintenance of leukemic stem cells. (Schepers et al. STAT5 is required for long-term maintenance of normal and leukemic human stem/progenitor cells. Blood (2007) vol. 110 (8) pp. 2880-2888) In contrast to STAT3 and STAT5, STAT1 negatively regulates cell proliferation and angiogenesis and thereby inhibits tumor formation. Consistent with its tumor suppressive properties, STAT1 and its downstream targets have been shown to be reduced in a variety of human tumors (Rawlings, J. The JAK/STAT signaling pathway. J of Cell Science. 2004; 117 (8):1281-1283, hereby fully incorporated by reference in its entirety for all purposes).

### DNA Damage and Apoptosis

The DNA damage response is a protective measure that allows cells to halt cell cycle progression and repair their damaged DNA before resuming passage through the cell cycle. If the DNA damage is irreparable, cells may exit the cell cycle and enter a state known as senescence or initiate an apoptotic program leading to individual cell death to preserve the genomic integrity and overall health of the entire organism. (Wade Harper et al., Molecular Cell, (2007) 28 p739-745, Bartek J et al., Oncogene (2007)26 p7773-9).

Several protein complexes within DNA damage response pathways act as sensors of DNA damage, or transducers and effectors of a DNA damage response. Depending on the nature of DNA damage for example; double stranded breaks, single strand breaks, single base alterations due to alkylation, oxidation, and the like cells activate distinct DNA response signaling pathways. For example, in response to double-strand DNA breaks a specific DNA damage sensor protein complexes assembles wherein activated ataxia telangiectasia mutated (ATM) and ATM- and Rad3 related (ATR) kinases phosphorylate and subsequently activate the checkpoint kinases Chk1 and Chk2. Both of these DNA damage activated kinases transduce and amplify the damage response by phosphorylating a multitude of downstream substrates. Chk1 and Chk2 also have overlapping and distinct roles in orchestrating the cell's response to DNA damage.

Activation of Chk2 kinase activity involves ATM mediated phosphorylation of threonine 68 and homo-dimerization (Reinhardt H C, Yaffe M B Curr Opin Cell Biol. 2009 April; 21(2):245-55, Antoni L, Sodha N, Collins I, Garrett M D Nat Rev Cancer. 2007 December; 7(12):925-36. This phosphorylation event initiates the DNA repair process of which there are at least twelve distinct mechanisms. The choice of which repair process to use depends on the type of genetic lesion and on the cell-cycle phase in which DNA damage occurs. For example, a DNA double-strand break (DSB) that occurs during the S and G2 phases is repaired by homologous recombination (Branzei and Foiani Nat Rev Mol Cell Biol. 2008 April; 9(4):297-308). If DNA repair is successful cell cycle progression may be resumed (Antoni et al., Nature reviews cancer (2007) 7, p925-936).

When DNA repair is no longer feasible, the cell may undergo apoptosis mediated by Chk2 through p53 independent and dependent pathways. Chk2 substrates that operate in a p53-independent manner include the E2F1 transcription factor, the tumor suppressor promyelocytic leukemia (PML), and the polo-like kinases 1 and 3 (PLK1 and PLK3). Once activated by Chk2, E2F1 drives the expression of a number of proapoptotic genes including caspases 3, 7, 8 and 9 as well as the pro-apoptotic Bcl-2 related proteins Bim, Noxa, and PUMA.

In its response to DNA damage, p53 activates the transcription of a many genes that regulate DNA repair, cell cycle arrest, senescence, and apoptosis. The overall function of p53 is to ensure faithful replication of the genome so that when cell division occurs genomic instability and attendant tumorigenic potential can be avoided. (Riley et al., Nature Reviews Molecular Cell Biology (2008) 9 p402-412). The diverse DNA damage and other cellular stress signals that activate p53 cause a rapid increase in p53 levels through a variety of post translational modifications. ATM, Chk1 and Chk2 phosphorylate amino acid residues within the amino terminal portion of p53. These phosphorylation events prevent MDM2 association with the p53 amino terminal domain necessary to promote 26S proteosome mediated degradation of p53. The subsequent stabilization of p53 allows p53 to transcriptionally upregulate multiple pro-apoptotic proteins as discussed below.

p53 may induce apoptosis in response to DNA damage, anoxia, and depravation of growth or other pro-survival signals. This p53-initiated apoptotic pathway may be termed the intrinsic apoptotic program as most activating stimuli originate within the cell. An alternate means of inducing apoptosis can initiate from outside the cell. This extrinsic apoptotic pathway is mediated by extracellular ligand binding to transmembrane death receptors. This extrinsic or receptor mediated apoptotic program ultimately converges with the intrinsic apoptotic program as discussed below (Sprick et al., Biochim Biophys Acta. (2004) 1644 p125-32).

Key regulators of the intrinsic apoptotic program are proteins of the Bcl-2 family. The founding member, the Bcl-2 proto-oncogene was first identified at the chromosomal breakpoint of t(14:18) associated with human follicular B cell lymphoma. Bcl-2 expression was observed to block cell death following multiple cytotoxic stimuli (Danial and Korsemeyer, Cell (2204) 116, p205-219). The Bcl-2 family has at least 20 members which are regulators of apoptosis, and most members regulate apoptosis by controlling mitochondrial permeability and downstream release of proapoptotic proteins from mitochondria.

The Bcl-2 family can be divided into 3 subclasses based on distinct structure and apoptotic function. Subclass 1 members include Bcl-2, Bcl-X_{L} and Mcl-1 are characterized by the presence of 4 Bcl-2 homology domains termed BH1, BH2, BH3 and BH4. The unique conformation conferred by the BH4 domain renders this subclass anti-apoptotic. The second subclass members Bax and Bak contain BH1-3 domains and are necessary for mitochondrial permeabilization. The third subclass, termed the BH3-only proteins includes Noxa, Puma, Bid, Bad, Bmf and Bim. BH3-only proteins promote apoptosis by directly binding and activating Bax and Bak or by inhibiting the anti-apoptotic members of subclass 1. Bax and Bak oligomerize and activate in the anti-apoptotic Bcl-2 family member inhibition. (Er et al., Biochimica et Biophysica Act (2006) 1757, p1301-1311, Fernandez-Luna Cellular Signaling (2008) Advance Publication Online).

Mitochondria play a central role in the intrinsic apoptotic program. Following a pro-death stimulus, Bax and/or Bak activate and permeabilize the outer mitochondrial membrane. Mitochondrial permeabilization serves two proapoptotic functions: cytochrome c release from the outer surface of the inner mitochondrial membrane and destruction of the mitochondrial membrane potential, an event known in the art as mitochondrial depolaraization. Mitochondrial depolarization and cytochrome c release halt cellular oxidative metabolize and help ensure that apoptotic death will be irreversible.

Once released into the cytosol, cytochrome c binds an adaptor protein known as adaptor apoptotic protease activating factor 1 (APAF1). The APAF1-cytochrome c complex oligomerizes and binds procaspase 9 to form a structure called the apoptosome. The apoptosome activates procaspase 9 by promoting proteolytic cleavage in trans of adjacent apoptosome-associated procaspase 9. Cleaved, activated caspase 9 then cleaves and activates a related family of downstream effector caspases such as caspases 3, 6, and 7. Active effector caspases cleave a host of intracellular substrates to dismantle the dying cells and package its contents into membrane bound vesicles for engulfment by surrounding cells.

The caspases are a family of cysteine aspartyl-specific proteases. Eleven caspase genes have been mapped in the human genome. As described above, both initiator and effector caspases reside in the cytosol of healthy cells as inactive zymogens. Effector caspases cleave most of the cellular substrates to produce the apoptotic phenotype and are activated by upstream initiator caspases in a rapid proteolytic cleavage cascade. One well characterized caspase substrate is poly(ADP-ribose) polymerase 1 (PARP). PARP cleavage produces two fragments both of which have apoptotic roles. (Soldani and Scovassi Apoptosis (2002) 7, p321).

A further level of apoptotic regulation is provided by Smac, a mitochondrial protein that is released from mitochondria following depolarization. Smac directly binds and inactivates a group of anti-apoptotic proteins termed inhibitors of apoptosis (IAPs) (Huang et al., Cancer Cell (2004) 5 μl-2). IAPs operate to block caspase activity in 2 ways; they bind directly to and inhibit caspase activity and in certain cases they may polyubiquitinate caspases and promote their degradation. The expression of X-linked inhibitor of apoptosis (XIAP) may be upregulated in certain human cancers.

The balance of pro- and antiapoptotic proteins is tightly regulated under normal physiological conditions. Any perturbation of this equilibrium may result in disease. An inability of tumor cells to undergo apoptosis promotes tumorigenesis. Cancer cells may become refractory to proapoptotic stimuli by over-expression or increased activity of anti-apoptotic proteins or reduced expression or activity of pro-apoptotic proteins.

Interrogation of the apoptotic machinery will also be performed with a combination of Cytarabine and Daunorubicin at clinically relevant concentrations based on peak plasma drug levels. The standard dose of Cytarabine, 100 mg/m2, yields a peak plasma concentration of approximately 40 nM, whereas high dose Cytarabine, 3 g/m2, yields a peak plasma concentration of 2 uM. Daunorubicin at 25 mg/m2 yields a peak plasma concentration of 50 ng/ml and at 50 mg/m2 yields a peak plasma concentration of 200 ng/ml. Our in vitro apoptosis assay will use concentrations of Cytarabine up to 2 uM, and concentrations of Daunorubicin up to 200 ng/ml.

### DNA Damage Response in Tumorigenesis and Cancer Treatment

The cellular DNA damage response (DDR) machinery is intimately linked with cancer as damage to DNA causes cancer. The DDR provides an intrinsic biological barrier against the development of cancer, and tumors develop when maintenance of genome integrity fails. Germline and somatic defects in the hierarchical DDR network—from sensors of diverse types of DNA lesions, damage signaling and mechanisms of checkpoint activation, to multiple DNA repair pathways—can predispose to cancer and fuel tumor progression, respectively. Recently, promising anticancer agents have emerged that target components of DNA damage signaling, the checkpoint machinery and DNA repair. Several are in preclinical development or clinical trials, either as monotherapy or to be combined with standard-of-care genotoxic therapies, to selectively target tumor cells. These developments move further towards the exciting promise of personalized therapy.

Constitutive activation of the DDR commonly occurs in premalignant and early cancerous lesions, but not in corresponding normal tissues. Among the sources of such DNA damage in nascent tumor cells is oncogene-induced DNA replication stress, telomere attrition and possibly increased levels of ROS. The resulting aberrant replication structures and DSBs activate the ATR and/or ATM-orchestrated DDR network, which provides an inducible barrier that constrains tumor progression at the early stages by inducing senescence or cell death. This causes a Darwinian struggle’ that may eventually select for genetic or epigenetic aberrations of activated DDR pathways, such as the ATM-CHK2-p53 cascade. Such a breach of this barrier would rescue the emerging malignant clones from senescence or cell death at the expense of genomic stability. Others have shown immunohistochemistry images of phosphorylated histone H2AX which indicates DDR activation, in human colorectal adenomat (a premaligant lesion) but not in normal colon.

Germline mutations in DDR genes predispose to familial cancer (such as BRCA1- or BRCA2-associated breast and ovarian tumors) and cause a range of cancer-prone genetic instability syndromes. Such mutations affect DNA damage sensors (NBS1: Nijmegen breakage syndrome), signaling kinases (ATM: ataxia-telangiectasia), effectors (p53: Li—Fraumeni syndrome) or repair (MMR: hereditary non-polyposis colorectal cancer; NER: xeroderma pigmentosum; interstrand crosslink repair: Fanconi anaemia). The impaired ability to maintain genetic stability can foster tumorigenesis, including subsequent somatically acquired genetic and epigenetic alterations in the DDR machinery that promote tumor survival and disease progression. However, such DDR defects also represent weaknesses of cancer cells that provide opportunities for cancer-selective therapeutic intervention.

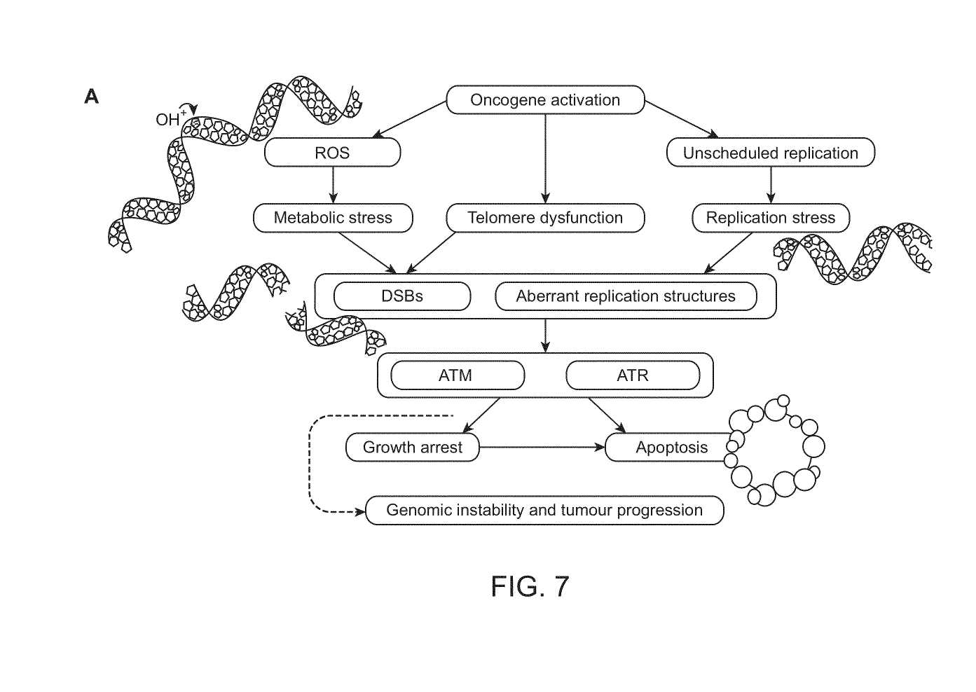

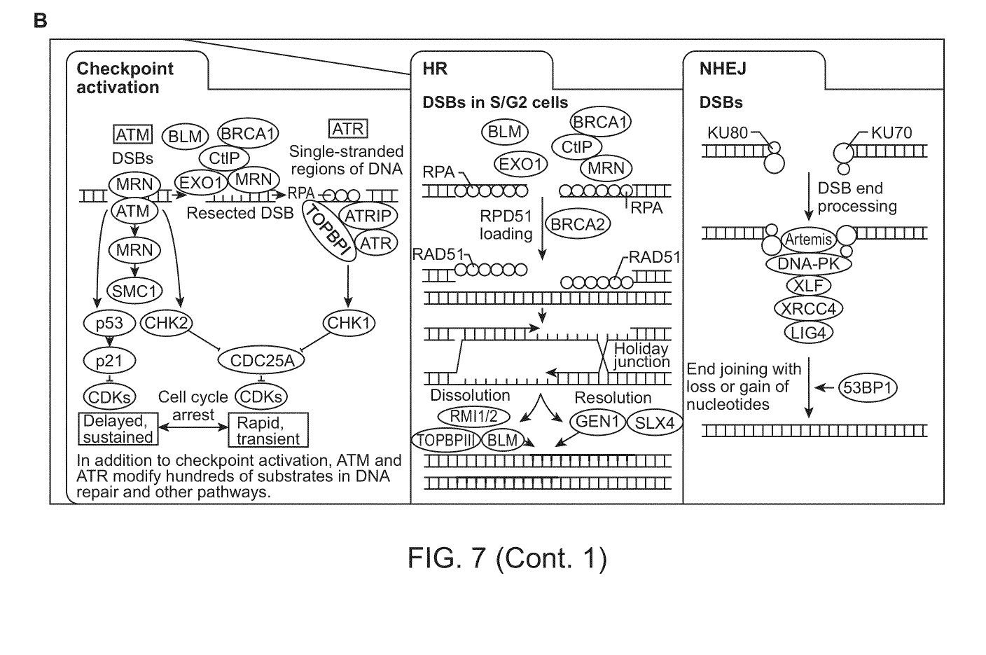

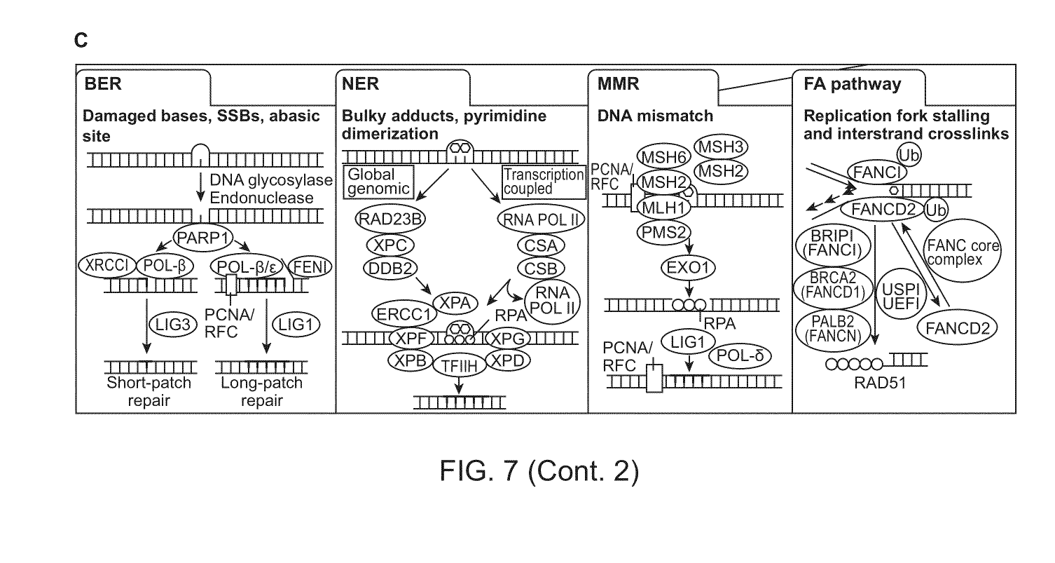

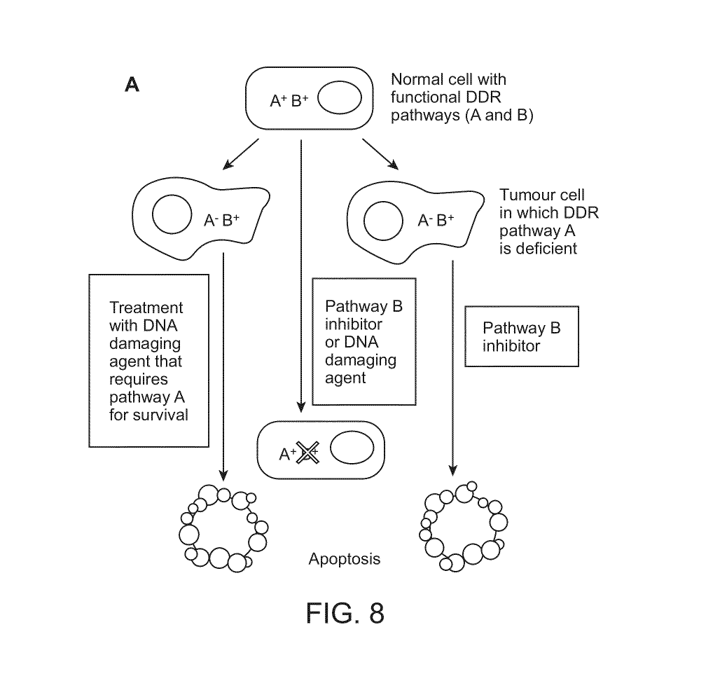

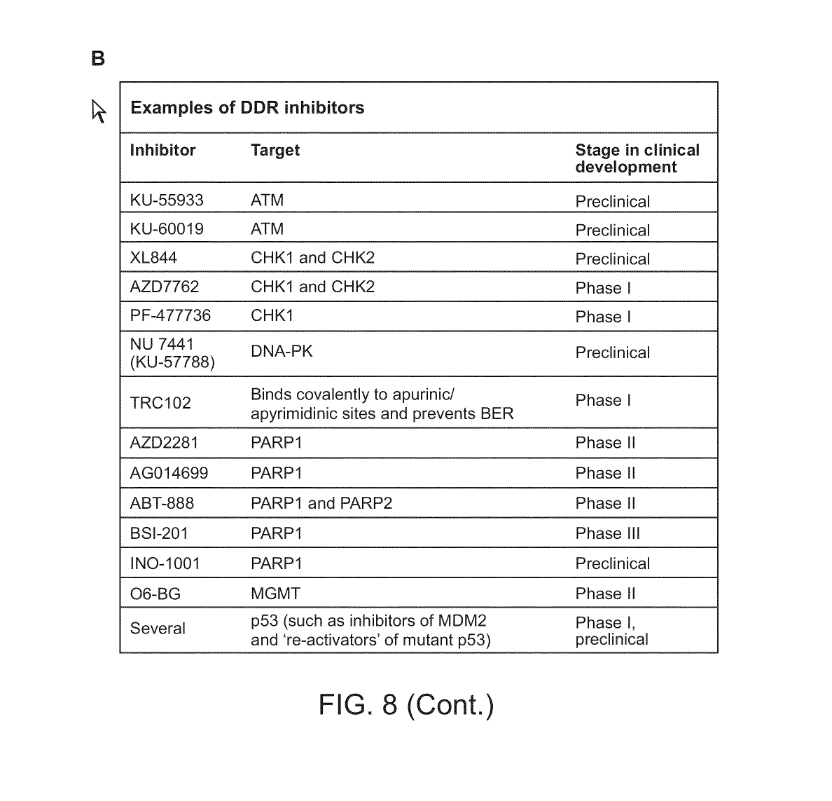

The impairment of the DDR machinery in tumors and the dependency of cancer cells on stress survival pathways (including ongoing repair of endogenous DNA damage) provides the rationale for targeting the DDR. The approach selectively targets tumor cells while sparing normal cells, which improves efficacy and reduces toxicity. The major strategy to achieve such selective tumor cell killing has been the principle of synthetic lethality: defects in either of two genes or proteins have no effect on survival but combining the two defects results in cell death (see FIGS. 7 and 8). The best example of this strategy is the PARP inhibitors, which selectively kill hereditary breast and ovarian cancers that rely on PARP for DNA break repair owing to loss-of-function mutations in BRCA1 or BRCA2. Another example is sensitization of partially checkpoint-defective cancers to radiotherapy or chemotherapy by inhibiting ATM or CHK1. DDR inhibitors show promise for treatment of diverse tumor types, both familial and sporadic, either as monotherapy or in combination to improve the efficacy of genotoxic radiotherapy and chemotherapy. Identification and validation of predictive biomarkers to select patients who would benefit most from these treatments and understanding the basis of potential resistance to such treatments are among the key goals in this rapidly evolving area of translational cancer research.

In some embodiments, the invention provides compositions and methods to measure genomic instability in cells associated with a condition and/or cells other than cells associated with a condition. In some embodiments, the genomic stability of one or more cells is determined by examining and profiling the activation level of one or more activatable elements in a DNA repair damage pathway. In some embodiments, the cells are further examined by determining and profiling the activation level of one or more activatable elements in a plurality of additional cellular pathways such as the pathways described herein, for example, signaling and cell cycle pathways. In some embodiments, the genomic stability of single cells is determined by examining and profiling the activation level of one or more activatable elements in a plurality of DNA repair damage pathways. In some embodiments, genomic instability can be measured using flow cytometry. In some embodiments, genomic instability can be measured using any suitable method known in the art to measure activation levels of activatable elements in single cells, including those described herein. Analysis of these measurements can be used as a diagnostic, prognostic, or theranostic indicator of the development or progression of disease. In some embodiments, assessment in the activation level of a carekeeper gene product in germline cells provides tools to identify subjects at high risk of tumor development and inform appropriate preventive interventions. In some embodiments, assessment in the activation level of a carekeeper gene product in somatic cells provides tool to inform therapeutic selection. In some embodiments, the methods comprise using the activation level of the activatable elements within the DNA damage repair pathways to create a response panel, wherein when the activation levels of the activatable elements are higher or lower than a predetermine threshold is indicative that a pathway is functional or not in the cell population. Correlations between the plurality of activation levels of the different activatable elements in the response panel can indicate whether a cell population from a patient favors a specific repair pathway and/or whether one or more pathways are functional or not in the cell population. The response panel can then be used, e.g., to predict outcome of a therapy or to choose a therapy or combination therapy. In some embodiments, genomic instability is measured by determining the functional consequences of genetic and epigenetic alterations (e.g., in genes and their expression) affecting proteins which are part of the DDR pathways. In some embodiments, deregulations of the DDR pathways that are caused by an alteration are measured functionally by the methods described herein. In some embodiments, deregulations of DDR pathways caused by germline mutation can be used as predisposition factor to cancer. In some embodiments, deregulations of DDR pathways caused by somatic mutations are used as an indicator of a treatment outcome, e.g., hypersensitivity to PARP inhibitors.

### Cell Cycle

The cell cycle, or cell-division cycle, is the process by which a cell duplicates its genome and synthesizes additional organelles and other cellular contents in preparation for division into two daughter cells. The cell cycle consists of five distinct phases: G0 phase, G1 phase, S (synthesis) phase, G2 phase (these four phases are collectively known as interphase) and M phase (mitosis). Two tightly coupled processes occur during M phase: mitosis, in which the cell's duplicated chromosomes are divided between two daughter cells ensuring that each daughter cell receives a full compliment of 23 chromosome pairs, and cytokinesis, in which the cell physically partitions its cytosol to form two distinct daughter cells. Activation of each cell cycle phase is sequential and dependent on the proper progression and completion of the previous phase. Cells may also temporarily or irreversibly stop dividing and exit the cell cycle. Such cells are said to have entered the state of quiescence termed G0 phase.

The cell cycle must be tightly regulated to ensure that each daughter cell receives a faithful copy of its genome. Cycling cells continuously monitor the genome to detect and repair genetic damage prior to cell division. The molecular events and signaling pathways that control cell cycle progression are ordered and directional; that is, each phase occurs in a sequential irreversible fashion.

Two key classes of regulatory molecules, cyclins and cyclin-dependent kinases (CDKs), drive cell cycle progression. Cyclin-CDK complexes phosphorylate numerous substrates to promote cell cycle progression. Cyclin forms the regulatory subunit and CDK the catalytic subunit of an active heterodimer; cyclins have no catalytic activity and CDKs are inactive in the absence of a partner cyclin. Periodic cyclin synthesis and 26S proteosome-mediated destruction activate sequential cyclin-CDK complexes that drive cell cycle progression while simultaneously ensuring that the progression is irreversible. Cell cycle phase specific cyclin-CDK complexes determine the downstream proteins targeted. Upon receiving a pro-mitotic extracellular signal, for example a growth factor binding to its cognate cell surface receptor, G1 cyclin-CDK complexes activate to prepare the cell for S phase. The G1 cyclin, cyclin D is synthesized in response to growth factor receptor stimulation. Cyclin D binds CDK4, forming the active cyclin D-CDK4 complex. This complex in turn phosphorylates, among other targets, the retinoblastoma protein (Rb). Hyperphosphorylated Rb dissociates from the E2F/DP1/Rb complex thus, activating the E2F transcription factor. E2F activation upregulates transcription of various genes required for further cell cycle progression such as the S phase cyclins cyclin E and cyclin A, as well as other molecules required for cell cycle progression such as DNA polymerases, thymidine kinase, and the like. See Chen H. Z. et al., Emerging roles of E2Fs in cancer: an exit from cell cycle control 9 Nature Rev. Cancer 785 (2009). The G1 cyclin-CDK complexes also promote the degradation of molecules that inhibit S phase entry by targeting them for ubiquitination and proteosomal degradation.

Synthesis of cyclin E and cyclin A activates S phase cyclin-CDK complexes that phosphorylate components of pre-replication complexes assembled on DNA replication origins during G1. This phosphorylation serves two purposes: to activate each preassembled pre-replication complex, and to prevent new pre-replication complexes from forming by sterically blocking replication origins. The coordinated activation of replication ensures that the cell's genome will be replicated only once replication must occur once per cell cycle to prevent aneuploidy, a condition in which cells possess aberrant numbers of whole and/or partial chromosomes. Aneuploidy disrupts expression of many genes and substantially impairs cell and organism survival. Active S phase cyclin-CDK complexes also induce synthesis of the mitotic B type cyclins to promote entry into M phase, or mitosis.

Mitotic cyclin-CDK complexes initiate mitosis by activating downstream proteins necessary for chromosome condensation and mitotic spindle assembly. One major role of the cyclinB-Cdc2 complex, the primary mitotic cyclin-CDK complex, is to activate the E3 ubiquitin ligase complex known as the anaphase-promoting complex (APC). The APC promotes mitotic entry by degrading structural proteins associated with the kinetochore, such as securin, that physically hold sister chromatid tetrad pairs together. Tetrad pairs must be separated during anaphase to ensure that each daughter cell receives the proper number of chromosomes. The APC also targets the mitotic cyclins for degradation. Mitotic cyclin degradation inactivates the cyclinB-cdc2 complex, and this inactivation is required so that telophase and cytokinesis can proceed thus completing one cell cycle.

P53 regulates cell cycle progression, in part, by inducing the expression of cyclin dependent kinase inhibitors (CDIs). The major p53 transcriptional target following DNA damage is the CDI p21. CDIs halt cell cycle progression by directly binding and inhibiting active cyclin-CDK complexes. In particular, p21 arrests the cell cycle during the G1 and S phases by inhibiting cyclinE/CDK2 and cyclinD/CDK4 complexes. Two gene families, the Cip/Kip family and the INK4a/ARF (Inhibitor of Kinase 4/Alternative Reading Frame) encode CDIs. The Cip/Kip family includes the p21 as well as the related gene products p27 and p57. Although the Cip/Kip family proteins are functionally similar, some family members have distinct modes of activation. For example, p27 is activated by Transforming Growth Factor β (TGF β), growth inhibitor pathway. The INK4a/ARF family includes p16INK4a that also binds CDK4 and arrests the cell cycle in the G1 phase. The related protein p14arf is transcriptionally upregulated in response to various forms of cellular stress and prevents p53 degradation by directly binding MDM2 and abrogating MDM2-mediated p53 proteosomal degradation.

DAPI (4′,6-Diamidino-2-phenylindole) is a blue fluorescent probe that fluoresces brightly when it is selectively bound to the minor groove of double stranded DNA where its fluorescence is approximately 20-fold greater than in the non-bound state. DAPI has an excitation maximum at 345 nm and an emission maximum at 455 nm. Cells stained with DAPI emit fluorescence in direct proportion to their DNA content. An exponentially growing population of cells will have a DNA content distribution containing an initial peak of G0/G1 cells, a valley of S Phase cells, and a second peak containing G2/M cells. Cells in the G2/M Phase have twice the DNA content as cells in the G0/G1 Phase. DAPI offers a rapid method for measuring the DNA content of cells and provides a convenient research tool to monitor cell cycle status and regulation.

In some embodiments, the kits of the present invention comprise one or binding elements to measure one or more activatable elements within a cell cycle pathway in response to a modulator that slows or stops the growth of cells and/or induces apoptosis of cells. In some embodiments, the kits further comprise the modulator that slows or stops the growth of cells and/or induces apoptosis of cells. In some embodiments, the activatable element is selected from the group consisting of, Cdk1, Cyclin B1, Histone H3, Cyclin D1, p15, p16, and p21. In some embodiments, the modulator that slows or arrests cell cycle progression, and/or induces apoptosis of cells is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, and Zolinza.

### Binding Element

In some embodiments of the invention, the activation level of an activatable element is determined. One embodiment makes this determination by contacting a cell from a cell population with a binding element that is specific for an activation state of the activatable element. The term “Binding element” includes any molecule, e.g., peptide, nucleic acid, small organic molecule which is capable of detecting an activation state of an activatable element over another activation state of the activatable element. Binding elements and labels for binding elements are shown in U.S. Ser. No. /048,886; 61/048,920 and 61/048,657.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

Methods of the present invention may be used to detect any particular activatable element in a sample that is antigenically detectable and antigenically distinguishable from other activatable elements which are present in the sample. For example, the activation state-specific antibodies of the present invention can be used in the present methods to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody.

The term “antibody” includes full length antibodies and antibody fragments, and may refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 61/048,886; 61/048,920 and 61/048,657 for more information about antibodies as binding elements.

Activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

The antigenicity of an activated isoform of an activatable element is distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pinl prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

In alternative embodiments of the instant invention, aromatic amino acids of protein binding elements may be replaced with other molecules. See U.S. Ser. Nos. 61/048,886; 61/048,920 and 61/048,657.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods of the present invention.

A variety of recognition structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In a some embodiment, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used in the present invention.

In some embodiments the binding element is a carbohydrate. As used herein the term carbohydrate is meant to include any compound with the general formula (CH₂0)ₙ. Examples of carbohydrates are di-, tri- and oligosaccharides, as well polysaccharides such as glycogen, cellulose, and starches.

In some embodiments the binding element is a lipid. As used herein the term lipid herein is meant to include any water insoluble organic molecule that is soluble in nonpolar organic solvents. Examples of lipids are steroids, such as cholesterol, and phospholipids such as sphingomeylin.

Examples of activatable elements, activation states and methods of determining the activation level of activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

### Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. Binding elements and labels for binding elements are shown in U.S. Ser. No. /048,886; 61/048,920 and 61/048,657.

A compound can be directly or indirectly conjugated to a label which provides a detectable signal, e.g. radioisotopes, fluorescers, enzymes, antibodies, particles such as magnetic particles, chemiluminescers, molecules that can be detected by mass spec, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments of the invention, these labels may be conjugated to the binding elements.

In some embodiments, one or more binding elements are uniquely labeled. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Labels include optical labels such as fluorescent dyes or moieties. Fluorophores can be either “small molecule” fluors, or proteinaceous fluors (e.g. green fluorescent proteins and all variants thereof).

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome-conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195.

Alternatively, detection systems based on FRET, discussed in detail below, may be used. FRET finds use in the instant invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

The methods and composition of the present invention may also make use of label enzymes. By label enzyme is meant an enzyme that may be reacted in the presence of a label enzyme substrate that produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes for use in the invention include, but are not limited to ¹⁴C, ³H, ³²P, ³³P, ³⁵S, ¹²⁵I and ¹³¹I. The use of radioisotopes as labels is well known in the art.

As mentioned, labels may be indirectly detected, that is, the tag is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide (Hopp et al., BioTechnology, 6:1204-1210 (1988)); the KT3 epitope peptide (Martin et al., Science, 255: 192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)) and the antibodies each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag that is a partner of a binding pair, as just described, is referred to herein as “indirect labeling”.

By “surface substrate binding molecule” or “attachment tag” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate (Field et al., Mol. Cell. Biol., 8:2159-2165 (1988)); the c-myc tag and the 8F9, 3C7, 6E10, G4, B7 and 9E10 antibody substrates thereto (Evan et al., Molecular and Cellular Biology, 5:3610-3616 (1985)); and the Herpes Simplex virus glycoprotein D (gD) tag and its antibody substrate (Paborsky et al., Protein Engineering, 3(6):547-553 (1990)). In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

### Alternative Activation State Indicators

An alternative activation state indicator useful with the instant invention is one that allows for the detection of activation by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allowing for a detectable signal being produced from a previously quenched label.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detect and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See U.S. patent Ser. No. 10/898,734 and Shulz et al., Current Protocols in Immunology, 2007, 78:8.17.1-20 which are incorporated by reference in their entireties.

In some embodiments, the present invention provides methods for determining the activation level on an activatable element for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

When using fluorescent labeled components in the methods and compositions of the present invention, it will recognize that different types of fluorescent monitoring systems, e.g., Cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells that may used as a modulator or as a population of reference cells. In some embodiments, the modulator or reference cells are first contacted with fluorescent-labeled binding elements (e.g. antibodies) directed against specific elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least one activatable element. In some embodiments, cells are analyzed on the basis of the activation level of at least two activatable elements.

In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

As will be appreciated, these methods provide for the identification of distinct signaling cascades for both artificial and stimulatory conditions in cell populations, such a peripheral blood mononuclear cells, or naive and memory lymphocytes.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3):188-195.).

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acids on a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell. See U.S. Pat. No. 5,744,934.

In some embodiments confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods of the invention include the use of liquid handling components. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems. See U.S. Ser. No. 61/048,657 which is incorporated by reference in its entirety.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader. See U.S. Ser. No. 61/048,657.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory. See U.S. Ser. No. 61/048,657 which is incorporated by reference in its entirety.

These robotic fluid handling systems can utilize any number of different reagents, including buffers, reagents, samples, washes, assay components such as label probes, etc.

Any of the steps above can be performed by a computer program product that comprises a computer executable logic that is recorded on a computer readable medium. For example, the computer program can execute some or all of the following functions: (i) exposing different population of cells to one or more modulators, (ii) exposing different population of cells to one or more binding elements, (iii) detecting the activation levels of one or more activatable elements, and (iv) making a diagnosis or prognosis based on the activation level of one or more activatable elements in the different populations.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of determining the status of an individual by accessing data that reflects the activation level of one or more activatable elements in the reference population of cells.

### Gating and Analysis

In some embodiments of the invention, different gating strategies can be used in order to analyze a specific cell population (e.g., only blasts) in a sample of mixed population after treatment with the modulator. These gating strategies can be based on the presence of one or more specific surface markers expressed on each cell type. In some embodiments, the first gate eliminates cell doublets so that the user can analyze singlets. The following gate can differentiate between dead cells and live cells and the subsequent gating of live cells classifies them into, e.g. myeloid blasts, monocytes and lymphocytes. A clear comparison can be carried out to study the effect of potential modulators, such as G-CSF on activatable elements in: ungated samples, myeloid blasts, monocytes, granulocytes, lymphocytes, and/or other cell types by using two-dimensional contour plot representations, two-dimensional dot plot representations, and/or histograms. For example, a comparison can be carried out to study the effect of a modulator of the Jak/Stat signaling pathway in different cell populations within a patient sample by using two-dimensional contour plot representations of Stat5 and Stat3 phosphorylation (downstream intracellular readouts for Jak kinases) (X and Y axis). The level of basal phosphorylation and the change in phosphorylation in both Stat3 and Stat5 in response to a modulator such as G-CSF can be compared. G-CSF mediates increases in both Stat3 and Stat5 phosphorylation and this signaling can occur concurrently (subpopulations with increases in both p-Stat 3 and p-Stat5) or individually (subpopulations with either an increase in p-Stat3 or pStat5 alone). The advantage of gating is to get a clearer picture and more precise results of the effect of various activatable elements on a specific cell sub-population such as blasts within a complex human sample.

In some embodiments, the present invention provides methods for classification, diagnosis, prognosis of a condition and/or prediction of outcome after administering a therapeutic agent to treat the condition by determining the activation levels of one or more activatable elements in a population of cells. The characterization of one or more pathways is performed by contacting a cell population with one or more modulators and determining the activation level of an activatable element of at least one cell in the cell population. The data can be analyzed using various metrics. Examples of metrics include: 1) measuring the difference in the log of the median fluorescence value between an unstimulated fluorochrome-antibody stained sample and a sample that has not been treated with a stimulant or stained (log (MFI_{Unstimulated Stained})−log (MFI_{Gated Unstained})), 2) measuring the difference in the log of the median fluorescence value between a stimulated fluorochrome-antibody stained sample and a sample that has not been treated with a stimulant or stained (log (MFI_{Stimulated Stained})−log (MFI_{Gated Unstained})), 3) Measuring the change between the stimulated fluorochrome-antibody stained sample and the unstimulated fluorochrome-antibody stained sample log (MFI_{stimulated Stained})−log (MFI_{Unstimulated Stained}), also called “fold change in median fluorescence intensity”, 4) Measuring the percentage of cells in a Quadrant Gate of a contour plot which measures multiple populations in one or more dimension 5) measuring WI of phosphor positive population to obtain percentage positivity above the background; and 6) use of multimodality and spread metrics for large sample population and for subpopulation analysis. Other possible metrics include third-color analysis (3D plots); percentage positive and relative expression of various markers; clinical analysis on an individual patient basis for various parameters, including, but not limited to age, race, cytogenetics, mutational status, blast percentage, CD34+ percentage, time of relapse, survival, etc. In alternative embodiments, there are other ways of analyzing data, such as third color analysis (3D plots), which can be similar to Cytobank 2D, plus third D in color. In another embodiment, a user may analyze the signaling in subpopulations based on surface markers. For example, the user could look at: “stem cell populations” by CD34+ CD38− or CD34+ CD33− expressing cells; or a subset of PMBCs expressing a genetic alteration and analyzing signaling in each subpopulation. In another alternative embodiment, a user may analyze the data based on intracellular markers, such as transcription factors or other intracellular proteins, based on a functional assay, or based on other fluorescent markers.

In some embodiments where flow cytometry is used, prior to analyzing of data the populations of interest and the method for characterizing these populations are determined. For instance, there are at least two general ways of identifying populations for data analysis: (i) “Outside-in” comparison of Parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific populations prior to classification. A possible drawback of this approach is that it creates populations which, at least initially, may require multiple transient markers to enumerate and may never be accessible with a single cell surface epitope. As a result, the biological significance of such populations can be difficult to determine. One advantage of this unconventional approach is the unbiased tracking of cell populations without drawing potentially arbitrary distinctions between lineages or cell types.

Each of these techniques capitalizes on the ability of flow cytometry to deliver large amounts of multiparameter data at the single cell level. For cells associated with a condition (e.g. neoplastic or hematopoetic condition), a third “meta-level” of data exists because cells associated with a condition (e.g. cancer cells) are generally treated as a single entity and classified according to historical techniques. These techniques have included organ or tissue of origin, degree of differentiation, proliferation index, metastatic spread, and genetic or metabolic data regarding the patient.

In some embodiments, the present invention uses variance mapping techniques for mapping condition signaling space. These methods represent a significant advance in the study of condition biology because it enables comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and reclusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g. two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the X² test. Similarly, if two variable factors within the experiment are thought to be related, the r² correlation coefficient from a linear regression is used to represent the degree of this relationship.

Examples of analysis for activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology February 2008. Methods for the analysis of multiple parameters are well known in the art. See U.S. Ser. No. 61/079,579 for gating analysis.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Ser. No. 61/079,537 for visualization tools.

The patients are stratified based on nodes that inform the clinical question using a variety of metrics. To stratify the patients a prioritization of the nodes can be made according to statistical significance (such as p-value or area under the curve) or their biological relevance.

### Methods

In some embodiments, the invention is directed to methods that allow for the determination of a physiological status of a cell. In some embodiments, the physiological status of a cell is determined by measuring DNA repair levels and/or a DNA damage response (DDR) in cells containing a genetic or epigenetic alteration that may be derived from a germline or somatic mutation. Thus, in some embodiments, determining the physiological status of a cell involves determining functionally DNA repair levels, DDR pathway deficiencies, and/or apoptotic pathway deficiencies in the cell. The methods described herein are suitable for any condition for which a correlation between the physiological status of a cell and the determination of a disease predisposition, diagnosis, prognosis, and/or course of treatment in samples from individuals may be ascertained. In some embodiments, this invention is directed to methods for analysis, drug screening, diagnosis, prognosis, and for methods of disease treatment and prediction. In some embodiments, the present invention involves methods of analyzing experimental data. In some embodiments, the physiological status of a cell population comprising a genetic alteration is used, e.g., in diagnosis or prognosis of a condition, patient selection for therapy, e.g., using some of the agents identified herein, to monitor treatment, modify therapeutic regimens, and/or to further optimize the selection of therapeutic agents which may be administered as one or a combination of agents. In some embodiments, the cell population is not associated and/or is not causative of the condition. In some embodiments, the cell population is associated with the condition but it has not yet developed the condition. The physiological status of a cell population can be determining by determining the activation level of at least one activatable element in response to at least one modulator in one or more cells belonging to the cell population.

The methods of the invention provide tools useful in the prevention of disease such cancer by identifying predispositions on which we can medically intervene, treatment of an individual afflicted with a condition, including but not limited to methods for assigning a risk group, methods of predicting an increased risk of relapse, methods of predicting an increased risk of developing secondary complications, methods of choosing a therapy for an individual, methods of predicting duration of response, response to a therapy for an individual, methods of determining the efficacy of a therapy in an individual, and methods of determining the prognosis for an individual. The physiological state of a cell population can serve as a prognostic indicator to predict the course of a condition, e.g. whether a person will develop a certain tumor or other pathologic conditions, the course of a neoplastic or a hematopoietic condition in an individual will be aggressive or indolent, thereby aiding the clinician in managing the patient and evaluating the modality of treatment to be used. In another embodiment, the present invention provides information to a physician to aid in the clinical management of a patient so that the information may be translated into action, including treatment, prognosis or prediction.

In some embodiments, the methods described herein are used to screen candidate compounds useful in the treatment of a condition or to identify new druggable targets.

In still another embodiment, the physiological status of cell population can be used to confirm or refute a diagnosis of a pre-pathological or pathological condition.

In instances where an individual has a known pre-pathologic or pathologic condition, the physiological status of cell population can be used to predict the response of the individual to available treatment options. In one embodiment, an individual treated with the intent to reduce in number or ablate cells that are causative or associated with a pre-pathological or pathological condition can be monitored to assess the decrease in such cells and the state of a cellular network over time. A reduction in causative or associated cells may or may not be associated with the disappearance or lessening of disease symptoms. If the anticipated decrease in cell number and/or improvement in the state of a cellular network do not occur, further treatment with the same or a different treatment regiment may be warranted.

In one embodiment, at least one cellular pathway abnormality that may characterize and underlie a known condition may be profiled in a first sample and compared to at least one cellular pathway abnormality associated with at least one known genetic alteration in a second sample. For example, DDR and/or apoptosis pathways may be interrogated and profiled as described herein using samples derived from patients known to carry BRCA1/2 mutations and samples derived from patients whose BRCA 1 and 2 genes are known to lack any genetic alterations. The patient samples known to carry BRCA1/2 mutations and the patient samples known to be free of BRCA 1/2 mutations may be further subdivided into two additional populations: samples derived from patients with a documented history of triple negative breast or ovarian cancer and samples derived from patients having no documented history of any form of breast or ovarian cancer. The DDR and/or apoptosis pathways from four distinct groups of patient samples may be interrogated and profiled. The DDR and/or apoptosis pathways from any group of patient samples may be determined in the presence or the absence of a modulator as described herein. Comparisons of the DDR and/or apoptosis pathways among all groups or combinations of groups of patient samples may be used to predict individual at high risk of developing breast cancer/ovarian cancers, a therapeutic response to any drug or other treatment, diagnose any condition, or screen candidate compounds useful in the treatment of a condition or to identify new druggable targets

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another cell population(s) that are associated with a good prognosis. If a beneficial, population of cells is observed, measures can be taken to further increase their numbers, such as the administration of growth factors. Alternatively, individuals may be monitored for the appearance or increase in cell number of another cells population(s) associated with a poor prognosis. In such a situation, renewed therapy can be considered including continuing, modifying the present therapy or initiating another type of therapy.

In some embodiment, the characterization of multiple DNA damage repair pathways are used to predict an individual at high risk of developing a condition (e.g., breast cancer/ovarian cancers), a therapeutic response to any drug or other treatment, diagnose any condition, or screen candidate compounds useful in the treatment of a condition or to identify new druggable targets. In some embodiments, the invention provides methods of classification, diagnosis, prognosis and/or prediction of an outcome of a condition in an individual by: a) contacting a cell population from the individual with a DNA damage or apoptosis inducing agent, wherein the cell population comprises a genetic and/or epigenetic alteration, wherein the alteration is associated with the development of the condition; b) characterizing a plurality of DNA damage repair pathways in one or more cells from the cell population by determining an activation level of at least one activatable element within the plurality of DNA damage repair pathways; c) determining whether the plurality of DNA damage pathways are functional in the individual based on the activation levels of the activatable elements; and d) making a decision regarding the classification, diagnosis, prognosis and/or prediction of an outcome of the condition in the individual, where the decision is based on the determination on step (c). In some embodiments, the methods further comprise a correlation between the activation levels of the activatable elements within the plurality of DNA damage repair pathways. Correlations between DDR nodes can indicate whether a cell population from a patient favors a specific repair pathway and/or whether one or more pathways are functional or not in the cell population. This information can be further correlated with apoptosis induced by the DNA damage or apoptosis inducing agent on the cell population. These correlations can be used, e.g., to predict outcome of a therapy or to choose a therapy or combination therapy. In some embodiments, the methods comprise using the activation level of the activatable elements within the plurality of DNA damage repair pathways to create a response panel, wherein when the activation levels of the activatable elements are higher or lower than a predetermine threshold is indicative that a pathway is functional or not in the cell population. Correlations between the plurality of activation levels of the different activatable elements in the response panel can indicate whether a cell population from a patient favors a specific repair pathway and/or whether one or more pathways are functional or not in the cell population. The response panel can then be used, e.g., to predict outcome of a therapy or to choose a therapy or combination therapy.

In some embodiments, the physiological status of a cell comprising a genetic alteration is determined by contacting the cell with a DNA damage or apoptosis inducing agent and determining an activation level of at least one activatable element within a DNA damage pathway, an apoptosis pathway, and/or a cell cycle pathway. In some embodiments a plurality of activatable elements is determined. The plurality of activatable elements can be within a single pathway or can be members of different pathways. In some embodiments, the physiological status of a cell is used for the classification, diagnosis, prognosis and/or prediction of an outcome of a condition in an individual.

In some embodiments, the characterization of a DNA damage pathway and/or apoptosis pathway is performed in cycling cells. Examples of cycling cells and how to measure activation on cycling cells can be found in U.S. application Nos. 61/423,918 and 12/713,165, incorporated herein by references in their entirety.

The invention provides methods of classification, diagnosis, prognosis and/or prediction of an outcome of a condition in an individual. In some embodiments, the methods comprise the steps of: a) contacting a cell population from a individual with a DNA damage or apoptosis inducing agent, where the cell population comprises a genetic alteration, and where the cell population is not associated and/or is not causative of the condition; b) determining an activation level of at least one activatable element within a DNA damage pathway, an apoptosis pathway, and/or a cell cycle pathway in one or more cells from the cell population; and c) making a decision regarding classification, diagnosis, prognosis and/or prediction of an outcome of the condition in the individual, where the decision is based on the activation levels of the at least one activatable element within the DNA damage pathway, an apoptosis pathway, and/or a cell cycle pathway.

In some embodiments, the invention provides methods of determine a signaling phenotype of a cell population, where the cell population comprises a genetic alteration. In some embodiments, the invention provides methods to determine a signaling phenotype of a cell population, where the cell population comprises no known genetic alteration and is not known to be associated with a condition. In other embodiments, the invention provides methods to determine a signaling phenotype of a cell population, where the cell population is derived from an individual having a documented history of a condition. In yet other embodiments, the invention provides methods to determine a signaling phenotype of a cell population, where the cell population comprises at least one known genetic alteration. In still other embodiments, the invention provides methods to determine a signaling phenotype of a cell population, where the cell population comprises at least one known genetic alteration and a documented history of a condition. In some embodiments, the methods comprise the steps of: a) subjecting any cell population described above to a plurality of modulators in separate cultures; b) characterizing at least one pathway in the cell population from a separate plurality of cultures by determining an activation level of at least one activatable element within the at least one pathway; c) creating a response panel comprising the characterization of the at least one pathway from the separate cultures; and d) determining a signaling phenotype, where the signaling phenotype is based on the response panel.

In any of the embodiments described herein the activation level of a plurality of activatable elements can be determined (sequentially or simultaneously) in response to one or more modulators.

In some embodiments, the genetic alteration is a germ line alteration. Examples of genetic alterations include, but are not limited to, alterations in APC, AXIN2, ARF, ATM, BLM, CDH1, GPC3, CYLD, EXT1, EXT2, PTCH, SUFU, FH, SDHB, SDHC, SDHD, VHL, TP53, WT1, STK11, PTEN, TSC1, TSC2, CDKN2A, CDK4, RB1, RAD50, NF1, BMPR1A, MEN1, SMAD4, BHD, HRPT2, NF2, MUTYH, ATM, BLM, BRCA1, BRCA2, FANCA, FANCC, FANCD2, FANCE, FANCF, FANCG, NBS1, RECQL4, WRN, MSH2, MLH1, MSH6, MDM2, MRE11, NBS1, RAS, RHO, RAN, RAB, PMS2, p53, XPA, XPC, ERCC2, ERCC3, ERCC4, ERCC5, DDB2, KIT, MET, PDGFRA, RET, and DNA replication factor C. In some embodiments, the genetic alteration is an alteration in a gene from Table 1.

Examples of DNA damage or apoptosis inducing agent include, but are not limited to, Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, temozolomide, teniposide, Thioguanine, topotecan, troxacitabine, Abraxane, Adriamycin, carboplatin, Cytoxan, Doxil, Ellence, fluorouracil, Gemzar, Ixempra, methotrexate, Mitomycin, mitoxantrone, Navelbine, Taxol, Taxotere, thiotepa, vincristine, Xeloda, Herceptin, Tykerb, Avastin, mitotic inhibitors, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Examples of activatable elements within a DNA Damage pathway include, but are not limited to, p-Chk1, p-Chk2, p-53, p-ATM, p-ATR, p-21, and p-H2AX. Examples of activatable elements within an apoptosis pathway include but are not limited to Cleaved PARP, Cleaved Caspase 3, Cleaved Caspase 8, BAX, Bak, Puma, Noxa, Bad, Bim, Bmf, and Cytochrome C. Examples of activatable elements within a cell cycle pathway include, but are not limited to Cdc25, p53, cCdk1, CyclinB1, Cyclin E, Cyclin A, CDK4, p16, p21, p-Histone H3 and Gadd45.

In some embodiments, the invention provides methods of classification, diagnosis, prognosis and/or prediction of an outcome of a condition in an individual by contacting a cell population comprising a genetic alteration from an individual with a DNA damage or apoptosis inducing agent and one or more additional modulators.

In some embodiments, the invention provides methods of determining a signaling phenotype of a cell population, wherein the cell population comprises a genetic alteration, by contacting a cell population comprising a genetic alteration from an individual with a DNA damage or apoptosis inducing agent and one or more additional modulators. In some embodiments, the signaling phenotype is then used for classification, diagnosis, prognosis and/or prediction of an outcome of a condition in an individual.

In any of the embodiments described herein, the methods provide for the determination of an activatable element within a pathway selected from the group consisting of drug conversion into an active agent, internal cellular pH, redox potential environment, phosphorylation state of ITIM; drug activation; and signaling pathways.

In any of the embodiments described herein, the methods provide for the determination of an activatable element within a pathway selected from the group consisting of Jak/Stat, PI3K/Akt, and MAPK pathways. In some embodiments, the activation levels of p-Akt, p-ERK, p-SyK, p38 and pS6 are determined in response to FLT3L, SCF, G-CSF, GM-CSF, SCF, SDF1a, LPS, PMA, Thapsigargin, and/or a combination thereof. In some embodiments, the activation levels of p-Stat3, p-Stat5, p-Stat1, and p-Stat6 is determined in response to IFNg, IFNa, IL-27, IL-3, IL-6, IL-10, GM-CSF and G-CSF.

In any of the embodiments described herein, the presence or absence of one or more cell surface markers, intracellular markers, or combination thereof is determined. Examples cell surface markers and intracellular markers include but are not limited to proteins, carbohydrates, lipids, nucleic acids and metabolites. In some embodiments, determining of the presence or absence of one or more cell surface markers or intracellular markers comprises determining the presence or absence of an epitope in both activated and non-activated forms of the cell surface markers or the intracellular markers. In any of the embodiments described herein, the classification, diagnosis, prognosis and/or prediction of outcome of the condition in an individual is based on both the activation levels of one or more activatable elements and the presence or absence of the one or more cell surface markers, intracellular markers, or combination thereof.

The invention provides methods for detecting p53 levels in a cell population. In some embodiments, the methods comprise the steps of a) subjecting the cell population to a modulator; b) contacting the cell population with a binding element specific for p53; and c) using flow cytometry to detect presence or absence of binding of the binding element to p53, where the presence or absence of binding of the binding element is indicative of the p53 levels in the population. In some embodiments, the binding element comprises an antibody, recombinant protein, or fluorescent dye. In some embodiments, the methods further comprise contacting the cell population with a second binding element specific for a different epitope of p53. In some embodiments, p53 is a mutated p53. In some embodiments, p53 is wild type p35. In some embodiments, the cell population is associated and/or causative of a condition. In some embodiments, the cell population is not associated and/or is not causative of a condition.

The invention provides methods for detecting apoptosis in a cell population. In some embodiments, the methods comprise the steps of a) subjecting the cell population to a cytotoxic agent or any other modulator; b) contacting the cell population with a binding element specific for cleaved caspase 3, cleaved caspase 7, cleaved caspase 8, cleaved PARP, cytochrome c, tBid, Puma, Noxa, Bad, phospho-Bad, or and combination of the preceding; and c) using flow cytometry to detect the presence or absence of binding of the binding element to polypeptide listed in b), where the presence or absence of binding of the binding element is indicative of the polypeptide levels in the population. In some embodiments, the binding element comprises an antibody, recombinant protein, or fluorescent dye. In some embodiments, the cell population is associated and/or causative of a condition. In some embodiments, the cell population is not associated and/or is not causative of a condition. In some embodiments the binding element is a nonspecific vital dye, for example Aqua Blue.

In some embodiments the modulator is a DNA damage or apoptosis inducing agent. In some embodiments the modulator is a cytotoxic agent whose mechanism of action is other than inducing DNA damage. Examples of DNA damage or apoptosis inducing agent include, but are not limited to, Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, Hydroxyurea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, temozolomide, teniposide, Thioguanine, topotecan, troxacitabine, Abraxane, Adriamycin, carboplatin, Cytoxan, Doxil, Ellence, fluorouracil, Gemzar, Ixempra, methotrexate, dexamethosone, Mitomycin, mitoxantrone, Navelbine, Taxol, Taxotere, thiotepa, vincristine, Xeloda, Herceptin, Tykerb, Avastin, mitotic inhibitors, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

In some embodiments, the levels of p53 are used in classifying, diagnosing, prognosing and/or predicting an outcome of a condition in an individual.

In some embodiments the levels of apoptotic or nonapoptotic cell death within a cell population induced by a modulator are used in classifying, diagnosing, prognosing and/or predicting an outcome of a condition in an individual.

In some embodiments the DDR profile is used in classifying, diagnosing, prognosing and/or predicting an outcome of a condition in an individual.

### Pathway Profiling

In some embodiments, the invention provides methods for measuring activity at multiple steps in a signaling pathway. For cells comprising genetic alterations with aberrant signaling activity, these methods may be used to determine the step or steps of the pathway at which signaling is disrupted. Identification of the disrupted steps may enable the selection of targeted therapeutics, as well as enable diagnosis, prognosis or outcome prediction in a condition. For example, the methods of the invention can distinguish between DNA damage-dependent cell cycle arrest and DNA damage-independent cell cycle arrest, and further can identify the stage of cell cycle arrest.

In response to double-stranded DNA breaks, the ataxia telangiectasia mutated (ATM) kinase is activated through autophosphorylation, and induces cell cycle arrest by acting on multiple targets (for review, see Riches, L. C., et al. Early events in the mammalian response to DNA double-strand breaks. Mutagenesis. (2008) 23:331-9). ATM is one of several kinases known to directly phosphorylate the histone variant H2AX, which nucleates a DNA damage response complex. ATM also phosphorylates Chk2 and p53. The Chk2 checkpoint kinase is central to transducing the DNA damage signal. P53 regulates both cell cycle arrest and apoptosis by transcriptionally upregulating the CDK inhibitor p21 and the proapoptotic proteins Puma and NOXA. P53 itself can directly induce apoptosis by binding to the mitochondria and triggering cytochrome c release.

In response to many forms of DNA damage, the BRCA1 and BRCA2 proteins are activated. BRCA 1 and BRCA2 are necessary for Rad 51 mediated homologous recombination. BRCA1 plays roles in the repair of many forms of DNA damage including double strand breaks, replication fork stalls, and aberrant DNA structures such as crosslinks caused by UV radiation and many forms of chemotherapy such as cisplatin. During the DDR, BRCA1 may become phosphorylated at serine 1524.

Cyclin B1 is produced during the G2 phase of the cell cycle and its accumulation drives the cell cycle into M phase. Therefore, low levels of Cyclin B1 mark G1 and S phases, high levels mark G2 phase, and higher levels mark M phase. Histone H3 (S28) becomes phosphorylated in M phase, making p-Histone H3(S28) a useful marker of cells in M phase and not G2. It is also possible to monitor the G2/M phases of the cell cycle by measuring the phosphorylation status of Cdk1, previously known as Cdc2, a cyclin-dependent protein kinase that controls the cell cycle entry from G2 to M phase. An inhibitory phosphorylation on Cdk1 is removed by CDC25C in the M phase transition, allowing G2 cells to be distinguished from M cells based on levels of p-Cdk1.

Treating a cell comprising a genetic alteration with a modulator that produces DNA damage and/or induces cell cycle arrest can be used to detect deficiencies in the DNA repair activity of the cell, even if the cell is not associated and/or causative of a condition. Multiparameter flow cytometry can be used to measure the levels of activated DNA damage response elements, p-H2AX(S139), p-p53(S15), p-Chk2(T68), p-ATR(S428), p-BRCA1(S1524), and p-ATM(S1981) and markers of cell cycle arrest, p-Cdk1(G2 phase), Cyclin B1 (G2-M), p-H3 (M) in single cells in response to modulators that produce DNA damage and/or induces cell cycle arrest and/or promotes cell death or apoptosis. Thus, the methods of the invention can be used to measure DNA repair levels in cell comprising a genetic alteration and, therefore, detect DNA repair deficiencies that might predispose patients to a condition (e.g. cancer) and/or predict a response to a therapy (e.g. cytotoxic therapies or targeted therapies). In some embodiments, the methods of the invention may be used to identify the specific step in a signaling pathway at which signaling is disrupted, for example in a disease, or by treatment with a modulator. In other embodiments, the methods of the invention may be used to identify the effects of modulator treatment on specific steps in a signaling pathway, including, but not limited to pathways disrupted in disease. In some embodiments, the signaling activity of different DNA damage repair pathways is used to of creating a response panel, wherein when the signaling activity of one activatable elements in a DNA damage repair pathway is higher or lower than a predetermine threshold is indicative that a pathway is functional or not in the cell population. Correlations between the plurality of signaling activity of the different activatable elements in the response panel can indicate whether a cell population from a patient favors a specific repair pathway and/or whether one or more pathways are functional or not in the cell population. The response panel can then be used, e.g., to predict outcome of a therapy or to choose a therapy or combination therapy. In some embodiments, the signaling activity of different DNA damage repair pathways is determined in single cells by suitable methods known in the art including those described herein.

### Conditions

The methods of the invention are applicable to any condition in an individual involving, indicated by, and/or arising from, in whole or in part, by altered physiological status in cells. The term “physiological status” includes mechanical, physical, and biochemical functions in a cell. In some embodiments, the physiological status of a cell is determined by measuring characteristics of at least one cellular component of a cellular pathway in cells. Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pgs. 232-253; Weinberg, Chapter 6, The biology of Cancer, 2007; and Blume-Jensen and Hunter, Nature, vol 411, 17 May 2001, p 355-365). In some embodiments, the physiological status of a cell is determined by measuring DNA repair levels in cells containing a genetic alteration. The genetic alteration may be a mutation in the BRCA1 and/or BRCA2 genes. Thus, in some embodiments, determining the physiological status of a cell involves determining DNA repair deficiencies in the cell. A condition involving or characterized by altered physiological status may be readily identified, for example, by determining the state of one or more activatable elements in cells from one or more populations, as taught herein. A condition involving or characterized by altered physiological status may be readily identified, for example, by determining the state of one or more activatable elements in cells from one or more populations, wherein the one or more cell populations are or are not associated or causative of the condition.

In certain embodiments of the invention, the condition is a neoplastic, immunologic or hematopoietic condition. In some embodiments, the neoplastic, immunologic or hematopoietic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma, sepsis, allergic diseases and disorders that include but are not limited to allergic rhinitis, allergic conjunctivitis, allergic asthma, atopic eczema, atopic dermatitis, and food allergy, immunodeficiencies including but not limited to severe combined immunodeficiency (SCID), hypereosiniphic syndrome, chronic granulomatous disease, leukocyte adhesion deficiency I and II, hyper IgE syndrome, Chediak Higashi, neutrophilias, neutropenias, aplasias, agammaglobulinemia, hyper-IgM syndromes, DiGeorge/Velocardial-facial syndromes and Interferon gamma-TH1 pathway defects, autoimmune and immune dysregulation disorders that include but are not limited to rheumatoid arthritis, diabetes, systemic lupus erythematosus, Graves' disease, Graves ophthalmopathy, Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens, and hematopoietic conditions that include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the neoplastic or hematopoietic condition is non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia. In some embodiments the condition is breast cancer. In a preferred embodiment the condition is triple negative breast cancer wherein the breast cancer tumor is characterized by genetic alterations in BRCA1 or BRCA2 genes, and the absence of the estrogen receptor, progesterone receptor, and HER2 receptor tyrosine kinase.

In some embodiments, the neoplastic or hematopoietic condition is non-B lineage derived. Examples of non-B lineage derived neoplastic or hematopoietic condition include, but are not limited to, Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B atypical immune lymphoproliferations.

In some embodiments, the neoplastic or hematopoietic condition is a B-Cell or B cell lineage derived disorder. Examples of B-Cell or B cell lineage derived neoplastic or hematopoietic condition include but are not limited to Chronic Lymphocytic Leukemia (CLL), B lymphocyte lineage leukemia, B lymphocyte lineage lymphoma, Multiple Myeloma, and plasma cell disorders, including amyloidosis and Waldenstrom's macroglobulinemia.

Other conditions within the scope of the present invention include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR overexpression and resulting ERK activation in glioma cancer (Thomas C Y. Int J Cancer Mar. 10, 2003; 104(1):19-27), expression of HER2 in breast cancers (Menard et al. Oncogene. Sep. 29 2003, 22(42):6570-8), and APC mutation and activated Wnt signaling in colon cancer (Bienz M. Curr Opin Genet Dev 1999 October, 9(5):595-603).

Diseases other than cancer involving altered physiological status are also encompassed by the present invention. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D. J Immunol Methods 2003 February; 273 (1-2):3-13). The invention is not limited to diseases presently known to involve altered cellular function, but includes diseases subsequently shown to involve physiological alterations or anomalies.

### Kits

In some embodiments the invention provides kits. In some embodiments, the invention provides kits for the classification, diagnosis, prognosis of a condition and/or prediction of outcome after administering a therapeutic agent to treat the condition, the kit comprising one or more modulators, inhibitors, specific binding elements for signaling molecules, and may additionally comprise one or more therapeutic agents. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the state-specific binding elements described herein, such as phospho-specific antibodies. A kit may also include other reagents that are useful in the invention, such as modulators, fixatives, containers, plates, buffers, therapeutic agents, instructions, and the like.

In some embodiments, the kit comprises one or more of antibodies which recognize dynamic state changes, protein modification, phosphorylation, methylation, acetylation, ubiquitination, SUMOylation, or cleavage of the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCγ□, PLCγ2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα□□, PKCβ□□, PKCθ□□□, PKCδ, CAMK, Elk, AFT, Myc, Egr-1, NFAT, ATF-2, Mdm2, p53, DNA-PK, Chk1, Chk2, ATM, ATR, β□catenin, CrkL, GSK3a, GSK3β, and FOXO. In some embodiments, the kit comprises one or more of the phospho-specific antibodies specific for the proteins selected from the group consisting of Erk, Syk, Zap70, Lck, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCγ, PLCγ2, STAT1, STAT 3, STAT 4, STAT 5, STAT 6, CREB, Lyn, p-S6, Cbl, NF-κB, GSK3β, CARMA/Bcl10, p-Chk1, p-Chk2, p-ATM, p-H2AX and Tcl-1. In some embodiments, the kit comprises one or more of the specific antibodies specific for the proteins selected from the group consisting of PARP, caspase 3 and p-53.

Kits provided by the invention may comprise one or more of the modulators described herein. In some embodiments, the kit comprises one or more modulators selected from the group consisting of SDF-1α, IFN-α, IFN-γ, IL-10, IL-6, IL-27, G-CSF, FLT-3L, IGF-1, M-CSF, SCF, PMA, Thapsigargin, H₂O₂, etoposide, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Kits provided by the invention may comprise one or more assays to determine the expression and/or function of one or more drug transporters.

In some embodiments, the kits of the invention enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entireties.

### EXAMPLES

### Example 1

### Analysis of BRCA1 and BRCA2 Protein Network in Single Cells

Patient Samples:

Sets of fresh or cryopreserved samples from patients can be analyzed. The sets can consist of peripheral blood mononuclear cell (PBMC) samples or bone marrow mononuclear cell (BMMC) samples derived from blood. All patients will be asked for consent for the collection and use of their samples for institutional review board (IRB)-approved research purposes. All clinical data will be de-identified in compliance with Health Insurance Portability and Accountability Act (HIPAA) regulations. Samples can include those collected from breast cancer patients with a mutation in BRCA1 or BRCA2, patients with a triple-negative carcinoma phenotype (negative for estrogen receptor, progesterone receptor, and human epidermal growth factor receptor 2) that are BRCA1 and BRCA2 wild type, and patients without breast cancer that lack BRCA1 and BRCA2 mutations. A study could be set up to analyze samples from patients in for groups: 1a) women carrying germline BRCA 1 or 2 mutations who have a history of either triple negative breast cancer or ovarian cancer; 1b) women carrying germline BRCA 1 or 2 mutations who do not have a history of cancer; 2a) women who are negative for BRCA 1 and 2 mutations and have a history of either triple negative breast cancer or ovarian cancer; and 2b) women who are negative for BRCA 1 and 2 mutations and who do not have a history of cancer.

Materials:

In this example, the following materials are used: Phosphate Buffered Saline (PBS) (MediaTech); Thawing media (PBS-CMF+10% FBS+2 mM EDTA); 70 um Cell Strainer (BD); 1 uL anti-CD45 Alexa 700 (Invitrogen) per sample; 1 ug/mL propidium iodide (PI) solution (Sigma) (7-AAD or an equivalent viability dye can also be used); RPMI+1% FBS; Media A (RPMI+1% FBS+Penn/Strep); Live/Dead Reagent, Amine Aqua (Invitrogen); 2 mL, 96-Deep Well, U-bottom polypropylene plates (Nunc); 300 uL 96-Channel Extended-Length D.A.R.T. tips for Hydra (Matrix); 16% Paraformaldehyde (Electron Microscopy Sciences); 100% Methanol (EMD); Transtar 96 dispensing apparatus (Costar); Transtar 96 Disposable Cartridges (Costar, Polystyrene, Sterile); Transtar reservoir (Costar); Foil plate sealers.

Cell Network Profiling Assays:

Cell network profiling assays can involve measuring protein levels and their post-translational modification by phosphorylation in different populations of cells at baseline and after perturbation with various modulators. The populations that can be analyzed include PMBCs such as B cells, T cells, dendritic cells, monocytes, macrophages, neutrophils, eosinophils, and basophils. Other cells such as epithelial cells can also be analyzed.

A pathway “node” is defined as a combination of a specific proteomic readout in the presence or absence of a specific modulator. Levels of signaling proteins, as well as expression of cell surface markers (including cell lineage markers, membrane receptors and drug transporters), are detected by multiparameter flow cytometry using fluorochrome-conjugated antibodies to the target proteins. Multiple nodes (including surface receptors and transporters), using multiple modulators can be assessed.

A minimum yield of 100,000 viable cells and 500 cells per gated sample in gate of interest can be used for each patient sample to be classified as evaluable.

Cyropreserved samples are thawed at 37° C., diluted in RPMI, 10% FBS (or RPMI 60% RBS) and live mononuclear cells are purified via ficoll densitry centrifugation, and washed again in RPMI 10% FBS. The cells are resuspended, filtered, and are washed in RPMI cell culture media, 1% FBS, then are stained with Live/Dead Fixable Aqua Viability Dye (Invitrogen, Carlsbad, Calif.) to distinguish non-viable cells. The viable cells are resuspended in RPMI, 10% FBS, aliquoted to ˜100,000 cells/condition, and are rested for 1-2 hours at 37° C. prior to cell-based functional assays or staining for phenotypic markers. Each condition can include 2 to 5 phenotypic markers (e.g., CD45, CD33), up to 3-5 intracellular stains, or up to 3-5 additional surface markers.

Treatment of Cells with a Modulator:

A concentration for each modulator that is five fold (5×) more than the final concentration is prepared using Media A as diluent. The 5× stimulants are arrayed in a standard 96 well v-bottom plate that correspond to the wells on the plate with cells to be stimulated. Fixative is prepared by dilution of stock 16% paraformaldehyde with PBS to a concentration that is 1.5×, and then placed in a 37° C. water bath. Once the plated cells have completed their incubation, the plate(s) are taken out of the incubator and place in a 37° C. water bath next to the pipette apparatus. Prior to addition of stimulant, each plate of cells is taken from the water bath and gently swirled to resuspend any settled cells. The stimulant is pipetted into the cell plate, which is then held over a vortexer set to “7” (˜2000 rpm) and mixed for 5 seconds, and followed by the return of the deep well plate to the water bath. For cells undergoing apoptosis inducing conditions, cells are re-stained with Aqua viability dye priori to fixation. Cells are washed with PBS+0.5% BSA prior to staining with Amine Aqua viability dye, then are washed with RPMI 10% prior to fixation. After centrifugation and fixation cells are typically in a 100 uL volume before fixation.

Fixing Cells:

200 μL of the fixative solution (final concentration is 1.6%) is dispensed into wells containing 100 uL of cells and then mixed on the titer plate shaker on high for 5 seconds. The plate is then covered with foil sealer and floated in 37° C. water bath for 10 min, followed by a 5 min spin at 1000×g at room temperature. Supernatant is removed using a 96 well plate aspirator (VP Scientific), and cells are resuspended in the residual volume by vortexing, achieving pellet dispersion prior to the methanol step (see cell permeabilization) to avoid clumping. Cell Permeabilization: Permeability agent (such as −20 C methanol) is added slowly using a pipette a 96-well V&P dispenser. Plate(s) are placed on ice until this step has been completed for all plates, after which plates are covered with a foil seal using the plate roller to achieve a tight fit. At this stage the plates may be stored at −80° C.

Incubation of Cells with Antibodies and Processing for Cytometry:

Cells are pelleted at 2000 rpm for 5 minutes. A vacuum aspirator is used to remove the resulting supernatant, and the plate is vortexed on the plate-vortexer for 5 to 10 seconds. Cells are washed with 1 mL FACS/stain buffer (PBS+0.1% Bovine serum albumen (BSA)+0.05% Sodium Azide). The plate is then spun, vortexed, and aspirated as above, which can be further repeated if desired. Using a single channel, multichannel or 96-well pipettor, 50-100 μL of FACS/stain buffer with the desired, previously optimized antibody cocktail, is added (staining volume can vary) followed by agitation. Samples are covered and incubated on plate shaker for 60 minutes at room temperature (staining duration and temperature can vary). During this incubation, the compensation plate is prepared: In a standard 96-well V-bottom plate, 20 μL of “diluted bead mix” (1 mL FACS buffer+1 drop anti-mouse Ig Beads+1 drop negative control beads) is added per well. Each well receives 5 μL of 1 fluorophor conjugated control IgG (such as Alexa488, PE, Pac Blue, Aqua, Alexa647, Alexa700). For the Aqua well, 200 uL of Aqua−/+ cells are added. Following these additions, the compensation plate is incubated at room temperature for 10 minutes, followed by a wash with 200 uL FACS/stain buffer, centrifugation at 2000 rpm for 5 minutes, and removal of supernatant. This wash, centrifugation, and removal step is then repeated, followed by resuspension in 200 uL FACS/stain buffer and transfer to a U-bottom 96-well plate. (As an alternative to the compensation plate, cells such as PBMC can be used for single color controls or fluorescence minus one (FMO) controls. Also, machine cytometers can be standardized with predefined voltages and compensation settings for specific combinations of fluorophores and Quality Controlled daily.) To the cell sample plate, after the above 60 minute incubation, 1 mL of FACS/stain buffer is added per well followed by a 5 minute incubation at room temperature. Cells are centrifuged, aspirated, and vortexed as above. 1 mL of FACS/stain buffer is again applied to each well, followed by incubation for 5 minutes. Centrifugation, aspiration, and vortexing are again repeated as above. Cells are fixed in 1.6% PFA for 5 minutes at room temperature, and are then centrifuged, aspirated, and vortexed and transferred to U-bottom plates or other appropriate plates) and covered with a foil seal. Cells are then stored at 4 C in the dark until being acquired on a flow cytometer. Cells are then analyzed using a flow cytometer, such as a LSRII (Becton Dickinson), with a high throughput screening (HTS) 96-well plate reader, all wells selected, and the following Loader Settings: 2 uL/sec flow rate; 40 uL sample volume; 250 uL/sec mixing speed; number of mixes set to 5; 800 uL wash volume; and standard mode (or alternative appropriate loader settings). When the plate has completed, a batch analysis in performed to ensure there are no clogs. Rainbow Calibration Particles (Spherotech) or other beads are used as cytometer controls to ensure proper machine function.

Data Acquisition and Cytometry Analysis:

Data can be acquired using FACS DIVA software on both LSR II and CANTO II Flow Cytometers (BD). For all analyses, dead cells and debris are excluded by FSC (forward scatter), SSC (side scatter), and Amine Aqua Viability Dye measurement. Populations of interest within the samples can be identified using markers known in the art. Activity of the DNA repair node after exposure to a BRCA mutation-targeted chemotherapy regime and/or other DNA damaging agents can be analyzed using markers with binding elements specific for components of the DNA repair pathway. Such components may include BRCA1, BRCA2, RAD51, and members of the BRCA1-associated genome surveillance complex (BASC), including MSH2, MSH6, MLH1, ATM, BLM, the RAD50-MRE11-NBS1 protein complex, DNA replication factor C, p-Chk2, cleaved-PARP, p-X2AX, ATM/ATR, p21, p53 and cell cycle.

These analyses can then be used to identify deficiencies in the DNA repair pathway of affected cells, as well as deficiencies in pathways associated with aberrantly responding proteins. Cells from patients having a BRCA gene mutation and cells responding to the BRCA mutation-targeted chemotherapy regime can be compared to wild type cells. Furthermore, cells with functional insufficiencies that, on one hand predispose patients to cancer can, on the other hand, be used to predict the response of a tumor to certain cytotoxic therapies and targeted therapies, such as BRCA mutation-targeted therapies.

### Example 2

### Analysis of p53 Levels

Background

The p53 tumor suppressor is a transcription factor that is a tightly regulated protein that is involved in cell cycle arrest and induction of apoptosis in genetically damaged cells. Mutations or deletions of the p53 gene may facilitate the transmission of genetic damage and the emergence of neoplastic clones with a survival advantage. Wild type p53 has a short half-life (due to continuous ubiquitylation and subsequent degradation by the 26S proteosome) and cannot be detected in the cell nucleus of most human cells. In contrast, mutated p53 has a prolonged half-life and becomes detectable by immunological techniques using p53 antibodies.

Summary of p53 Functional Regulation

When a cell is subjected to stress, p53 is stabilized in the nucleus, where it initiates cellular responses through a transcriptional program by which distinct target genes whose function is primarily to prevent proliferation of damaged cells.

The function of p53 is tightly controlled by its interaction with negative regulators including MDM2, which induces p53 degradation and prevents its accumulation in normal cells. This interaction can be disrupted when the cell detects DNA damage or other stresses, resulting in stabilization and activation of p53.

Active p53 is subject to a diverse array of covalent post-translational modifications, which have a profound influence on the expression of p53 target genes.

Phosphorylation and acetylation of p53 generally result in its stabilization and accumulation in the nucleus, followed by activation. Significant redundancies are observed in that the same p53 site is phosphorylated by several different protein kinases and distinct protein kinases also phosphorylate several sites on p53.

Mutant p53 proteins generally show intense phosphorylation and acetylation at sites that are well known to stabilize wild-type p53, and so could facilitate accumulation of dysfunctional mutant p53 in the nucleus, where it can act as an oncogene.

Overexpression of MDM2 E3 ubiquitin ligase is observed in many tumor types and results in the aberrant deactivation of p53.

In normal cells, p53 post-translational modification is induced by numerous carcinogens. Evidence indicates that normal cells and cancer cells show a markedly different response to ultraviolet-light exposure.

The functional domains of p53 are shown in FIG. 1 (Reference Bode and Dong, Nat Rev. Cancer Vol 4, p793 (2004)).

Functional Classes of p53 Mutations

p53 function can be disrupted in different ways: 1) disruption of the p53 allele, 2) mutations in the p53 gene that generate a dysfunctional or absent protein, 3) indirect mechanisms such as altered expression of other gene products that disrupt p53 function such as MDM2 amplification or loss of Arf.

Notably, most mutations in p53 are found in the DNA binding core domain encompassing residues 98-292 that abrogate the transcriptional activity of p53. Another consequence of these mutations is p53 protein accumulation and escape from down-regulation.

The Role of p53 Protein in CLL

17p abnormalities and TP53 mutations are poor prognostic indicators in CLL.

p53 is located on chromosome 17p13.1. Known to be altered in a number of hematological malignancies, p53 mutations are low in lymphoid malignancies except for non-hodgkins lymphoma, progressive CLL and B cell chronic prolymphocytic leukemia. Furthermore, p53 positivity is a hallmark of the stabilized mutated form of the protein. When the percentage of p53 positivity was correlated with the clinical stage of the disease, the prevalence of p53 positive cases increased significantly as the disease progressed; Binet stage A (8/100 7.4%) to Binet stage B (12/49 24.4%) and to Binet stage C (7/25 29.2%). P53 correlated with the phase of the disease showing low expression at diagnosis (8/112 7.1%) and a higher level as the disease progressed (7/35 20%) (Cordone et al., Blood (1998) Vol. 91 p. 4342). Importantly, when the entire p53 cDNA was sequenced in 15 samples, there was an association between protein expression and mutations in the gene.

Furthermore, studies have shown that p53 protein expression was shown to be associated with advanced clinical stage, progressive disease, poor response to therapy and short survival (Cordone et al., Blood (1998) Vol. 91 p. 4342, Grever et al., J. Clin. Oncol. (2007) Vol. 25 p. 799, Schlette et al., Leukemia and Lymphoma (2009) Vol. 50 p. 1597).

Single Cell Network Profiling (SCNP) Test for p53 Expression

Patient Samples:

Sets of fresh or cryopreserved samples from patients can be analyzed. The sets can consist of peripheral blood mononuclear cell (PBMC) samples or bone marrow mononuclear cell (BMMC) samples derived from blood. All patients will be asked for consent for the collection and use of their samples for institutional review board (IRB)-approved research purposes. All clinical data will be de-identified in compliance with Health Insurance Portability and Accountability Act (HIPAA) regulations. Samples can be from patients with chronic lymphocytic leukemia (CLL) at different stages of the disease, as well as from non-CLL patients.

Data Acquisition and Cytometry Analysis:

Using methods presented in Example 1, levels and activity of p53 and related proteins can be analyzed in individual cells. The test uses two p53 antibodies that recognize distinct p53 epitopes, as well as other antibodies specific for members of the pathways in which p53 participates, including cell cycle arrest, apoptosis, senescence, and DNA repair. Markers can have binding elements for different epitopes of p53; such additional pathway participants can include MDM2 and p-Arf. Markers may be selected that can distinguish between functional and non-functional protein targets.

The use of two antibodies that recognize distinct p53 epitopes, used together or separately, provides internal controls in the test. During the development stages of the test, a comparison will be conducted with p53 levels detected by SCNP versus sequencing the p53 cDNA to detect mutations in the gene. The SCNP test will allow p53 protein to be measured in single cells. For example in CLL samples B cells can be analyzed. In the same CLL sample p53 levels can be measured in other cell types, such as T cells and myeloid cells. p53 levels can also be measured simultaneously with other modulated signaling assays in distinct cell subsets within a CLL sample. SCNP therefore avoids measuring p53 levels in a mixture of cells. This will be advantageous for monitoring levels of p53 expression in specific cell subsets within diagnostic CLL patient samples as well as changes in p53 that occur in the same patient over time. Measurements of p53 by SCNP can be made in other hematologic malignancies as well as in other tumor types.

Activity of p53 and specific members of pathways in which p53 participates, including cell cycle arrest, apoptosis, senescence, and DNA repair can also be assessed by monitoring changes in protein levels in response to modulators within single cells, either by changes in levels of a protein of interest or in the levels of secondary proteins regulated by the protein of interest.

The SCNP test described herein can be used to monitor functional wild type p53 in leukemic cells. It is known that drugs such as etoposide stabilize p53 through ATM-mediated phosphorylation of serine 15 and we have this assay worked out. If mutant p53 protein is not detected then cells would be treated with a modulator to evaluate p-p53(S15), which would indicate the presence of functional wild type protein. There are many other sites for post translational modification of p53 known in the art that can be used in the methods described herein. Levels of MDM2, post translational modifications of MDM2 and p-Arf levels could also be measured by SCNP either alone or combined with each other or with other modulated signaling assays.

Comparison of results from leukemic cells of patients with different stages of CLL to non-leukemic cells of CLL and non-CLL patients can be used as a diagnostic for disease progression, as well as a means to monitor the degree to which the disease is controlled on a therapeutic regimen. Furthermore, characterization of the pathways involving p53 in leukemic cells in the presence or absence of a modulator can be used to predict their response to targeted cytotoxic therapies.

### Example 3

### Genomic Instability Analysis

Genomic instability is a hallmark of cancer. Germline mutations in DNA repair genes and/or genes that primarily function to maintain genomic stability may drive cancer development by increasing the spontaneous mutation rate. Examples of cancers associated with germline mutations associated include Hereditary non-polyposis colorectal carcinoma, Bloom's syndrome, Ataxia-telangiectasia (ATM mutation), BRCA-associated breast and ovarian cancers, Fanconi anaemia, Retinoblastoma. Somatic mutations in DNA repair genes and/or genes that function to maintain genomic stability and arise in the course of cancer genesis and progression, e.g., p53. Genomic Instability may be the basis for increased sensitivity/resistance to DNA damaging agents and can be exploited by synthetic lethality.

Synthetic Lethality exploits the dependence of the cancer cell upon a particular repair pathway, due to inactivating mutation in alternative(s). For example, cells that have a deficiency in homologous recombination (HR) repair pathway might be sensitive to PARP inhibitors. Healthy cells are spared due to functional alternative pathway.

Gene sequencing is the more common way to identify gene mutations but may underestimate gene inactivation. For example, epigenetic mechanisms alter gene expression but will not be detected, not all mutations have known functional significance e.g.; BRCA, and the accumulated effects of mutations in multiple genes may have functional effects that mutational analyses cannot identify. This example provides (i) functional assessment of carekeeper genes in germline cells that can provide tools to identify subjects at high risk for tumor development and inform appropriate preventive interventions, and (ii) functional assessment of carekeeper genes in somatic cells can provide tools to inform therapeutic selection. Table 2 below shows exemplary readouts that can be used to make these functional assessments.

TABLE 2

| Pathway | Target/Readout | Pathway | Target |
|---|---|---|---|
| NHEJ Repair | pDNA-PKcs | Damage Response | p-H2AX |
| NHEJ Repair | pKu70 | Damage Response | p-Chk2 |
| NHEJ Repair | pKu80 | Damage Response | p-Chk1 |
| NHEJ Repair | pAkt | Damage Response | p-ATM |
| NHEJ Repair | p53BP1 | Damage Reponse | p-ATR |
| HR Repair | pBRCA1 | Damage Response | p-p53 |
| HR Repair | pRad51, pRad52 | Apoptosis | MCL-1 |
| HR Repair | pRad51, pRad52 | Apoptosis | Cleaved Caspase 3 |
| HR Repair | pRPA32(RPA2) | Apoptosis | Ceaved PARP |
| PART activity | PARP, PAR | Apoptosis | Viability Dye |

### Generation and Validation of Tools to Examine DDR and Defective Signaling

DNA DSB response pathways were measured in cell lines controls using the antibodies staining methods and modulator conditions described in Example 1. Table 3 below describes the DNA damage pathways of a B-lymphoblastoid cell lines.

TABLE 3
B-lymphoblastoid cell lines for assaying DNA Damage Repair Pathways

| Gene | Pathway | +/+ | +/− | −/− |
|---|---|---|---|---|
| ATM | DNA Damage Sensing | ✓ | ✓ | ✓ |
| NBS-1 | DNA Damage Sensing | ✓ | | ✓ |
| ATR | HR | ✓ | | ✓ |
| BRCA1 | HR | ✓ | ✓ | |
| BRAC2 | HR | ✓ | | ✓ |
| BLM-1 | HR | ✓ | | ✓ |
| FANCD2 | HR/Fork Collapse | ✓ | | ✓ |

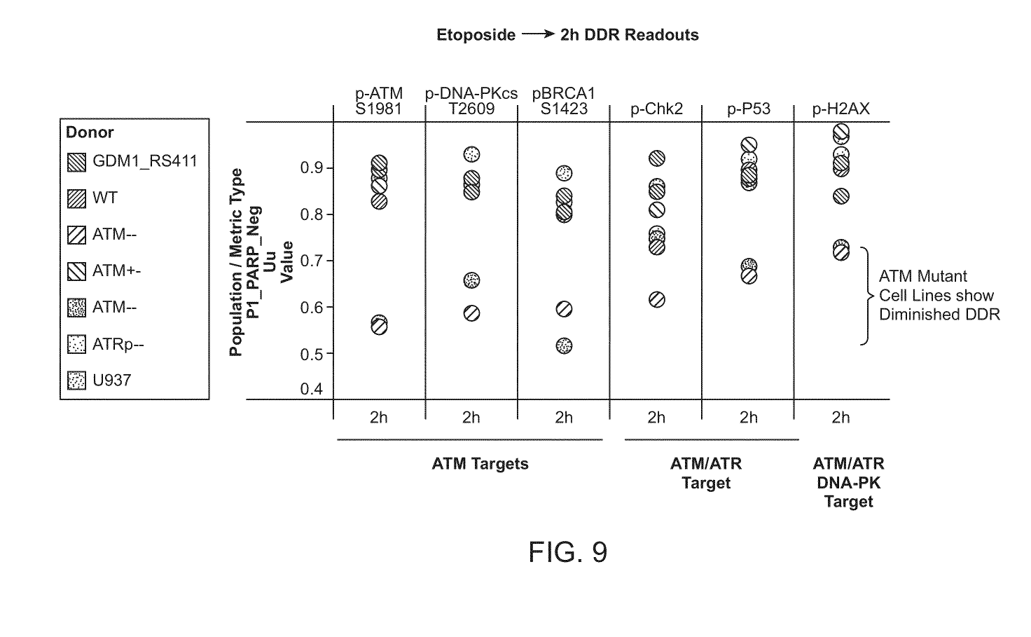

Other cell lines used were GDM1 (Chemorefractory AML cell line), U937 (Chemosensitive AML cell line) and RS411 (Chemosensitive B-ALL cell line). RS411 and GDM1 cells showed induction of DDR nodes in response to 2 h treatment with Etoposide. FIG. 9 shows that an ATM mutant cell line displayed attenuated DNA damage response to a 2 h treatment with etoposide. The experiments were repeated and found reproducible at least three times at different dates. Thus using the control cell lines, these results demonstrate feasibility and repeatability of DNA Damage readout assays (data not shown).

### Examination of the Sensitivities of AML Samples to PARP Inhibition, in Single Agent and Combination Studies

DNA DSB response pathways were measured in clinical samples as described above. Table 4 below summarized the clinical samples used.

TABLE 4

| | Early DDR | Early DDR | Apoptosis |
|---|---|---|---|
| Sample Category | Data (2 h) | Data (6 h) | Data (24 h) |
| AML Pediatric (0-21) | 5 | 4 | 3 |
| AML Adult (18-60) | 7 | 7 | 4 |
| AML Elderly (56+) | 15 | 7 | 10 |
| Cell Line Controls | 8 | 8 | 8 |

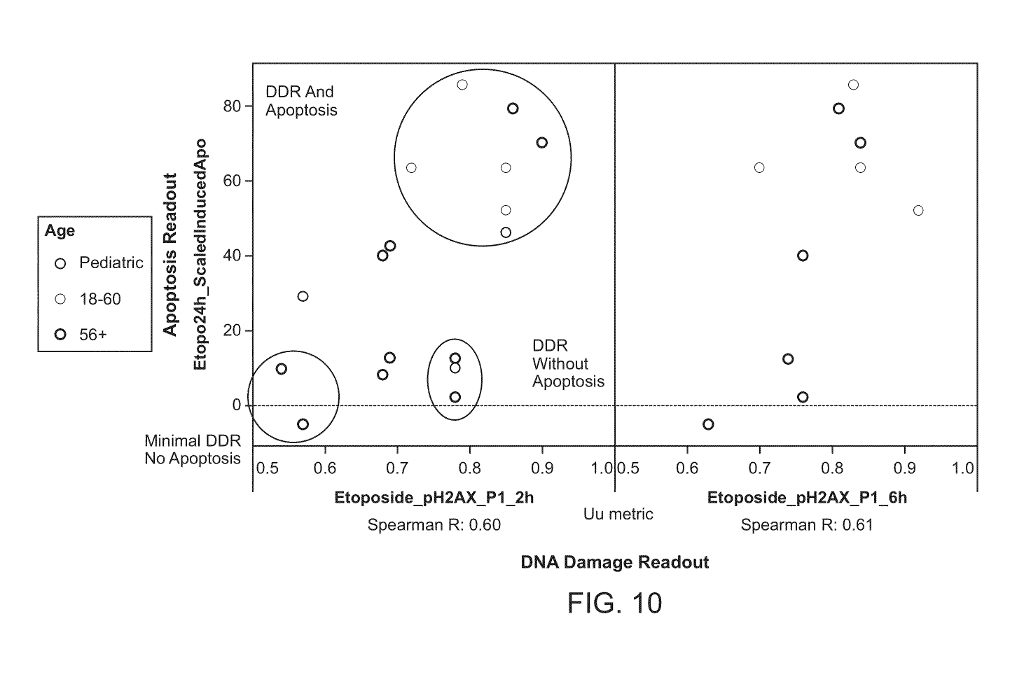

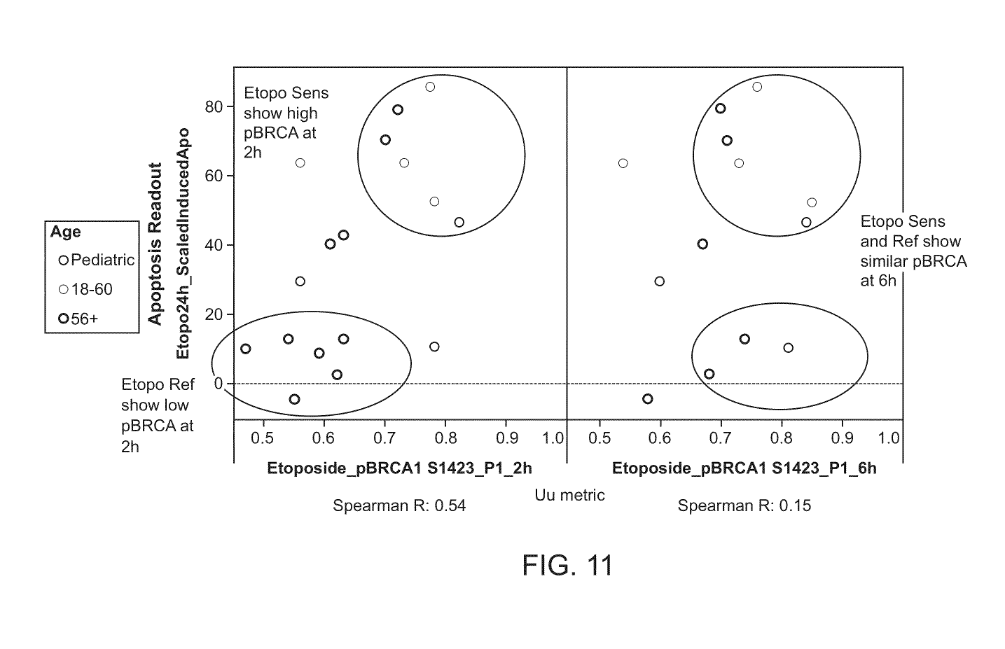

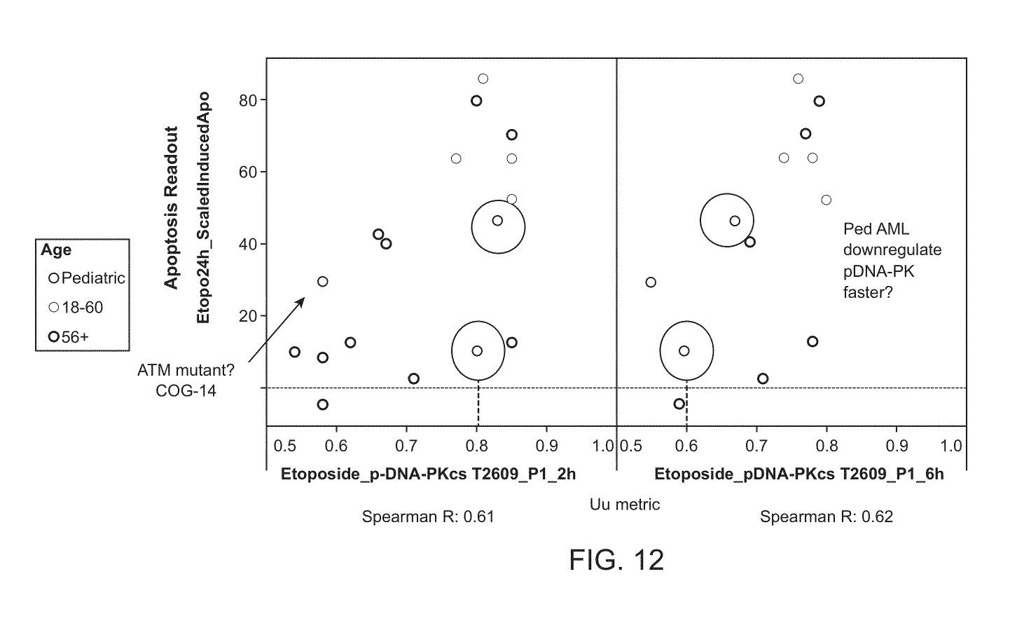

Etoposide-induced pATM (DNA DSB response) does not differentiate sensitive or resistant patient samples (data not shown). FIG. 10 shows that Etoposide induced DNA damage (pH2AX) and apoptosis identified distinct subgroups in AML Samples. FIG. 11 shows that Etoposide-induced HR (pBRCA1) and apoptosis identify distinct subgroups in AML samples. Etoposide refractory samples showed lower pBRCA at 2 hrs. This method allows the examination of individual points in the samples for signaling through other DNA DSB repair nodes. FIG. 12 shows induced phosphorylation of NHEJ (pDNA-PKcs) identifies etoposide sensitive samples. The difference in the kinetics of the samples suggests that pediatric AML samples might downregulate pDNA-PK faster than adult AML samples. The observed profiles suggest that some of the samples might have mutations in ATM. FIG. 13 pediatric AML display distinct kinetics of etoposide induced pDNA-PK downregulation.

Correlations between DDR nodes in AML samples (2 h, 6 h) highlight connections between repair pathways. We observed higher correlations between HR (also called HRR) and DNA DSB response (p-BRCA1, pATM) and between NHEJ and DNA damage (p-DNAPK, p53PB1, pH2AX). There were some weaker correlations observed between the 2 major DNA DSB repair pathways, NHEJ Repair (pDNA-PK, p53BP1) vs. HR (pBRCA). There were some other weaker correlations observed at 6 h vs. 2 h, e.g., differential regulation at later timepoints. Table 5 below shows some of the correlations observed.

TABLE 5

| | | 2 h | 6 h |
|---|---|---|---|
| Response 1 | Response 2 | R² | R² |
| pBRCA1 S1423 | p-ATM S1981 | 0.80 | 0.78 |
| pH2AX | p-DNA-PKcs T2609 | 0.79 | 0.67 |
| p-53BP1 S1778 | p-DNA-PKcs T2609 | 0.78 | 0.53 |
| pH2AX | p-53BP1 S1778 | 0.74 | 0.64 |
| p-53BP1 S1778 | p-Chk2 | 0.73 | 0.48 |
| p-53BP1 S1778 | p-ATM S1981 | 0.71 | 0.72 |
| p-53BP1 S1778 | pBRCA1 S1423 | 0.64 | 0.58 |
| pH2AX | pBRCA1 S1423 | 0.63 | 0.65 |
| p-ATM S1981 | p-DNA-PKcs T2609 | 0.62 | 0.42 |
| pH2AX | p-ATM S1981 | 0.62 | 0.58 |
| pBRCA1 S1423 | p-DNA-PKcs T2609 | 0.55 | 0.19 |

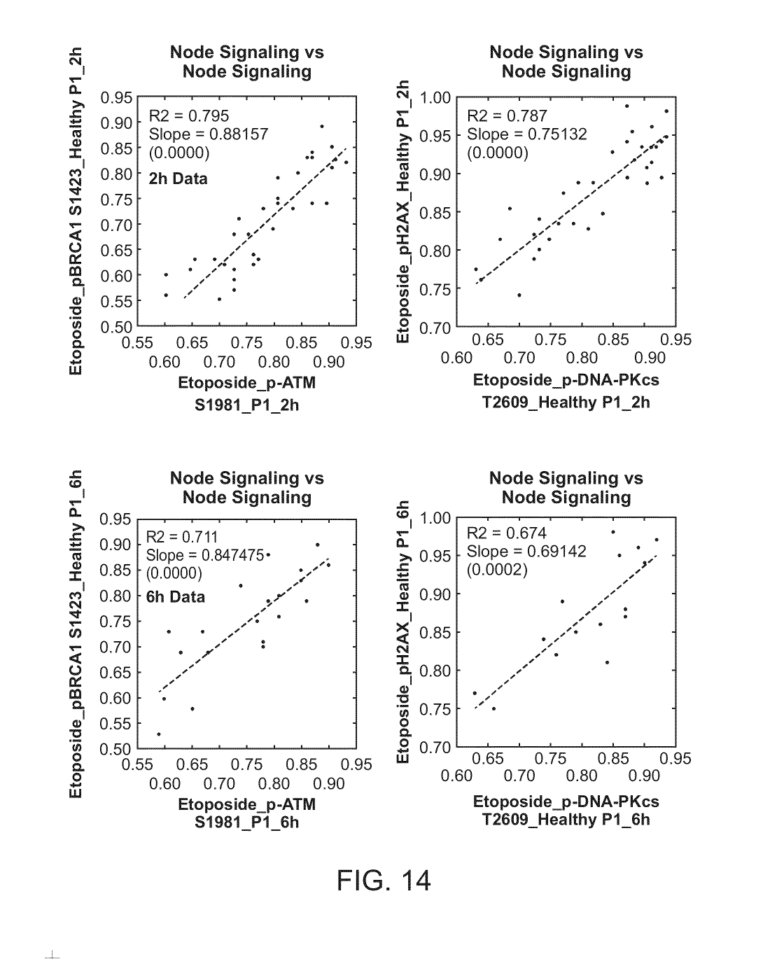

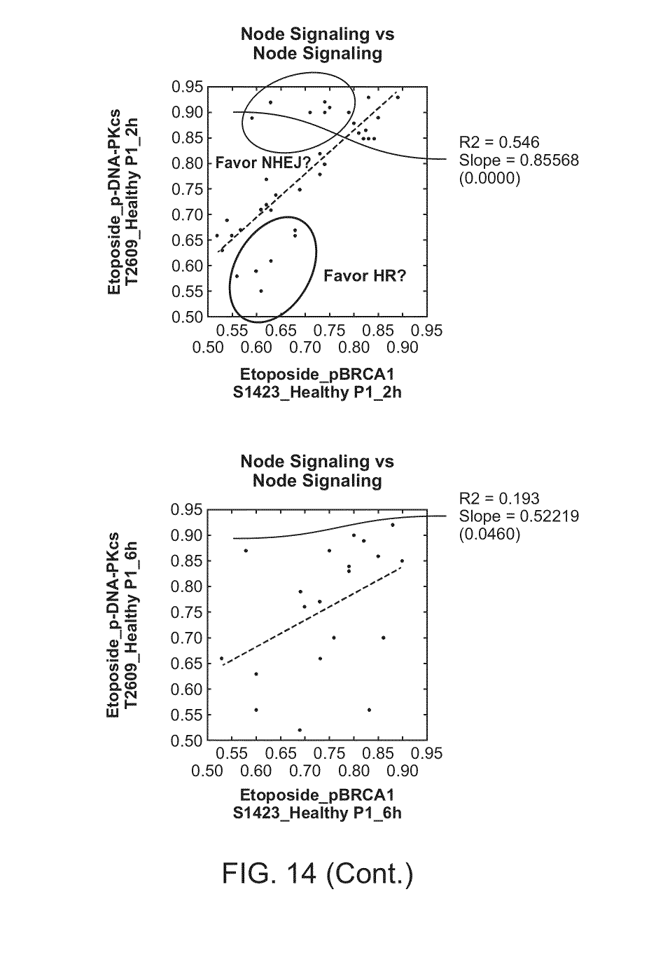

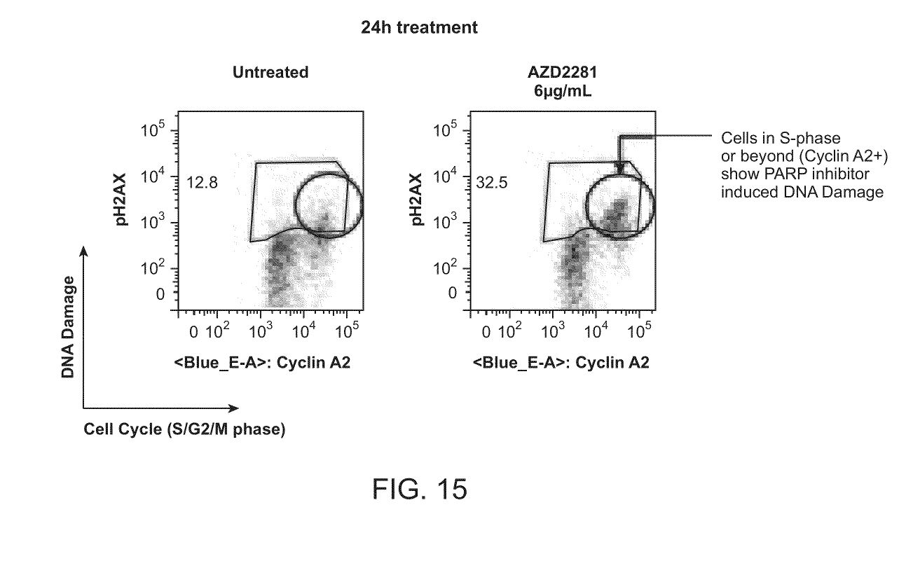

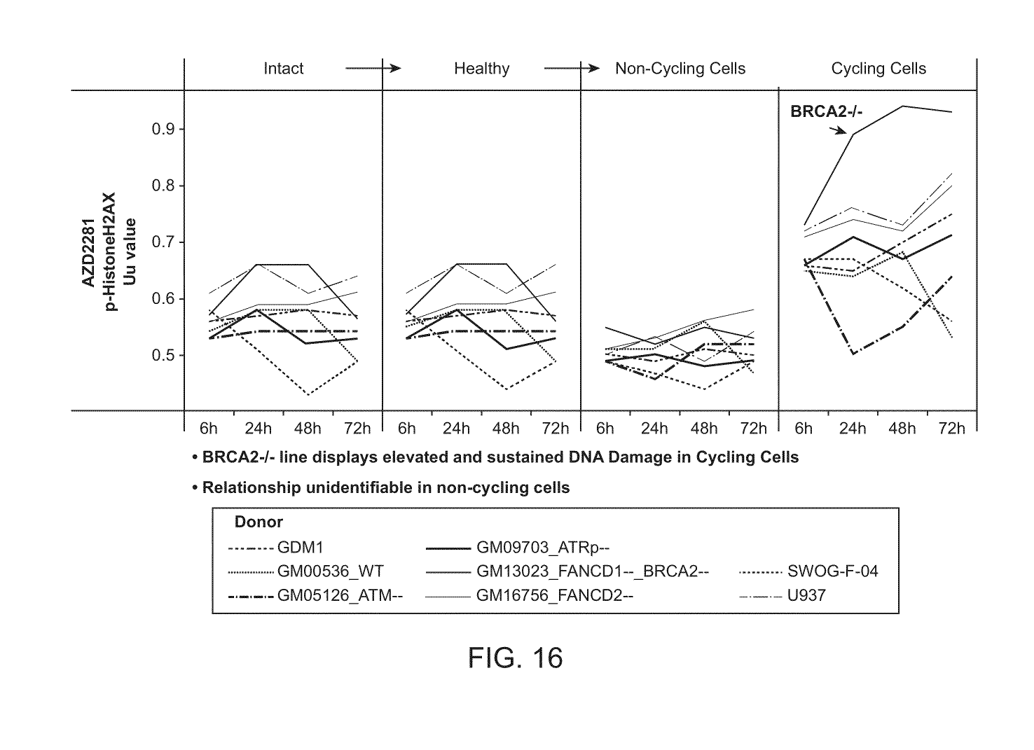

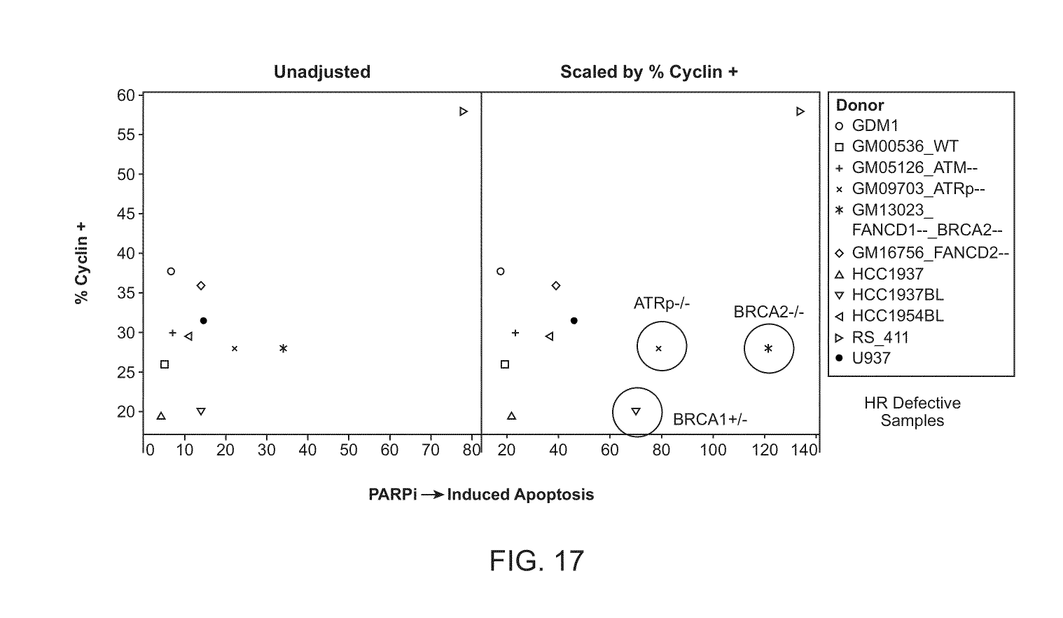

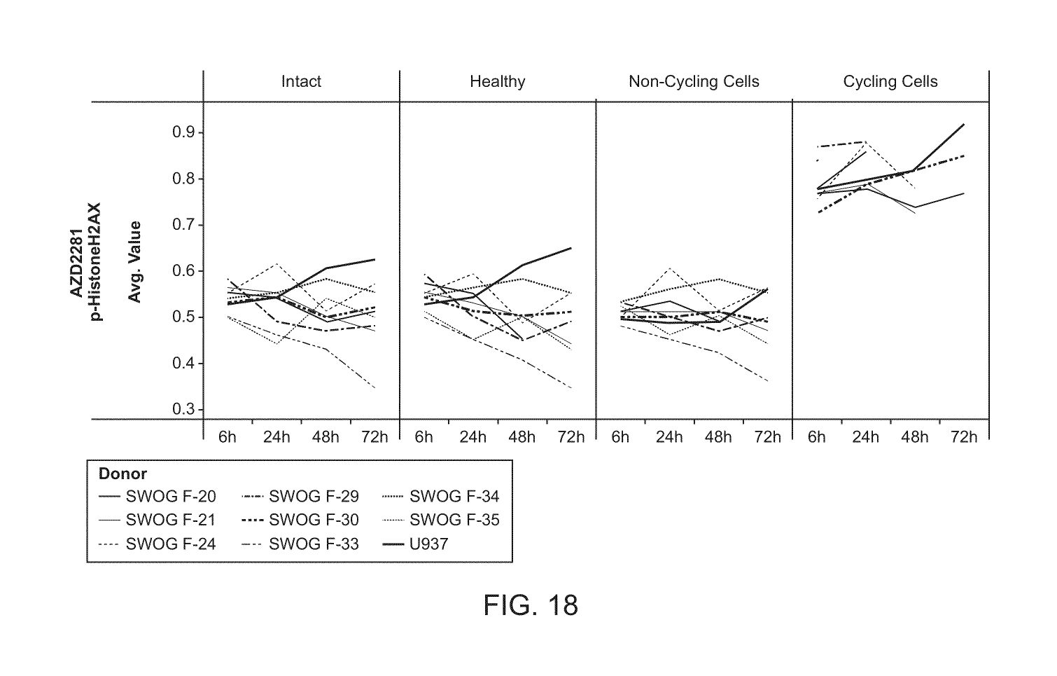

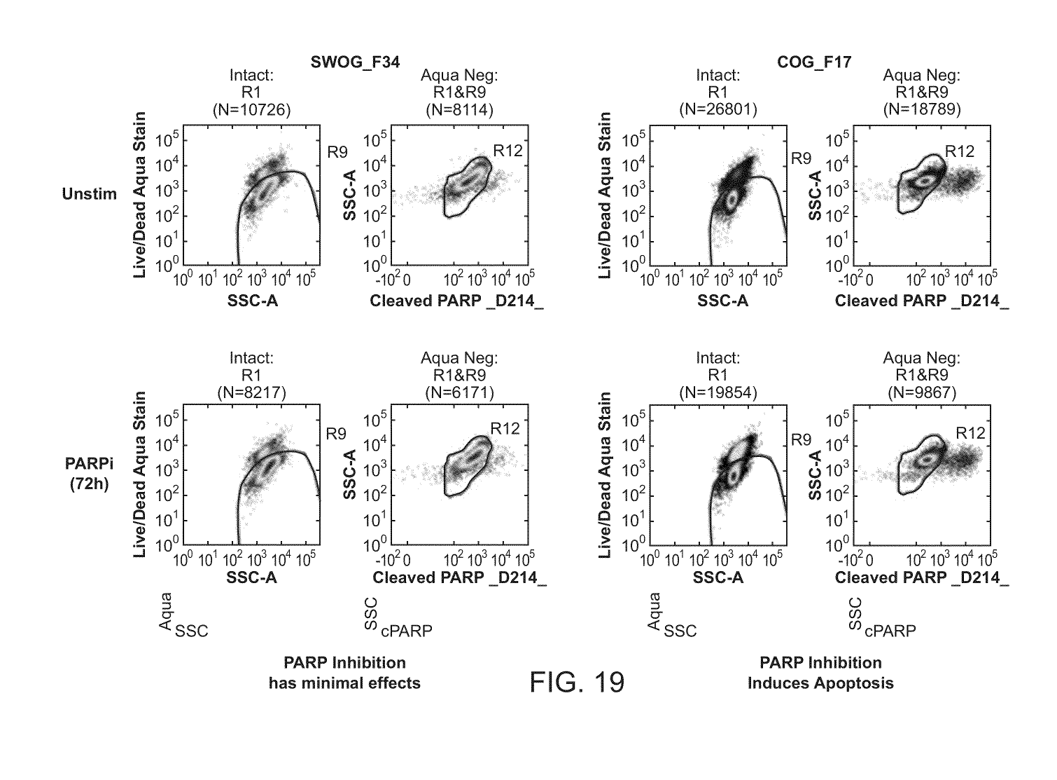

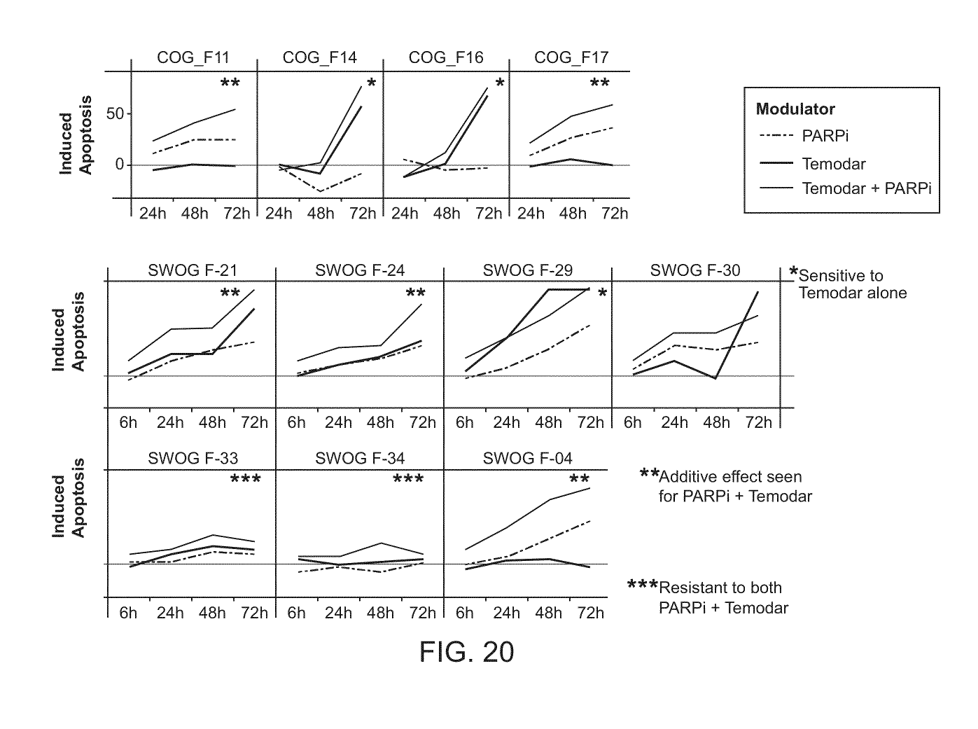

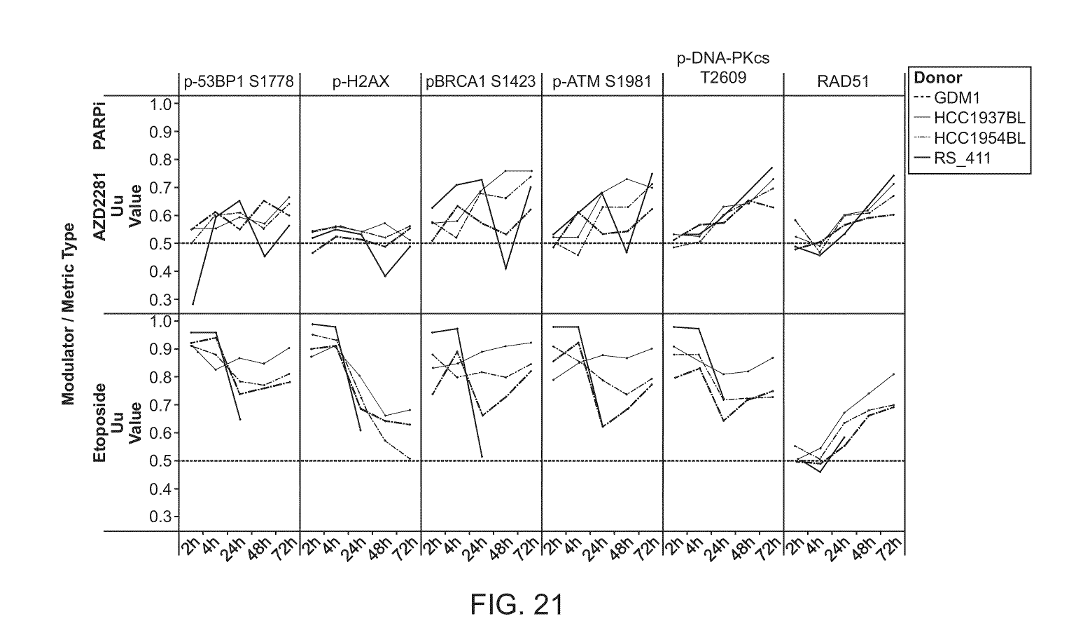

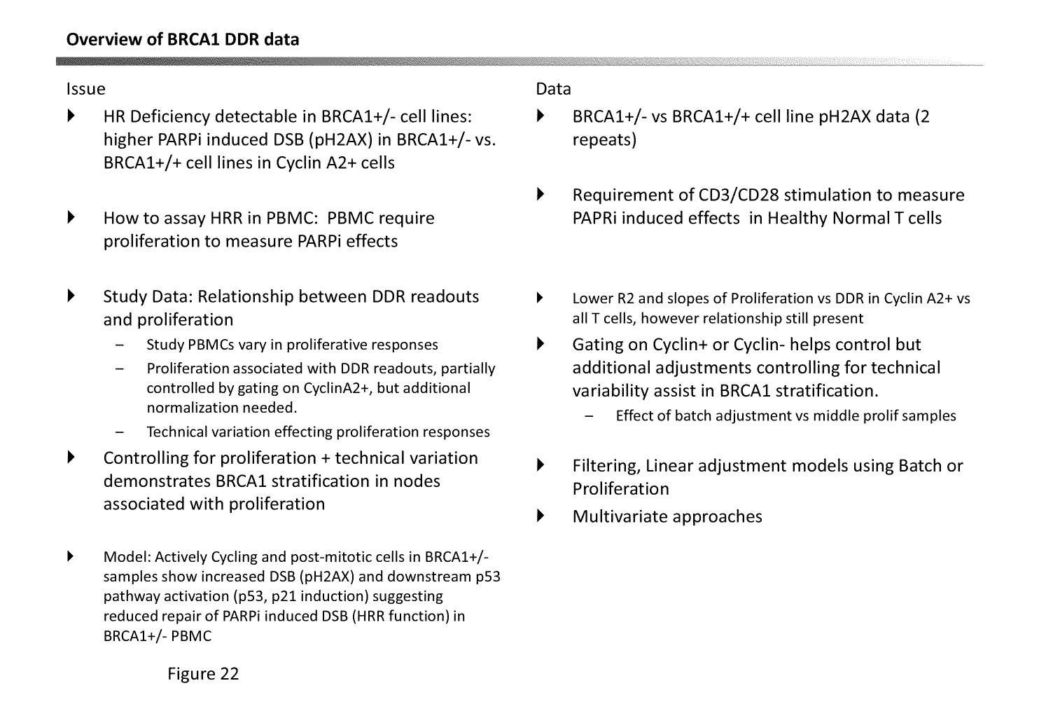

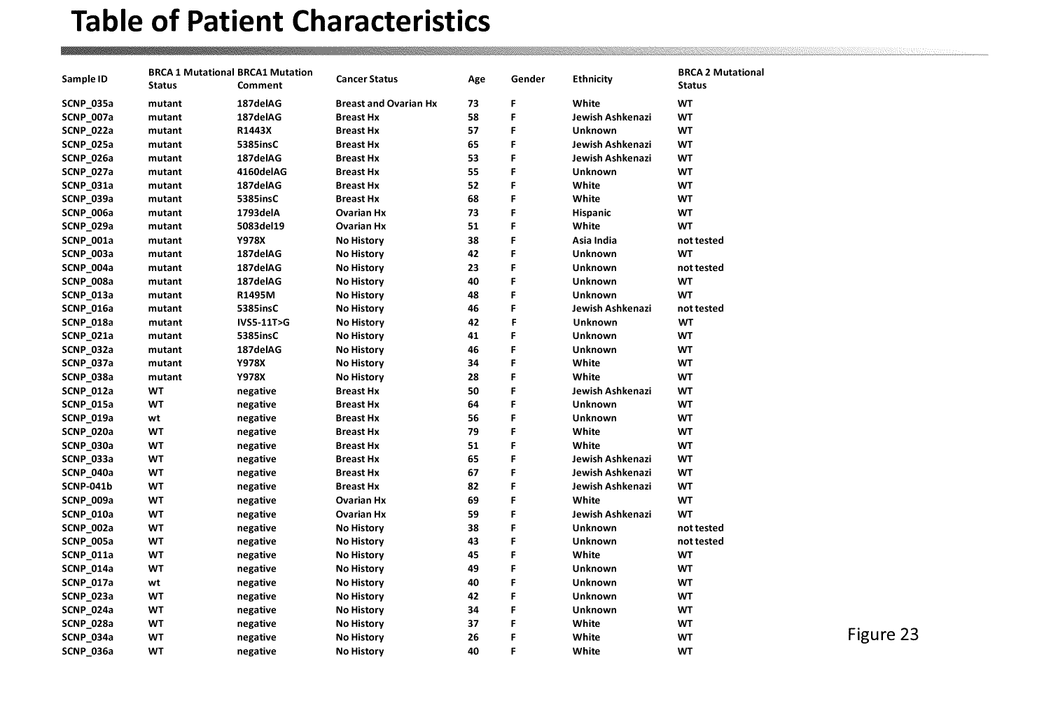

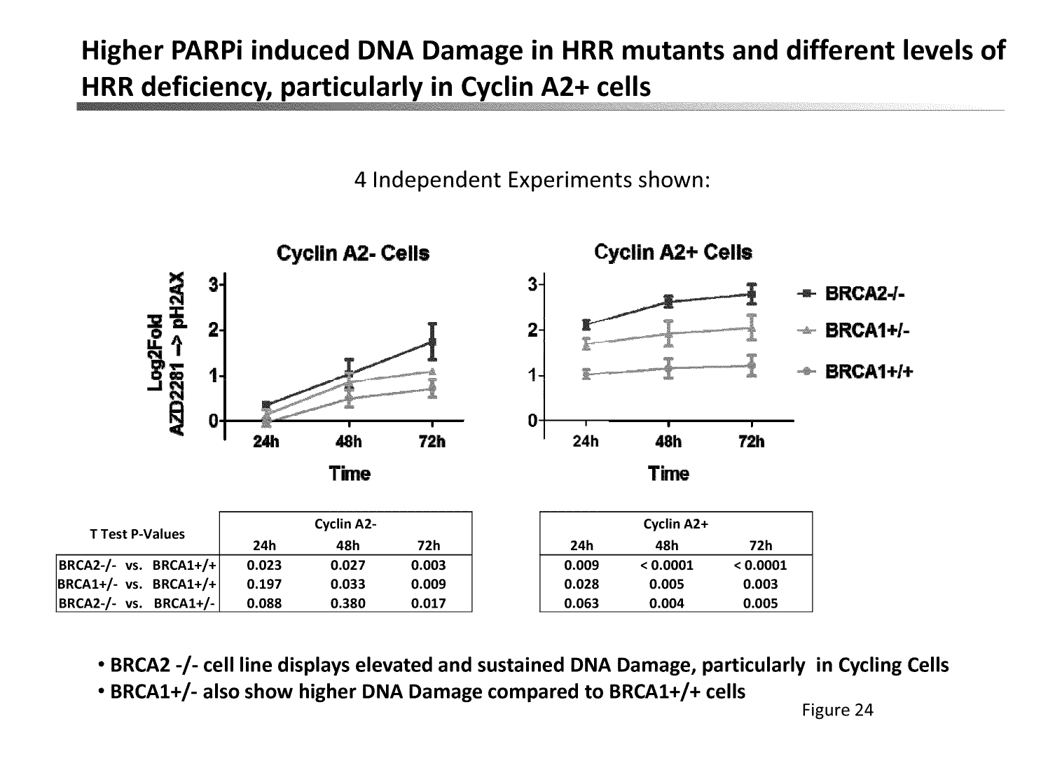

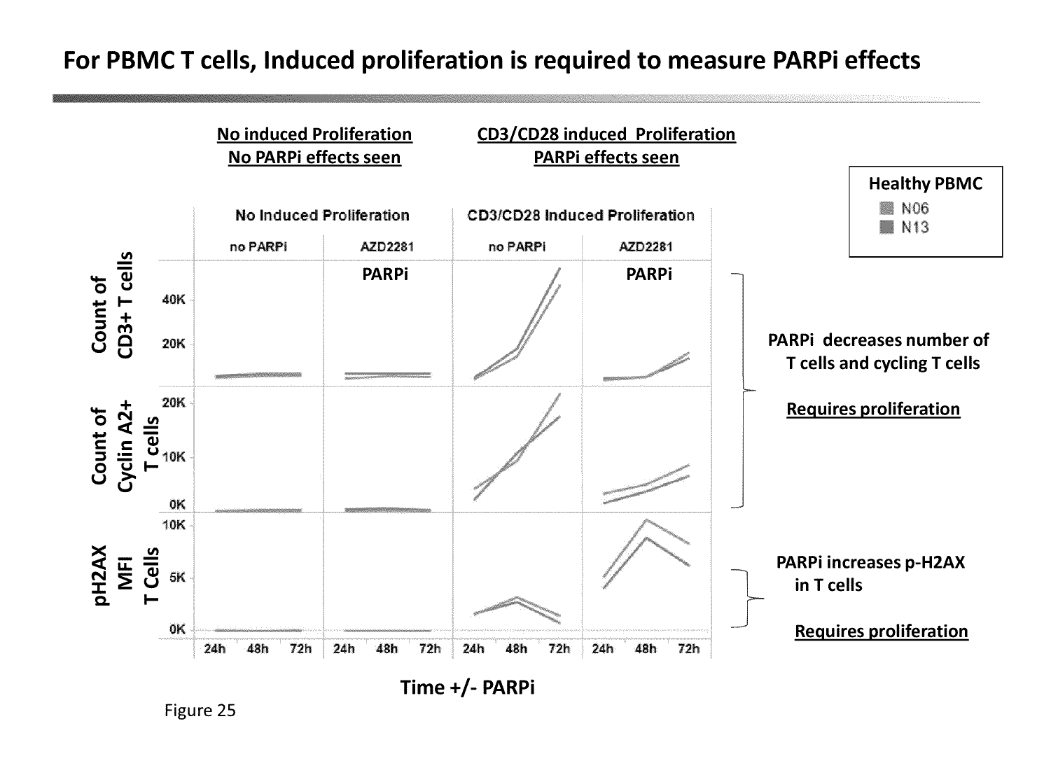

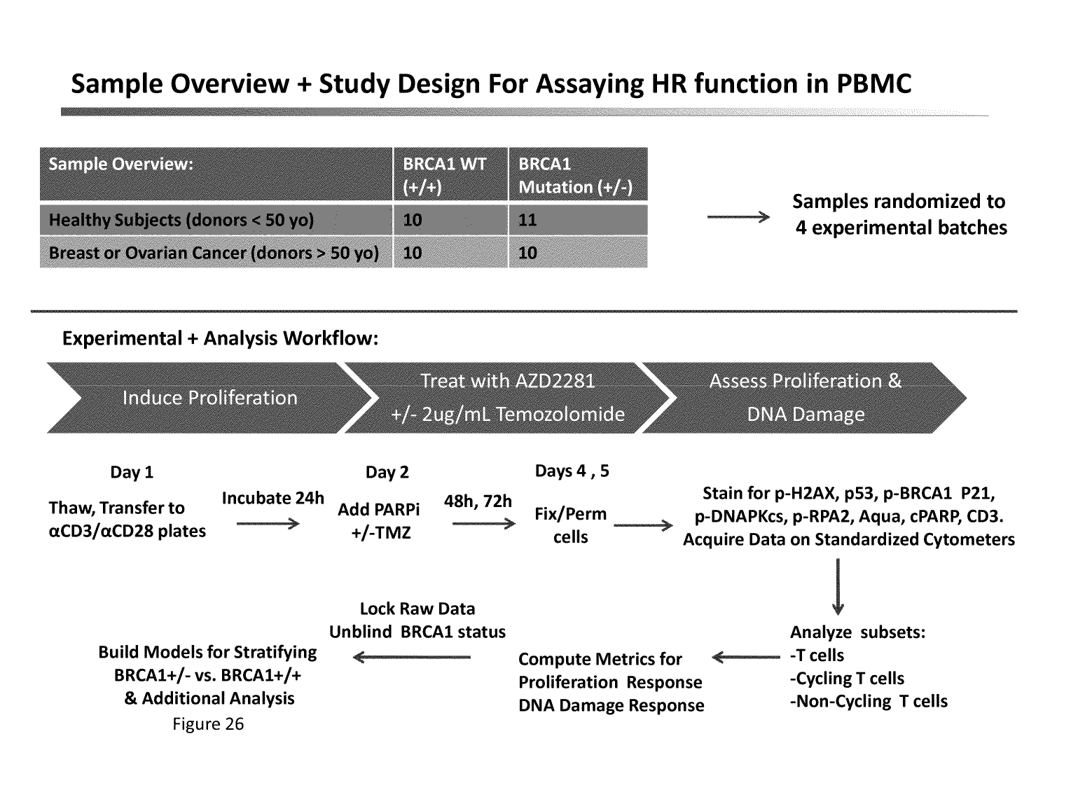

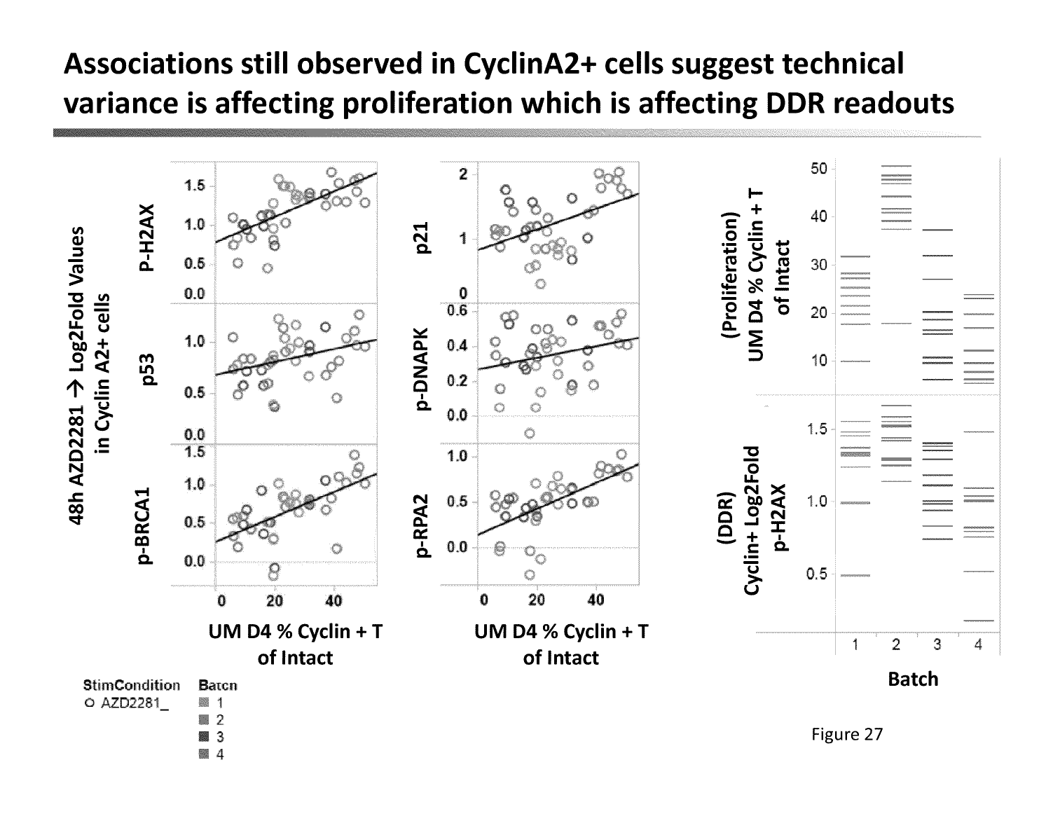

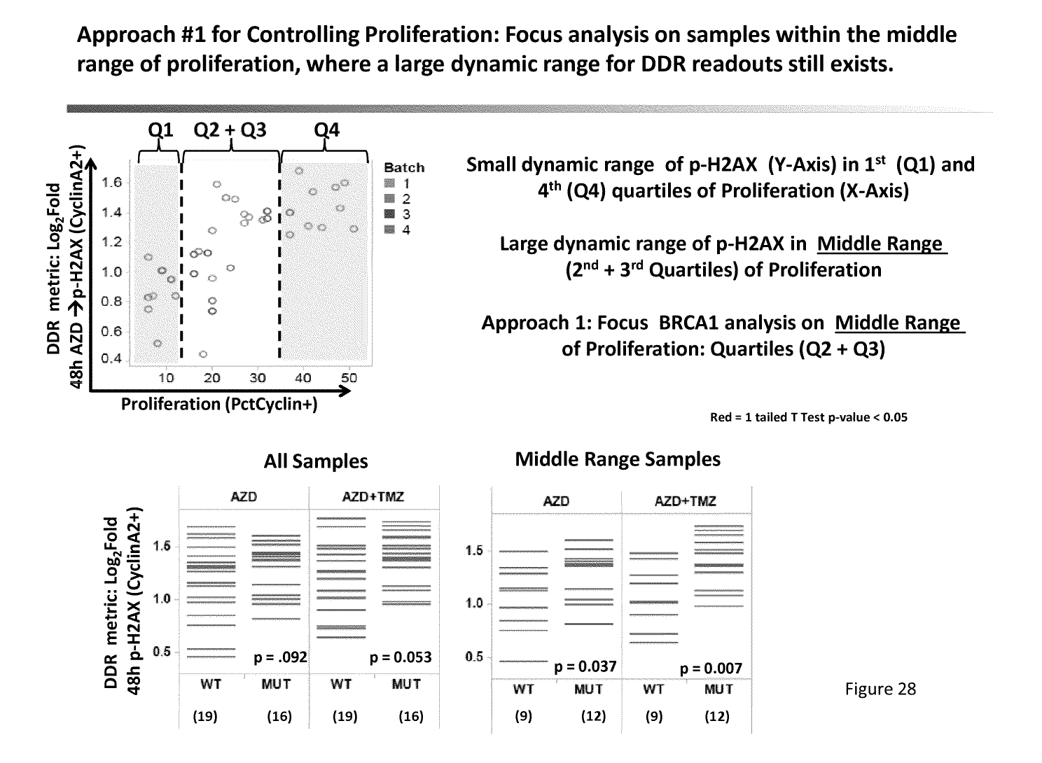

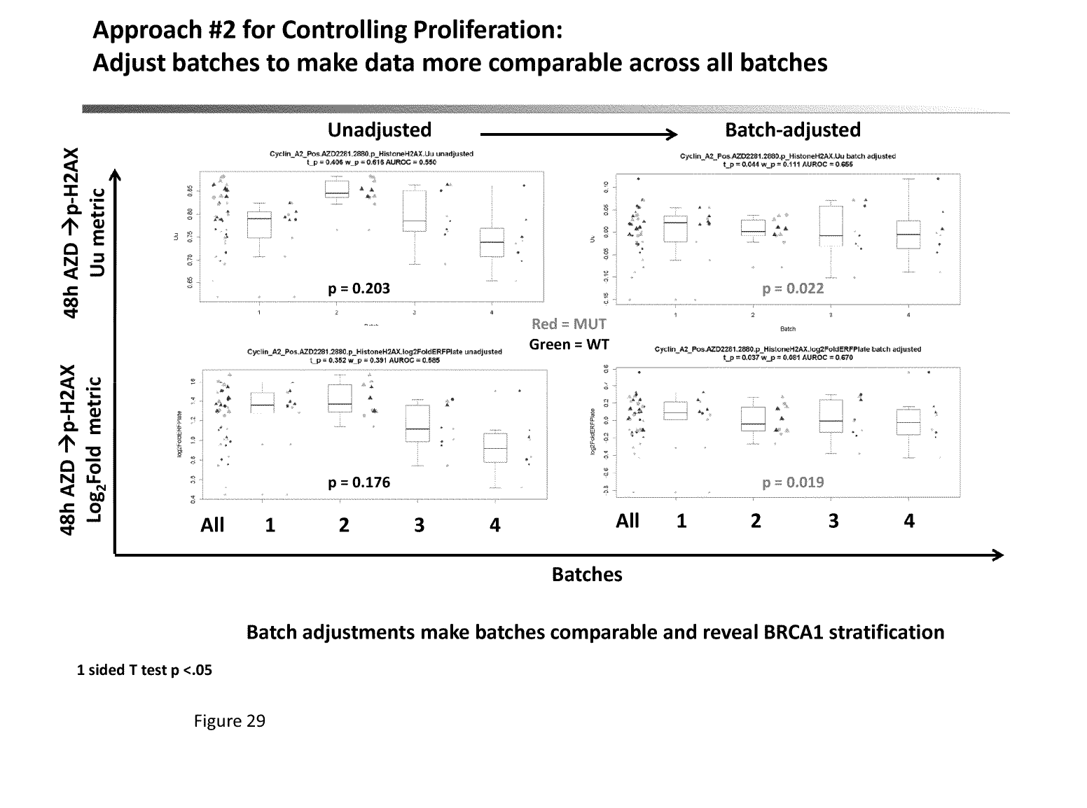

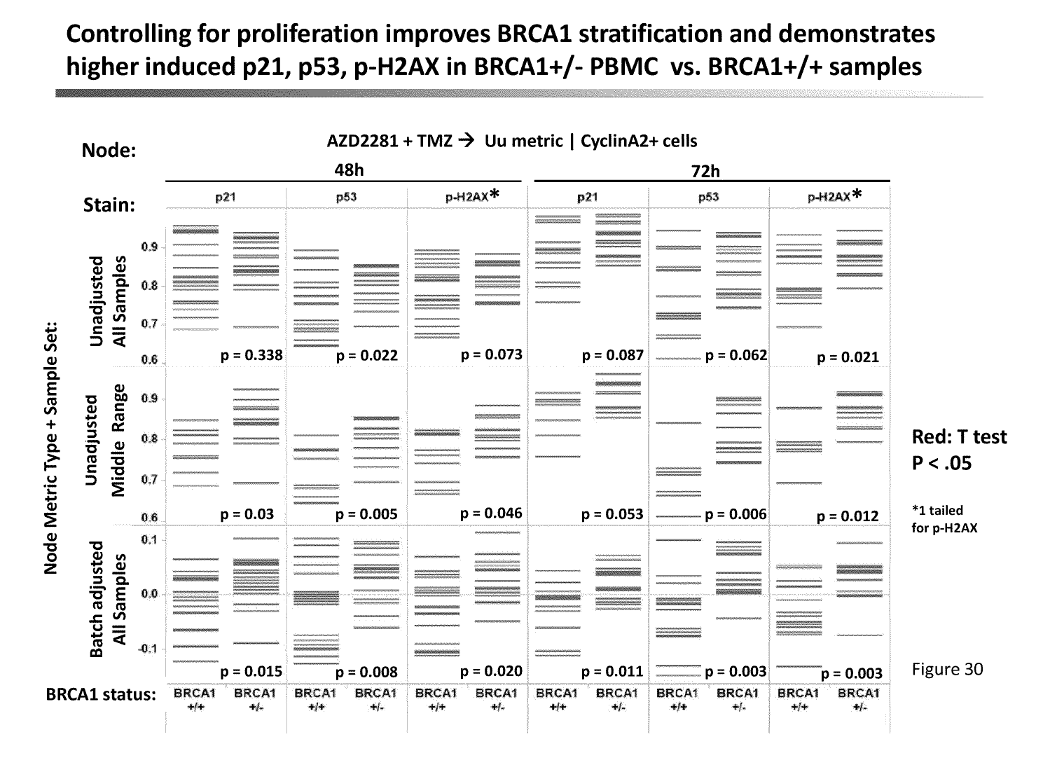

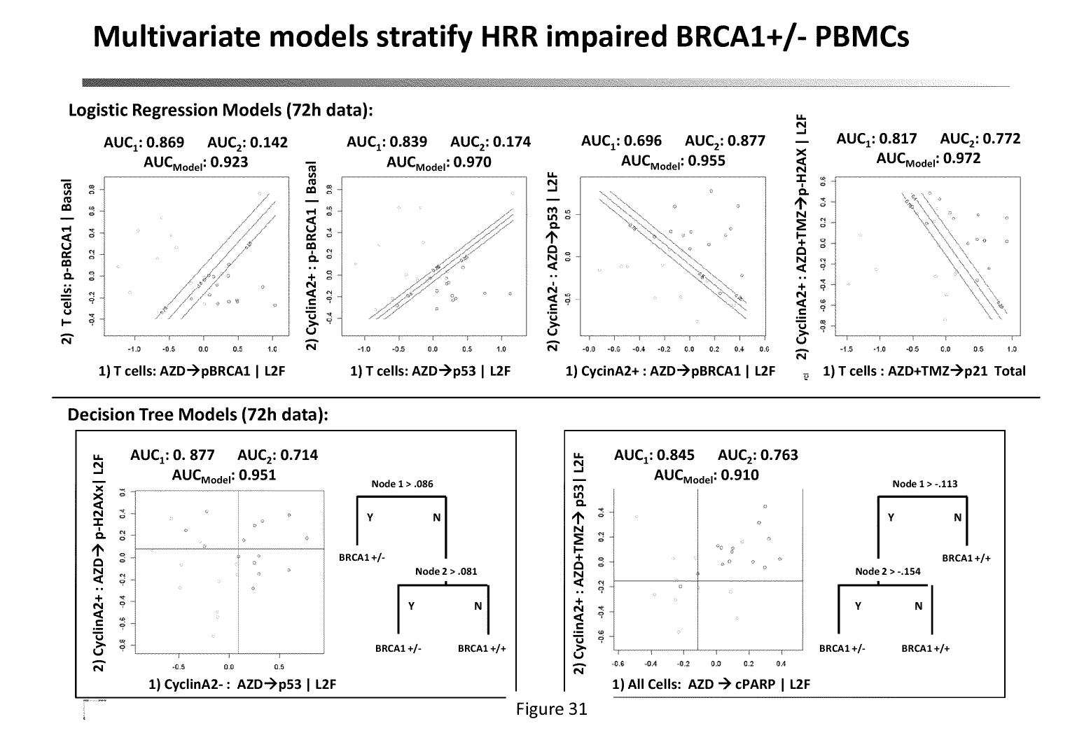

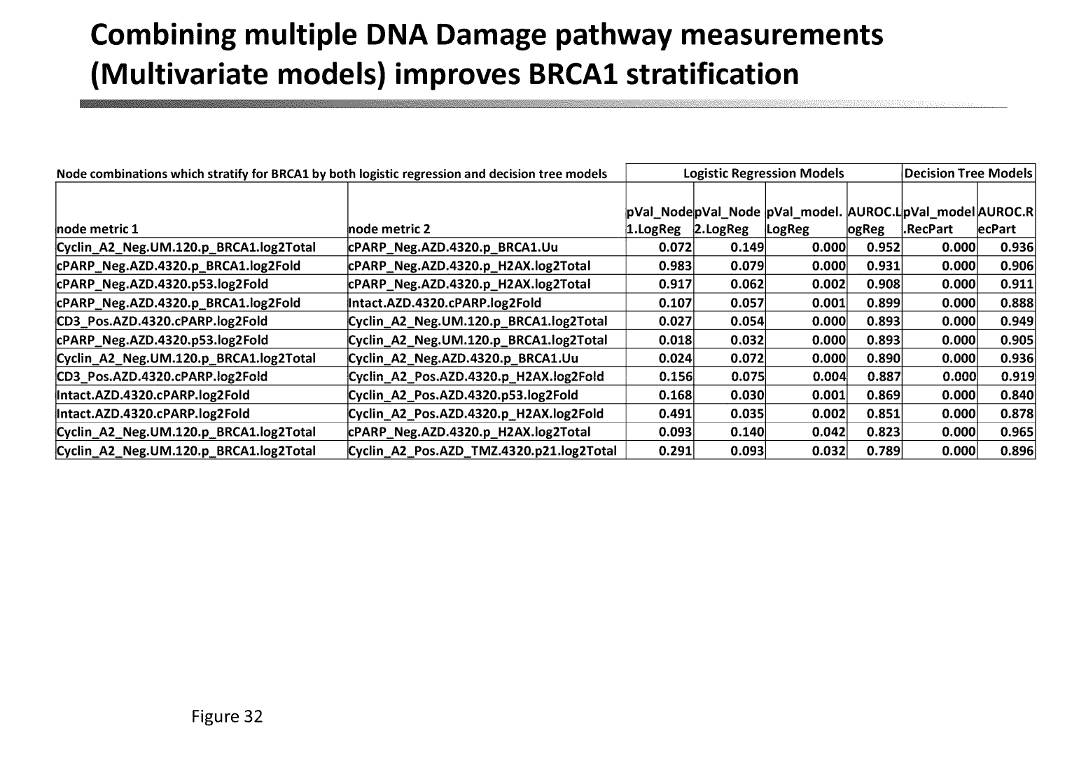

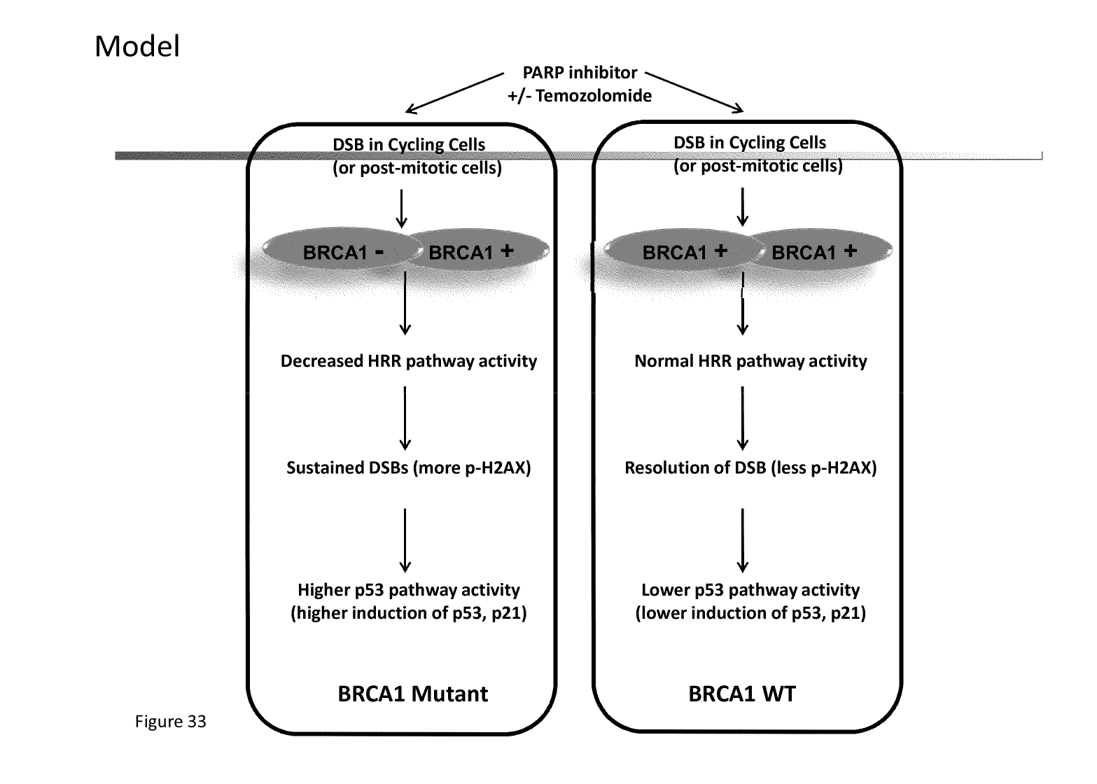

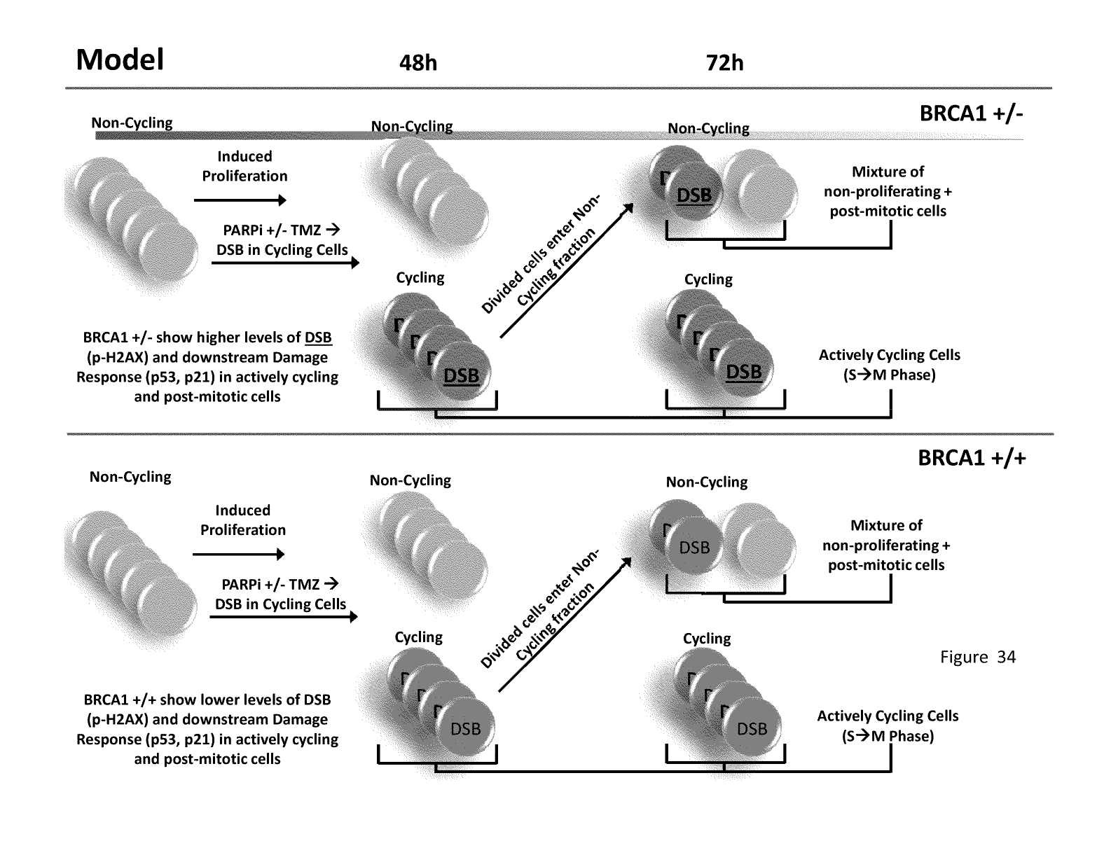

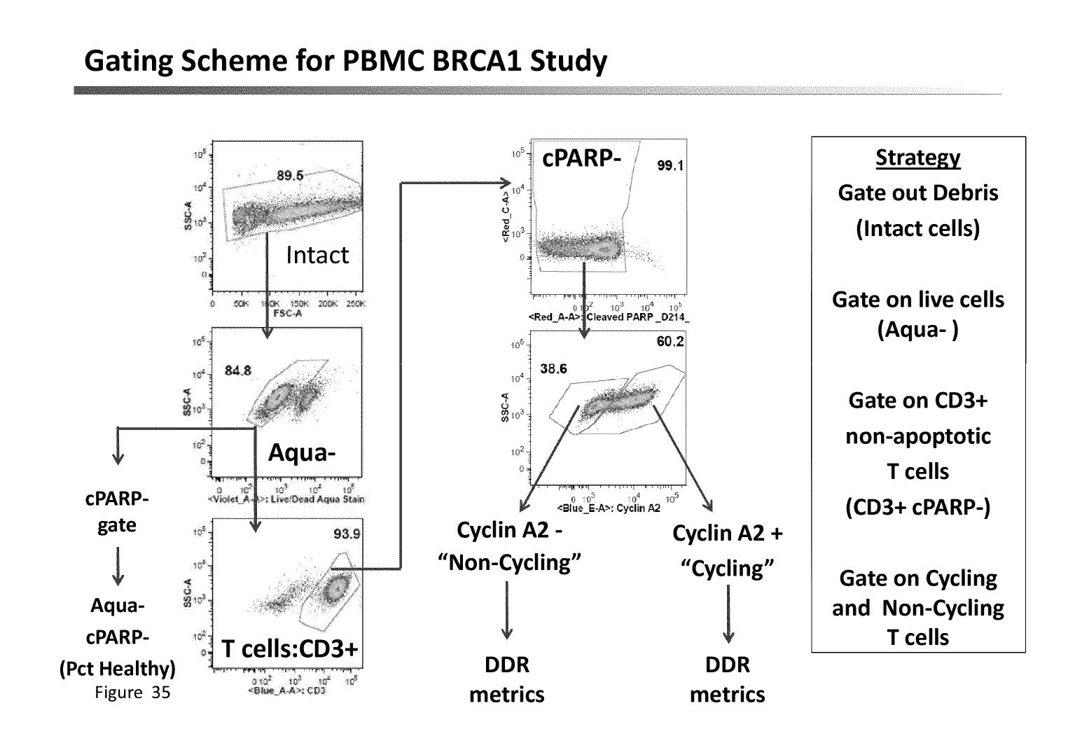

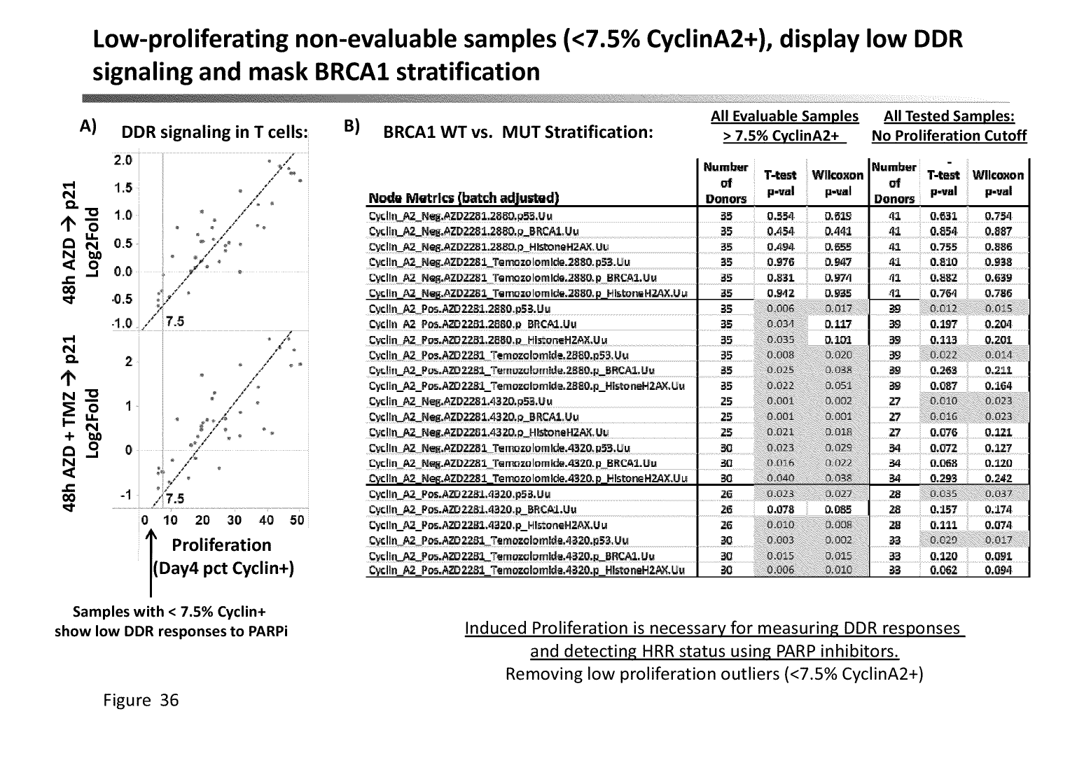

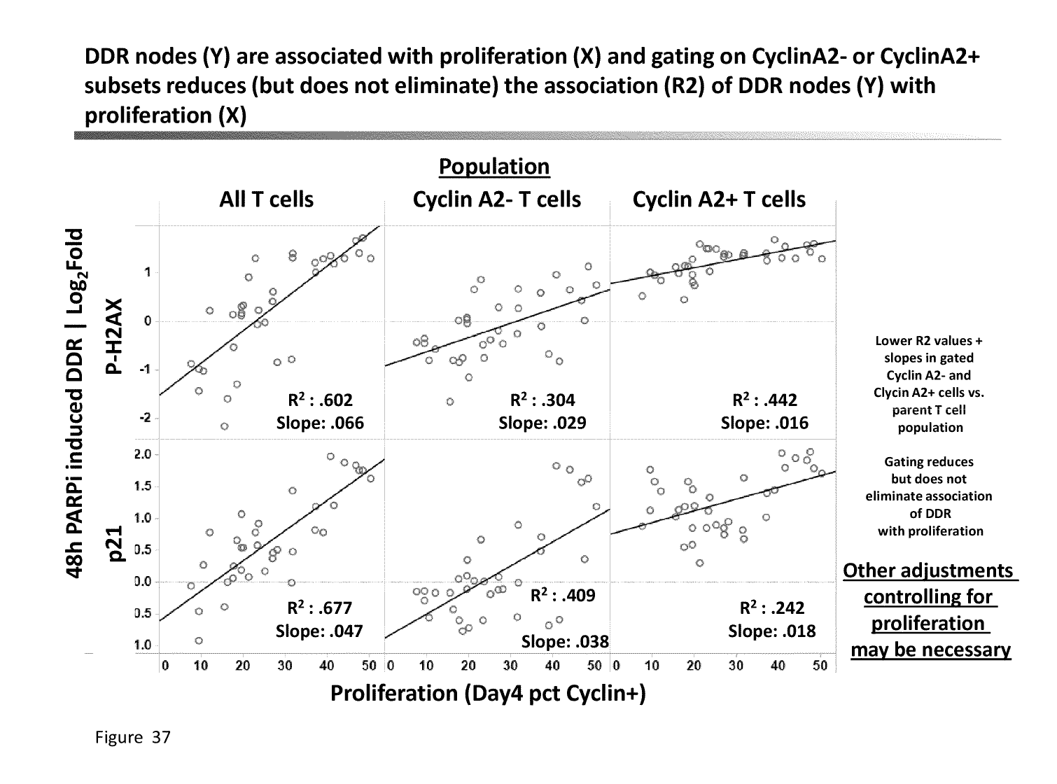

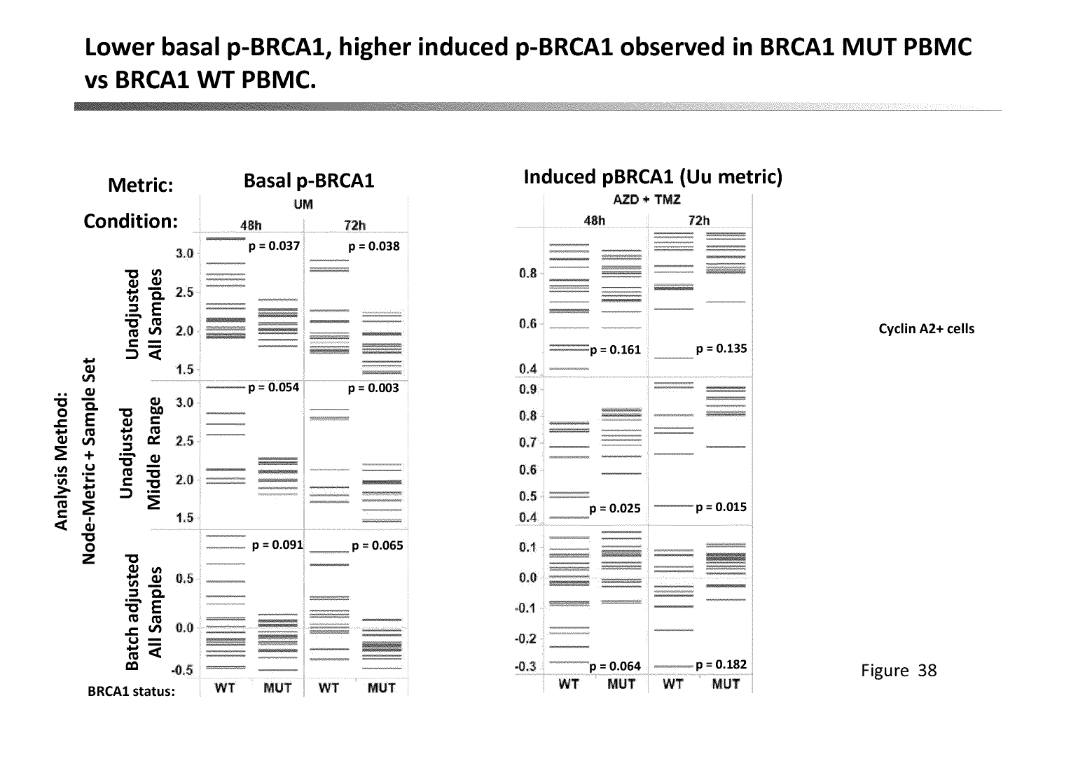

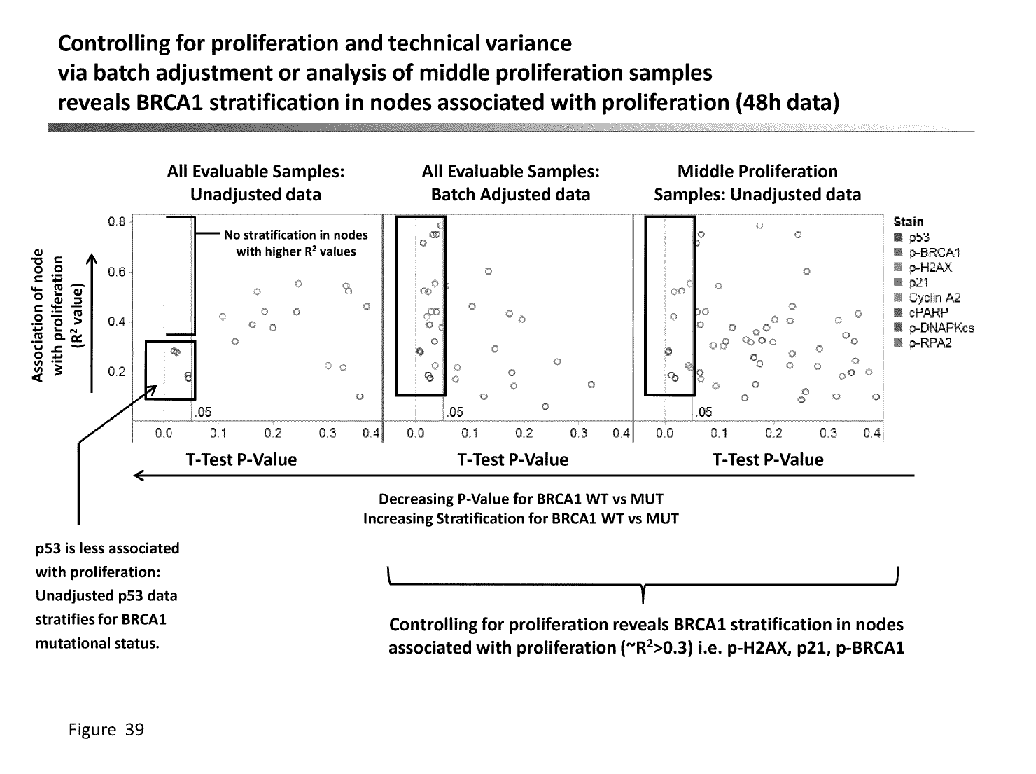

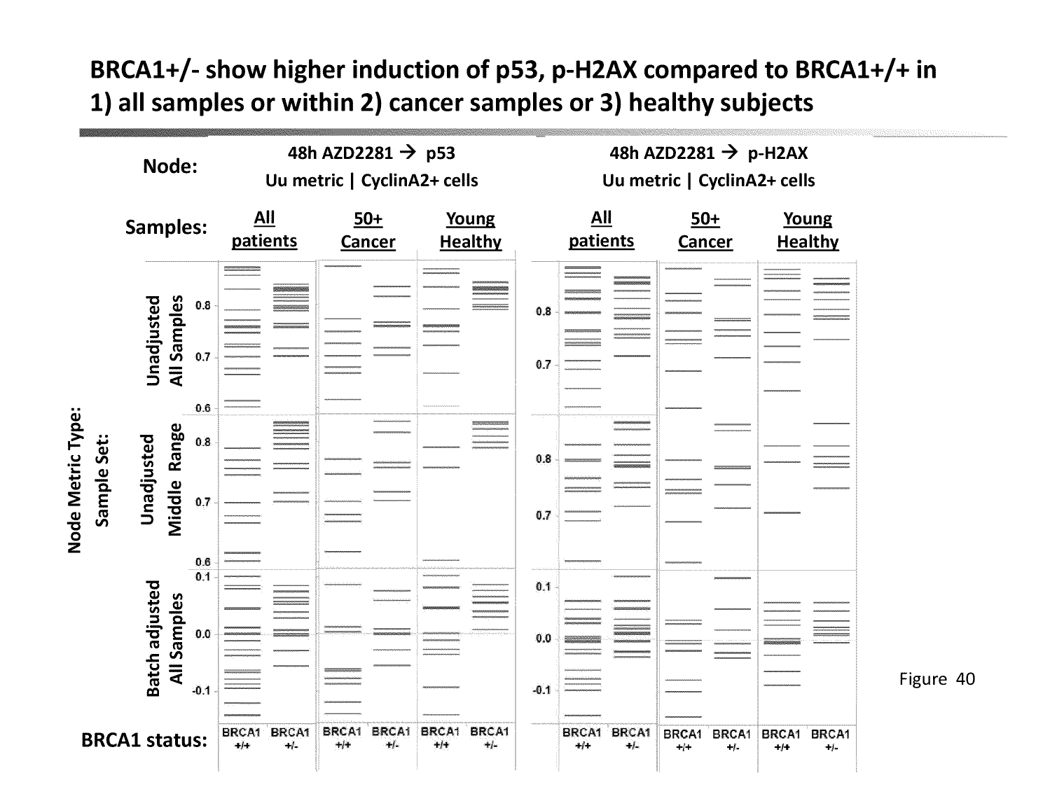

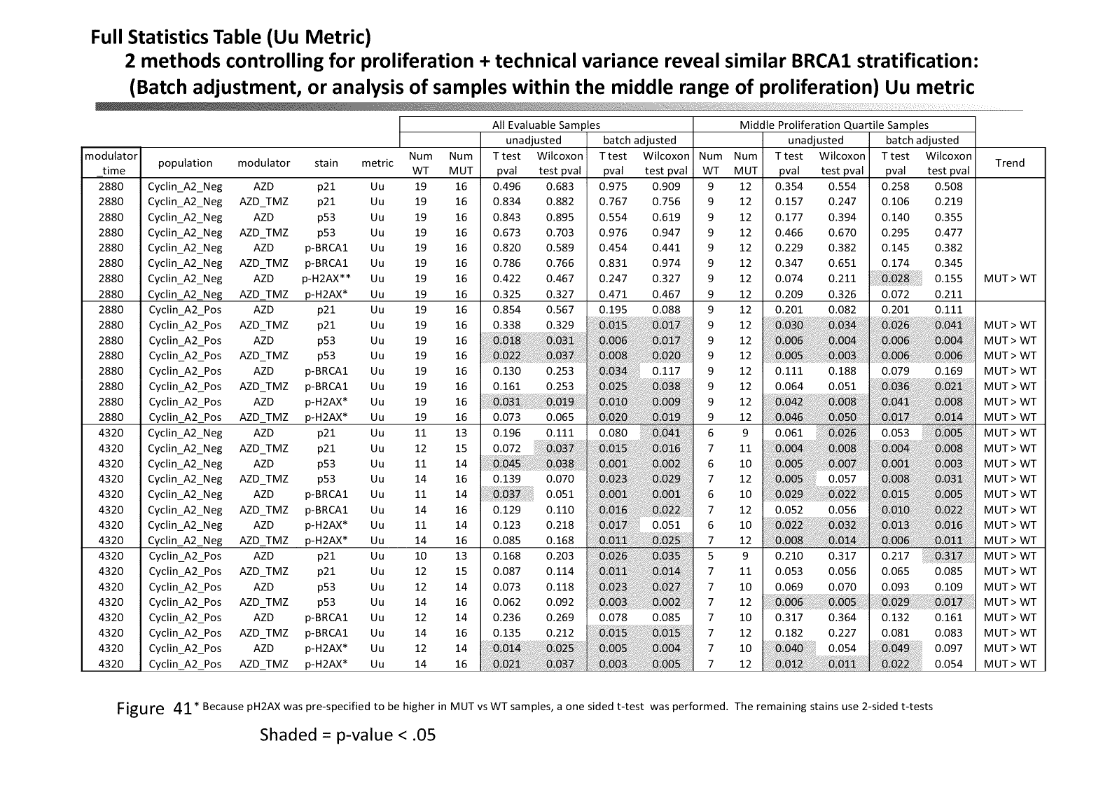

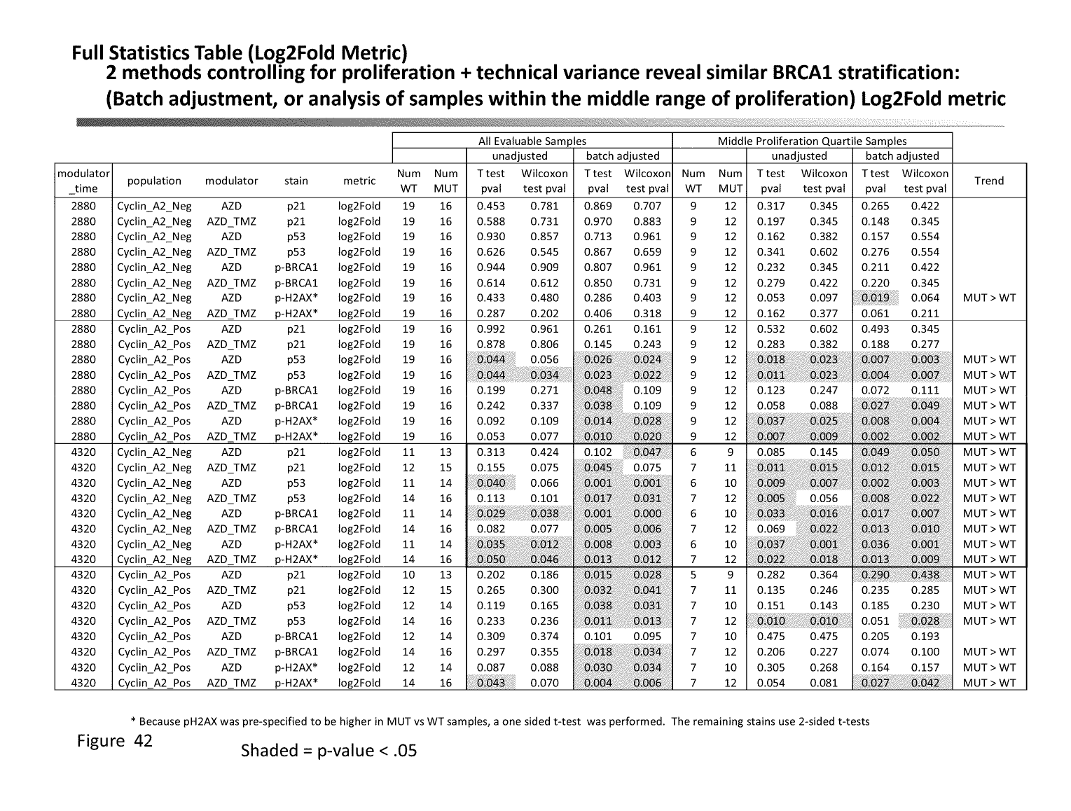

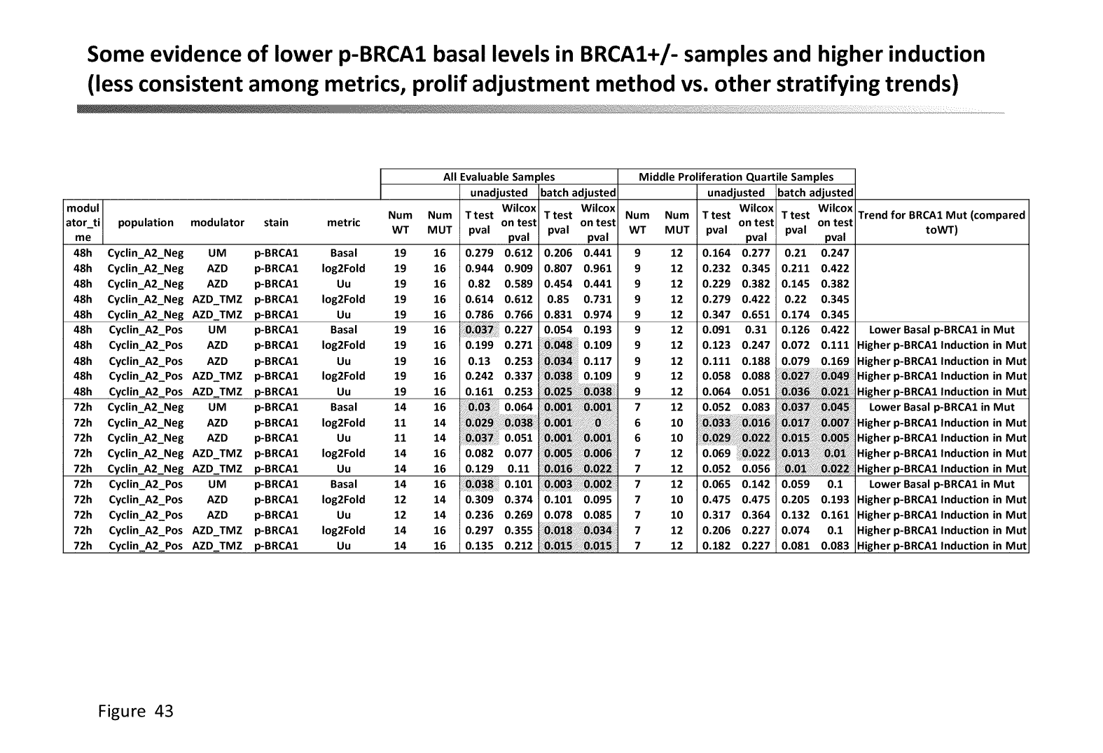

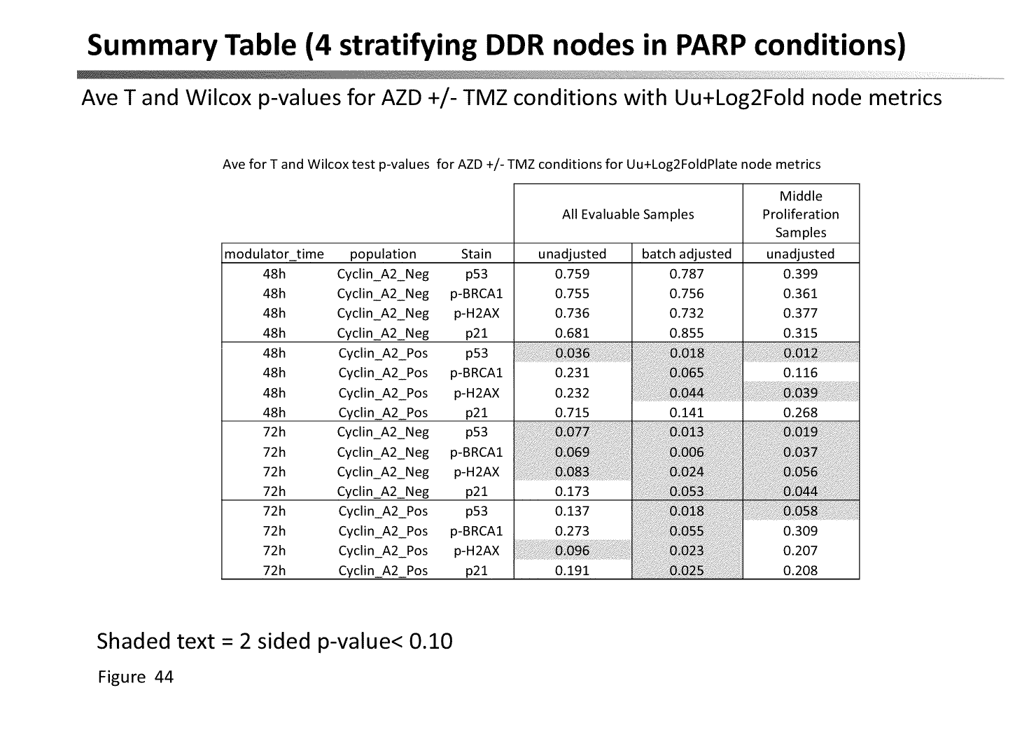

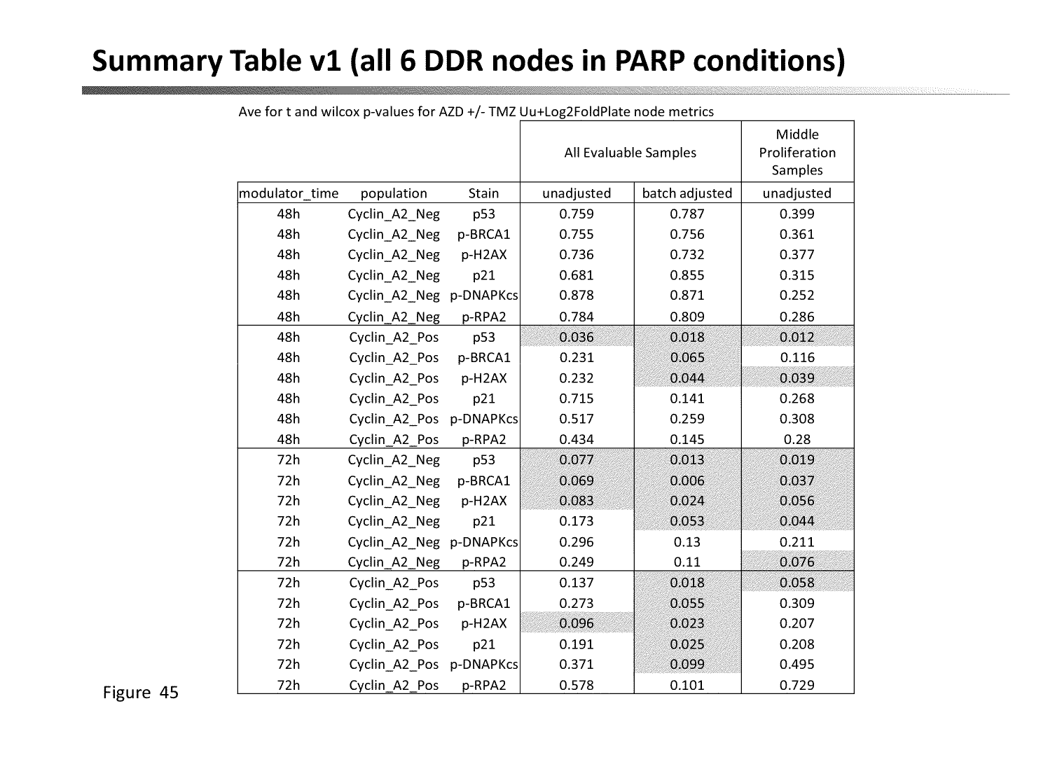

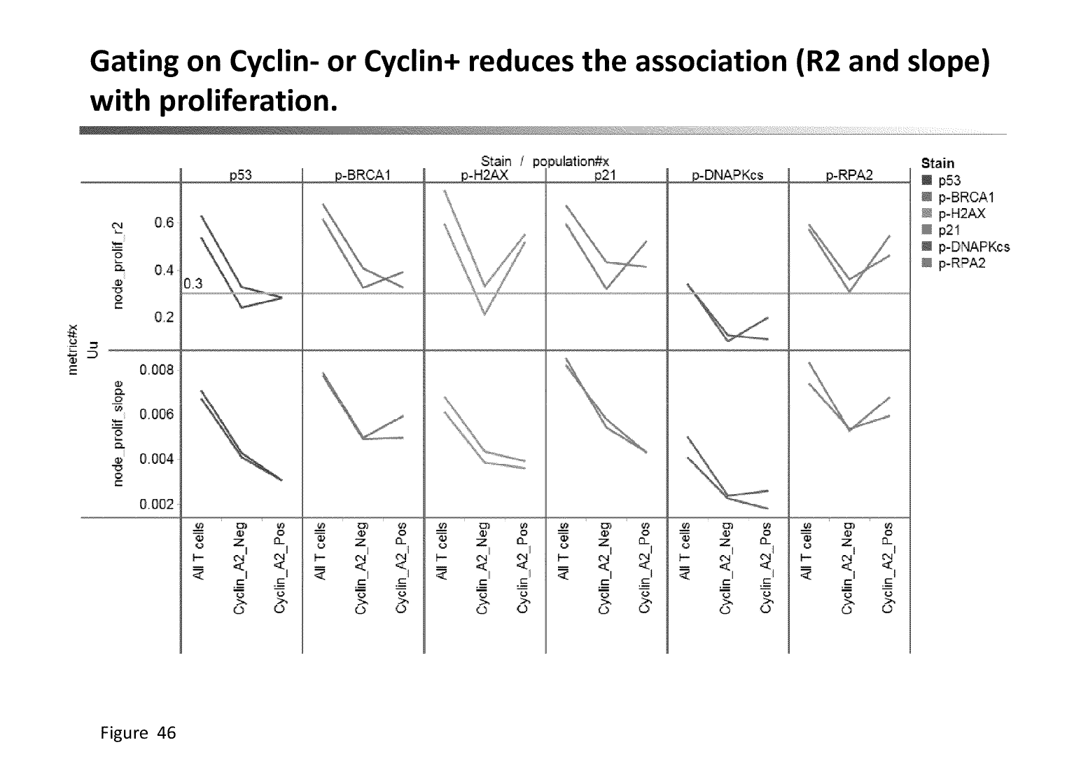

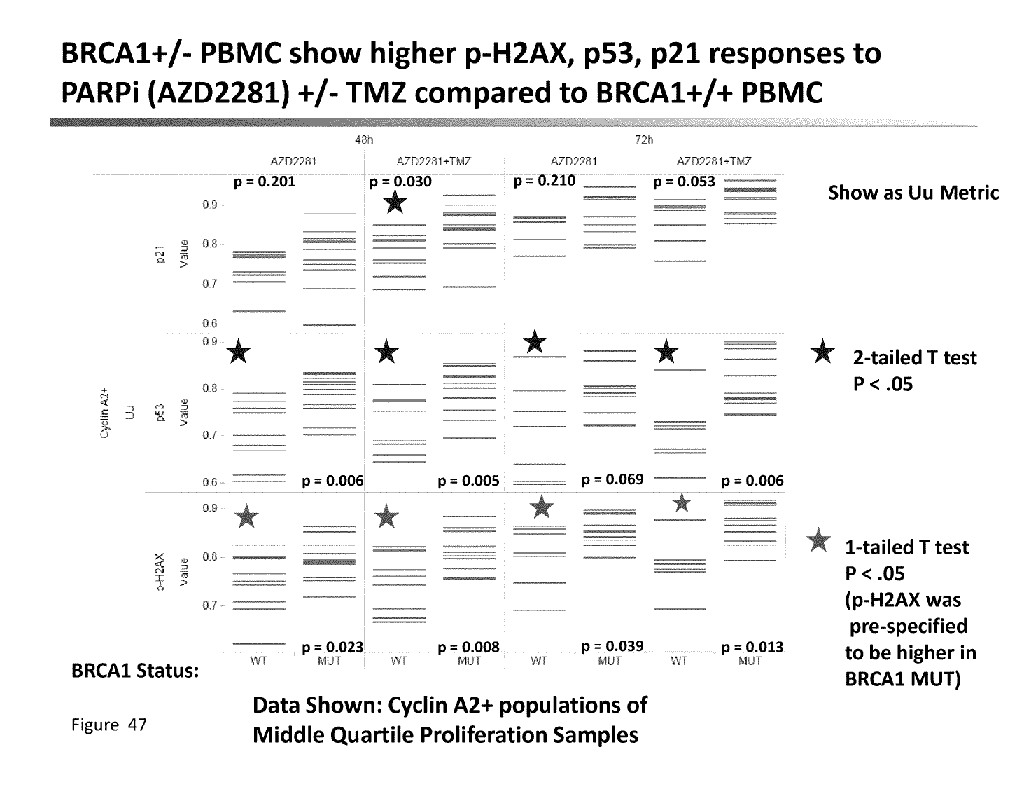

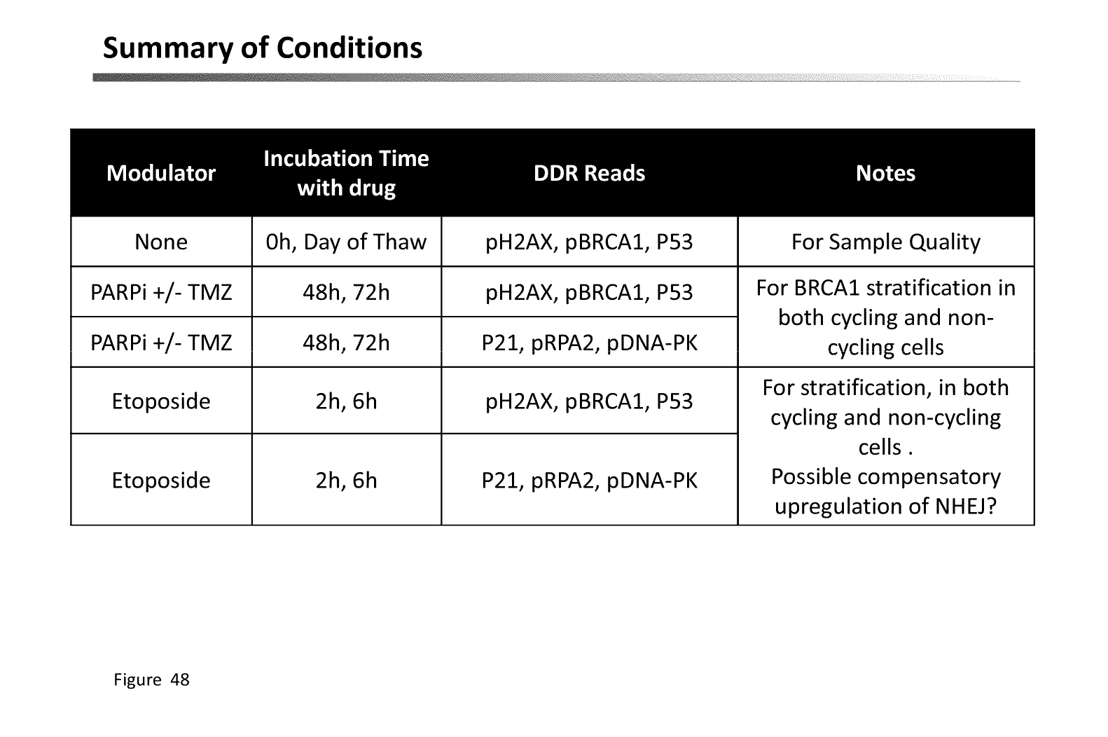

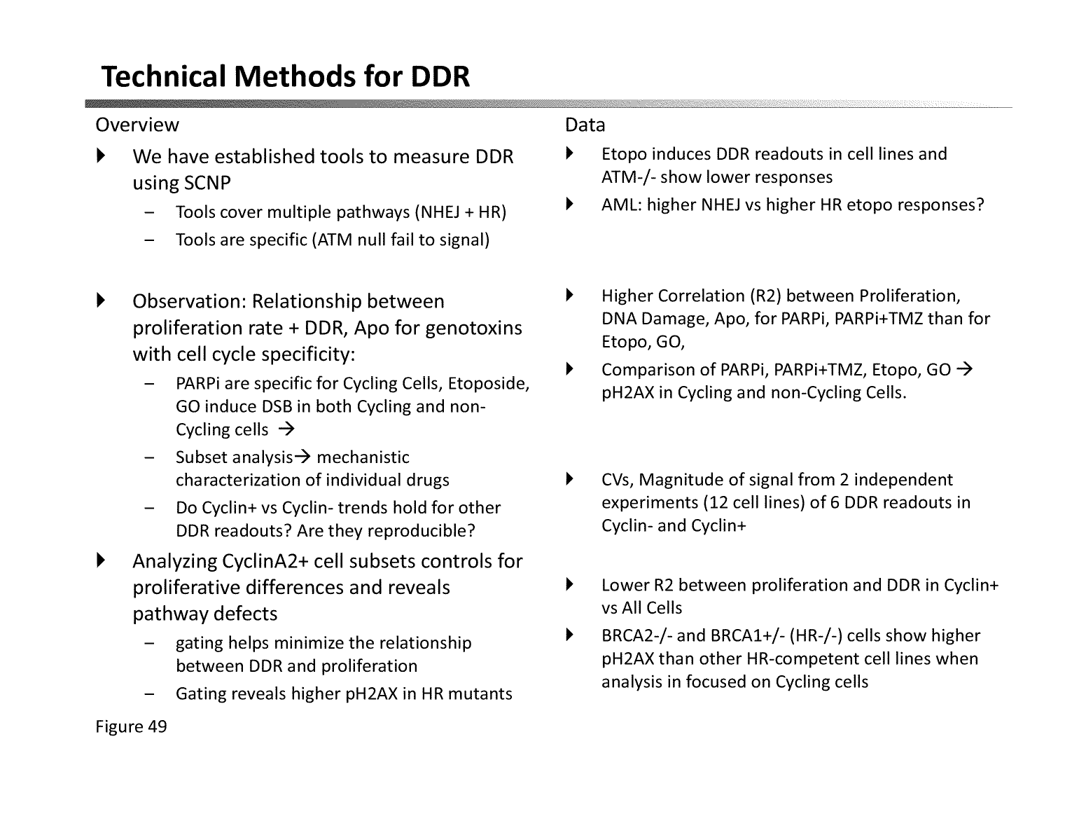

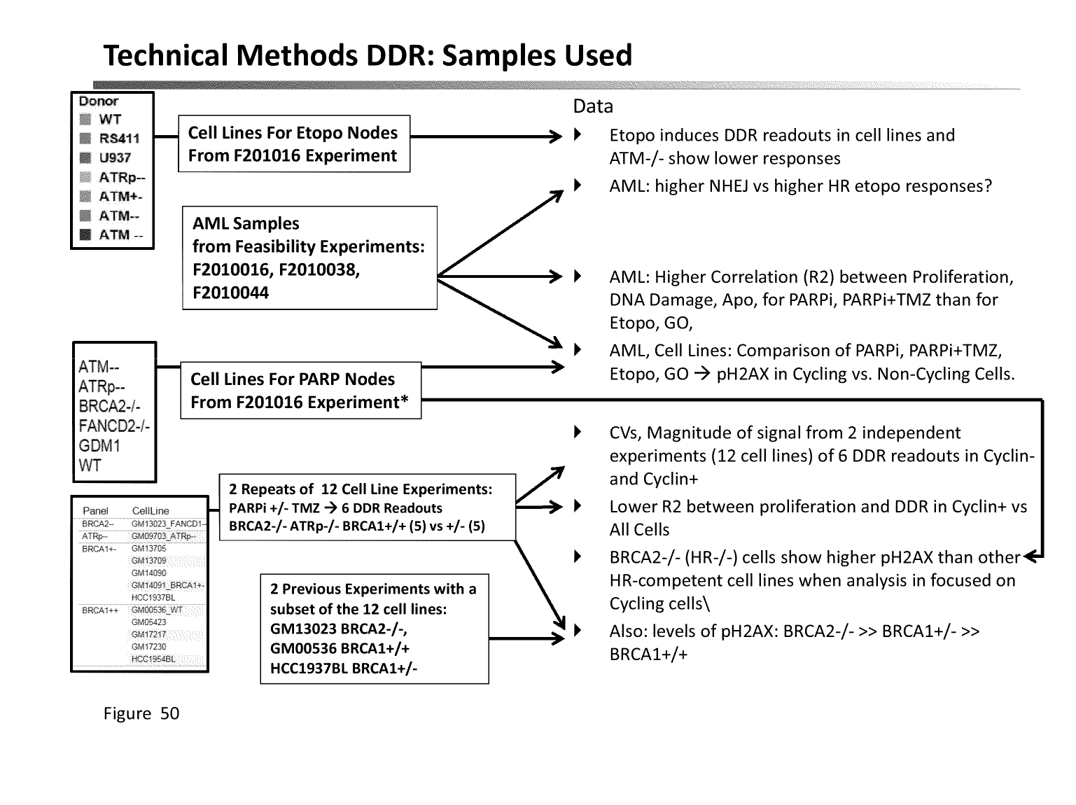

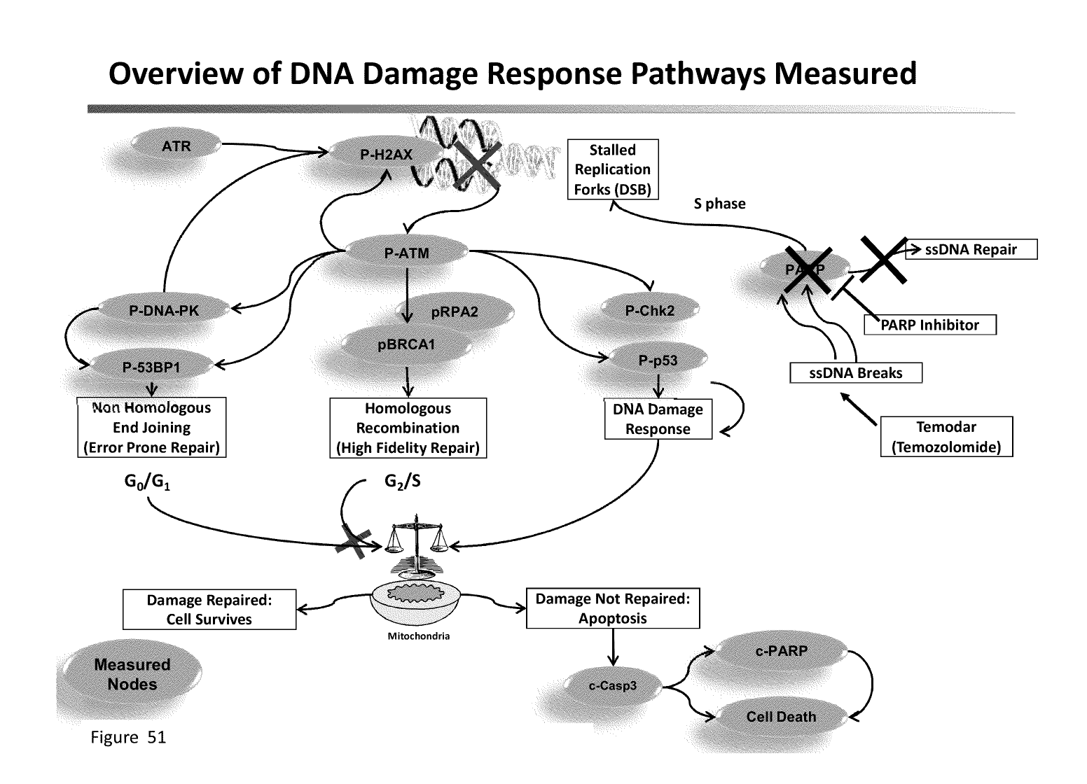

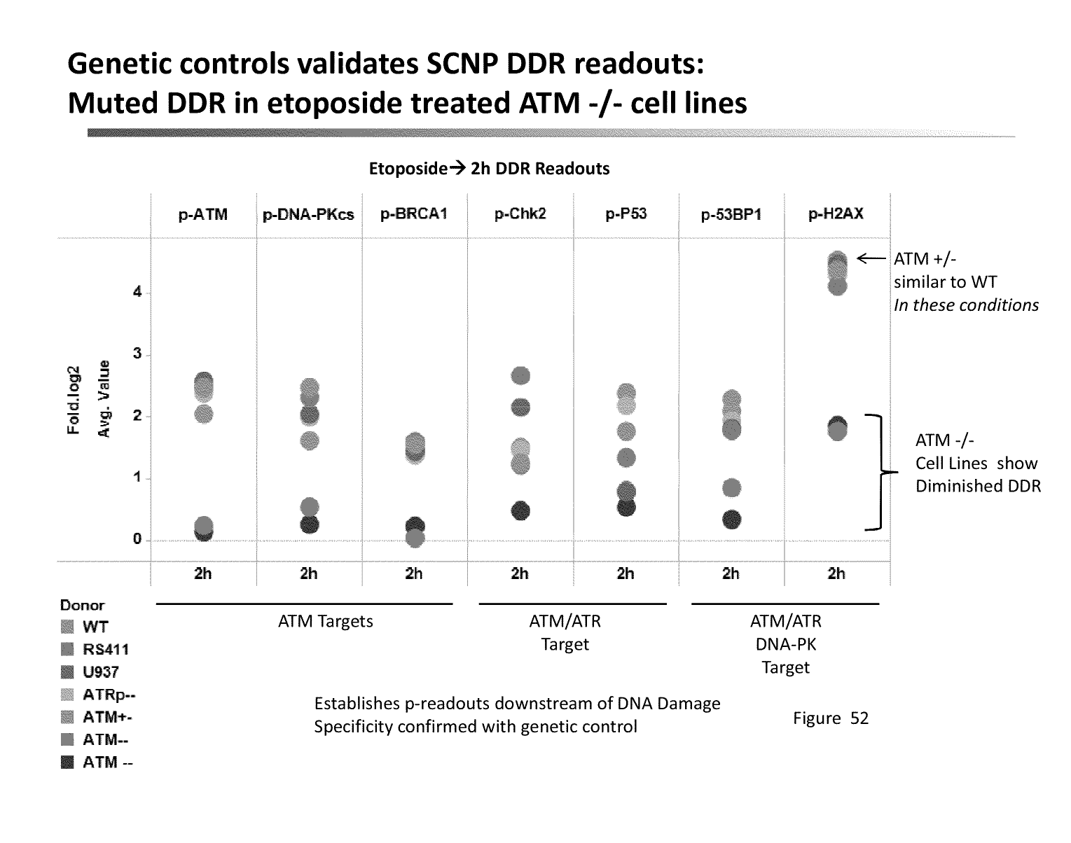

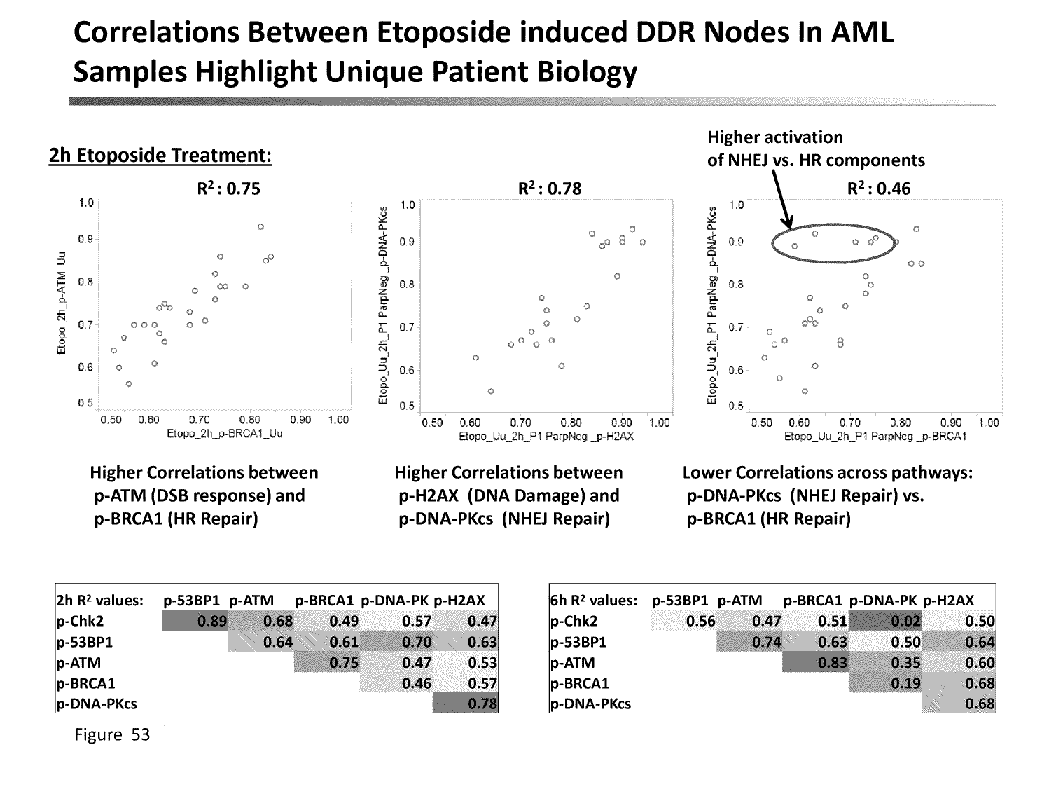

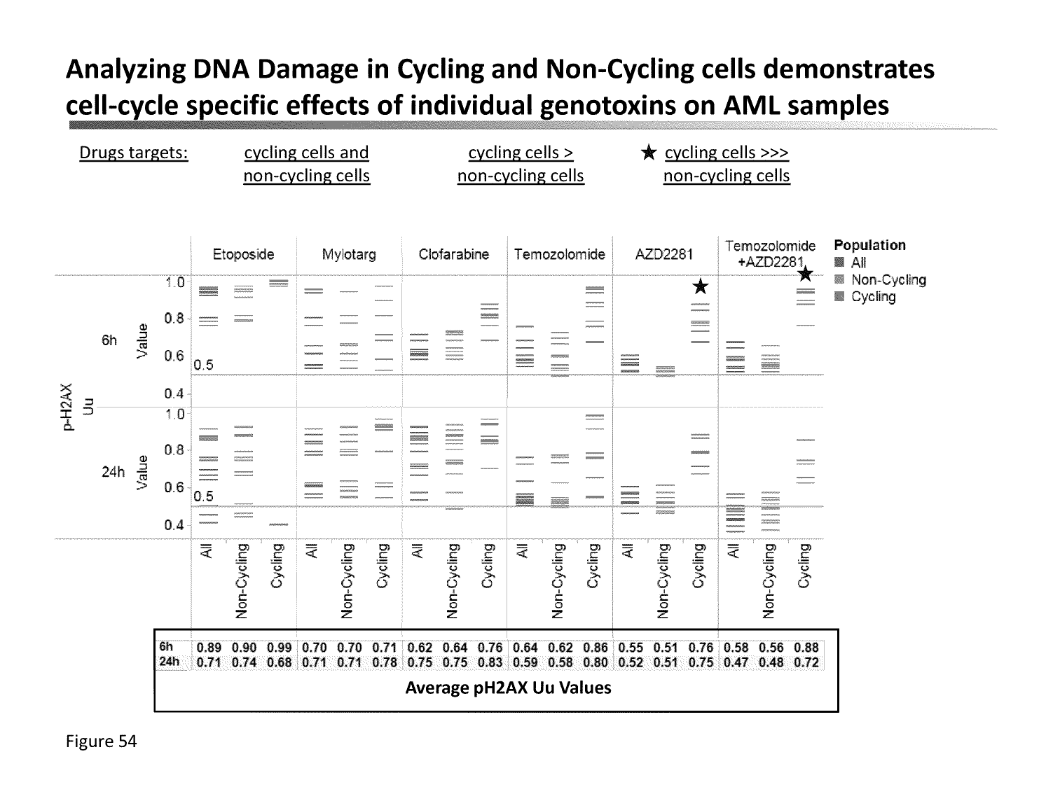

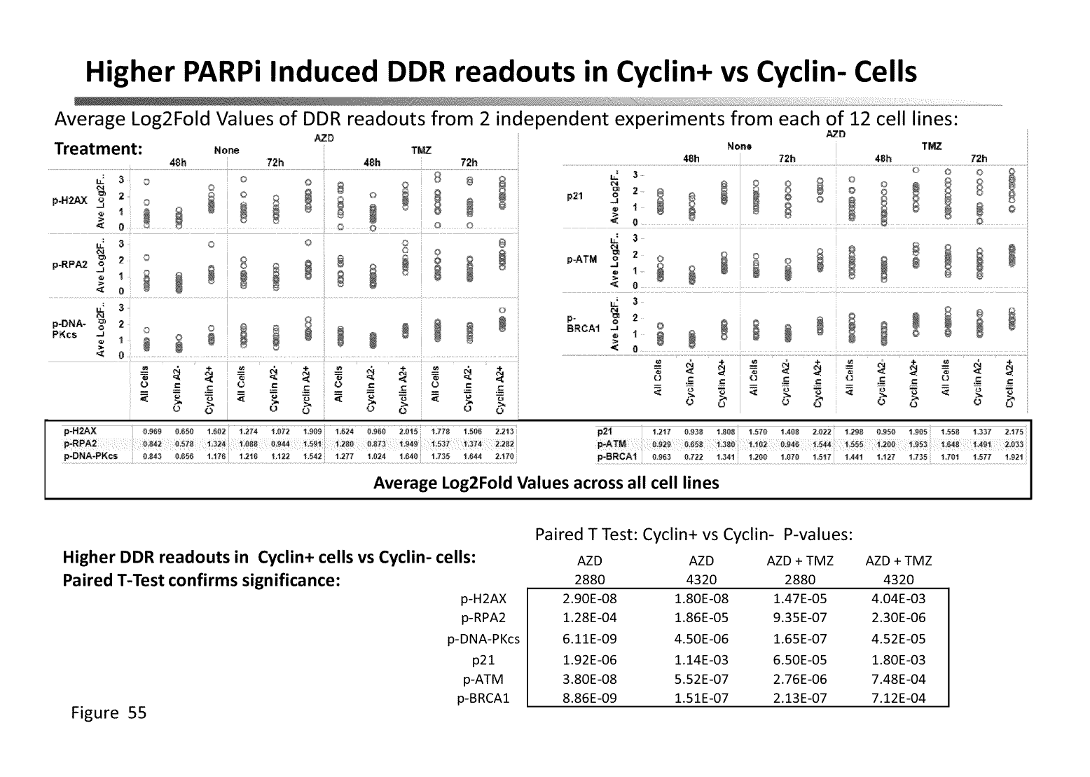

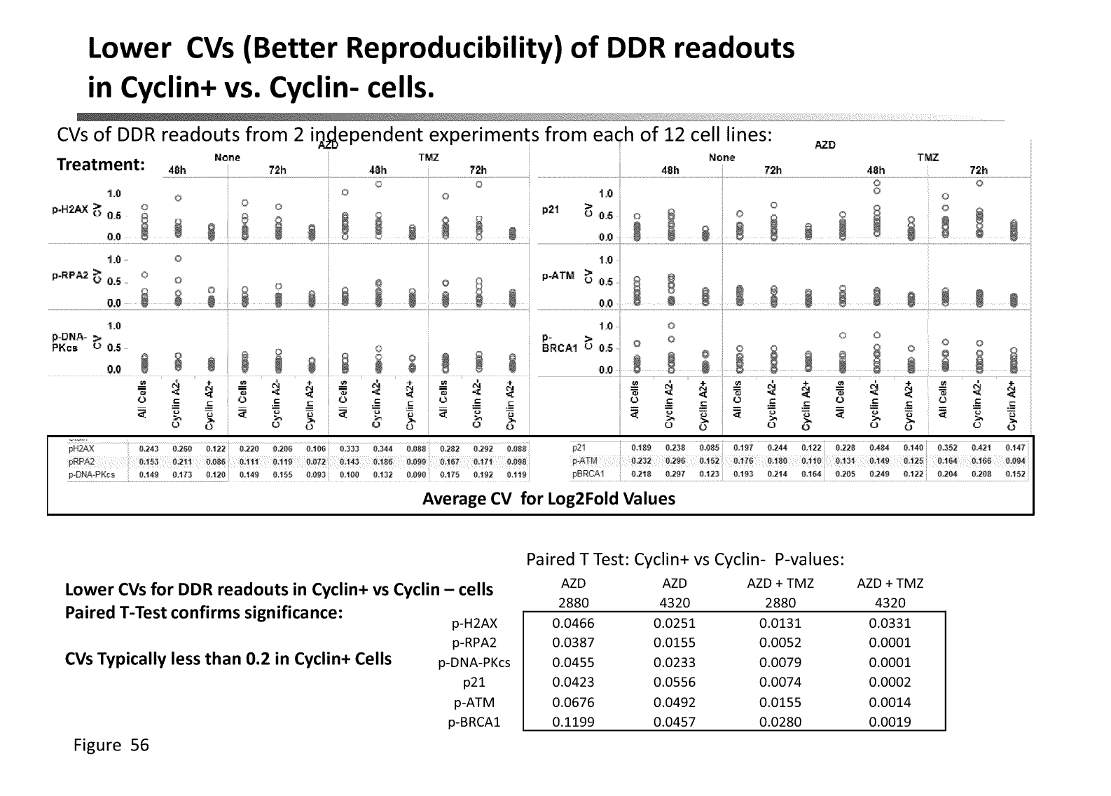

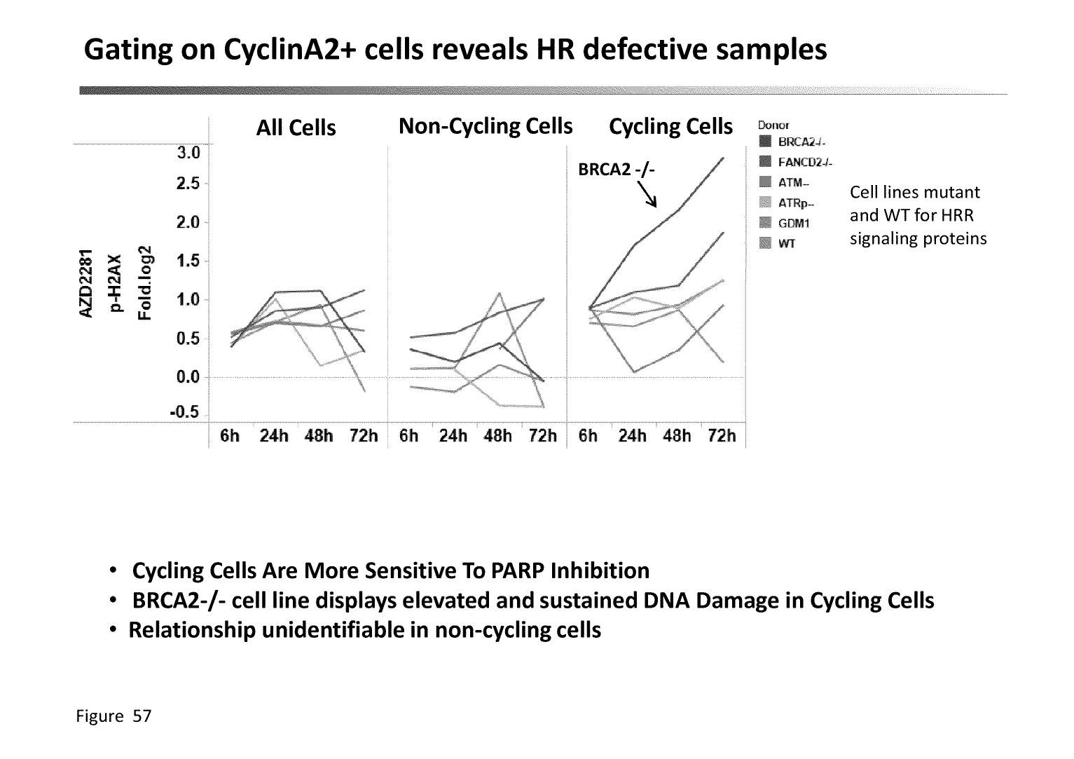

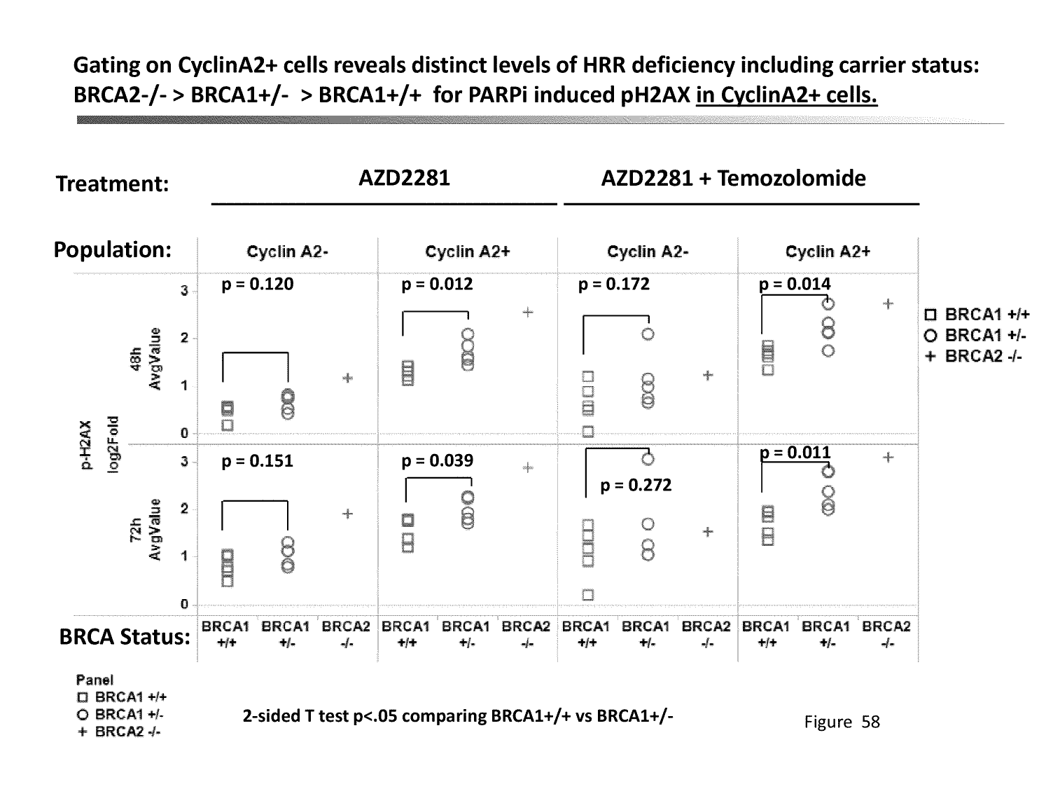

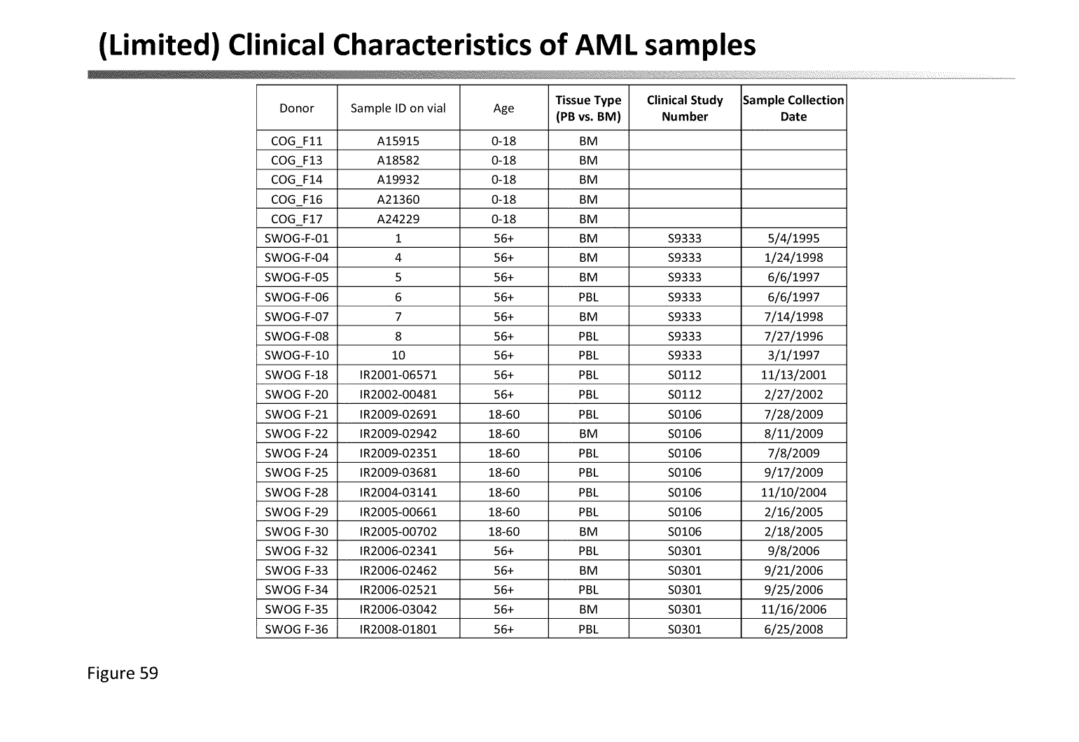

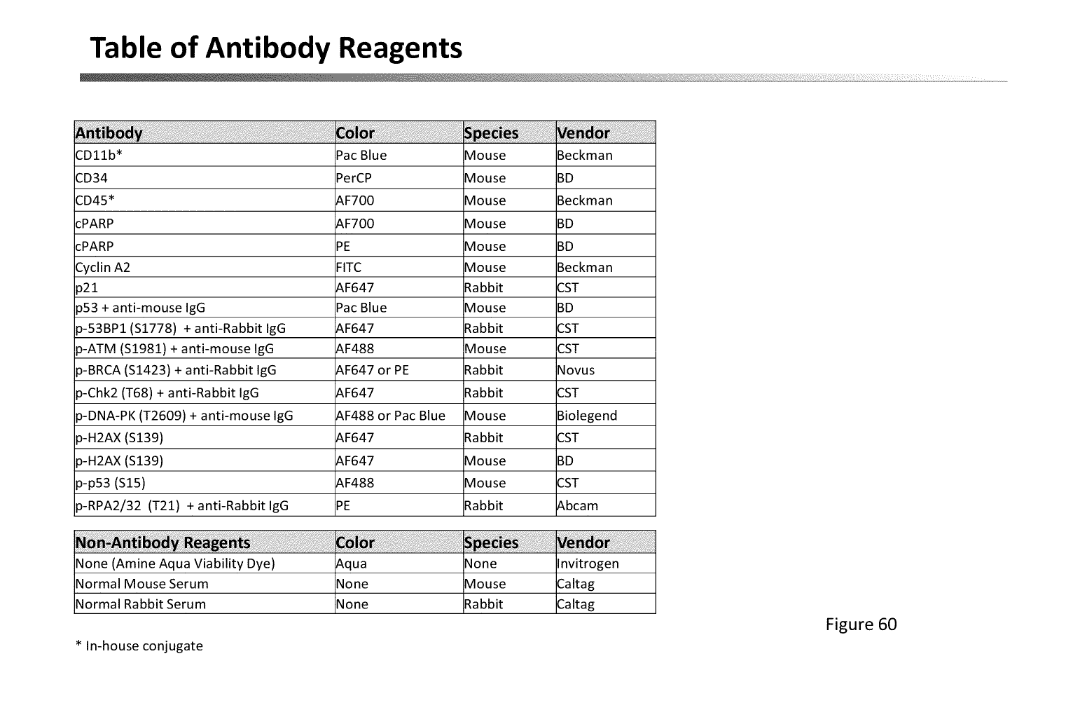

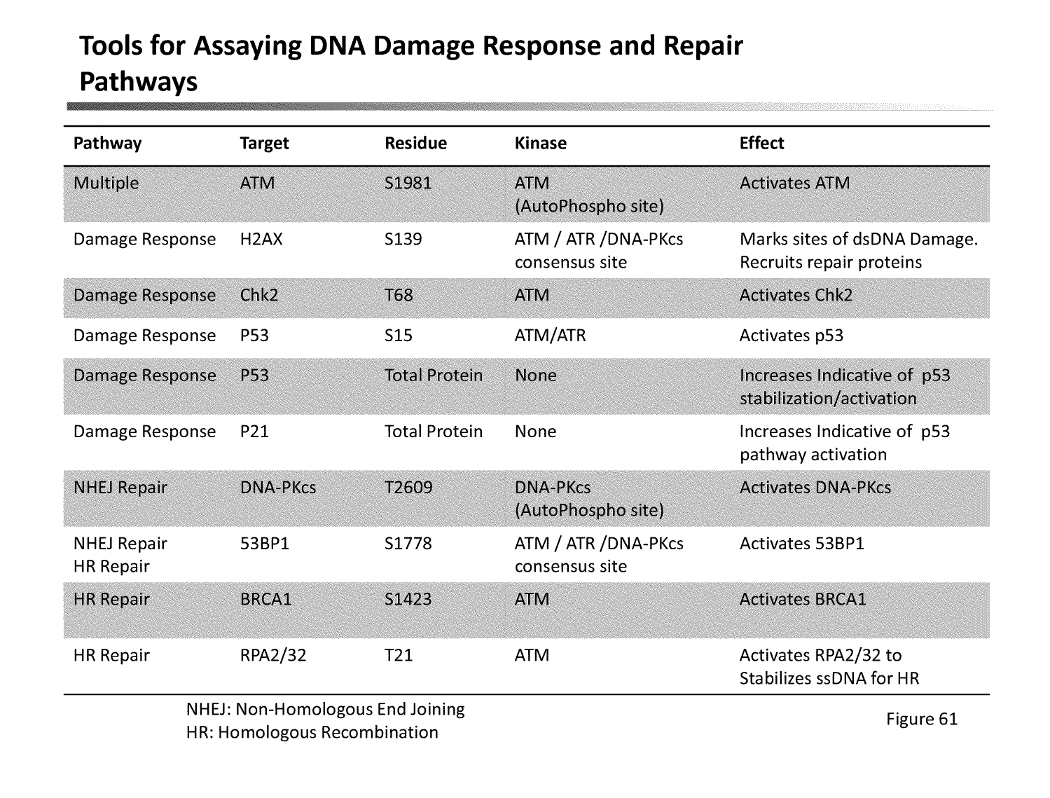

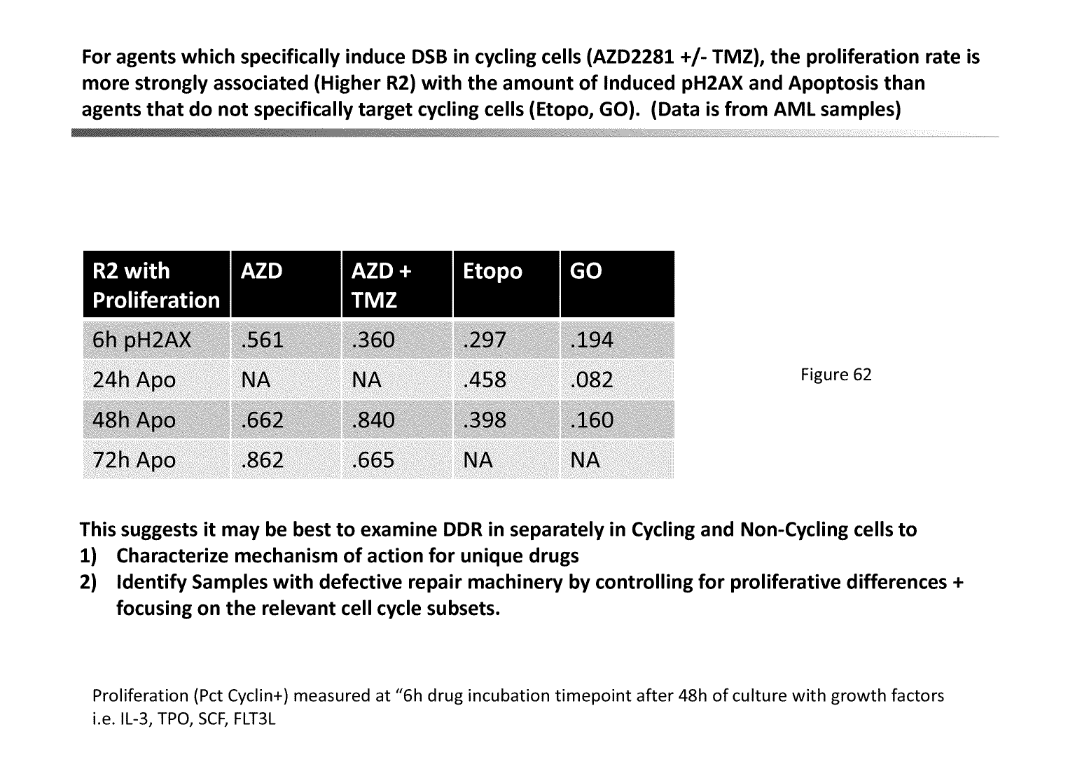

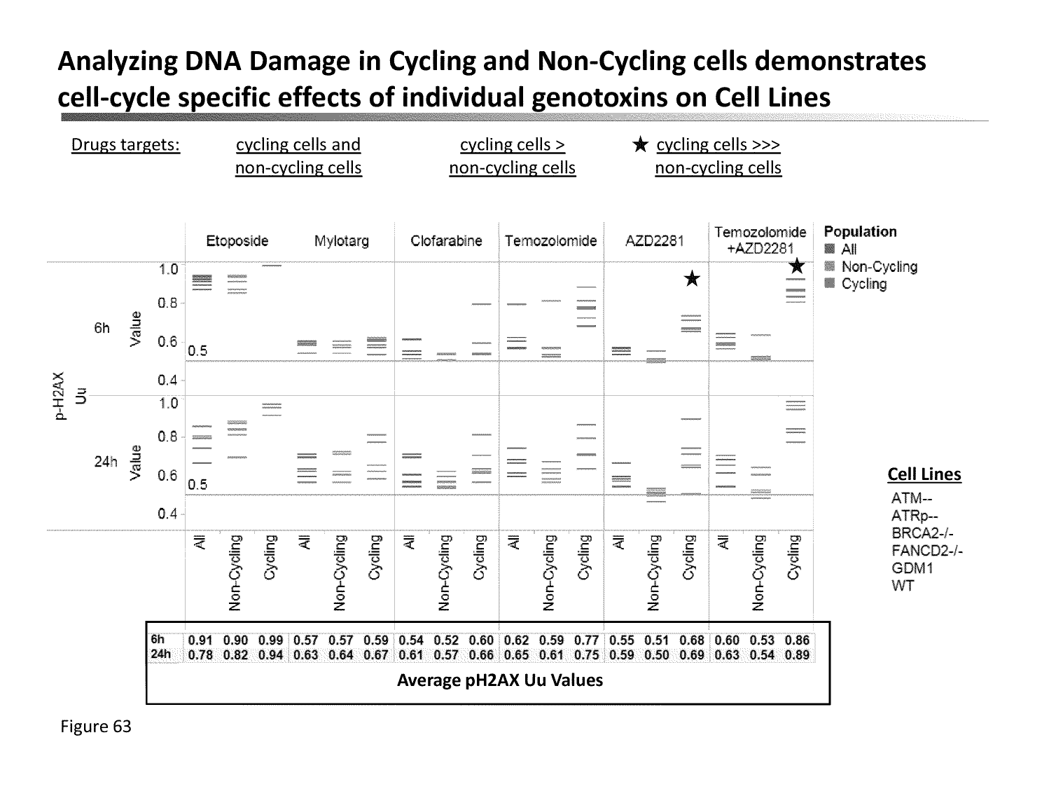

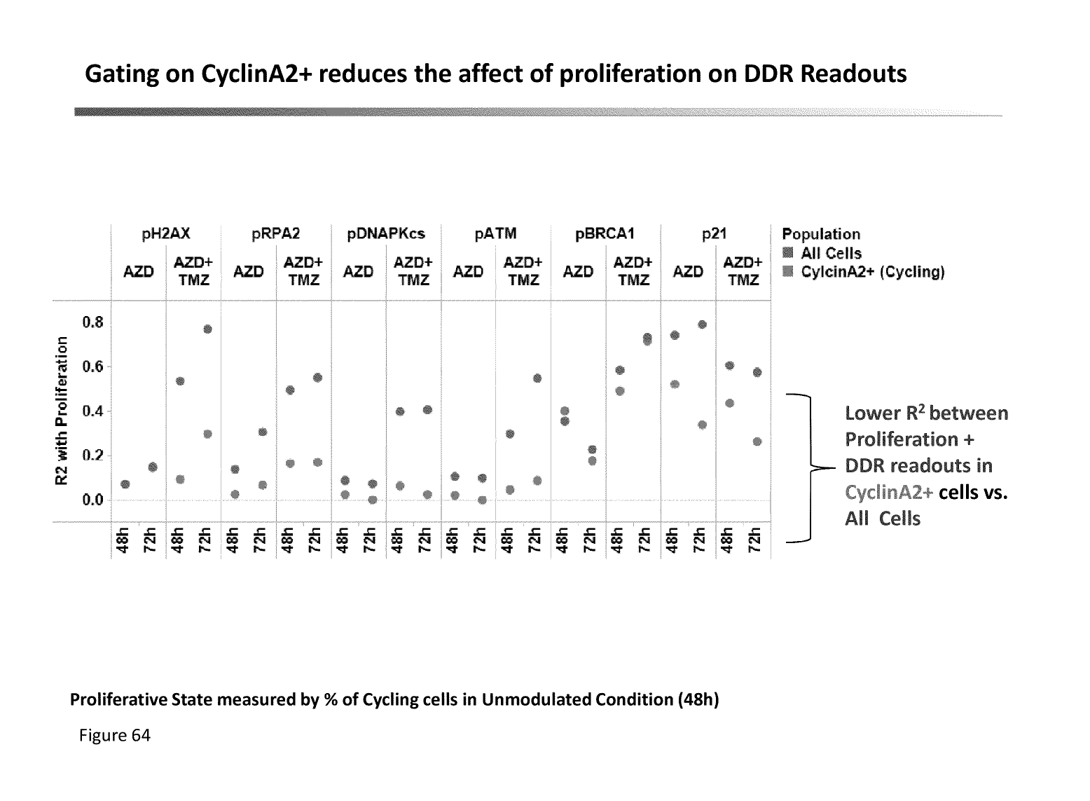

Correlations between DDR nodes among non-apoptotic cells in AML samples (2 h, 6 h) highlight unique patient biology that might indicate that some cells might favor a DNA damage repair pathway over others (FIG. 14). For example some patients' cells might favor NHEJ pathway over HR pathway or vice versa.

In summary, DDR nodes can identify etoposide sensitive AML. This point is summarized in table 6 below (X=not correlated with Etoposide induced Apoptosis. Check=correlated with Etoposide induced Apoptosis).

TABLE 6

| | 2 h Data identifies | 6 h Data identifies |
|---|---|---|
| DDR Readout | Sensitive Samples | Sensitive Samples |
| DSB DNA Damage (pH2AX) | ✓ | ✓ |
| DSB Damage Response (pATM) | X | X |
| HR Repair (pBRCA1) | ✓ | X |
| NHEJ Repair (DNA-PKcs) | ✓ | ✓ |

Cell lines GDM1 (Etopo Refractory) and RS411 (Etopo Sensitive) show similar patterns of DDR responses as AML samples (not shown). This method can be used to identify network relationships between DDR readouts and apoptosis. Among other things one can study: (i) kinetics of DDR responses, (ii) correlations between pathways, (iii) balance of NHEJ vs. HR pathways, and (iv) evaluate in the context of specific age groups, patients.

### Examination of the Sensitivities of AML Samples to PARP Inhibition, in Single Agent and Combination Studies

The methods described herein allow for the identification of PARP inhibitor (PARPi) sensitive samples, the correlation with node readouts—synthetic lethal mutations, and the identification of outliers and subgroups

FIG. 15 shows that treatment with PARP inhibitors induces DNA damage in cycling cells. Cells in S-phase or beyond, measured by the levels of Cyclin A2+, show PARP inhibitor induced DNA damage. Focusing on cycling cells reveals PARPi induced DNA damage in cell lines (FIG. 15 and data not shown). BRCA2−/− cell line displays elevated and sustained DNA damage in cycling cells. This relationship was unidentifiable in non-cycling cells (FIG. 16). FIG. 17 shows that adjusting apoptosis by % Cyclin+reveals PARPi induced apoptosis in HR defective cell lines. Since PARPi targets cycling cells and the individual samples have different percentages of cycling cells, there is a need to have ability to control for these differences to identify HR defective samples.

FIG. 18 shows that focusing on cycling cells reveal PARPi induced DNA damage: AML samples. FIG. 19 shows that SCNP detects AML samples sensitive to in vitro PARP inhibitor treatment.

Temodar (Temozolomide; TMZ) induces SSB and PARP repairs SSB. The combination of Temodar+PARPi amplifies DSBs in cycling cells. This combination treatment is useful for multiple purposes such as: (i) identification of HR defective samples, and (ii) groundwork for rational therapy combination. PARPi+Temodar combination induced apoptosis in cell lines (data not shown). There was an additive effect seen for PARPi+Temodar (Temozolomide). In addition, apoptosis results correlated with DNA Damage. FIG. 20 shows that PARPi+Temodar Combination induced apoptosis in AML samples and that there were unique patient trends of Temodar and PARP sensitivity observed. There were samples sensitive to Temodar alone, samples for which an additive effect was seen for PARPi+Temodar, and samples resistant to both PARPi+Temodar. FIG. 21 shows that SCNP can detect activation of multiple DDR pathways after PARP inhibition.

The methods described herein can be used to investigate ability to functionally identify HR defective/PARPi sensitive clinical samples. There is a significant clinical need and potential opportunity. These methods can be used to identify responsive populations to PARPi in triple negative breast cancer patients for example. Functional assessment provides opportunity to inform therapeutic selection, enriching for responsive patients with HR mutant disease. The methods also allow investigating the ability to functionally detect HR pathway mutations in clinical samples, ultimately to identify individuals at risk for tumor development. This will allow identifying functional HR pathway mutations that may be undetected by genomic studies.

### Example 4

### Germline Homologous Recomination (HR) Deficiency

This example is an overview of the samples tested in Example 5. Inherited alterations in BRCA1/2 genes increase genomic instability and cancer susceptibility. DNA sequencing detects BRCA1/2 mutations, but has the following limitations; 1) mutations may have unknown functional significance, 2) epigenetic alterations and mutations in other Homologous Recombination (HR) pathway components are not detected, and 3) the combined effects of pathway mutations are not understood. Thus a functional assessment of HR competence at the single cell level remains is still an unmet need as BRCA1/2 sequencing does not holistically inform on functionality of the HR pathway. Single Cell Network Profiling (SCNP) is a multiparametric flow cytometry-based assay that simultaneously measures, at the single cell level, extracellular surface markers and functional changes in intracellular signaling in response to extracellular modulators (S. M. Kornblau, M. D. Minden, D. B. Rosen, S. Putta, A. Cohen, T. Covey, D. C. Spellmeyer, W. J. Fantl, U. Gayko, A. Cesano, Dynamic single-cell network profiles in acute myelogenous leukemia are associated with patient response to standard induction therapy, Clinical cancer research: an official journal of the American Association for Cancer Research, 16 (2010) 3721-3733). In this study, we tested the ability of SCNP to detect and quantify functional changes in HR signaling using peripheral blood mononuclear cell (PBMC) samples from BRCA1 mutation carriers (MUT) and wild type (WT) subjects.

Methods: HR pathway activity was examined in PBMCs from BRCA1 MUT (n=21) or WT (n=20) subjects. Cell lines carrying BRCA MUT or WT genes were used as controls. PBMCs were stimulated with anti-CD3 and anti-CD28 for 24 hours to induce T cell proliferation then treated with PARP inhibitor (PARPi) AZD2281+/−Temozolomide (TMZ) for 48 h or 72 h to induce DNA damage. DDR responses were measured in both CyclinA2− and CyclinA2+ T cell subsets. Measurements included induced levels of p21, p53 and phosphorylation (p-) of p-H2AX, p-DNA-PKcs, p-RPA2/32, and p-BRCA1.

Results: As expected based on the mechanism of action of PARPi, higher levels of induced p-H2AX and p53 were observed in CyclinA2+ cells of BRCA1 MUT versus WT cell line controls. In PBMCs, T cell proliferation (% CyclinA2+) was positively associated with PARPi induced DDR readouts. After controlling for proliferation, statistically significant differences in PARPi induced DDR signaling were observed between BRCA1 MUT and WT samples in many simultaneously assessed readouts including p-H2AX, p53 and p21 (increased in MUT), particularly in CyclinA2+ cells. MUT BRCA1 samples displayed lower basal p-BRCA1 but higher induced p-BRCA1 levels compared to BRCA WT samples.

Conclusions: SCNP is able to detect and quantify functional differences between PBMC samples from BRCA1 MUT (haploinsufficient) and WT donors by quantitatively assessing DDR signaling in CyclinA2+ T cells. Once verified on a larger data set, the assay could form the basis for the development of screening tests to identify subjects at higher risk of developing cancer or stratification tests to inform on cancer patient selection for treatment with PARP inhibitors.

### Example 5

### HR Deficiences in BRCA 1

FIGS. 22 to 49 are relevant to this example. Background: Inherited alterations in BRCA1/2 increase genomic instability and cancer susceptibility. Molecular sequencing can detect BRCA1/2 mutations, however the value of these data is limited as 1) many detected mutations are of unknown functional significance, 2) non-coding BRCA1/2 alterations and genetic/epigenetic alterations in other Homologous Recombination Repair (HR) pathway components are not detected, and 3) the combined effects of any detected mutations are not understood. To assess the functional impact of alterations in the entire HR pathway, a functional assessment of HR competence is needed. Here we test the principle of detecting functional changes in the HR signaling network, by successfully applying SCNP to detect germline mutations in the BRCA1 gene using peripheral blood mononuclear cells (PBMCs) from BRCA1 mutation carriers.

The DNA damage response (DDR), through the action of sensors, transducers and effectors, orchestrates the appropriate recognition, repair of DNA damage and resolution of stalled DNA replication. This process is coordinated through complex interplay with the cell cycle, apoptosis, ubiquitination and cell survival signaling. Two major mechanisms for the repair of DNA double strand (ds) breaks are homologous recombination repair and non-homologous end joining (HR and NHEJ, respectively). HR predominates in cells that are replicating DNA and is less-error prone as HR uses the identical homologous sister chromatid as a sequence template to repair DNA ds breaks [Kass E M, Jasin M (2010) Collaboration and competition between DNA double-strand break repair pathways. FEBS Letters 584: 3703-3708.1]. NHEJ, which is predominately used in resting cells in the G0/G1 phases of the cell cycle, is a more error-prone mechanism of repairing DNA ds breaks via ligation of DNA ends without a template [Kass E M, Jasin M (2010) Collaboration and competition between DNA double-strand break repair pathways. FEBS Letters 584: 3703-3708 and Mladenov E, Iliakis G (2011) Induction and repair of DNA double strand breaks: the increasing spectrum of non-homologous end joining pathways. Mutation research 711: 61-72.].

BRCA1 and BRCA2 are genes coding for DNA repair proteins involved in HR [Tutt A, Ashworth A (2002) The relationship between the roles of BRCA genes in DNA repair and cancer predisposition. Trends in molecular medicine 8: 571-576; 4. Powell S N, Kachnic L A (2003) Roles of BRCA1 and BRCA2 in homologous recombination, DNA replication fidelity and the cellular response to ionizing radiation. Oncogene 22: 5784-5791; and 5. Liu Y, West S C (2002) Distinct functions of BRCA1 and BRCA2 in double-strand break repair. Breast cancer research: BCR 4: 9-13.]. The majority (approximately 84%) of hereditary breast and ovarian cancer results from inherited germline mutations in BRCA1 (52%) and BRCA2 (32%) [Ford D, Easton D F, Stratton M, Narod S, Goldgar D, et al. (1998) Genetic heterogeneity and penetrance analysis of the BRCA1 and BRCA2 genes in breast cancer families. The Breast Cancer Linkage Consortium. American journal of human genetics 62: 676-689 and Frank T S, Deffenbaugh A M, Reid J E, Hulick M, Ward B E, et al. (2002) Clinical characteristics of individuals with germline mutations in BRCA1 and BRCA2: analysis of 10,000 individuals. Journal of clinical oncology: official journal of the American Society of Clinical Oncology 20: 1480-1490]. The risk of ovarian cancer due to inherited BRCA1 mutations is 28% [Whittemore A S, Gong G, Itnyre J (1997) Prevalence and contribution of BRCA1 mutations in breast cancer and ovarian cancer: results from three U.S. population-based case-control studies of ovarian cancer. American journal of human genetics 60: 496-504] to 44% [Ford D, Easton D F, Bishop D T, Narod S A, Goldgar D E (1994) Risks of cancer in BRCA1-mutation carriers. Breast Cancer Linkage Consortium. Lancet 343: 692-695] by age 70, compared to the general population risk of <1%. Germline mutations in BRCA1 and BRCA2 are associated with a 45% to 87% risk of breast cancer by age 70 [Ford D, Easton D F, Stratton M, Narod S, Goldgar D, et al. (1998) Genetic heterogeneity and penetrance analysis of the BRCA1 and BRCA2 genes in breast cancer families. The Breast Cancer Linkage Consortium. American journal of human genetics 62: 676-689, Ford D, Easton D F, Bishop D T, Narod S A, Goldgar D E (1994) Risks of cancer in BRCA1-mutation carriers. Breast Cancer Linkage Consortium. Lancet 343: 692-695 and Antoniou A, Pharoah P D, Narod S, Risch H A, Eyfjord J E, et al. (2003) Average risks of breast and ovarian cancer associated with BRCA1 or BRCA2 mutations detected in case Series unselected for family history: a combined analysis of 22 studies. American journal of human genetics 72: 1117-1130.]. Most importantly, hereditary breast cancer occurs at a far earlier age than the nonhereditary (sporadic) form.

In BRCA1-mutation carriers, loss of heterozygosity at the BRCA1 locus impairs DNA double-strand break repair by HR. This promotes the use of error-prone mechanisms such as non-homologous end joining (NHEJ) to repair DNA double strand breaks that arise during replication, eventually resulting in DNA deletions or translocations (genomic instability).

A high percentage (70-89%) of triple negative breast cancers (lacking estrogen, progesterone and Her-2 receptors) harbor mutations in BRCA1 [Anders C K, Carey L A (2009) Biology, metastatic patterns, and treatment of patients with triple-negative breast cancer. Clin Breast Cancer 9 Suppl 2: 573-81; Atchley D P, Albarracin C T, Lopez A, Valero V, Amos C I, et al. (2008) Clinical and pathologic characteristics of patients with BRCA-positive and BRCA-negative breast cancer. J Clin Oncol 26: 4282-4288; Dawson S J, Provenzano E, Caldas C (2009) Triple negative breast cancers: clinical and prognostic implications. Eur J Cancer 45 Suppl 1: 27-40; Rowe D L, Ozbay T, O'Regan R M, Nahta R (2009) Modulation of the BRCA1 protein and induction of apoptosis in triple negative breast cancer cell lines by the polyphenolic compound curcumin. Breast Cancer 3: 61-75.] and are non-responsive to hormonal therapy or Herceptin treatment [Arslan C, Dizdar O, Altundag K (2009) Pharmacotherapy of triple-negative breast cancer. Expert Opin Pharmacother 10: 2081-2093; Breuer A, Kandel M, Fisseler-Eckhoff A, Sutter C, Schwaab E, et al. (2007) BRCA1 germline mutation in a woman with metaplastic squamous cell breast cancer. Onkologie 30: 316-318; Collins L C, Martyniak A, Kandel M J, Stadler Z K, Masciari S, et al. (2009) Basal cytokeratin and epidermal growth factor receptor expression are not predictive of BRCA1 mutation status in women with triple-negative breast cancers. Am J Surg Pathol 33: 1093-1097; Reis-Filho J S, Tutt A N (2008) Triple negative tumours: a critical review. Histopathology 52: 108-118.]. Associated deficits in the DDR render these tumors particularly sensitive to targeted therapies, such as PARP inhibitors, which significantly increase the amount of DNA damage requiring BRCA/HR mediated repair and result in substantial error-prone NHEJ mediated repair of DNA damage in the presence of dysfunctional BRCA/HR [Rottenberg S, Jaspers J E, Kersbergen A, van der Burg E, Nygren A O, et al. (2008) High sensitivity of BRCA1-deficient mammary tumors to the PARP inhibitor AZD2281 alone and in combination with platinum drugs. Proc Natl Acad Sci USA 105: 17079-17084] Helleday Mol Oncol 2011, Patel A G, Kaufman S H PNAS 2011. Currently individuals with a family history of breast cancer are tested for the presence of BRCA1 and 2 mutations using a molecular test and if positive they are provided information regarding the option to undergo preventive bilateral mastectomy and oophorectomy [Burke W, Daly M, Garber J, Botkin J, Kahn M J, et al. (1997) Recommendations for follow-up care of individuals with an inherited predisposition to cancer. II. BRCA1 and BRCA2. Cancer Genetics Studies Consortium. Jama 277: 997-1003; Friebel T M, Domchek S M, Neuhausen S L, Wagner T, Evans D G, et al. (2007) Bilateral prophylactic oophorectomy and bilateral prophylactic mastectomy in a prospective cohort of unaffected BRCA1 and BRCA2 mutation carriers. Clin Breast Cancer 7: 875-882; and. Ray J A, Loescher L J, Brewer M (2005) Risk-reduction surgery decisions in high-risk women seen for genetic counseling. J Genet Couns 14: 473-484; Rebbeck T R (2002) Prophylactic oophorectomy in BRCA1 and BRCA2 mutation carriers. Eur J Cancer 38 Suppl 6: S15-17.]. However, 11-30% of breast cancer patients have triple negative tumors and do not have germ line alterations in the BRCA1 gene, [Anders C K, Carey L A (2009) Biology, metastatic patterns, and treatment of patients with triple-negative breast cancer. Clin Breast Cancer 9 Suppl 2: S73-81; and Atchley D P, Albarracin C T, Lopez A, Valero V, Amos C I, et al. (2008) Clinical and pathologic characteristics of patients with BRCA-positive and BRCA-negative breast cancer. J Clin Oncol 26: 4282-4288.] but their tumors are phenotypically similar in terms of biology and therapeutic response to those in patients carrying germ line alterations in the BRCA1 gene, [Reis-Filho J S, Tutt A N (2008) Triple negative tumours: a critical review. Histopathology 52: 108-118; Alli E, Sharma V B, Sunderesakumar P, Ford J M (2009) Defective repair of oxidative DNA damage in triple-negative breast cancer confers sensitivity to inhibition of poly(ADP-ribose) polymerase. Cancer Res 69: 3589-3596; and Diaz L K, Cryns V L, Symmans W F, Sneige N (2007) Triple negative breast carcinoma and the basal phenotype: from expression profiling to clinical practice. Adv Anat Pathol 14: 419-430.] suggesting a potential functional abnormality in the BRCA-signaling pathway not identified by BRCA1 mutation in these BRCA WT patients.

Hence, DNA sequencing can detect BRCA1/2 mutations, but has the following limitations: 1) mutations may have unknown functional significance, 2) epigenetic alterations and mutations in other HR pathway components are not detected, and 3) the combined effects of pathway mutations are not understood. Thus, a functional assessment of HR competence at the single cell level is still an unmet need. Single Cell Network Profiling (SCNP) is a multiparametric flow cytometry-based assay that simultaneously measures, at the single cell level, extracellular surface markers and functional changes in intracellular signaling in response to extracellular modulators (S. M. Kornblau, M. D. Minden, D. B. Rosen, S. Putta, A. Cohen, T. Covey, D. C. Spellmeyer, W. J. Fantl, U. Gayko, A. Cesano, Dynamic single-cell network profiles in acute myelogenous leukemia are associated with patient response to standard induction therapy, Clinical cancer research: an official journal of the American Association for Cancer Research, 16 (2010) 3721-3733). In this study, we tested the ability of SCNP to detect and quantify functional changes in HR signaling using peripheral blood mononuclear cell (PBMC) samples from BRCA1 mutation carriers (MUT) and wild type (WT) subjects. See overview at FIG. 22.

Material and Methods

Patient Samples

Peripheral blood specimens were obtained from healthy subjects (age less than 50) or patients with breast or ovarian cancer (age greater than 50) and genotyped for BRCA1 at the North Shore Long Island Jewish Medical Center. Patients consented to the collection of biospecimens for biology studies. Patient characteristics are shown in FIG. 23. Informed consent was obtained in accordance with the Declaration of Helsinki, and the institutional review boards of participating institutions approved this study.

### Cell Lines

The following cell lines were obtained from Coriell Cell Repositories (Camden, N.J.), or ATCC and cultured in complete RPMI-1640 supplemented with 10-15% FCS as shown in Table 7 below:

| | | Cultured in | | |
|---|---|---|---|---|
| ID | Cell Type | CompleteMedia | Donor | Mutation |
| HCC1937BL | B-Lymphocyte | RPMI 10% FCS | BR cancer patient | BRCA1 +/− |
| GM00536 | B-Lymphocyte | RPMI 15% FCS | Healthy Donor | Not Tested (healthy) |
| GM13023 | B-Lymphocyte | RPMI 15% FCS | Fanconi's Anemia | BRCA2−/− |

Flow Cytometric Profiling of Cells

SCNP assays were performed in a similar manner as described in the patent applications incorporated above, the previous examples and as described previously in the following reference [Rosen D B, Putta S, Covey T, Huang Y W, Nolan G P, et al. (2010) Distinct patterns of DNA damage response and apoptosis correlate with Jak/Stat and PI3kinase response profiles in human acute myelogenous leukemia. PLoS One 5: e12405]. Aliquots of cryopreserved cells were thawed at 37° C., washed, resuspended in RPMI-1640 medium supplemented with 60% fetal bovine serum (FBS), and mononuclear cells isolated via ficoll density gradient. After a second washing step with RPMI-1640 medium supplemented with 60% FBS, cells were washed in RPMI-1640 supplemented with 10% FBS, counted, filtered, re-suspended in RPMI-1640 10% FBS, then aliquoted (100,000 cells/condition for primary cells or 50,000 cells/condition for cell lines) to tissue culture plates (Costar, Sigma Aldrich) which were previously coated with or without anti-CD3 (ebiosciences, San Diego Calif.) for primary cells, or left uncoated for cell lines. Plates were coated with 0.125 μg/mL anti-CD3 overnight at 4° C., then washed in PBS followed by RPMI 10% FCS, according to the manufacturer's instruction. Cells were then treated with 2 μg/mL soluble anti-CD28 (eBiosciences, San Diego Calif.) for PBMCs or media alone for cell lines. Cells were incubated at 37° C. for 24 h before addition of 6 μg/mL AZD2281 (PARPi)+/−2 μg/mL Temozolomide (TMZ) for 24-72 h. Following incubation with drugs, cells were stained with amine aqua viability dye (Invitrogen, Carlsbad, Calif., USA) to distinguish non-viable cells, fixed with 1.6% paraformaldehyde for 10 minutes at 37° C., pelleted, permeabilized with 100% ice-cold methanol, and stored at −80° C.

For antibody staining, cells were washed with FACS buffer (PBS, 0.5% BSA, 0.05% NaN3), pelleted, and stained with unlabeled antibody cocktails followed by fluorochrome conjugated secondary antibodies, then blocked with normal rabbit serum and normal mouse serum (Caltag, Life Technologies, Clarsbad Calif.) and stained with cockails of fluorochrome-conjugated antibodies (See FIG. 60 for a list of antibody reagents). These cocktails included antibodies against CD3 (BD Biosciences, San Jose, Calif.) to distinguish T cells, cleaved PARP (BD Biosciences) to distinguish apoptotic/non-apoptotic cells, CyclinA2 (Beckman Coulter, Miami Fla.) to identify cells between S and M cell cycle phases and 3 antibodies against intracellular DDR signaling molecules for an 8-color flow cytometry assay.

Data was acquired on an LSR II and/or CANTO II flow cytometer using the FACS DIVA software (BD Biosciences, San Jose, Calif.). All flow cytometry data were analyzed with FlowJo (TreeStar Software, Ashland, Oreg.) or WinList (Verity House Software, Topsham, Me.). Dead cells and debris were excluded by forward and side scatter properties combined with amine aqua viability dye exclusion. T cells were identified by expression of CD3 (BD Biosciences) and lack of cleaved PARP. Within T cells, Cyclin A2+ (cycling) and Cyclin A2− (non-cycling) subsets were then gated using CyclinA2 (Beckman Coulter, Miami, Fla.) as show in FIG. 35. For a given population and treatment condition, 100 cells were required to compute DDR metrics. The experiment was performed over 4 weeks with 10-11 samples analyzed per week (4 batches). The specific modulation conditions and DDR readouts were chosen based on foundational experiments measuring DDR responses of 5 BRCA1 WT (+/+) versus 5 MUT (+/−) cell lines (DDR paper ref) or PBMCs from healthy donors of unknown BRCA1 status.

Metrics:

The data was analyzed using various metrics, such Log 2Fold, Uu, Total, and Ua, to understand cell populations. See U.S. Ser. No. 61/515,660, which is hereby incorporated by reference in its entirety, for definitions of various metrics.

Statistics

Statistics: Based on cell line experiments, we prespecified that p-H2AX would be higher in BRCA1 MUT samples so 1-sided T-test or Wilcoxon tests were performed for p-H2AX univariate nodes. For all other readouts 2-sided T or Wilcoxan tests were performed. Statistical significance was defined at p<0.05.

Methods of Analyzing DNA Damage+Repair by Controlling for Proliferation:

Batch Adjustment Methods:

Linear regression methods for Computing R2 and p-Values with Proliferation.

Multivariate Analysis Methods:

Nodes for multivariate model building were selected as follows: from the 512 distinct node-metrics collected, random forest techniques were used to identify the top 50 nodes based on rank importance, nodes were then filtered for inclusion based on interquartile range (IQR>X for log 2Fold, Total nodes, IQR>Y for Uu, Ua nodes) correlation (R2<X), and node-pairs were filtered for inclusion based on correlations (R2<X) before bivariate logistic regression or decision tree (using recursive partitioning) modeling was performed.

Results:

BRCA2−/− and BRCA1+/− cell lines demonstrate defective HR pathway activity compared to BRCA1+/+ cell lines.

To develop methods for functionally characterizing HR pathway activity, we first turned to cell lines where genetic models of BRCA1 mutation exist. We analyzed 3 cell lines of distinct HR status including a BRCA1+/+ cell line, a BRCA1+/− cell line, and a BRCA2−/− cell line in 4 independent experiments. While non-cycling cells could only distinguish these three cell lines at the latest timepoint tested (72 h), we clearly observed three levels of p-H2AX at all timepoints tested (24 h-72 h) in cycling cells with BRCA1+/+ showing lowest, BRCA1+/− showing intermediate, and BRCA2−/− showing highest levels of induced p-H2AX in response to AZD2281, suggesting that quantitative measurements of DNA Damage can functionally identify HR defencies in heterozygous BRCA1+/− samples (FIG. 24).

PBMC require proliferation to measure the effects of PARPi.

The observation that PARPi induce more DNA Damage in cycling cells, made it appear that 1) proliferation is an important factor in measuring PARPi effects and that 2) proliferation would need to be induced in resting primary cells. To further develop conditions for assaying HR function in primary cells, we used PBMCs from healthy donors to test the requirement of proliferation for measuring the effects of PARPi. Healthy PBMC were incubated with or without anti-CD3, anti-CD28 crosslinking antibodies to induce activation and proliferation of T lymphocytes for 24 h before addition of PARPi AZD2281 or media alone. As shown in FIG. 25, in the absence of CD3/CD28 crosslinking, no increases were observed in the number of total T cells or CyclinA2+ T cells over time and PARPi treatment had no effect on T cell p-H2AX levels. In contrast, in the presence of CD3/CD28 crosslinking, increased Total and CyclinA2+ T cell numbers were observed and PARPi treatment both decreased the proliferative response and increased T cell p-H2AX levels at all timepoints tested (FIG. 25), suggesting that induction of proliferation in primary PBMC may be a requirement for measuring HR function using PARPi.

Study Design for Testing HR Pathway Activity in PBMC of Known BRCA1 WT or BRCA1 MUT Status

To assay HR pathway activity in proliferating PBMC (n=41) previously genotyped for BRCA1 status, we used the study design shown in FIGS. 26 and 48 and the gating scheme shown in FIG. 35. 20 BRCA1 WT and 21 BRCA1 MUT samples (patient characteristics shown in FIG. 23) were collected from either healthy subjects younger than 50 years (10 BRCA1 WT, 11 BRCA1 MUT) or patients with a history of breast or ovarian cancer older than 50 years (10 BRCA1 WT, 10 BRCA1 MUT) and randomized into 4 separate experimental batches to be assayed in 4 sequential weeks. Importantly, BRCA1 status was blinded until completion of experimental work and computation of raw DDR metrics. In each experimental batch, PBMC were thawed (Day1) and mononuclear cells were stimulated for 24 h with anti-CD3, anti-CD28 antibodies before addition of AZD2281, AZD2281+ TMZ or media alone (Day 2), cultured for an additional 48 h (Day4) or 72 h (Day5), then assayed for DDR readouts in multiple populations including total T cells, Cyclin A2− T cells and Cyclin A2+ T cells as shown in FIG. 26. 35/41 PBMC samples responded to CD3, CD28 stimulation with “proliferation” measures (herein defined as the percentage of Cyclin A2+ T cells out of total intact cells on Day 4 in conditions lacking genotoxic agents) greater than 7.5% and were considered evaluable for the study. Samples with less than 7.5% proliferation demonstrated low signaling for DDR metrics, confirming the requirement of induced proliferation for robust DDR responses and BRCA1 stratitfication (FIG. 36).

Proliferation is associated with DDR readouts in primary PBMCs

In agreement with Example 6, associations were observed between “proliferation” and DDR readouts with higher associations (R2 values and slopes) between proliferation and DDR metrics from the total T cell population compared to associations with DDR metrics from either CyclinA2− or CyclinA2+ subsets. See FIGS. 37 and 46. While specific analysis of Cyclin A2− or CyclinA2+ subsets partially controlled for the effect of proliferation (reduced R2 and slopes between proliferation and DDR metrics), linear regression still demonstrated significant association between most DDR readouts in CyclinA2+ cells (p-H2AX, p53, p21, p-BRCA1) and proliferation (data not shown), suggesting additional control for proliferation may be needed to measure HR deficiency.

To further understand how proliferation affects DDR readouts and whether any technical variation existed between the four experimental batches, we examined the relationships between DDR metrics in CyclinA2+ cells, proliferation, and batch run. As shown in FIG. 27, a positive association was seen between DDR metrics and proliferation with different ranges of proliferation seen between batches, suggesting that technical batch-to-batch variability is affecting proliferation, which may be effecting DDR readouts.

BRCA1 MUT PBMC demonstrate elevated levels of PARPi induced DSBs compared to BRCA1 WT PBMC, particularly in analyses controlling for proliferation.

To ensure our analysis of BRCA1 biology was not confounded by technical or proliferative variation, we tested two methods for controlling proliferation and technical variability. First we analyzed proliferation across all samples and asked where along this proliferation measure does a dynamic range for DDR signaling exist. Samples were arbitrarily divided into four quartiles based on proliferation and analyzed for 48 h PARPi induced p-H2AX levels in CyclinA2+ cells, While samples in the lowest (1st quartile) and highest (4th quartile) quartiles demonstrated low dynamic ranges characterized by either low (1st quartile) or high (4th quartile) p-H2AX induction, samples within the 2nd and 3rd quartiles (middle range of proliferation; 12-32% CyclinA2+ cells) showed a large dynamic range for induction of p-H2AX (FIG. 28). While analysis of all evaluable samples for BRCA1 stratification only demonstrated (non-significant) trends of increased p-H2AX in BRCA1 MUT samples, analysis of middle proliferation samples showed significantly elevated p-H2AX levels in BRCA1 MUT vs. BRCA1 WT in response to treatment with PARPi alone (p=0.037) or PARPi+TMZ (p=0.007).

The second method aimed to control for proliferation by normalizing DDR metrics across experimental batches, thereby removing technical variability between batches.

The second method aimed to control for proliferation by controlling for technical variability between experimental batches.

As different ranges of induced signaling were observed between batches (likely caused by different amounts of induced proliferation between batches) we performed batch adjustments for each DDR readout to give equal averages across the 4 individual batches (FIG. 29). Like analysis of samples only within the middle range of proliferation, analysis of batch-normalized DDR metrics showed elevated PARPi induced p-H2AX in BRCA1 MUT samples compared to than BRCA1 WT samples in Cyclin A2+ cells using metrics designed to measure the absolute magnitude of p-H2AX induction (log 2Fold metric) or percentage of pH2AX+ cells (Uu metric) (FIG. 29).

Similarly, analysis of CyclinA2+ cells at either 48 h or 72 h in conditions with PARPi with or without TMZ consistently showed increased induction of p-H2AX, p53, and p21 in BRCA1 MUT samples compared to BRCA1 WT samples with improved and statistically significant BRCA1 stratification seen with adjustments controlling for proliferation (either batch normalization or analysis of middle proliferation samples only) (FIGS. 30, 41, and 42). Interestingly, BRCA1 WT samples demonstrated higher basal p-BRCA1 levels and lower induction of p-BRCA1 compared to BRCA1 MUT samples. FIGS. 43 and 38.

At 48 h BRCA1 stratification was only consistently observed in cycling cells, however by 72 h, non-cycling cells consistently demonstrated BRCA1 stratification mirroring the stratification seen at 48 h in cycling cells. At 72 h in non-cycling cells, statistically higher induction of p-H2AX, p-BRCA1, p53 and p21 was observed in BRCA1 MUT samples compared to BRCA1 WT samples in analyses controlling for proliferation (FIGS. 41 and 42). For a summary of additional BRCA stratification trends, see FIGS. 44, 45, and 47.

To better characterize which nodes require additional analyses controlling for proliferation, we examined the a) association (R2 value) of proliferation with 48 h DDR nodes and b) the ability of each node to stratify for BRCA1 status using i) unadjusted DDR values, or to control for proliferation, using ii) batch adjusted DDR values or iii) unadjusted DDR values only from middle proliferation samples. While p53 nodes showed lower associations (R2<0.3) with proliferation and significant (p<0.05) BRCA1 stratification using unadjusted data, p-H2AX, p-BRCA1, and p21 demonstrated higher associations with proliferation (R2>0.3) and required methods normalizing for proliferation (either batch adjustment or analyses of middle proliferation samples) for significant BRCA1 stratification (FIG. 39), confirming the need to control for proliferation differences among samples in nodes affected by proliferation to measure HR pathway status of individual samples.

Similar trends for BRCA1 stratification exist in both Young Healthy donors and Patients with Breast or Ovarian Cancer.

We next examined whether the BRCA1 MUT vs WT stratification trends observed was present within the subgroups of either healthy subjects or breast or ovarian cancer samples. Similar BRCA1 WT vs. MUT trends were seen in subgroup analyses of either young healthy subjects and breast/ovarian cancer patients with higher p-H2AX, p53, p21, and p-BRCA1 induction observed in BRCA1 MUT samples within both subgroups, confirming that these data reflect the mutation status of the cells rather than demographic differences in DDR signaling (FIG. 40).

Combinations of DDR Readouts Improve BRCA1 Stratification

To capitalize on the multidimensional nature of SCNP data, we constructed multivariate models for BRCA1 mutational status combining individual DNA damage pathway measurements using logistic regression or decision tree (recursive partitioning)-based methods. Multivariate analyses identified node-pairs which were stratifying for BRCA1 status using either logistic regression or decision tree modeling or using both modeling techniques (FIG. 31). As shown in FIG. 32, both methods computed multiple node-pairs showing improved stratification of PBMC samples for BRCA1 mutational status compared to analysis of single nodes alone. Combinations of readouts such as basal BRCA1, PARPi induced p-BRCA1, p-H2AX, p53 and p21 and even PARPi induced apoptosis [as measured by increased cleaved PARP (cPARP) readouts from total intact cells] resulted in multiple models significantly stratifying for BRCA1 mutational status with AUCROCs values up to 0.972.

Discussion

Genomic instability is a hallmark of cancer. Quantitative functional analysis of an individual's ability to maintain genomic stability could significantly improve risk assessment for cancer development in healthy patients and substantially characterize the DNA repair capacity in cancer patients as a method for determining individualized therapeutic strategy. By simultaneously measuring DNA Damage, activation of DSB repair pathways, and p53 pathway activity components in single cells and distinct cell cycle subsets, we herein show that SCNP offers a novel in vitro approach to functionally identify impaired HR repair machinery in samples with BRCA1 haploinsufficiency using peripheral blood.

The data from the current study support three major conclusions:

First, PARP inhibitors, which induce DNA damage in specific cell cycle phases, require cell proliferation to have genotoxic effects. Further, our data suggest that proliferation levels affect the extent of PARPi induced DNA Damage and downstream activation of DNA repair/damage response pathways in individual samples.

Second, analysis of DNA repair capacity using PARPi most clearly identifies HR deficient samples in analyses controlling for proliferation. In these experiments, proliferation was controlled for by 1) subset analysis of CyclinA2− vs CyclinA2+ cells, 2) analysis of samples within a specific range of proliferation (to maintain a dynamic range for DNA damage measurements), or 3) controlling for experimental batch run where (proliferative differences were observed between batches).

Third, functional profiling of DNA damage pathways in primary PBMC identifies BRCA1+/− samples as having impaired HR as compared with BRCA1+/+ samples. Impaired HR was characterized by increased levels of DNA damage (p-H2AX) and downstream activation of repair proteins (p-BRCA1) or damage response pathways (p21, p53).

These data support a model by which BRCA1+/− mutated cells display a dampened HR capability for repairing PARPi induced DSBs in cycling cells (as evidenced by increased p-H2AX levels following PARPi treatment compared to BRCA1+/+ samples), resulting in heightened activation of downstream p53 mediated DSB response pathways (as evidenced by elevated levels of p53 and downstream p53 target p21 in BRCA1+/− samples compared to BRCA1+/+ samples (FIG. 33). Interestingly, our data show elevated DSB and downstream response (p-H2AX. p53, p21) in BRCA1+/− samples uniquely in cycling cells at 48 h, consistent with PARPi induced DSB occurring in cycling cells, but also in non-cycling cells by 72 h. This suggests a model in which cells which were cycling at 48 h and incurring DSB damage eventually finish cell division and at 72 h are in a non-cycling state (FIG. 33). Thus at 72 h the non-cycling fraction reflects a mixture of post-mitotic and truly non-cycling (just cycled and never cycled). Additional markers tracking cell division such as CFSE, which could separate these 2 populations could prove useful in further characterizing HR pathway function and identifying functionally HR deficient samples.

SCNP can detect functional differences in PBMC samples from BRCA1 heterozygous mutational carriers compared to BRCA1 WT samples by quantitatively assessing DDR signaling in proliferating T cells. While this study contains a limited number of samples, the concordance of results (higher p-H2AX in BRCA1+/− samples, particularly in CyclinA2+ cells) between cell lines models and primary cells is quite encouraging. The assay can form the basis for the development of screening tests to identify subjects at higher risk of developing cancer or stratification tests to inform on patient selection for treatment with PARP inhibitors.

### Example 6

### DNA Damage Response (DDR) Pathways

FIGS. 50-65 are relevant to this example. See FIGS. 49 and 50 for an overview. As explained above, the DDR pathway uses HR and NHEJ to repair DNA damage [Rosen D B, Putta S, Covey T, Huang Y W, Nolan G P, et al. (2010) Distinct patterns of DNA damage response and apoptosis correlate with Jak/Stat and PI3kinase response profiles in human acute myelogenous leukemia. PLoS One 5: e12405] and is also involved in the repair of interstrand DNA cross-links (ICL) in conjunction with the Fanconi anemia pathway [S. Jalal, J. N. Earley, J. J. Turchi, DNA repair: from genome maintenance to biomarker and therapeutic target, Clinical cancer research: an official journal of the American Association for Cancer Research, 17 (2011) 6973-6984]. NHEJ, which is predominately used in resting cells in the G0/G1 phases of the cell cycle, is a more error-prone mechanism of repairing DNA ds breaks via ligation of DNA ends without a template [Rosen D B, Putta S, Covey T, Huang Y W, Nolan G P, et al. (2010) Distinct patterns of DNA damage response and apoptosis correlate with Jak/Stat and PI3kinase response profiles in human acute myelogenous leukemia. PLoS One 5: e12405 and Stelzer G T, Goodpasture L (2000) Use of multiparameter flow cytometry and immunophenotyping for the diagnosis and classification of acute myeloid leukemia. In: Stewart C C, Nicholson J K A, editors. Immunophenotyping. Wilmington Del.: Wiley-Liss. pp. 215-238].

The capacity of an individual to repair DNA Damage has significant clinical implications which include effects on: 1) aging, 2) the risk of development of various illnesses including cancer and neurologic diseases and 3) the response of cancer cells to DNA-damaging therapies (S. Jalal, J. N. Earley, J. J. Turchi, DNA repair: from genome maintenance to biomarker and therapeutic target, Clinical cancer research: an official journal of the American Association for Cancer Research, 17 (2011) 6973-6984). Multiple assays analyzing DNA repair capacity have yielded useful information, but they have limitations in applicability, reproducibility, and interpretation (S. Jalal, J. N. Earley, J. J. Turchi, DNA repair: from genome maintenance to biomarker and therapeutic target, Clinical cancer research: an official journal of the American Association for Cancer Research, 17 (2011) 6973-6984). With this in mind, we set out to develop improved quantitative methods for assessing induced double strand DNA Damage and activation of the two major DSB repair pathways in response to genotoxic stresses.

Single cell network profiling (SCNP) using multiparametric flow cytometry has emerged as versatile tool to quantitatively and simultaneously study the function of several specific biological pathways and signaling networks at the single cell level (A. Ashworth, A synthetic lethal therapeutic approach: poly(ADP) ribose polymerase inhibitors for the treatment of cancers deficient in DNA double-strand break repair, Journal of clinical oncology: official journal of the American Society of Clinical Oncology, 26 (2008) 3785-3790 and S. Rottenberg, J. E. Jaspers, A. Kersbergen, E. van der Burg, A. O. Nygren, S. A. Zander, P. W. Derksen, M. de Bruin, J. Zevenhoven, A. Lau, R. Boulter, A. Cranston, M. J. O'Connor, N. M. Martin, P. Borst, J. Jonkers, High sensitivity of BRCA1-deficient mammary tumors to the PARP inhibitor AZD2281 alone and in combination with platinum drugs, Proc Natl Acad Sci USA, 105 (2008) 17079-17084.) By characterizing cellular signaling responses following exposure to extracellular modulators, signaling network integrity and dysfunction can be revealed to identify properties not seen in resting cells. We have recently demonstrated that dynamic single cell network profiles can quantitatively measure DNA damage (p-H2AX) in primary clinical samples, such as newly diagnosed adult AML [D. B. Rosen, J. A. Cordeiro, A. Cohen, N. Lacayo, D. Hogge, R. E. Hawtin, A. Cesano, Assessing signaling pathways associated with in vitro resistance to cytotoxic agents in AML, Leukemia research, (2012)]. Herein, we demonstrate the usefulness of SCNP to 1) reproducibly characterize the activation of HR and NHEJ repair pathways in response to DNA Damage, 2) mechanistically characterize distinct classes of genotoxins with regard to cell cycle specificity of, and 3) identify samples with defective repair by analyzing induced DNA Damage within distinct cell cycle subsets.

Materials and Methods

Patient Samples

Peripheral blood or bone marrow specimens were obtained from patients with AML enrolled in the following clinical studies performed by either the South Western Oncology Group (SWOG) or the Childerns' Oncology Group (COG): S9333, S0112, S0106, S0301 and AAML03P1. See FIG. 59. All patients consented to the collection of biospecimens for biology studies. Informed consent was obtained in accordance with the Declaration of Helsinki, and the institutional review boards of participating institutions approved this study.

Cell Lines

U937, GDM-1, HCC1937BL, HCC19654BL & RS4;11 cell lines were obtained from ATCC and maintained in RPMI-1640 supplemented with 10% FCS. GM03189, GM03323, GM00536, GM01526, GM09703, GM13023, GM16756, GM14091, GM13705, GM13709, GM14090, GM05423, GM17230, GM17217 cell lines were obtained from Coriell Cell Repositories and cultured in complete RPMI-1640 supplemented with 15% FCS. Individual cell lines harbored mutations in ATM, BRCA2, BRCA1 as outlined in Table 8 below/

TABLE 8

| | | | | | | |
|---|---|---|---|---|---|---|
| | Cell | | | Age | | |
| Cell Line | Type | Description | Mutations | (YR) | Race | Sex |

Panel 1: Etoposide Nodes showing lack of response of ATM−− cells to DDR readouts (Tool validation)

| | | | | | | |
|---|---|---|---|---|---|---|
| U937 | Myeloid | Monocytic Lymphoma | | 37 | Caucasian | M |
| RS4; 11 | B-Cell | B-ALL | | 32 | Caucasian | F |
| GM03189 | B-Cell | AT patient | ATM −/− del8266AT, 4bpins1141 | 7 | Caucasian | M |
| GM03323 | B-Cell | Healthy Donor | ATM +/− sibling to GM03189 | 6 | Caucasian | M |
| GM00536 | B-Cell | Healthy Donor | ATM +/+ | 27 | Caucasian | M |
| GM01526 | B-Cell | AT patient | ATM −/− 2T-->C | 28 | Caucasian | F |
| GM09703 | B-Cell | Seckel Syndrome | Unknown; ATR−/− phenocopy | 14 | Caucasian | F |

Panel 2: PARP Nodes Testing pH2AX in CyclinA2− and + cells, and showing stratification of BRCA2−/−

| | | | | | | |
|---|---|---|---|---|---|---|
| GM13023 | B-Cell | Fanconi's Anemia | BRCA2−/− | 2 | Caucasian | M |
| GM16756 | B-Cell | Fanconi's Anemia | FANCD2−/− 376A > G, 3707G > A | 7 | Unknown | M |
| GM09703 | B-Cell | Seckel Syndrome | Unknown; ATR−/− phenocopy | 14 | Caucasian | F |
| GM00536 | B-Cell | Healthy Donor | ATM +/+ | 27 | Caucasian | M |
| GM01526 | B-Cell | AT patient | ATM −/− 2T-->C | 28 | Caucasian | F |

Panel 3: PARPi experiments Testing Repeatability of DDR nodes + Showing Stratification of BRCA2−/−
vs BRACA1+/− vs BRCA1+/+ samples

| | | | | | | |
|---|---|---|---|---|---|---|
| HCC1937BL | B-Cell | Breast Cancer | BRCA1 +/− 5382insC | 24 | Caucasian | F |
| GM14091 | B-Cell | Breast Cancer | BRCA1 +/− 5382insC | 46 | Caucasian | F |
| GM13705 | B-Cell | Breast Cancer | BRCA1 +/− 3875del4 | 38 | Caucasian | F |
| GM13709 | B-Cell | Breast Cancer | BRCA1 +/− 2187delA | 32 | Caucasian | F |
| GM14090 | B-Cell | Breast Cancer | BRCA1 +/− 185delAG | 43 | Caucasian | F |
| HCC1954BL | B-Cell | Breast Cancer | BRCA1 +/+ | 61 | East | F |
| | | | | | Indian | |
| GM00536 | B-Cell | Healthy Donor | Not tested (healthy) | 27 | Caucasian | M |
| GM05423 | B-Cell | Healthy Donor | Not tested (healthy) | 32 | Caucasian | F |
| GM17230 | B-Cell | Healthy Donor | Not tested (healthy) | 37 | Caucasian | F |
| GM17217 | B-Cell | Healthy Donor | Not tested (healthy) | 37 | Caucasian | F |
| GM13023 | B-Cell | Fanconi's Anemia | BRCA2−/− | 2 | Caucasian | M |
| GM09703 | B-Cell | Seckel Syndrome | Unknown; ATR−/− phenocopy | 14 | Caucasian | F |

Flow Cytometric Profiling of Cells

SCNP assays were performed in a similar manner as described in the patent applications incorporated above, the previous examples and as described previously in the following reference [Rosen D B, Putta S, Covey T, Huang Y W, Nolan G P, et al. (2010) Distinct patterns of DNA damage response and apoptosis correlate with Jak/Stat and PI3kinase response profiles in human acute myelogenous leukemia. PLoS One 5: e12405]. Aliquots of cryopreserved cells were thawed at 37° C., washed, resuspended in RPM I-1640 medium supplemented with 60% fetal bovine serum (FBS), and mononuclear cells isolated via ficoll density gradient. After a second washing step with RPMI-1640 60% FBS, cells were washed in RPMI-1640 10% FBS, counted, filtered, re-suspended in RPMI-1640 10% FBS, then aliquoted (100,000 cells/condition for primary cells or 50,000 cells/condition for cell lines) and rested for 30 min at 37° C. For experiments measuring multiple DDR readouts after etoposide treatment, AML samples or cell lines (cell line panel 1 in Table 8) were treated with 30 μg/mL etoposide for 2 h or 6 h. For experiments measuring p-H2AX responses in All Cells, CyclinA2+ cells or CyclinA2− cells and comparing proliferation with DNA Damage and Apoptosis across drugs, AML samples were treated for 48 h with 20 ng/mL of growth factors [IL-3 (BD Biosciences), SCF (R&D Systems), FLT3L (ebiosciences) and TPO (R&D Systems)] to induce proliferation then AML samples and cell lines (cell line panel 2 in Table 8) were challenged with 30 μg/mL etoposide, 6 μg/mL AZD2281, 1 μg/mL Gemtuzumab Ozogamicin, 0.25 μM Clofarabine, 10 μg/mL Temozolomide, or the combination of 6 μg/mL AZD2281+10 μg/mL Temozolomide for 6-72 h. For experiments showing reproducibility and dynamic ranges of multiple DDR readouts in Cyclin A2− and CyclinA2+ cells, experiments showing the effect of gating on cyclinA2+ cells on associations between DDR readouts with proliferation and experiments showing stratification of BRCA1+/+ vs BRCA1+/− or BRCA2−/− cell lines (cell line panel 3 in Table 8), cells were treated with 6 μg/mL AZD2281+/−2 μg/mL Temozolomide for 48-72 h.

Following incubation with drugs, cells were stained with amine aqua viability dye (Life Technologies, Carlsbad, Calif., USA) to distinguish non-viable cells, fixed with 1.6% paraformaldehyde for 10 minutes at 37° C., pelleted, permeabilized with 100% ice-cold methanol, and stored at −80° C. For antibody staining cells were washed with FACS buffer (PBS, 0.5% BSA, 0.05% NaN3), pelleted, and stained with unlabeled antibody cocktails followed by fluorochrome conjugated secondary antibodies, then blocked with normal rabbit serum and normal mouse serum (Caltag-Life Technologies, Carlsbad, Calif., USA) and stained with cockails of fluorochrome-conjugated antibodies (See FIG. 60 for a list of antibody reagents). These cocktails included antibodies against 2-5 cell surface markers for cell population leukemic cell gating of AML cells (e.g. CD11b, CD34, and CD45) and up to 3 antibodies against intracellular signaling molecules for an 8-color flow cytometry assay.

Data was acquired on an LSR II and/or CANTO II flow cytometer using the FACS DIVA software (BD Biosciences, San Jose, Calif.). All flow cytometry data were analyzed with FlowJo (TreeStar Software, Ashland, Oreg.) or WinList (Verity House Software, Topsham, Me.). Dead cells and debris were excluded by forward and side scatter properties combined with amine aqua viability dye exclusion. For AML samples, non-apoptotic leukemic cells were identified based on expression of CD45 and side-scatter properties and lack of apoptosis marker cleaved PARP as previously described [Rosen D B, Putta S, Covey T, Huang Y W, Nolan G P, et al. (2010) Distinct patterns of DNA damage response and apoptosis correlate with Jak/Stat and PI3kinase response profiles in human acute myelogenous leukemia. PLoS One 5: e12405 and Stelzer G T, Goodpasture L (2000) Use of multiparameter flow cytometry and immunophenotyping for the diagnosis and classification of acute myeloid leukemia. In: Stewart C C, Nicholson J K A, editors. Immunophenotyping. Wilmington Del.: Wiley-Liss. pp. 215-238.] and Cyclin A2 staining discriminated Cyclin A2+ vs Cyclin A2− cells. For cell lines, forward scatter, side scatter, amine aqua, and cleaved PARP similarly identified live non-apoptotic cells and Cyclin A2 discriminated Cyclin A2+ vs Cyclin A2− cells.

Metrics

For measures of apoptosis, the percentage of induced apoptotis/cell death (apoptosis) was calculated as: [LiveUntreated−LiveDrug treated]/[LiveUntreated], with “live” cells defined as the percentage of leukemic cPARP-/aqua dye-cells. This metric ranges from values of 0 to 100% for each sample and normalizes for differences in sample quality or spontaneous apoptosis. Metrics for quantifying DDR (Log 2Fold and Uu) have been described previously (A. Cesano, D. B. Rosen, P. O'Meara, S. Putta, U. Gayko, D. C. Spellmeyer, L. D. Cripe, Z. Sun, H. Uno, M. R. Litzow, M. S. Tallman, E. Paietta, Functional pathway analysis in acute myeloid leukemia using single cell network profiling assay: Effect of specimen source (bone marrow or peripheral blood) on assay readouts, Cytometry Part B: Clinical Cytometry, 82B (2012) 158-172). Also, see U.S. Ser. No. 61/515,660. Briefly, the Log 2Fold metric measures the magnitude of the responsiveness of a cell population where a value of zero indicates lack of induced signaling while a positive value indicates an increase in signaling response of a population. The “Uu” Metric measures the proportion of responsive cells by comparing the overlap and rank-order of the modulated and unmodulated populations on a cell-by-cell basis and ranges from zero to one where 0.5 indicates no change, values>0.5 indicate cells have increased in signal, and values<0.5 indicate that cells have decreased in signal vs. an unmodulated population. The U metric is useful for comparing which could otherwise have data on a normalized scale zero to one scale and was used here to compare the proportion of responding cells within Cyclin A2− or Cyclin A2+ populations in response to a panel of genotoxins.

Results

Development of Single Cell Assays to Measure Induction of DNA Damage and ATM Mediated Activation of DNA Damage Repair Pathways

To quantitatively assess induction of DNA damage and activation of DNA damage repair (DDR) pathways at the single cell level, we first identified conditions and reagents capable of measuring activation motifs within the two major double strand break repair pathways [Non-Homologous End Joining (NHEJ) and Homologous Recombination Repair (HR)]. Using topoisomerase II inhibitor etoposide to induce DNA Damage and DDR pathway activation, we tested phosphorylation sites on multiple proteins within NHEJ and HR (FIGS. 51 and 61) In a panel of cell lines, etoposide induced a range of phosphorylation levels for all proteins tested. Of note, ATM−/− cell lines demonstrated a minimal response in DDR readouts uniquely downstream of ATM (p-ATM, p-DNA-PKcs, p-BRCA1) while showing detectable but muted activation of DDR readouts phosphorylated by multiple kinases (p-H2AX) compared to other cell lines tested, validating the specificity of these DDR tools with genetic controls lacking functional ATM kinase (FIG. 52).

Assessment of DNA Damage and Activation of DNA Damage Repair Pathways in Primary AML Samples

To examine clinically relevant samples for DDR pathway activity, we turned to AML, where the standard induction therapy regimen consists of genotoxic agents that induce DSB. We tested activation of DDR pathways in response to in vitro etoposide treatment to assess the integrity and relationships of these repair pathways in primary AML samples. As shown in FIG. 53, we detected co-activation of repair proteins within unique pathways with higher correlations observed within HR and DNA DSB response proteins (p-BRCA1, pATM) or within NHEJ and DNA damage readouts (p-DNAPK, p53PB1, pH2AX) and weaker correlations seen across the NHEJ Repair (pDNA-PK, p53BP1) vs. HR (pBRCA) readouts. Analyzed at the individual sample level, the data show subgroups of AML with higher activation of either p-DNA-PKcs or p-BRCA1 suggesting individual leukemia samples may selectively initiate either NHEJ or HR mediated repair (FIG. 53).

Measuring cell cycle specificity and the effect of cell proliferation demonstrates mechanistic differences between individual genotoxins.

Next, we wanted to use these AML samples to mechanistically characterize distinct classes of genotoxins besides etoposide. Because some of these agents, such as PARP inhibitors, require cell proliferation for accumulation of DSB (by a process in which unresolved single strand breaks are converted to double strand breaks during DNA synthesis), primary AML samples were cultured with growth factors (SCF, FLT3L, TPO, IL-3) for 48 h to induce cell proliferation then treated with genotoxic agents for 6-72 h. To assess cell proliferation, we quantified the percentage of leukemic cells positive for Cyclin A2 (Pct Cyclin+), a marker expressed between the S and M phases of the cell cycle, on the day of drug addition. This measure of proliferation (Pct Cyclin+ or “proliferation”) was then compared with induced DNA Damage (p-H2AX) after 6 h or induced apoptosis/cell death after 24-72 h of treatment with individual genotoxins. As shown in Table 62, proliferation was more strongly associated (Higher R2) with both induced p-H2AX and with apoptosis/cell death for conditions with PARPi which specifically induce DSB in cycling cells (AZD or AZD+TMZ; average R2: 0.695, 0.622, respectively) than for conditions with etoposide or G0 (average R2: 0.384, 0.145, respectively).

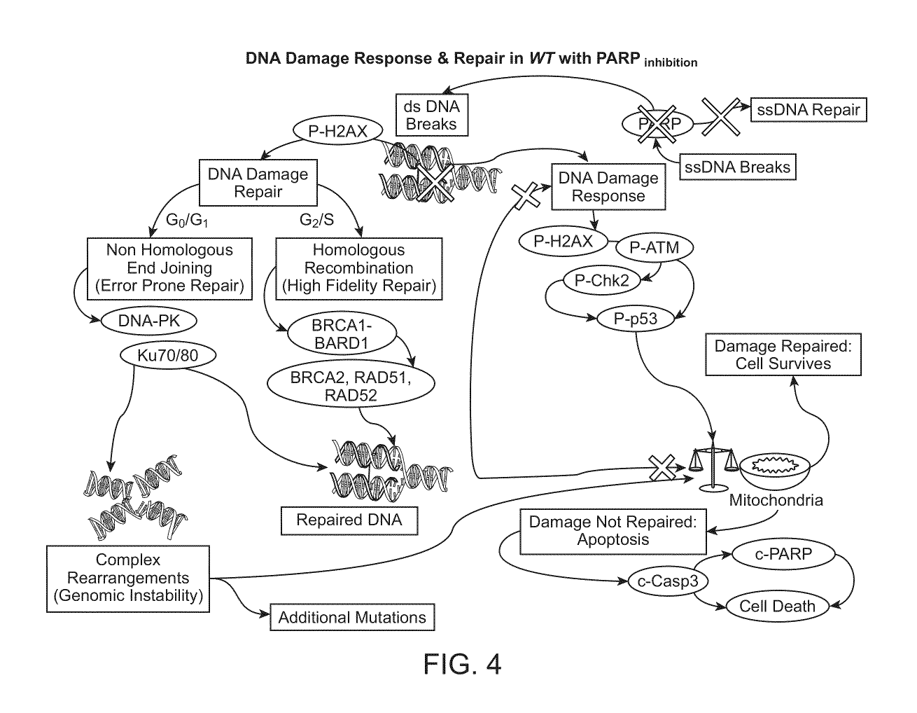

To further mechanistically characterize the cell cycle specificity of unique genotoxic agents in primary clinical samples, we treated AML samples with a panel of genotoxins and measured the induction of p-H2AX in CyclinA2− (non-cycling) or Cyclin A2+ (cycling) cells. For etoposide and GO (Mylotarg), similar levels of p-H2AX were induced in either non-cycling or cycling cells (FIG. 4) in agreement with the lower associations observed between proliferation and p-H2AX or apoptosis for these agents. While, clofarabine and temozolomide treatment induced higher p-H2AX in cycling cells with measurable but lower p-H2AX induction in non-cycling cells, conditions with PARPi AZD2281 induced pH2AX specifically in cycling cells (FIG. 54), consistent with the higher relationships observed between proliferation and pH2AX or apoptosis for conditions with PARPi. To confirm the cell cycle specificities observed for individual genotoxins, we tested a panel of cell lines for p-H2AX induction in cycling or non-cycling cells treated with the same agents. With the exception of lower overall levels of GO induced p-H2AX in the cell lines vs. the AML samples (likely explained by the lack of target CD33 expression in the B cell lines tested) we observed similar cell cycle specificities for the unique genotoxins in cell lines, confirming the mechanistics insights observed in primary cells (FIG. 63).

Cyclin A2+ cells demonstrate more robust and reproducible activation of multiple DNA Damage repair proteins compared to Cyclin A2− cells in response to PARPi treatment.

With the mechanistic insight that certain genotoxins, such as PARP inhibitors, display cell cycle specificity and differentially induce DNA damage in cycling vs. non-cycling cells, we set out to characterize the activation of additional DNA Damage repair proteins in cycling and non-cycling cells in response to treatment with PARPi+/−temozolomide. We treated a panel of 12 EBV transformed B cell lines with PARP+/−temozolomide for 48-72 h and analyzed 6 DDR proteins including p-H2AX, p-RPA2, p-DNAPKcs, p-ATM, p-BRCA1 and p21 (total protein). As shown in FIG. 55, we consistently observed statistically higher induction of all 6 DDR readouts in cycling vs non-cycling cells in all timepoints (48 h, 72 h) and treatment conditions (+/−TMZ) tested (paired t-test p-values ranged from p=0.004 to p=8.86×10-9). Within the cycling population, p-H2AX, and p21 showed higher induction of signal while p-DNA-PKcs showed lower induction, particularly in response to PARPi alone. In addition, higher levels of DDR responses were observed in conditions with temozolomide and in later (72 h) time points of drug treatment in both non-cycling and cycling cells.

To assess the reproducibility of DDR readouts, we computed the coefficient of variation (CV) between two independent experiments using the same cell lines. As shown in FIG. 56, we consistently observed better reproducibility for DDR readouts (lower CVs, all <0.2, typically <0.15) in the cycling population compared to the non-cycling population in both timepoints (48 h, 72 h) and treatment conditions (AZD+/−TMZ) with statistical significance seen (p<0.05 paired t-test) for 21/24 conditions tested.

Analyzing DNA Damage repair proteins in CyclinA2+ cells controls for proliferative differences and reveals DNA Damage repair pathway defects.

Based on the increased signal intensities and better reproducibility observed for DDR readouts in cycling cells along with the thought that gating on cycling cells could help normalize for proliferative differences in the frequency of CyclinA2+ cells, we asked whether an analysis of DNA Damage specifically in cycling cells could help identify samples with defective DDR machinery. First, to ask whether specifically analyzing DDR in cycling cells reduces the effect of proliferation on DDR measurements, we used cell lines to compare the association (R2 values) between proliferation (as measured by the frequency of CyclinA2+ cells) and DDR readouts from a) cycling cells only, or b) all live cells. Using linear regression, we consistently observed lower R2 values and slopes between proliferation and DDR readouts from cycling cells (ave R20.194) compared to DDR readouts from all cells (ave R20.403), suggesting that gating on cycling cells reduces the effect of proliferation on DDR readouts (FIG. 64).

To examine how specifically analyzing DDR in cycling cells can enable identification of samples with DNA repair defects, we examined the kinetics (6 h-72 h) of AZD2281 induced p-H2AX induction in cycling vs. non-cycling cells of another panel of cell lines, including a BRCA2−/− mutant. While p-H2AX levels in all cells or non-cycling cells failed to clearly identify the BRCA2−/− as having an abnormal ability to repair AZD2281 induced DSB, p-H2AX levels in cycling cells were clearly elevated in BRCA2−/− cells compared to the other cell lines tested suggesting that specific analysis of DSB in cycling cells aids in identifying HR pathway defects (FIG. 57).

To further test this system, we next asked whether we could detect functional HR pathway deficiency in heterozygous BRCA1 mutated cells using 5 EBV-transformed B lymphoblast cell lines from patients with BRCA1-mutated breast cancer where peripheral blood B cells should be BRCA1+/−. To model wild type BRCA1 (BRCA1+/+), we used 5 EBV-transformed B lymphoblast cell lines from patients with BRCA1-WT breast cancer or from healthy donors. We next tested the functional integrity of the HR pathway in these BRCA+/+ and BRCA+/− lines and in BRCA2−/− mutant cells by treating cells with PARP inhibitor AZD2281 (AZD)+/− alkylating agent Temozolomide (TMZ) and measuring i) induction of double strand DNA Damage (as measured by p-H2AX), ii) activation of downstream DNA Damage repair proteins from both HR and NHEJ (as measured by p-BRCA1, p-RPA2 or p-DNAPKcs, respectively) and iii) activation of p53 pathway activity (as measured by p53 and p21 total protein levels). While homozygous BRCA2−/− cells showed the highest PARPi induced p-H2AX levels in cycling cells, the 5 heterozygous BRCA1+/− cell lines also displayed significantly (p<0.05) higher levels of PARPi induced p-H2AX compared to the 5 BRCA1+/+ lines in cycling cells but not in non-cycling cells at all timepoints and treatment conditions tested (FIG. 58). For example, 48 h treatment with AZD+TMZ resulted in higher p-H2AX levels in BRCA1+/−vs. BRCA1+/+ samples (2.22±0.161 vs. 1.65±0.085, respectively, p: 0.014) in Cyclin A2+ cells with no significant difference observed for BRCA1+/− vs BRCA1+/+ samples in Cyclin A2− cells (1.14±0.258 vs. 0.650±0.195, respectively, p: 0.172), consistent with the proposed mechanism of unresolved SSB becoming DSB during S-phase in cells treated with PARPi requiring repair by BRCA1 and the HR pathway.

Discussion

By simultaneously measuring DNA Damage and activation of multiple DSB repair pathway components in single cells and distinct cell cycle subsets, data from the current study support three major conclusions: First, using novel methods to measure single cell activation and phosphorylation of DNA repair proteins from multiple repair pathways including NHEJ (p-DNA-PK, p-53BP1) and HR (p-RPA2, p-BRCA1) with flow cytometry, measurements which previously required bulk cell lysis and detection via western blot, these experiments demonstrate that primary cancer samples differ in their relative activation of HR vs NHEJ components in a measurable fashion.

Second analysis of induced DNA Damage in Cyclin A2+ vs CyclinA2− cell cycle subsets mechanistically characterizes genotoxins and distinguishes genotoxins which require proliferation and are cell cycle specific from those that induce DNA Damage in all cell cycle phases. Such mechanistic characterization could be quite useful in a pre-clinical setting, particularly in light of recent failed clinical trials with supposed “PARP” inhibitors, drugs which would have clearly benefited from a more mechanistic pre-clinical analysis (H. Ledford, Drug candidates derailed in case of mistaken identity, Nature, 483 (2012) 519 and A. G. Patel, S. B. De Lorenzo, K. S. Flatten, G. G. Poirier, S. H. Kaufmann, Failure of Iniparib to Inhibit Poly(ADP-Ribose) Polymerase In Vitro, Clinical Cancer Research, 18 (2012) 1655-1662).

Lastly, it is possible to functionally identify samples with defective (BRCA2−/−) or impaired (BRCA1+/−) HR repair machinery using SCNP. Our data suggest that for genotoxins which require cell proliferation, such as PARPi induced DNA Damage and DDR responses are associated with cell proliferation. Importantly, analysis of CyclinA2+ cells helps control for proliferative effects and shows more robust, reproducible measures of PARPi induced DNA Damage or activation of DSB repair pathways with clearer identification of samples with defective (BRCA2−/−)- or impaired (BRCA1+/−), compared to an analysis of Cyclin A2− cells. This is particularly important when thinking about the development of clinically predictive or prognostic assays which require a high level of technical robustness.

Our findings are consistent with pre-clinical data in which PARP inhibition was noted to be most effective in BRCA-deficient or mutant samples presumably by a mechanism in which PARP inhibitors significantly increase the amount of DNA damage requiring BRCA/HR mediated repair and result in substantial error-prone NHEJ mediated repair of DNA damage in the presence of dysfunctional BRCA/HR (H. Farmer, N. McCabe, C. J. Lord, A. N. Tutt, D. A. Johnson, T. B. Richardson, M. Santarosa, K. J. Dillon, I. Hickson, C. Knights, N. M. Martin, S. P. Jackson, G. C. Smith, A. Ashworth, Targeting the DNA repair defect in BRCA mutant cells as a therapeutic strategy, Nature, 434 (2005) 917-921; A. Ashworth, A synthetic lethal therapeutic approach: poly(ADP) ribose polymerase inhibitors for the treatment of cancers deficient in DNA double-strand break repair, Journal of clinical oncology: official journal of the American Society of Clinical Oncology, 26 (2008) 3785-3790; S. Rottenberg, J. E. Jaspers, A. Kersbergen, E. van der Burg, A. O. Nygren, S. A. Zander, P. W. Derksen, M. de Bruin, J. Zevenhoven, A. Lau, R. Boulter, A. Cranston, M. J. O'Connor, N. M. Martin, P. Borst, J. Jonkers, High sensitivity of BRCA1-deficient mammary tumors to the PARP inhibitor AZD2281 alone and in combination with platinum drugs, Proc Natl Acad Sci USA, 105 (2008) 17079-17084; C. E. Strom, F. Johansson, M. Uhlen, C. A. Szigyarto, K. Erixon, T. Helleday, Poly (ADP-ribose) polymerase (PARP) is not involved in base excision repair but PARP inhibition traps a single-strand intermediate, Nucleic acids research, 39 (2011) 3166-3175 and A. G. Patel, J. N. Sarkaria, S. H. Kaufmann, Nonhomologous end joining drives poly(ADP-ribose) polymerase (PARP) inhibitor lethality in homologous recombination-deficient cells, Proceedings of the National Academy of Sciences of the United States of America, 108 (2011) 3406-3411.)

Another recently described method for assessing HR function in single cells, which has generated useful data in cell lines, measures repair and reactivation of an HR substrate reporter gene (DR-GFP) [K. Nakanishi, F. Cavallo, E. Brunet, M. Jasin, Homologous recombination assay for interstrand cross-link repair, Methods in molecular biology, 745 (2011) 283-291], however the requirement of transient transfection in this system challenges the potential clinical utility of this approach [S. Jalal, J. N. Earley, J. J. Turchi, DNA repair: from genome maintenance to biomarker and therapeutic target, Clinical cancer research: an official journal of the American Association for Cancer Research, 17 (2011) 6973-6984]. Alternatively, the experiments herein described do not require transfection but rather use treatment with specific genotoxins to functionally measure DNA repair capability.

As the ultimate capacity of cells to respond to genotoxins and repair damaged DNA is affected by multiple distinct molecular mechanisms and/or individual mutations, SCNP greatly facilitates the recognition and quantification of the functional consequences of these alterations converging at the downstream level of pathway and network responses. This approach may provide additional functionally relevant information compared to sequencing-based mutational analysis of DNA repair genes alone, such as BRCA1/2 sequencing, where many mutations have unknown function, epigenetic and/or non-coding alterations may not be detected and the combined effects of pathway mutations are not understood.

These data demonstrate the utility of SCNP for functionally assaying multiple DNA Damage associated readouts in single cells of distinct cell cycle subsets to characterize unique genotoxins and identify samples with defective repair capacity. As multivariate analyses of SCNP data have proven useful in prediction of clinical outcome in other oncology settings [S. M. Kornblau, M. D. Minden, D. B. Rosen, S. Putta, A. Cohen, T. Covey, D. C. Spellmeyer, W. J. Fantl, U. Gayko, A. Cesano, Dynamic single-cell network profiles in acute myelogenous leukemia are associated with patient response to standard induction therapy, Clinical cancer research: an official journal of the American Association for Cancer Research, 16 (2010) 3721-3733 and A. Cesano, E. Evensen, J. Ptacek, J. Cordeiro, R. E. Hawtin, J. R. Ware, I. Nichele, M. T. Scupoli, BCR Responsiveness is Associated with Time to First Treatment (TTFT) in B-Cell Chronic Lymphocytic Leukemia (B-CLL): Results From a Single Cell Network Profiling (SCNP) Verification Study, ASH Annual Meeting Abstracts, 118 (2011) 2834], future experiments are warranted characterizing genotoxins or clinical samples by combining multiple DNA damage associated pathway readouts in a multivariate approach to capitalize on the multidimensional nature of this data.

Our data demonstrate the capability to quantitatively measure functional activation of NHEJ and HR pathways relevant to genotoxin responses and cancer predisposition. As mutations in additional DNA repair pathways [such as nucleotide excision repair (NER), base excision repair (BER), and mismatch repair (MMR)] are also associated with cancer disposition and/or drug sensitivity (S. Jalal, J. N. Earley, J. J. Turchi, DNA repair: from genome maintenance to biomarker and therapeutic target, Clinical cancer research: an official journal of the American Association for Cancer Research, 17 (2011) 6973-6984) the development of functional assays for these additional repair pathways would also be worthwhile and clinically relevant. Our cell line data suggest that DNA repair deficiencies, including HR haploinsufficiency, are detectable through functional assays. This assay could form the basis for the development of screening tests to identify subjects at higher risk of developing cancer or stratification tests to inform on cancer patient selection for treatment with PARP inhibitors.

### Example 7

### Functional Characterization of KIT and FcεR1 Receptor Mutations in Mast Cell Leukemia (MCL) Using Single Cell Network Profiling (SCNP)

Background: A recent report described an imatinib/dasatinib resistant MCL patient (pt) with mutations in KIT (V654A) and FcεR1 (L188F) receptors [Spector et al, Leukemia 2011, incorporated by reference]. The pt did not respond to cytarabine-based induction therapy combined with dasatinib or to post-induction imatinib. The functional consequence of the receptor mutations on downstream signaling networks, and sensitivity to alternative therapeutics, was unknown. SCNP was applied to interrogate signaling networks downstream of these mutations and network sensitivity to targeted therapeutics by examining: 1) Basal and modulated signaling using stem cell factor (SCF) or α-IgE; 2) Effect on basal and modulated signaling of: a) KIT inhibitors imatinib, dasatinib and nilotinib; b) PI3K inhibitor GDC-0941; and c) SYK inhibitor fostamatinib R406.

Methods: Cryopreserved MCL BMMCs were processed with healthy donor BMMCs and fresh healthy donor basophils as controls. BMMCs were modulated with SCF for 5 and 15 min+/−KIT or PI3K inhibitor. MCL BMMCs and healthy donor basophils were modulated with α-IgE for 5 min+/−fostamatinib. Signaling in the KIT and FcεR1 pathways were quantified through measurement of p-AKT, p-ERK, or p-S6 or p-ERK, p-PLCγ2 and p-SYK levels respectively in the MCL population defined by CD45, CD34, CD33, and CD117.

Results: Consistent with previous reports, the V654A KIT mutation did not result in constitutive activation of the PI3K or MAPK pathways in MCL blasts, but was associated with dysfunctional SCF modulated signaling. Specifically, SCNP identified SCF induced p-AKT levels at 5 min, higher (2×) compared to CD34+/CD117+ healthy donor control cells, and sustained to 15 min with no simultaneous induction of p-ERK or p-S6. Consistent with the clinically observed imatinib resistance of the MCL case, in vitro AKT induction was unaffected by the presence of KIT inhibitors, but sensitive to the PI3K inhibitor GDC-0941. Of note, KIT inhibitors and GDC-0941 blocked SCF induced signaling in the healthy BMMC control. Despite robust p-ERK induction in the healthy donor basophil control sample after α-IgE modulation of the FcεR1 receptor, and inhibition by fostamatinib treatment, no basal or α-IgE modulated FcεR1 receptor signaling was detected in MCL BMMC cells.

Conclusions: SCNP can functionally characterize signaling and drug resistance profiles in MCL BMMCs and can potentially inform on therapeutic selection. These data demonstrate the ability in one assay to: 1) Profile disease-associated signaling; 2) Profile mutation-associated signaling at the level of the individual cell subpopulation across multiple nodes; and 3) Identify drug resistance and sensitivity profiles.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of classification, diagnosis, prognosis and/or prediction of an outcome of a condition in an individual, said method comprising:
a) contacting a cell population from said individual with a DNA damage or apoptosis inducing agent, wherein said cell population comprises a genetic and/or epigenetic alteration, wherein said alteration is associated with the development of said condition;
b) characterizing a plurality of DNA damage repair pathways in one or more cells from said cell population by determining an activation level of at least one activatable element within said plurality of DNA damage repair pathways;
c) determining whether said plurality of DNA damage pathways are functional in said individual based on the activation levels of said activatable elements; and
d) making a decision regarding classification, diagnosis, prognosis and/or prediction of an outcome of said condition in said individual, wherein said decision is based on said determination on step (c).

2. The method of claim 1 further comprising performing a molecular analysis to detect said genetic alteration is said cell population.

3. The method of claim 1, wherein said DNA damage or apoptosis inducing agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, temozolomide, teniposide, Thioguanine, topotecan, troxacitabine, Abraxane, Adriamycin, carboplatin, Cytoxan, Doxil, Ellence, fluorouracil, Gemzar, Ixempra, methotrexate, Mitomycin, mitoxantrone, Navelbine, Taxol, Taxotere, thiotepa, vincristine, Xeloda, Herceptin, Tykerb, Avastin, mitotic inhibitors, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

4. The method of any of the preceding claims, wherein said step (c) further comprises a correlation between the activation levels of said activatable elements within said plurality of DNA damage repair pathways.

5. The methods of claim 4 further comprising correlating the said activation levels of said activatable elements within said plurality of DNA damage repair pathways with apoptosis induced by said DNA damage or apoptosis inducing agent on said cell population.

6. The method of any preceding claim wherein said condition is selected from the group consisting of acute leukemia, myelodysplastic syndrome and myeloproliferative neoplasms.

7. The method of claim 6 wherein the individual has a predefined clinical parameter.

8-34. (canceled)

35. A method of classification, diagnosis, prognosis and/or prediction of an outcome of a condition in an individual, said method comprising:
a) contacting a cell population from said individual with a DNA damage or apoptosis inducing agent, wherein said cell population comprises a genetic and/or epigenetic alteration, wherein said alteration is associated with the development of said condition, and wherein said cell population is not associated and/or is not causative of said condition;
b) determining an activation level of at least one activatable element within a DNA damage pathway, an apoptosis pathway, and/or a cell cycle pathway in one or more cells from said cell population; and
c) making a decision regarding classification, diagnosis, prognosis and/or prediction of an outcome of said condition in said individual, wherein said decision is based on said activation levels of said at least one activatable element within said DNA damage pathway, an apoptosis pathway, and/or a cell cycle pathway.

36. The method of claim 35 further comprising performing a molecular analysis to detect said genetic alteration is said cell population.

37. The method of claim 35, wherein said DNA damage or apoptosis inducing agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, temozolomide, teniposide, Thioguanine, topotecan, troxacitabine, Abraxane, Adriamycin, carboplatin, Cytoxan, Doxil, Ellence, fluorouracil, Gemzar, Ixempra, methotrexate, Mitomycin, mitoxantrone, Navelbine, Taxol, Taxotere, thiotepa, vincristine, Xeloda, Herceptin, Tykerb, Avastin, mitotic inhibitors, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

38. The method of claim 35, wherein said at least one activatable element within said DNA Damage pathway is selected from the group consisting of p-Chk1, p-Chk2, p-53, p-ATM, and p-H2AX.

39. The method of claim 35, wherein said activatable element within said apoptosis pathway is selected from the group consisting of Cleaved PARP, Cleaved Caspase 3, Cleaved Caspase 8, BAX, Bak, and Cytochrome C.

40. The method of claim 35, wherein said at least one activatable element within a cell cycle pathway is selected from the group consisting of Cdc25, p-p53, cCdk1, CyclinB1, p16, p21, p-Histone H3 and Gadd45.

41. The method of claim 35, further comprising determining guides selection of a therapeutic treatment for said individual.

42-57. (canceled)

58. A method of determine a signaling phenotype of a cell population, wherein said cell population comprises a genetic/epigenetic alteration of interest, said method comprising:
a) subjecting said cell population comprising said genetic alteration to a plurality of modulators in separate of cultures;
b) characterizing at least one pathway in said cell population from separate plurality of cultures by determining an activation level of at least one activatable element within said at least one pathway;
c) creating a response panel for said comprising said characterization of said at least one pathway from said separate cultures; and
d) determining a signaling phenotype, wherein said signaling phenotype is based on said response panel.

59. The method of claim 58 further comprising performing a molecular analysis to detect said genetic alteration is said cell population.

60. The method of claim 58, wherein said genetic alteration is a germ line alteration,

61. The method of claim 58, wherein said genetic alteration is an alteration in a gene selected from the group consisting of APC, AXIN2, ARF, ATM, BLM, CDH1, GPC3, CYLD, EXT1, EXT2, PTCH, SUFU, FH, SDHB, SDHC, SDHD, VHL, TP53, WT1, STK11, PTEN, TSC1, TSC2, CDKN2A, CDK4, RB1, RAD50, NF1, BMPR1A, MEN1, SMAD4, BHD, HRPT2, NF2, MUTYH, ATM, BLM, BRCA1, BRCA2, FANCA, FANCC, FANCD2, FANCE, FANCF, FANCG, NBS1, RECQL4, WRN, MSH2, MLH1, MSH6, MDM2, MRE11, NBS1, RAS, RHO, RAN, RAB, PMS2, p53, XPA, XPC, ERCC2, ERCC3, ERCC4, ERCC5, DDB2, KIT, MET, PDGFRA, RET, and DNA replication factor C.

62. The method of claim 58, wherein said genetic alteration in a gene from Table 1.

63. The method of claim 58, wherein said genetic alteration is in a BRCA gene.

64-101. (canceled)
